(19) United States
(12) Patent Application Publication
(10) Pub. No.: US 2024/0246943 A1
(43) Pub. Date: Jul. 25, 2024

(54) MULTIFACETED APPROACH TO NOVEL INTERLEUKIN-6 INHIBITORS

(71) Applicant: UNIVERSITY OF FLORIDA RESEARCH FOUNDATION, INCORPORATED, Gainesville, FL (US)

(72) Inventors: Chenglong Li, Gainesville, FL (US); Daniel Schultz, Gainesville, FL (US); Zhihang Shen, Gainesville, FL (US)

(73) Assignee: University of Florida Research Fooundation, Incorporated, Gainesville, FL (US)

(21) Appl. No.: 18/287,690

(22) PCT Filed: Apr. 20, 2022
(86) PCT No.: PCT/US2022/025656

§ 371 Date: Oct. 20, 2023

Related U.S. Application Data
(60) Provisional application No. 63/177,779, filed on Apr. 21, 2021.
(60) Provisional application No. 63/307,012, filed on Feb. 4, 2022.

(51) Int. Cl. C07D 413/12; C07D 401/12; C07D 403/12; C07D 405/14; C07D 487/04; C07D 493/04; C07D 493/14

(52) U.S. Cl. CPC C07D 413/12; C07D 401/12; C07D 403/12; C07D 405/14; C07D 487/04; C07D 493/04; C07D 493/14

(57) ABSTRACT

The instant disclosure describes compounds having IL-6 modulating activity, pharmaceutical compositions and kits thereof, and methods of treating diseases, disorders or symptoms thereof mediated by IL-6.

### CROSS-REFERENCE TO RELATED APPLICATIONS

This application claims priority to U.S. Provisional Application No. 63/177,779, filed Apr. 21, 2021, and to U.S. Provisional Application No. 63/307,012, filed Feb. 4, 2022, each of which is incorporated herein by reference in their entireties.

### BACKGROUND

Interleukin-6 (IL-6) is a cytokine produced by a large variety of cell types and can exert its effects on virtually all cells. IL-6 is involved in a broad array of biological activities such as immune responses, apoptosis, and proliferation [Kishimoto T, Akira S, Taga T. Interleukin-6 and its receptor: a paradigm for cytokines. Science 1992, 258, 593-597]. However, when IL-6 is dysregulated or constantly expressed at a high level, it will cause numerous pathological conditions (FIG. 1) [Bernd S, May S. IL-6 as a drug discovery target. DDT 1998, 5, 202-213]. For example, uncontrolled and prolonged activation of inflammation, referred to as chronic inflammation, is a hallmark of many diseases including cancer, and one of the most abundant cytokines associated with this condition is IL-6 [Mantovani A, Paola A, Antonio S, et al. Cancer-related inflammation. Nature 2008, 454, 436-444]. Because of these two opposite effects, IL-6 is also recognized as a “wolf in sheep's clothing”. One possible reason for this difference may result from the concentration of IL-6 present in plasma. In healthy persons, the IL-6 level is as low as the detection limit of 1 pg/ml [Stefan R J. IL-6 trans-signaling via the soluble IL-6 receptor: importance for the pro-inflammatory activities of IL-6. Int J Biol Sci 2012, 8, 1237-1247], while in pathological conditions, the level of plasma IL-6 can surge by one hundred thousand fold to as high as the low microgram range (Table 1) [Nowell M A, Richards P J, Horiuchi S, et al. Soluble IL-6 receptor governs IL-6 activity in experimental arthritis: blockade of arthritis severity by soluble glycoprotein 130. J Immunol 2003, 171, 3202-3209; Waage A, Brandtzaeg P, Halstensen A, et al. The complex pattern of cytokines in serum from patients with meningococcal septic shock. JExp Med 1989, 169, 333-338].

IL-6 was first identified by its ability to activate the maturation of B cells to antibody-secreting plasma cells [Okada M. B cell growth factors and B cell differentiation factor from human T hybridomas. Two distinct kinds of B cell growth factor and their synergism in B cell proliferation. J Exp Med 1983, 157, 583-590]. It has been referred to by several names such as B-cell stimulatory factor-2 (BCSF-2), interferon-2 (IFN-2), hepatocyte stimulating factor (HSF), macrophage granulocyte inducer type 2 (MGI-2A) or thrombopoietin [Kishimoto T. The biology of interleukin-6. Blood 1989, 74, 1-10], which also illustrates its versatile functions. A 1.9 Å crystal structure of IL-6 was reported by Somers and his coworkers in 1997 [Somers W, Stahl M, Seehra, J. 1.9 Å crystal structure of interleukin 6: implications for a novel mode of receptor dimerization and signaling. The EMBO Journal 1997, 16, 989-997; Xu G-Y, Yu H-A, Hong J, et al. Solution structure of recombinant human interleukin-6. J Mol Biol 1997, 268, 468-481]. It is a 26-kDa glycoprotein that contains 212 amino acids arranged in a five helix bundle, with four of them forming a classical four-helix bundle with a conformation termed “up-up-down-down” [Zaccaro L, Bucci E, Vitale M, et al. Synthetic peptides mimicking the interleukin-6/gp 130 interaction: a two-helix bundle system. Design and conformational studies. J Pept Sci. 2003, 9, 90-105; Boulanger M J, Chow D C, Brevnova E E, et al. Hexameric structure and assembly of the interleukin-6/IL-6 alpha-receptor/gp130 complex. Science 2003, 300, 2101-2104].

TABLE 1
IL-6 levels in healthy individuals and in selected disease states.

| Condition | Plasma IL-6 conc. |
|---|---|
| Healthy individual | 1 pg/ml |
| Rheumatoid arthritis | 150 ng/ml |
| Septic condition | low μg/ml |

Il-6 activates a cell surface signaling assembly composed of IL-6, the IL-6 α-receptor (IL-6Ra), and the shared signaling receptor gp130. IL-6 first binds to IL-6Ra to form a binary complex, then recruits gp130 to form the IL-6/IL-6Ra/gp130heterotrimer. Furthermore, homodimerization of the IL-6/IL-6Ra/gp130 heterotrimer occurs by interactions between IL-6 of one trimer and the D1 domain of gp130 of the other trimer, forming a hexamer [Boulanger M J, Chow D C, Brevnova E E, et al. Hexameric structure and assembly of the interleukin-6/IL-6 alpha-receptor/gp130 complex. Science 2003, 300, 2101-2104; Eric A, Marie-Christophe B, Jean-Michel D. Interleukin-6: From identification of the cytokine to development of targeted treatments. Joint Bone Spine 2010, 77, 532-536]. The reciprocal homodimerization of the IL-6/IL-6Ra/gp130 trimer triggers the activation of associated Janus kinase (JAK). Then, JAK causes autophosphorylation as well as the phosphorylation of gp130. After that, the phosphorylated positions on JAK and gp130 serve as the docking sites of downstream effector signal transducer and activator of transcription 3 (STAT3) SH2 domain, followed by reciprocal dimerization of phosphorylated STAT3, nuclear translocation, DNA binding and cancer gene transcription [Darnell J E. STATs and gene regulation. Science, 1997, 277, 1630-1635; Jinxia D, Fedora G, Nouri N. Small Molecule Inhibitors of Stat3 Signaling Pathway. Curr Cancer Drug Targets, 2007, 7, 91-107]. Other than the IL-6/gp130/STAT3 signaling pathway, IL-6 can also activate the PI3K/AKT pathway and the Raf/MEK/ERK pathway. The existence of these different pathways activated by IL-6 appears to be highly cell type-dependent [Haegeman G. Structural analysis of the sequence coding for an inducible 26-kDa protein in human fibroblasts. Eur J Biochem 1986, 159, 625-632; Yamasaki K. Cloning and expression of the human interleukin-6 (BSF-2/IFN beta 2) receptor. Science 1988, 241, 825-828].

The extracellular structure and assembly of the IL-6/IL-6Ra/gp130complex was solved in 2003 by the Garcia group [Boulanger M J, Chow D C, Brevnova E E, et al. Hexameric structure and assembly of the interleukin-6/IL-6 alpha-receptor/gp130 complex. Science 2003, 300, 2101-2104], and the full-length transmembrane IL-6/IL-6Ra/gp130 cytokine receptor complex was solved by the same group in 2011 [Patrick J, Georgios S, Amanda J, et al. structural snapshots of full-length Jak1, a transmembranegp130/IL-6/IL-6Ra cytokine receptor complex, and the receptor-Jak1 holocomplex. Structure 2011, 19, 45-55]. The IL-6 extracellular signaling portion is a symmetric hexamer, which contains two IL-6, two IL-Ra and two gp130 subunits assembling sequentially and cooperatively. There are five main binding epitopes involved in this complex, namely site I, site II (IIa and IIb) and site III (IIIa and IIIb). [Boulanger M J, Chow D C, Brevnova E E, et al. Hexameric structure and assembly of the interleukin-6/IL-6 alpha-receptor/gp130 complex. Science 2003, 300, 2101-2104].

Site I is formed when IL-6 binds to IL-6Ra. Binding occurs between the D3 domain of IL-6Ra and the A and D helices of IL-6. It covers an area of 1200 Å². The key residues in this binding site are Phe229 and Phe279 on the IL-6Ra D3 domain, Arg179 and Lys171 on IL-6. Site II is formed when the IL-6/IL-6Ra complex binds to gp130. Site IIa consists of the elbow region of D2 and D3 on gp130 and the A and C helices on IL-6. It covers an area of 1272 Å², where Phe169 on the gp130 elbow region and Arg24, Lys27, Arg30 and Asp34 contribute the most to the binding surface. Site IIb consists of the D3 domain on IL-6Ra and the D3 domain on gp130, which covers a surface area of 1078 Å². Site III is formed when two IL-6/IL-6Ra/gp130 trimers dimerize with each other. Site IIIa consists of the tip of the IL-6 four-helix bundle and the bottom R sheet of the D1 domain of gp130, which covers an area of 1276 Å². The N-terminal peptide of gp130 (from Leu2 to Cys6) is solvent-exposed and inserts in a groove on the surface of IL-6 formed by the A, B and C, D inter-helical loops. Trp157 on IL-6 is also found to be critical, contributing 21% of the total buried surface area. This finding is consistent with previous results obtained through mutagenesis that this aromatic signature residue is of great importance in Site IIIa [Paonessa G. Graziani R, De S A, et al. Two distinct and independent sites on IL-6 trigger gp 130 dimer formation and signaling. EMBO J 1995, 14, 1942-1951; Barton V A, Hudson K R, Heath J K. Identification of Three Distinct Receptor Binding Sites of Murine Interleukin-11. J Biol Chem 1999, 274, 5755-5761]. Site IIIb involves the interaction between the tip of D1 domain on gp130 and the D2 domain of IL-6Ra, which covers a surface area of 473 Å².

Other than IL-6, there are seven other members in the IL-6 family of cytokines, which are IL-11, leukemia inhibitory factor (LIF), oncostatin M (OSM), cardiotrophin-1 (CT-1), ciliary neurotrophic factor (CNTF), cardiotrophin-like cytokine (CLC) and IL-27 [Ursula A W, Jacqueline M S. The gp130 Receptor Cytokine Family: Regulators of Adipocyte Development and Function. Curr Pharm Des. 2011, 4, 340-346]. They are grouped together because all of these cytokines require the signal receptor subunit gp130 in the signal transduction process, so they are also referred to as gp130 related cytokines. These cytokines act in a pleiotropic manner, and are also redundant in regulating various biological processes [Ernst M, Jenkins B J. Acquiring signalling specificity from the cytokine receptor gp130. Trends in genetics 2004, 20, 23-32]. The IL-6 family of cytokines participate in signal transduction mainly through the JAK/STAT pathway and the most common STAT activated is STAT3 [Heinrich P C, Behrmann I, Haan S, et al. Principles of interleukin (IL)-6-type cytokine signalling and its regulation. Biochem J 2003, 374, 1-20].

As mentioned above, as all members in the IL-6 family of cytokines transduce their signals through glycoprotein 130 (gp130), they still require other receptors to form the signal transduction complex and the stoichiometry varies. For IL-6 and IL-11, both initially bind to the IL-6 receptor a or IL-11 receptor a [White U A, Stewart W C, Mynatt R L, et al. Neuropoietin attenuates adipogenesis and induces insulin resistance in adipocytes. J Biol Chem 2008, 283, 22505-22512], respectively. The dimer then recruits and associates with gp130 to form a trimer, followed by homodimerization of two of these trimers for signal transduction. For LIF and CT-1, both require the LIF receptor (LIFR) to form a complex with gp130 to mediate signal transduction [Gearing D P, Thut C J, VandeBos T, et al. Leukemia inhibitory factor receptor is structurally related to the IL-6 signal transducer, gp130. EMBO J 1991, 10, 2839-2848; Pennica D, Shaw KJ, Swanson T A, et al. Cardiotrophin-1. Biological activities and binding to the leukemia inhibitory factor receptor/gp130 signaling complex. J Biol Chem 1995, 270, 10915-10922]. In addition to LIFR, CT-1 has also been reported to recruit another as yet unidentified alpha receptor [Robledo O, Guillet C, Chevalier S, et al. Hepatocyte-derived cell lines express a functional receptor for cardiotrophin-1. Eur Cytokine Netw 1997, 8, 245-52]. For CNTF and CLC, both require CNTFRa and LIFR to form a complex with gp130 for signal transduction [Davis S, Aldrich T H, Stahl N, et al. LIFR beta and gp130 as heterodimerizing signal transducers of the tripartite CNTF receptor. Science 1993, 260, 1805-1808; Elson G C, Lelievre E, Guillet C, et al. CLF associates with CLC to form a functional heteromeric ligand for the CNTF receptor complex. Nat Neurosci 2000, 3, 867-872]. For OSM, it has been reported to either utilize the LIFR complex or alternatively utilize an OSM receptor (OSMR) to form a complex with gp130 during signaling [Ichihara M, Hara T, Kim H, et al. Oncostatin M and leukemia inhibitory factor do not use the same functional receptor in mice. Blood 1997, 90,165-173], although some studies indicate that OSM primarily signals via the OSMR and not the LIFR. For IL-27, its cell signaling engages a gp130/WSX-1 heterodimeric receptor complex [Pflanz S, Hibbert L, Mattson J, et al. WSX-1 and glycoprotein 130 constitute a signal-transducing receptor for IL-27. J Immunol 2004, 172, 2225-2231].

From this discussion, it is apparent that IL-6 and IL-11 signal transduction require the dimerization of two gp130 subunits, while in signaling by other IL-6 family members, only one gp130 is involved. As a result, the binding site III in IL-6 signaling is unique from LIF, CNTF, CLC, CT-1, OSM and IL-27. It may share some similarity with IL-11 signaling, although the complex crystal structure of this signaling complex remains unknown.

IL-6 was first discovered as an inflammatory cytokine and plays an important role in the immune response. Tissue damage or pathogen invasion is always associated with inflammation, which is necessary to help remove necrotic debris. Macrophages are recruited to the infected area in the inflammatory response, which produces reactive oxygen species (ROS) and proteases [Ma J, Chen T, Mandelin J. Regulation of macrophage activation. Cell Mol Life Sci 2003, 60, 2334-2346]. This response helps to kill or degrade the pathogens. Under such a harsh microenvironment, normal cells would also be killed in the absence of a mediator to protect these cells. IL-6, together with various other inflammatory mediators, not only plays an important role in driving inflammatory mechanisms, but also acts as the protector for both cells in the immune system and the injured or infected tissues [Shinozaki M, Hirahashi J, Lebedeva T, et al. IL-15, a survival factor for kidney epithelial cells, counteracts apoptosis and inflammation during nephritis. J Clin Invest 2002, 109, 951-960; Tourbah A, Linnington C, Bachelin C, et al. Inflammation promotes survival and migration of the CG4 oligodendrocyte progenitors transplanted in the spinal cord of both inflammatory and demyelinated EAE rats. J Neurosci Res 1997, 50, 853-861]. However, this protection can go too far and, in some cases, it also extends to cells that strayed from normal cell cycle regulatory pathways [Hideshima T, Nakamura N, Chauhan D, et al. Biologic sequelae of interleukin-6 induced P13-K/Akt signaling in multiple myeloma. Oncogene 2001, 20, 5991-6000], which may eventually cause cancer. Strong support for the role of IL-6 in inflammation and cancer comes from research showing that IL-6 was produced by stroma and inflammatory cells within the tumor microenviroment. The abundant IL-6 then plays its role in activating JAK/STAT3 signal transduction and promotes cell proliferation. In inflammatory cells and tumor cells, extremely high levels of activated STAT3 were observed particularly at the invasive edge of tumors [Bromberg J, Wang T. Inflammation and Cancer: IL-6 and STAT3 Complete the Link. Cancer Cell 2009, 15, 79-80]. Another piece of evidence showing the role of IL-6 in protecting cancer cells comes from the fact that IL-6 is found to mediate many unwanted effects, such as making cancer cells resistant to chemotherapeutic drugs [Hodge D R, Xiao W, Wang L H, et al. Activating mutations in STAT3 and STAT5 differentially affect cellular proliferation and apoptotic resistance in multiple myeloma cells. Cancer Biol Ther 2004, 3, 188-194; Frassanito M A, Cusmai A, Iodice G, et al. Autocrine interleukin-6 production and highly malignant multiple myeloma: relation with resistance to drug-induced apoptosis. Blood 2001, 97, 483-489].

So far, researchers have found that there are numerous cell types which can respond to IL-6; the cell types include but are not limited to, lung, heart, ovary, kidney, liver, macrophages, astrocytes, endometrial stromal cells, monocytes, amnion-derived cells, Kupffer cells, osteoblasts, microglia, multiple myeloma, Leydig cell precursors (testes), mast cells, fibroblasts (dental pulp, gingival, nasal turbinate, polyps, synovial), human endothelial cells, and prostatic intraepithelial neoplasia cells [Suganuma M, Okabe S, Kurusu M, et al. Discrete roles of cytokines, TNF-alpha, IL-1, IL-6 in tumour promotion and cell transformation. Int J Oncol 2002, 20, 131-136]. Low level production of IL-6 typically occurs in these cells. However in a situation like inflammation, IL-6 is constantly expressed at a high level, which drives inflammation itself but also increases the risk of tumorigenesis in these types of cells, mainly through the IL-6/JAK/STAT3 signal transduction pathway. Indeed, various types of cancers are found to be associated with the IL-6/JAK/STAT3 signal transduction pathway, which includes multiple myeloma, chronic lymphocytic leukemia (CLL), acute myelogenous leukemia (AML), large granular lymphocyte leukemia (LGL), lung cancer, breast cancer, renal cancer, prostate cancer, pancreatic carcinoma, melanoma, colon carcinoma, gastric carcinoma, cervical cancer, ovarian cancer, liver cancer, and head and neck cancers [David R H, Elaine M H, William L F. The role of IL-6 and STAT3 in inflammation and cancer. Eur J Cancer 2005, 2502-2512].

As discussed above, IL-6 belongs to the gp130-related cytokine family. It plays an important role in the IL-6/gp130/STAT3 signaling pathway, which was found to be protumorigenic in many cancers. IL-6 first binds to the IL-6 receptor a (IL-6Ra), which recruits gp130 on the cell membrane; then dimerization of two of the IL-6/IL-6Ra/gp130 trimers allows signal transduction via gp130 cytoplasmic domains. This activates JAK then STAT3 (Tyr705 phosphorylation), leading to STAT3 nuclear translocation, DNA binding, and the transcription of multiple oncogenes. To date, very few small molecules have been identified as IL-6/gp130 inhibitors. MDL-A is a natural product that was found to specifically interfere with the IL-6/gp130 interaction surface. However, it's no longer produced in nature (due to mutation in the bacterial strain) and synthetic complexity together with its relatively low binding affinity limits its potential as a lead compound. Although some MDL-A analogues were designed and made, their tedious synthesis and relatively weak potency make them less promising. Raloxifene and bazedoxifene were found to be IL-6/gp130 inhibitors, but they are already FDA approved drugs known as selective estrogen receptor modulators (SERMs) used for the treatment of postmenopausal osteoporosis, which may cause side effects when used to treat cancer. SC144 is another potent small molecule found to bind to gp130, but it inhibits both IL-6 and LIF induced STAT3 nuclear translocation, indicating it may not be a specific IL-6/gp130 inhibitor. Therefore, looking for novel small molecule IL-6/gp130 inhibitors with high potency and specificity as anti-cancer agents is highly desirable.

### BRIEF SUMMARY OF THE INVENTION

This invention is directed towards compounds, compositions, and methods of treating disease, disorders and conditions in a subject, including, inflammation, cancer, and autoimmune diseases by use of the compounds and compositions thereof. The invention also includes methods of making the compounds and compositions herein, using processes, chemical reactions, and intermediate compounds, reagents, and conditions as described herein.

It is understood that the embodiments of the invention discussed below with respect to the preferred variable selections can be taken alone or in combination with one or more embodiments, or preferred variable selections, of the invention, as if each combination were explicitly listed herein.

In one aspect, provided herein is a compound of Formula (F), or a salt, solvate, hydrate, polymorph, co-crystal, tautomer, stereoisomer, isotopically labeled derivative, or prodrug thereof:

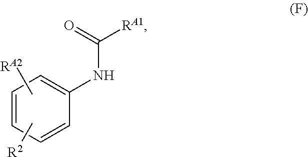

wherein R^{A1}, R^{A2}, and R² are as defined herein.

In one aspect, provided herein is a compound of Formula (A), or a salt, solvate, hydrate, polymorph, co-crystal, tautomer, stereoisomer, isotopically labeled derivative, or prodrug thereof:

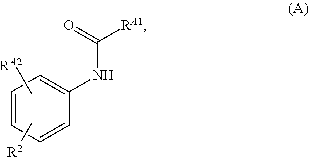

wherein R^{A1}, R^{A2}, and R² are as defined herein.

In another aspect, provided herein is a compound of Formula (B), or a salt, solvate, hydrate, polymorph, co-crystal, tautomer, stereoisomer, isotopically labeled derivative, or prodrug thereof:

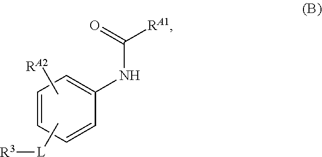

wherein R^{A1}, R^{A2}, L, and R³ are as defined herein.

In another aspect, provided herein is a compound of Formula (C), or a salt, solvate, hydrate, polymorph, co-crystal, tautomer, stereoisomer, isotopically labeled derivative, or prodrug thereof:

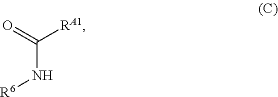

wherein R^{A1} and R⁶ are as defined herein.

In another aspect, provided herein is a compound of Formula (D), or a salt, solvate, hydrate, polymorph, co-crystal, tautomer, stereoisomer, isotopically labeled derivative, or prodrug thereof:

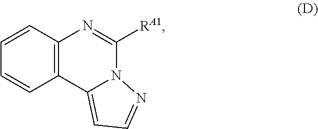

wherein R^{A1} is as defined herein.

In another aspect, provided herein is a compound of Formula (E), or a salt, solvate, hydrate, polymorph, co-crystal, tautomer, stereoisomer, isotopically labeled derivative, or prodrug thereof:

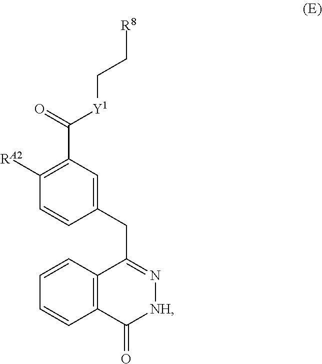

wherein R⁸, R^{A2}, and Y¹ are as defined herein.

In another aspect, provided herein is a pharmaceutical composition comprising a compound provided herein (e.g., a compound of Formula (A), (B), (C), (D), (E), or (F)), or a salt thereof, and a pharmaceutically acceptable carrier. In another aspect, provided herein is a pharmaceutical composition comprising a compound provided herein (e.g., a compound of Formula (A), (B), (C), (D), (E), or (F)), or a salt, solvate, hydrate, polymorph, co-crystal, tautomer, stereoisomer, isotopically labeled derivative, or prodrug thereof, and a pharmaceutically acceptable carrier. In some embodiments, the pharmaceutical composition further comprises an additional therapeutic agent.

In another aspect, provided herein is a kit comprising an effective amount of a compound provided herein (e.g., a compound of Formula (A), (B), (C), (D), (E), or (F)), or a salt thereof, or a pharmaceutical composition thereof, and instructions for administering the compound to a subject in need thereof. In another aspect, provided herein is a kit comprising an effective amount of a compound provided herein (e.g., a compound of Formula (A), (B), (C), (D), (E), or (F)), or a salt, solvate, hydrate, polymorph, co-crystal, tautomer, stereoisomer, isotopically labeled derivative, or prodrug thereof, or a pharmaceutical composition thereof, and instructions for administering the compound to a subject in need thereof.

In one aspect, provided herein is a method of inhibiting IL-6 signaling comprising administering a compound provided herein (e.g., a compound of Formula (A), (B), (C), (D), (E), or (F)), or a salt thereof, or a pharmaceutical composition thereof.

In another aspect, provided herein is a method of inhibiting IL-6/gp130 comprising administering a compound provided herein (e.g., a compound of Formula (A), (B), (C), (D), (E), or (F)), or a salt thereof, or a pharmaceutical composition thereof.

In another aspect, provided herein is a method of treating inflammatory disease in a subject in need thereof, the method comprising administering to the subject a compound provided herein (e.g., a compound of Formula (A), (B), (C), (D), (E), or (F)), or a salt thereof, or a pharmaceutical composition thereof.

In another aspect, provided herein is a method of treating cancer in a subject in need thereof, the method comprising administering to the subject a compound provided herein (e.g., a compound of Formula (A), (B), (C), (D), (E), or (F)), or a salt thereof, or a pharmaceutical composition thereof.

In another aspect, provided herein is a method of treating an autoimmune disease in a subject in need thereof, the method comprising administering to the subject a compound provided herein (e.g., a compound of Formula (A), (B), (C), (D), (E), or (F)), or a salt thereof, or a pharmaceutical composition thereof.

### BRIEF DESCRIPTION OF THE DRAWINGS

The present disclosure is further described below with reference to the following non-limiting examples and with reference to the following figures, in which:

FIG. 1. depicts results of bazedoxifene and LS-28-3, and the confirmatory binding of LS-28-3 to gp130 in MDA-MB-231 breast cancer cell lysates using a 1:2000 ratio of pronase:protein with a proteolysis time of 20 minutes. These results suggest the direct binding of LS-28-3 to gp130 stabilizes its structure and therefore protects it from proteolytic digestion.

FIG. 2. depicts results of bazedoxifene and LS-28-3, and the confirmatory binding of LS-28-3 to gp130 in MIAPaCA2 pancreatic cancer cell lysates using a 1:2000 ratio of pronase:protein with a proteolysis time of 20 minutes. These results suggest the direct binding of LS-28-3 to gp130 stabilizes its structure and therefore protects it from proteolytic digestion.

FIG. 3. depicts MTT assay results in MDA-MB-231 cells using LLM-418, bazedoxifene, LS-28-3, and COMP-D (LS-101-D).

FIG. 4. depicts cell proliferation and MTT assay results in AML-12 hepatocyte cells using LLM-418, bazedoxifene, and LS-28-3.

FIG. 5. depicts T47D breast cancer cells stimulated with LLM418-MCBu, then cytokine. The results indicate that IL-6 inhibitor LLM418-MCBU selectively inhibits IL-6 induced p-STAT3 versus IFN-7 induced p-STAT1 and LIF induced p-STAT3 in T47D breast cancer cell line.

FIG. 6 depicts T47D breast cancer cells stimulated with LLM418-CBU, then cytokine. The results indicate that IL-6 inhibitor LLM418-CBU inhibits IL-6 induced p-STAT3 but also inhibits IFN-7 induced p-STAT1 and LIF induced p-STAT3 in T47D breast cancer cell line.

FIG. 7 depicts T47D breast cancer cells stimulated with IL-6 inhibitor LLM418-Et or IL-6 inhibitor LLM418-iPR, then cytokine. The results indicate that IL-6 inhibitors LLM418-Et and LLM418-iPR inhibit both IL-6 induced p-STAT3 and IFN-7 induced p-STAT1 in T47D breast cancer cell line.

FIG. 8 depicts TNBC, BRCA-mutant, and T47D cells stimulated with LLM418-MCBU, then cytokine. The results indicate that IL-6 inhibitor LLM418-MCBU inhibits pSTAT3 in TNBC cell line, BRCA-mutant cell line, and T47D cell line.

FIG. 9 depicts cell viability of HCC1937 BRCA-mutant TNBC cells treated with LLM418-MCBU and PARP inhibitor talazoparib. The results indicate that IL-6 inhibitor LLM418-MCBU and talazoparib exhibits synergy (CI<1.0) in inhibiting cell viability.

### DETAILED DESCRIPTION

### Definitions

As used herein, the following terms have the meanings ascribed to them unless specified otherwise.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, I N 1972). The disclosure additionally encompasses compounds as individual isomers substantially free of other isomers, and alternatively, as mixtures of various isomers. The compounds herein may also contain linkages (e.g., carbon-carbon bonds) wherein bond rotation is restricted about that particular linkage, e.g., restriction resulting from the presence of a ring or double bond. Accordingly, all cis/trans and E/Z isomers are expressly included in the present disclosure. The compounds herein may also be represented in multiple tautomeric forms; in such instances, the present disclosure expressly includes all tautomeric forms of the compounds and oligonucleotides described herein, even though only a single tautomeric form may be represented. All such isomeric forms of such compounds herein are expressly included in the present disclosure. The term “isomers” is intended to include diastereoisomers, enantiomers, regioisomers, structural isomers, rotational isomers, tautomers, and the like. For compounds that contain one or more stereogenic centers, e.g., chiral compounds, the methods of the present disclosure may be carried out with an enantiomerically enriched compound, a racemate, or a mixture of diastereomers. All isomers of compounds delineated herein are expressly included in the present disclosure.

Furthermore, the compounds of the disclosure include olefins having either geometry: “Z” refers to what is referred to as a “cis” (same side) conformation whereas “E” refers to what is referred to as a “trans” (opposite side) conformation. With respect to the nomenclature of a chiral center, the terms “d” and “l” configuration are as defined by the IUPAC Recommendations. As to the use of the terms, diastereomer, racemate, epimer and enantiomer, these will be used in their normal context to describe the stereochemistry of preparations.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

The term “isotopes” refers to variants of a particular chemical element such that, while all isotopes of a given element share the same number of protons in each atom of the element, those isotopes differ in the number of neutrons.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁-6, C₁-5, C₁-4, C₁-3, C₁-2, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

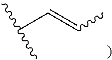

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroCₗ₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroCₗ₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R—, —NR^{bb}C(═O)N(R^{bb})₂, —C(═NR^{bb})R—, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{dd} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{dd} groups are joined to form a 3-14 membered heterocyclyl or 5-14 membered heteroaryl ring.

In certain embodiments, each carbon atom substituent is independently halogen, substituted (e.g., substituted with one or more halogen) or unsubstituted C₁₋₆ alkyl, —OR^{aa}, SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R—, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono-or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), (3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{aa})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methoxy, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3″′-[N-(imidazolylmethyl)]trityl Ether (IDTr-OR), 4,4′-Dimethoxy-3″′-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

A “leaving group” (LG) is an art-understood term referring to an atomic or molecular fragment that departs with a pair of electrons in heterolytic bond cleavage, wherein the molecular fragment is an anion or neutral molecule. As used herein, a leaving group can be an atom or a group capable of being displaced by a nucleophile. See e.g., Smith, March Advanced Organic Chemistry 6th ed. (501-502). Exemplary leaving groups include, but are not limited to, halo (e.g., fluoro, chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, −OP(R^{cc})₂, −OP(R^{aa})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Additional examples of suitable leaving groups include, but are not limited to, halogen alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some embodiments, the leaving group is a sulfonic acid ester, such as toluenesulfonate (tosylate, -OTs), methanesulfonate (mesylate, -OMs), p-bromobenzenesulfonyloxy (brosylate, -OBs), —OS(═O)₂(CF₂)₃CF₃ (nonaflate, —ONf), or trifluoromethanesulfonate (triflate, -OTf). In some embodiments, the leaving group is a brosylate, such as p-bromobenzenesulfonyloxy. In some embodiments, the leaving group is a nosylate, such as 2-nitrobenzenesulfonyloxy. In some embodiments, the leaving group is a sulfonate-containing group. In some embodiments, the leaving group is a tosylate group. In some embodiments, the leaving group is a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

These and other exemplary substituents are described in more detail in the Detailed Description, Examples, and Claims. The disclosure is not limited in any manner by the above exemplary listing of substituents.

The term “carbohydrate” refers to sugars or polymers of sugars. In some embodiments, the carbohydrate is a monosaccharide, a disaccharide, or a polysaccharide. In some embodiments, the carbohydrate is a monosaccharide or a disaccharide. In some embodiments, the carbohydrate is made up of one or more monosaccharide units having at least 4, 5 or 6 carbon atoms (which may be linear, branched or cyclic) with an oxygen, nitrogen or sulfur atom bonded to each carbon atom; or a compound having as a part thereof a carbohydrate moiety made up of one or more monosaccharide units. Without limitations, the term “carbohydrate” is intended to include monomeric sugar alcohols, polysaccharides, oligosaccharides and other carbohydrate polymers. The sugar may be optionally substituted. Further, the sugar can have the L- or the D-conformation. Exemplary carbohydrates include, but are not limited to, erythrose, threose, ribose, arabinose, xylose, lyxose, ribulose, xylulose, allose, altrose, glucose, mannose, gulose, idose, galactose, telose, galactosamine, N-acetylgalactose, glucosamine, N-acetylglucosamine, sialic acid, talose, psicose, fructose, sorbose, tagatose, fucose, fuculose, rhamonse, sedoheptulose, octose, sulfoquinovose, nonose (neuraminic acid), sucrose, lactulose, lactose, maltose, trehalose, cellobiose, kojibiose, nigerose, isomaltose, P,P-Trehalose, a,P-Trehalose, sophorose, laminaribiose, gentibiose, turanose, maltulose, palatinose, gentibiulose, mannobiose, melibiose, rutinose, rutinulose, xylobiose, raffinose, melezitose, acarbose, stachyose, and any combinations thereof. In some embodiments, the carbohydrate is pyranose selected from the group consisting of allose, altrose, glucose, mannose, gulose, idose, galactose, and telose.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”. Preferred enantiomerically enriched compounds have an enantiomeric excess of 50% or more, more preferably the compound has an enantiomeric excess of 60%, 70%, 80%, 90%, 95%, 98%, or 99% or more. In preferred embodiments, only one enantiomer or diastereomer of a chiral compound is administered to cells or a subject.

The term “co-crystal” refers to a crystalline structure comprising at least two different components (e.g., a compound disclosed herein and an acid), wherein each of the components is independently an atom, ion, or molecule. In certain embodiments, none of the components is a solvent. In certain embodiments, at least one of the components is a solvent. A co-crystal of a compound disclosed herein and an acid is different from a salt formed from a compound disclosed herein and the acid. In the salt, a compound disclosed herein is complexed with the acid in a way that proton transfer (e.g., a complete proton transfer) from the acid to a compound disclosed herein easily occurs at room temperature. In the co-crystal, however, a compound disclosed herein is complexed with the acid in a way that proton transfer from the acid to a compound disclosed herein does not easily occur at room temperature. In certain embodiments, in the co-crystal, there is no proton transfer from the acid to a compound disclosed herein. In certain embodiments, in the co-crystal, there is partial proton transfer from the acid to a compound disclosed herein. Co-crystals may be useful to improve the properties (e.g., solubility, stability, and ease of formulation) of a compound disclosed herein.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrug” includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included. In aspects, the compounds of the disclosure are prodrugs of any of the formulae herein.

The terms “composition” and “formulation” are used interchangeably.

As used herein, “inhibiting” encompasses preventing, reducing, and halting progression. For example, in the context of IL-6, the term refers to a reduction in the activity of the cytokine. In some embodiments, the term refers to a reduction of the level of activity, e.g., IL-6 activity, to a level that is statistically significantly lower than an initial level, which may, for example, be a baseline level of activity. In some embodiments, the term refers to a reduction of the level of activity, e.g., IL-6 activity, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of activity.

As used herein, “activating” encompasses permitting, increasing, and enhancing progression.

The term “modulate” refers to increases or decreases in the activity of a cell in response to exposure to a compound of the disclosure.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high-performance liquid chromatography. Particularly, in embodiments the compound is at least 85% pure, more preferably at least 90% pure, more preferably at least 95% pure, and most preferably at least 99% pure.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

A “peptide” is a sequence of at least two amino acids. Peptides can consist of short as well as long amino acid sequences, including proteins.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, D-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “protein” refers to series of amino acid residues connected one to the other by peptide bonds between the alpha-amino and carboxy groups of adjacent residues.

Amino acids may be referred to herein by either their commonly known three letter symbols or by the one-letter symbols recommended by the IUPAC-IUB Biochemical Nomenclature Commission.

As to amino acid sequences, one of skill will recognize that individual substitutions, deletions or additions to a peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I. The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 50 to 350 amino acids long. Typical domains are made up of sections of lesser organization such as stretches of D-sheet and D-helices. “Tertiary structure” refers to the complete three-dimensional structure of a polypeptide monomer. “Quaternary structure” refers to the three-dimensional structure formed by the noncovalent association of independent tertiary units. Anisotropic terms are also known as energy terms.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “target tissue” refers to any biological tissue of a subject (including a group of cells, a body part, or an organ) or a part thereof, including blood and/or lymph vessels, which is the object to which a compound, particle, and/or composition of the disclosure is delivered. A target tissue may be an abnormal or unhealthy tissue, which may need to be treated. A target tissue may also be a normal or healthy tissue that is under a higher than normal risk of becoming abnormal or unhealthy, which may need to be prevented. In certain embodiments, the target tissue is the liver. In certain embodiments, the target tissue is the lung. A “non-target tissue” is any biological tissue of a subject (including a group of cells, a body part, or an organ) or a part thereof, including blood and/or lymph vessels, which is not a target tissue.

The term “administer,” “administering,” or “administration” refers to implanting, absorbing, ingesting, injecting, inhaling, or otherwise introducing a compound described herein, or a composition thereof, in or on a subject. Examples of routes of administration which can be used include injection (subcutaneous, intravenous, parenterally, intraperitoneally, intrathecal), topical, oral, inhalation, rectal and transdermal.

The terms “condition,” “disease,” and “disorder” are used interchangeably.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population. In some embodiments, the subject is at risk of developing a disease or condition due to environmental factors (e.g., exposure to the sun).

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for inhibiting IL-6. In certain embodiments, a therapeutically effective amount is an amount sufficient for treating cancer. In certain embodiments, a therapeutically effective amount is an amount sufficient for inhibiting IL-6 and treating cancer.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for inhibiting IL-6. In certain embodiments, a prophylactically effective amount is an amount sufficient for preventing cancer. In certain embodiments, a prophylactically effective amount is an amount sufficient for inhibiting IL-6 and preventing cancer.

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); liver cancer (e.g., hepatocellular cancer (HCC), malignant hepatoma); and prostate cancer (e.g., prostate adenocarcinoma).

The terms “inflammatory disease” and “inflammatory condition” are used interchangeably herein, and refer to a disease or condition caused by, resulting from, or resulting in inflammation. Inflammatory diseases and conditions include those diseases, disorders or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent. Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

Additional exemplary inflammatory conditions include, but are not limited to, inflammation associated with acne, anemia (e.g., aplastic anemia, haemolytic autoimmune anaemia), asthma, arteritis (e.g., polyarteritis, temporal arteritis, periarteritis nodosa, Takayasu's arteritis), arthritis (e.g., crystalline arthritis, osteoarthritis, psoriatic arthritis, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, Type II diabetes mellitus), a skin condition (e.g., psoriasis, eczema, burns, dermatitis, pruritus (itch)), endometriosis, Guillain-Barre syndrome, infection, ischaemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenic purpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), lupus, multiple sclerosis, morphea, myeasthenia gravis, myocardial ischemia, nephrotic syndrome, pemphigus vulgaris, pernicious aneaemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, reperfusion injury, regional enteritis, rheumatic fever, systemic lupus erythematosus, schleroderma, scierodoma, sarcoidosis, spondyloarthopathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis. In certain embodiments, the inflammatory disorder is selected from arthritis (e.g., rheumatoid arthritis), inflammatory bowel disease, inflammatory bowel syndrome, asthma, psoriasis, endometriosis, interstitial cystitis and prostatistis. In certain embodiments, the inflammatory condition is an acute inflammatory condition (e.g., for example, inflammation resulting from infection). In certain embodiments, the inflammatory condition is a chronic inflammatory condition (e.g., conditions resulting from asthma, arthritis and inflammatory bowel disease). The compounds may also be useful in treating inflammation associated with trauma and non-inflammatory myalgia. The compounds disclosed herein may also be useful in treating inflammation associated with cancer.

Immune disorders, such as auto-immune disorders, include, but are not limited to, arthritis (including rheumatoid arthritis, spondyloarthopathies, gouty arthritis, degenerative joint diseases such as osteoarthritis, systemic lupus erythematosus, Sjogren's syndrome, ankylosing spondylitis, undifferentiated spondylitis, Behcet's disease, haemolytic autoimmune anaemias, multiple sclerosis, amyotrophic lateral sclerosis, amylosis, acute painful shoulder, psoriatic, and juvenile arthritis), asthma, atherosclerosis, osteoporosis, bronchitis, tendonitis, bursitis, skin condition (e.g., psoriasis, eczema, burns, dermatitis, pruritus (itch)), enuresis, eosinophilic disease, gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), and disorders ameliorated by a gastroprokinetic agent (e.g., ileus, postoperative ileus and ileus during sepsis; gastroesophageal reflux disease (GORD, or its synonym GERD); eosinophilic esophagitis, gastroparesis such as diabetic gastroparesis; food intolerances and food allergies and other functional bowel disorders, such as non-ulcerative dyspepsia (NUD) and non-cardiac chest pain (NCCP, including costo-chondritis)).

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

In certain embodiments, the inflammatory disorder and/or the immune disorder is a gastrointestinal disorder. In some embodiments, the gastrointestinal disorder is selected from gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)). In certain embodiments, the gastrointestinal disorder is inflammatory bowel disease (IBD).

In certain embodiments, the inflammatory condition and/or immune disorder is a skin condition. In some embodiments, the skin condition is pruritus (itch), psoriasis, eczema, burns or dermatitis. In certain embodiments, the skin condition is psoriasis. In certain embodiments, the skin condition is pruritis.

As used herein, LMM-418 refers to the compound N-(2-(1H-pyrazol-5-yl)phenyl)-4-(2-(piperidin-1-yl)ethoxy)benzamide, having the structural formula:

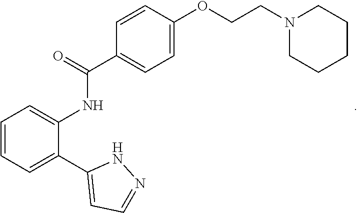 [SMILES: O=C(Nc1ccccc1-c1ccn[nH]1)c1ccc(OCCN2CCCCC2)cc1]

### Compounds

Provided herein are compounds (e.g., compounds of Formula (A), (B), (C), (D), (E), and (F)), and salts, solvates, hydrates, polymorphs, co-crystals, tautomers, stereoisomers, isotopically labeled derivatives, and prodrugs thereof

Compounds of the disclosure can be made by means known in the art of organic synthesis. Methods for optimizing reaction conditions, if necessary minimizing competing by-products, are known in the art. Reaction optimization and scale-up may advantageously utilize high-speed parallel synthesis equipment and computer-controlled microreactors (e.g., Design and Optimization in Organic Synthesis, 2nd Edition, Carlson R, Ed, 2005; Elsevier Science Ltd.; Jahnisch, K et al., Angew. Chem. Int. Ed. Engl. 2004 43: 406; and references therein). Additional reaction schemes and protocols may be determined by the skilled artesian by use of commercially available structure-searchable database software, for instance, SciFinder® (CAS division of the American Chemical Society) and CrossFire Beilstein® (Elsevier MDL), or by appropriate keyword searching using an internet search engine such as Google® or keyword databases such as the US Patent and Trademark Office text database.

As can be appreciated by the skilled artisan, methods of synthesizing the compounds of the formulae herein will be evident to those of ordinary skill in the art, including in the schemes and examples herein. Additionally, the various synthetic steps may be performed in an alternate sequence or order to give the desired compounds. In addition, the solvents, temperatures, reaction durations, etc. delineated herein are for purposes of illustration only and one of ordinary skill in the art will recognize that variation of the reaction conditions can produce the desired compounds of the present disclosure.

In some embodiments, the compound is a compound of any of the formulae herein (e.g., Formula (A), (B), (C), (D), (E), or (F)), or a salt, solvate, hydrate, polymorph, co-crystal, tautomer, stereoisomer, isotopically labeled derivative, or prodrug thereof, wherein the compound is not a compound disclosed in International Patent Application Publication Number WO2019/165158, or a pharmaceutically acceptable salt, solvate, hydrate or prodrug thereof. In some embodiments, the compound is a compound of any of the formulae herein (e.g., Formula (A), (B), (C), (D), (E), or (F)), or a salt, solvate, hydrate, polymorph, co-crystal, tautomer, stereoisomer, isotopically labeled derivative, or prodrug thereof, wherein the compound is not:
N-(5-fluoro-2-(1H-pyrrol-1-yl)phenyl)-4-(2-(piperidin-1-yl)ethoxy)benzamide;
N-(5-fluoro-2-(1H-pyrrol-1-yl)phenyl)-4-(3-(piperidin-1-yl)propoxy)benzamide;
N-(5-fluoro-2-(1H-pyrrol-1-yl)phenyl)-4-phenethoxybenzamide;
N-(5-fluoro-2-(1H-pyrrol-1-yl)phenyl)-4-(3-phenylpropoxy)benzamide;
N-(5-fluoro-2-(1H-pyrrol-1-yl)phenyl)-4-(4-phenylbutoxy)benzamide;
N-(3-(1H-pyrazol-5-yl)phenyl)-4-(3-(piperidin-1-yl)propoxy)benzamide;
N-(3-(1H-pyrazol-5-yl)phenyl)-4-(2-(piperidin-1-yl)ethoxy)benzamide;
N-(3-(1H-pyrazol-5-yl)phenyl)-3-(3-(piperidin-1-yl)propoxy)benzamide;
N-(3-(1H-pyrazol-5-yl)phenyl)-3-(2-(piperidin-1-yl)ethoxy)benzamide;
1-(3-(1H-pyrazol-5-yl)phenyl)-3-(3-(3-(piperidin-1-yl)propoxy)phenyl)urea;
1-(3-(1H-pyrazol-5-yl)phenyl)-3-(3-(2-(piperidin-1-yl)ethoxy)phenyl)urea;
N-(2-(1H-pyrazol-5-yl)phenyl)-4-(3-(piperidin-1-yl)propoxy)benzamide;
N-(2-(1H-pyrazol-5-yl)phenyl)-4-(2-(piperidin-1-yl)ethoxy)benzamide;
N-(3-(1H-pyrazol-5-yl)phenyl)-3-(piperidin-1-yl)benzamide;
N-(3-(1H-pyrazol-5-yl)phenyl)-3-(piperidin-1-ylmethyl)benzamide;
N-(2-(4-oxoazetidin-2-yl)phenyl)-4-(2-(piperidin-1-yl)ethoxy)benzamide;
4-(2-(piperidin-1-yl)ethoxy)-N-(2-(4-thioxoazetidin-2-yl)phenyl)benzamide;
N-(2-(5-oxopyrrolidin-2-yl)phenyl)-4-(2-(piperidin-1-yl)ethoxy)benzamide;
4-(2-(piperidin-1-yl)ethoxy)-N-(2-(5-thioxopyrrolidin-2-yl)phenyl)benzamide;
N-(2-(6-oxopiperidin-2-yl)phenyl)-4-(2-(piperidin-1-yl)ethoxy)benzamide;
4-(2-(piperidin-1-yl)ethoxy)-N-(2-(6-thioxopiperidin-2-yl)phenyl)benzamide;
N-(2-(2-oxohexahydropyrimidin-4-yl)phenyl)-4-(2-(piperidin-1-yl)ethoxy)benzamide;
4-(2-(piperidin-1-yl)ethoxy)-N-(2-(2-thioxohexahydropyrimidin-4-yl)phenyl)benzamide;
N-(2-(2-oxoimidazolidin-4-yl)phenyl)-4-(2-(piperidin-1-yl)ethoxy)benzamide;
4-(2-(piperidin-1-yl)ethoxy)-N-(2-(2-thioxoimidazolidin-4-yl)phenyl)benzamide;
N-(2-(4-oxo-1,3-diazetidin-2-yl)phenyl)-4-(2-(piperidin-1-yl)ethoxy)benzamide;
4-(2-(piperidin-1-yl)ethoxy)-N-(2-(4-thioxo-1,3-diazetidin-2-yl)phenyl)benzamide;
N-(2-(1,2-oxazetidin-3-yl)phenyl)-4-(2-(piperidin-1-yl)ethoxy)benzamide;
N-(2-(1,2-thiazetidin-3-yl)phenyl)-4-(2-(piperidin-1-yl)ethoxy)benzamide;
N-(2-(isoxazolidin-3-yl)phenyl)-4-(2-(piperidin-1-yl)ethoxy)benzamide;
N-(2-(isothiazolidin-3-yl)phenyl)-4-(2-(piperidin-1-yl)ethoxy)benzamide;
N-(2-(1,2-oxazinan-3-yl)phenyl)-4-(2-(piperidin-1-yl)ethoxy)benzamide;
N-(2-(1,2-thiazinan-3-yl)phenyl)-4-(2-(piperidin-1-yl)ethoxy)benzamide;
N-(2-(5-methyl-2,6-dioxo-1,2,3,6-tetrahydropyrimidin-4-yl)phenyl)-4-(2-(piperidin-1-yl)ethoxy)benzamide;
N-(2-(2,6-dioxo-1,2,3,6-tetrahydropyrimidin-4-yl)phenyl)-4-(2-(piperidin-1-yl)ethoxy)benzamide;
N-(2-(5-oxopyrrolidin-3-yl)phenyl)-4-(2-(piperidin-1-yl)ethoxy)benzamide;
4-(2-(piperidin-1-yl)ethoxy)-N-(2-(5-thioxopyrrolidin-3-yl)phenyl)benzamide;
N-(2-(6-oxopiperidin-3-yl)phenyl)-4-(2-(piperidin-1-yl)ethoxy)benzamide;
4-(2-(piperidin-1-yl)ethoxy)-N-(2-(6-thioxopiperidin-3-yl)phenyl)benzamide;
N-(2-(5-oxo-2,5-dihydroisoxazol-3-yl)phenyl)-4-(2-(piperidin-1-yl)ethoxy)benzamide;
N-(3-(2,4-dioxo-1,2,3,4-tetrahydro-7H-pyrrolo[2,3-d]pyrimidin-7-yl)phenyl)-4-(2-(piperidin-1-yl)ethoxy)benzamide;
N-(2-(7-oxo-4,5,6,7-tetrahydro-1H-indol-2-yl)phenyl)-4-(2-(piperidin-1-yl)ethoxy)benzamide;
N-(2-(1H-pyrrolo[2,3-b]pyridin-2-yl)phenyl)-4-(2-(piperidin-1-yl)ethoxy)benzamide;
N-(3-(2,4-dioxo-1,2,3,4-tetrahydroquinazolin-8-yl)phenyl)-4-(2-(piperidin-1-yl)ethoxy)benzamide;
N-(3-(2,4-dioxo-1,2,3,4-tetrahydrofuro[3,2-d]pyrimidin-7-yl)phenyl)-4-(2-(piperidin-1-yl)ethoxy)benzamide;
N-(3-(2-oxo-2,3-dihydro-1H-benzo [d]imidazol-4-yl)phenyl)-4-(2-(piperidin-1-yl)ethoxy)benzamide;
N-(2-(4,7-dioxo-4,7-dihydro-1H-indol-3-yl)phenyl)-4-(2-(piperidin-1-yl)ethoxy)benzamide;
N-(2-(5,6-dimethyl-4,7-dioxo-4,7-dihydro-1H-indol-3-yl)phenyl)-4-(2-(piperidin-1-yl)ethoxy)benzamide;
N-(2-(7-oxo-4,5,6,7-tetrahydro-1H-indol-3-yl)phenyl)-4-(2-(piperidin-1-yl)ethoxy)benzamide;
N-(2-(4,7-dioxo-4,5,6,7-tetrahydro-1H-indol-3-yl)phenyl)-4-(2-(piperidin-1-yl)ethoxy)benzamide;
N-(2-(7-oxo-1,4,5,7-tetrahydropyrano[3,4-b]pyrrol-3-yl)phenyl)-4-(2-(piperidin-1-yl)ethoxy)benzamide;
N-(2-(7-oxo-4,5,6,7-tetrahydro-1H-pyrrolo[2,3-c]pyridin-3-yl)phenyl)-4-(2-(piperidin-1-yl)ethoxy)benzamide;
N-(2-(7-oxo-6,7-dihydro-1H-pyrrolo[2,3-c]pyridin-3-yl)phenyl)-4-(2-(piperidin-1-yl)ethoxy)benzamide;
N-(2-(7-oxo-1,7-dihydropyrano[3,4-b]pyrrol-3-yl)phenyl)-4-(2-(piperidin-1-yl)ethoxy)benzamide;
N-(2-(2-oxo-2,3-dihydro-1H-imidazol-4-yl)phenyl)-4-(2-(piperidin-1-yl)ethoxy)benzamide;
1-(2-(1H-pyrazol-5-yl)phenyl)-3-(3-(2-(piperidin-1-yl)ethoxy)phenyl)urea;
1-(2-(5-methyl-2,6-dioxo-1,2,3,6-tetrahydropyrimidin-4-yl)phenyl)-3-(3-(2-(piperidin-1-yl)ethoxy)phenyl)urea;
1-(2-(2,6-dioxo-1,2,3,6-tetrahydropyrimidin-4-yl)phenyl)-3-(3-(2-(piperidin-1-yl)ethoxy)phenyl)urea;
1-(2-(1H-pyrazol-5-yl)phenyl)-3-(4-(2-(piperidin-1-yl)ethoxy)phenyl)urea;
N-(2-(4-fluoro-1H-pyrazol-5-yl)phenyl)-4-(2-(piperidin-1-yl)ethoxy)benzamide;
N-(2-(3,4-difluoro-1H-pyrazol-5-yl)phenyl)-4-(2-(piperidin-1-yl)ethoxy)benzamide;
N-(2-(3-fluoro-1H-pyrazol-5-yl)phenyl)-4-(2-(piperidin-1-yl)ethoxy)benzamide;
N-(2-(4-chloro-1H-pyrazol-5-yl)phenyl)-4-(2-(piperidin-1-yl)ethoxy)benzamide;
N-(2-(3,4-dichloro-1H-pyrazol-5-yl)phenyl)-4-(2-(piperidin-1-yl)ethoxy)benzamide;
N-(2-(3-chloro-1H-pyrazol-5-yl)phenyl)-4-(2-(piperidin-1-yl)ethoxy)benzamide;
N-(2-(3-methoxy-1H-pyrazol-5-yl)phenyl)-4-(2-(piperidin-1-yl)ethoxy)benzamide; 5-(2-(4-(2-(piperidin-1-yl)ethoxy)benzamido)phenyl)-1H-pyrazol-3-yl acetate;
N-(2-(4-methoxy-1H-pyrazol-5-yl)phenyl)-4-(2-(piperidin-1-yl)ethoxy)benzamide; 5-(2-(4-(2-(piperidin-1-yl)ethoxy)benzamido)phenyl)-1H-pyrazol-4-yl acetate;
N-(2-(4-acetamido-1H-pyrazol-5-yl)phenyl)-4-(2-(piperidin-1-yl)ethoxy)benzamide;
N-(2-(4-(dimethylamino)-1H-pyrazol-5-yl)phenyl)-4-(2-(piperidin-1-yl)ethoxy)benzamide;
N-(2-(3-(dimethylamino)-1H-pyrazol-5-yl)phenyl)-4-(2-(piperidin-1-yl)ethoxy)benzamide;
N-(4-methyl-2-(1H-pyrazol-5-yl)phenyl)-4-(2-(piperidin-1-yl)ethoxy)benzamide;
N-(4,5-dimethoxy-2-(1H-pyrazol-5-yl)phenyl)-4-(2-(piperidin-1-yl)ethoxy)benzamide;
N-(4,5-dihydroxy-2-(1H-pyrazol-5-yl)phenyl)-4-(2-(piperidin-1-yl)ethoxy)benzamide;
N-(5-bromo-2-(1H-pyrazol-5-yl)phenyl)-4-(2-(piperidin-1-yl)ethoxy)benzamide;
4-(2-(piperidin-1-yl)ethoxy)-N-(2-(4-(trifluoromethyl)-1H-pyrazol-5-yl)phenyl)benzamide;
4-(2-(piperidin-1-yl)ethoxy)-N-(2-(3-(trifluoromethyl)-1H-pyrazol-5-yl)phenyl)benzamide;
N-(3,5-dibromo-2-(1H-pyrazol-5-yl)phenyl)-4-(2-(piperidin-1-yl)ethoxy)benzamide;
N-(3-bromo-2-(1H-pyrazol-5-yl)phenyl)-4-(2-(piperidin-1-yl)ethoxy)benzamide;
N-(2-(3-acetamido-1H-pyrazol-5-yl)phenyl)-4-(2-(piperidin-1-yl)ethoxy)benzamide;
N-(2-(3-amino-1H-pyrazol-5-yl)phenyl)-4-(2-(piperidin-1-yl)ethoxy)benzamide;
   or a pharmaceutically acceptable salt, solvate, hydrate or prodrug thereof.

In some embodiments, the compound is a compound of any of the formulae herein (e.g., Formula (A), (B), (C), (D), (E), or (F)), or a salt, solvate, hydrate, polymorph, co-crystal, tautomer, stereoisomer, isotopically labeled derivative, or prodrug thereof, wherein the compound is not:

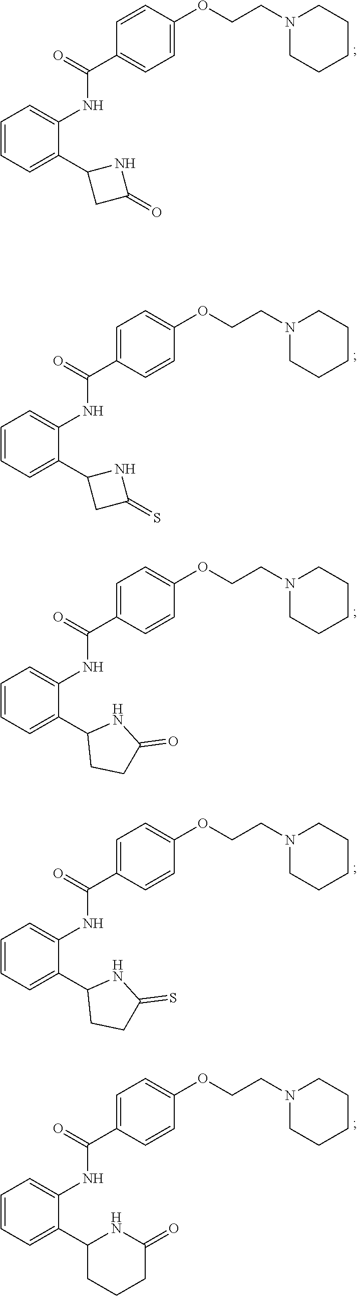 [SMILES: O=C1CC(c2ccccc2NC(=O)c2ccc(OCCN3CCCCC3)cc2)N1 ; O=C(Nc1ccccc1C1CC(=S)N1)c1ccc(OCCN2CCCCC2)cc1 ; O=C1CCC(c2ccccc2NC(=O)c2ccc(OCCN3CCCCC3)cc2)N1 ; O=C(Nc1ccccc1C1CCC(=S)N1)c1ccc(OCCN2CCCCC2)cc1 ; O=C1CCCC(c2ccccc2NC(=O)c2ccc(OCCN3CCCCC3)cc2)N1]

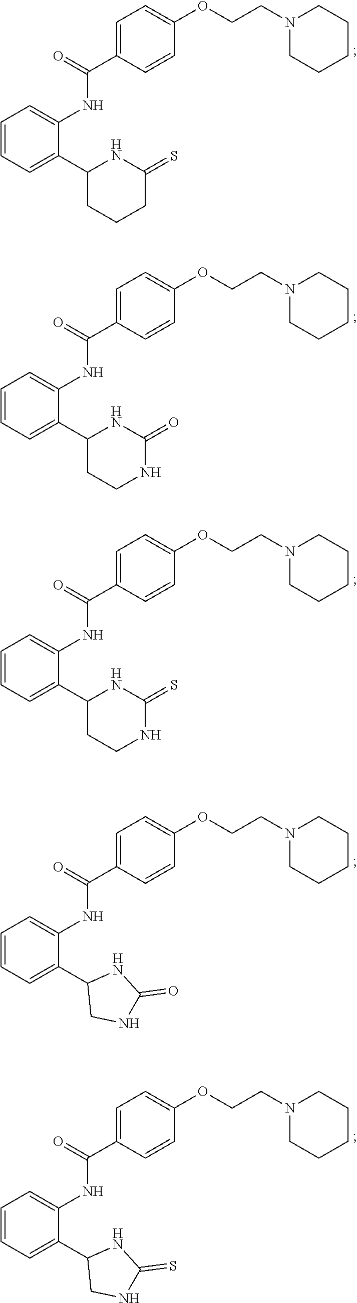 [SMILES: O=C(Nc1ccccc1C1CCCC(=S)N1)c1ccc(OCCN2CCCCC2)cc1 ; O=C1NCCC(c2ccccc2NC(=O)c2ccc(OCCN3CCCCC3)cc2)N1 ; O=C(Nc1ccccc1C1CCNC(=S)N1)c1ccc(OCCN2CCCCC2)cc1 ; O=C1NCC(c2ccccc2NC(=O)c2ccc(OCCN3CCCCC3)cc2)N1 ; O=C(Nc1ccccc1C1CNC(=S)N1)c1ccc(OCCN2CCCCC2)cc1]

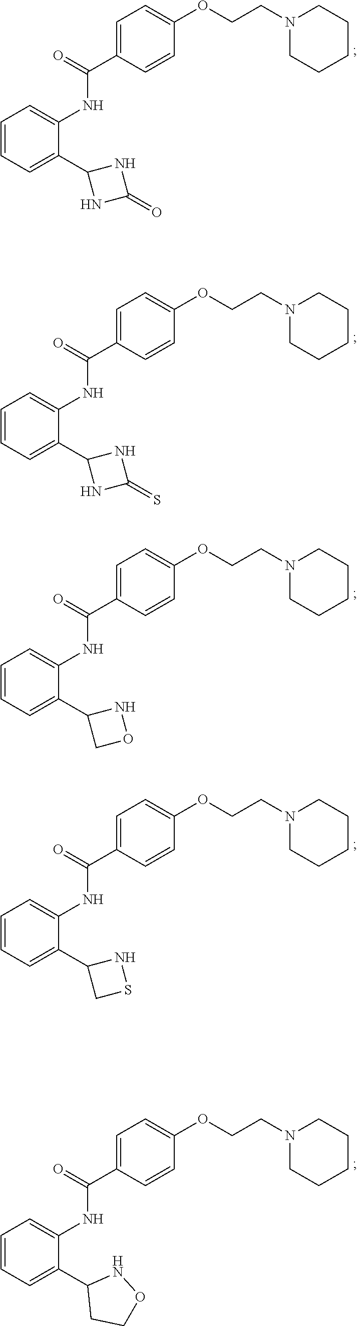 [SMILES: O=C1NC(c2ccccc2NC(=O)c2ccc(OCCN3CCCCC3)cc2)N1 ; O=C(Nc1ccccc1C1NC(=S)N1)c1ccc(OCCN2CCCCC2)cc1 ; O=C(Nc1ccccc1C1CON1)c1ccc(OCCN2CCCCC2)cc1 ; O=C(Nc1ccccc1C1CSN1)c1ccc(OCCN2CCCCC2)cc1 ; O=C(Nc1ccccc1C1CCON1)c1ccc(OCCN2CCCCC2)cc1]

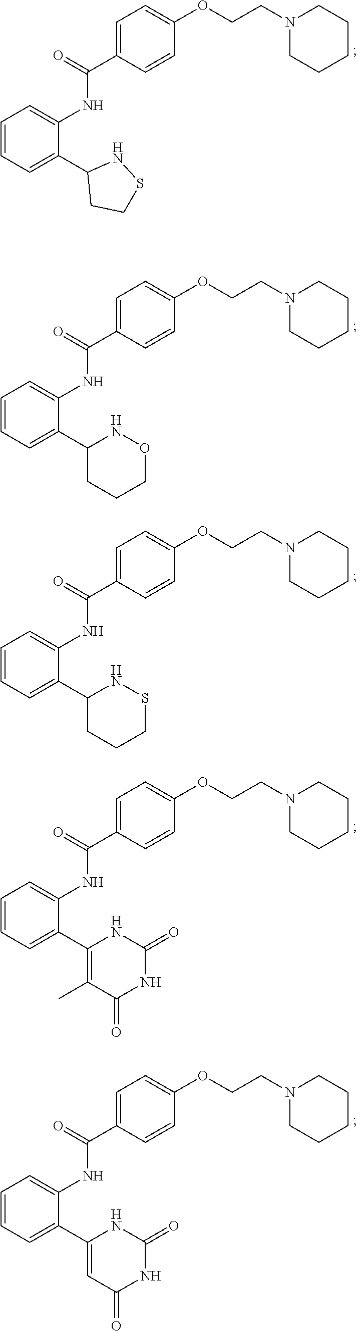

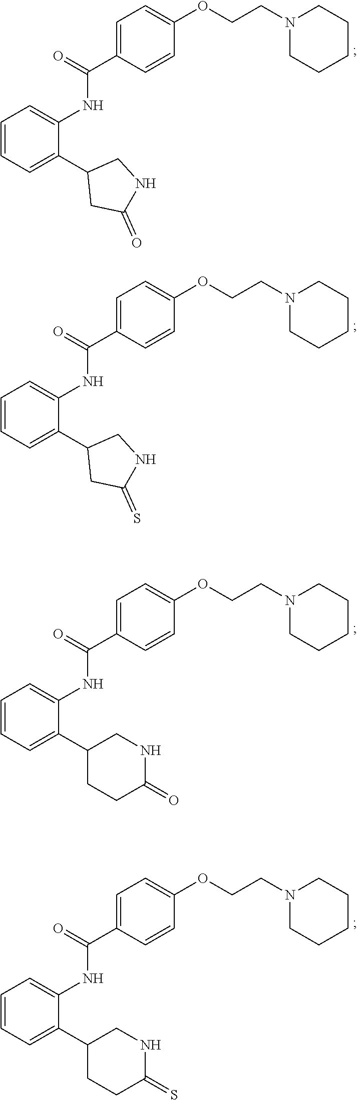

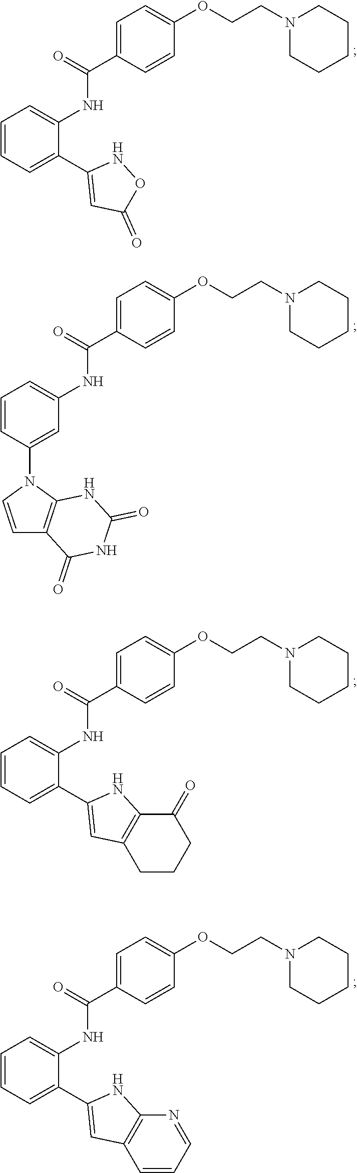

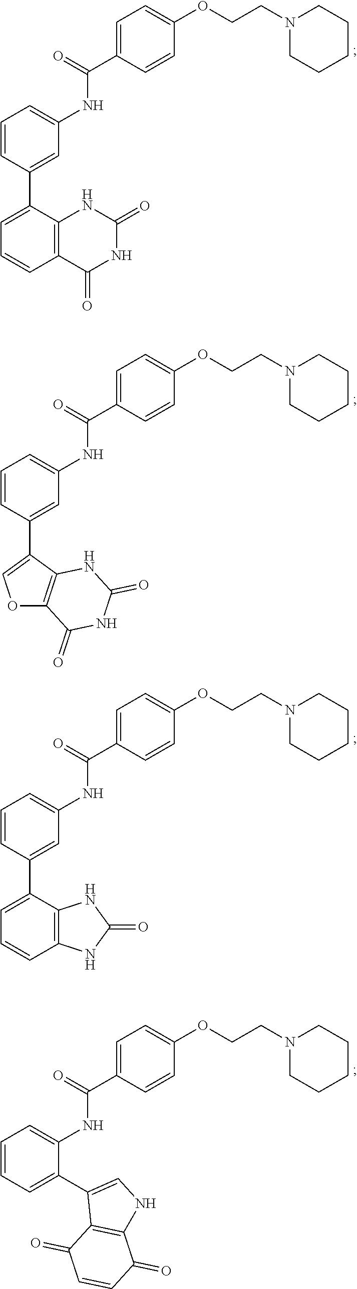 [SMILES: O=C(Nc1cccc(-c2cccc3c(=O)[nH]c(=O)[nH]c23)c1)c1ccc(OCCN2CCCCC2)cc1 ; O=C(Nc1cccc(-c2coc3c(=O)[nH]c(=O)[nH]c23)c1)c1ccc(OCCN2CCCCC2)cc1 ; O=C(Nc1cccc(-c2cccc3[nH]c(=O)[nH]c23)c1)c1ccc(OCCN2CCCCC2)cc1 ; O=C(Nc1ccccc1-c1c[nH]c2c1C(=O)C=CC2=O)c1ccc(OCCN2CCCCC2)cc1]

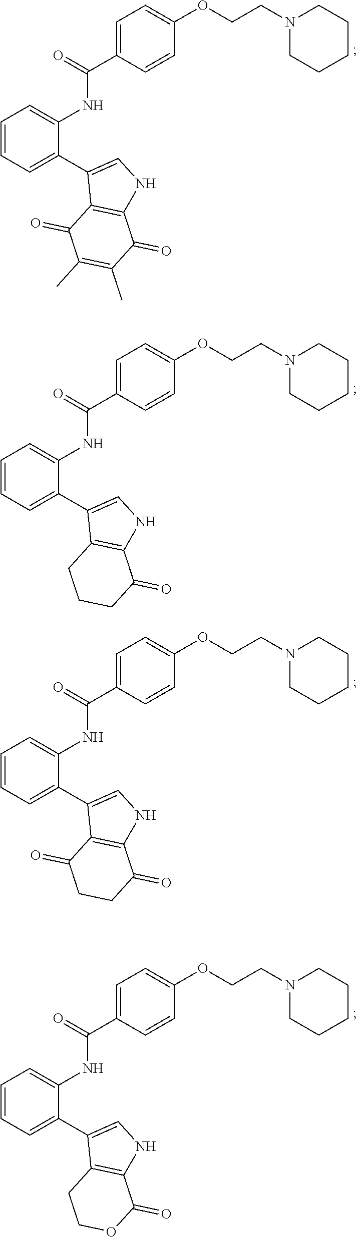

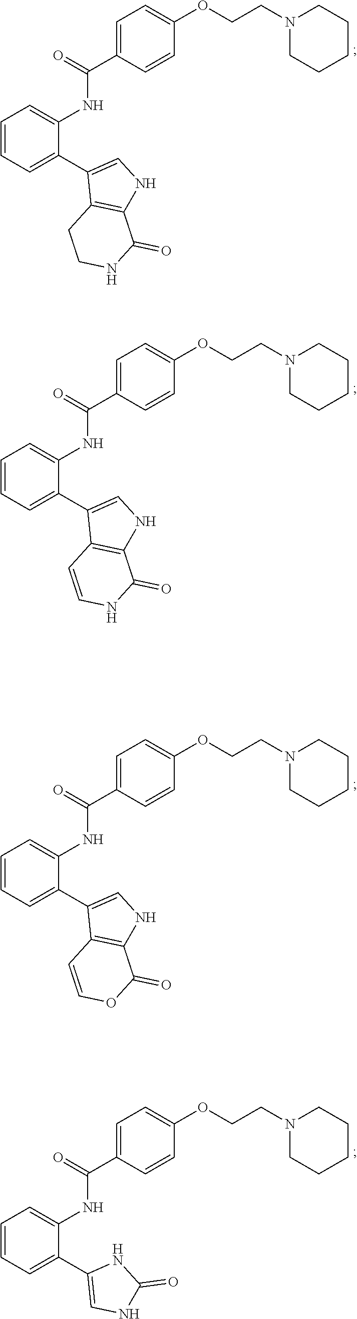 [SMILES: O=C(Nc1ccccc1-c1c[nH]c2c1CCNC2=O)c1ccc(OCCN2CCCCC2)cc1 ; O=C(Nc1ccccc1-c1c[nH]c2c(=O)[nH]ccc12)c1ccc(OCCN2CCCCC2)cc1 ; O=C(Nc1ccccc1-c1c[nH]c2c(=O)occc12)c1ccc(OCCN2CCCCC2)cc1 ; O=C(Nc1ccccc1-c1c[nH]c(=O)[nH]1)c1ccc(OCCN2CCCCC2)cc1]

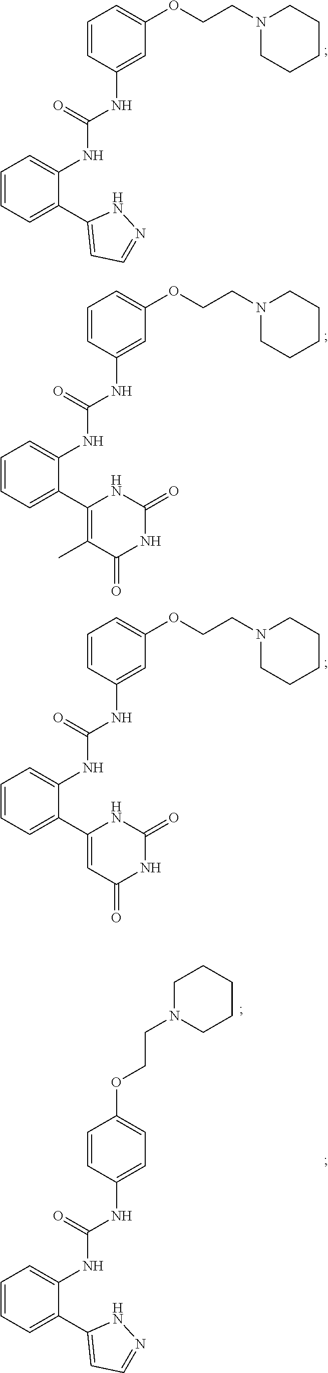

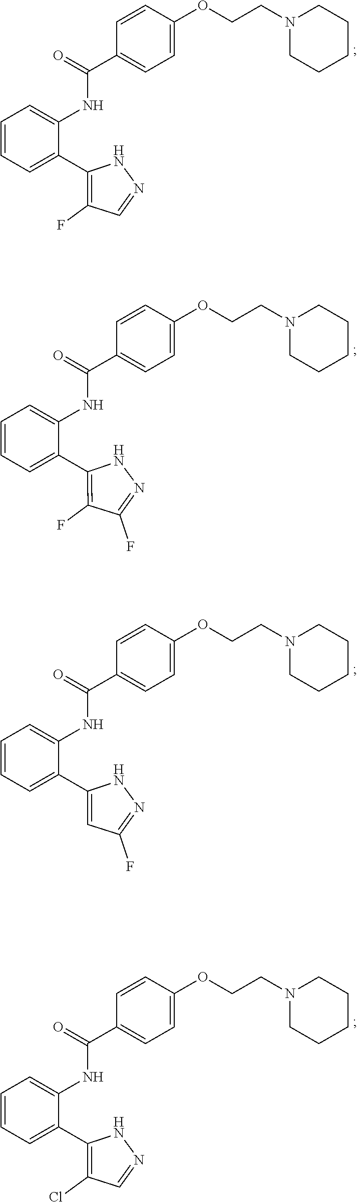 [SMILES: O=C(Nc1ccccc1-c1[nH]ncc1F)c1ccc(OCCN2CCCCC2)cc1 ; O=C(Nc1ccccc1-c1[nH]nc(F)c1F)c1ccc(OCCN2CCCCC2)cc1 ; O=C(Nc1ccccc1-c1cc(F)n[nH]1)c1ccc(OCCN2CCCCC2)cc1 ; O=C(Nc1ccccc1-c1[nH]ncc1Cl)c1ccc(OCCN2CCCCC2)cc1]

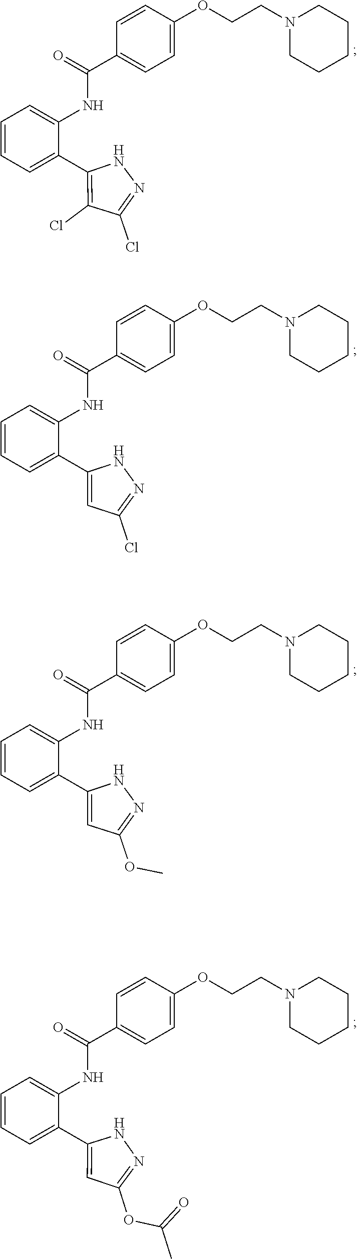 [SMILES: O=C(Nc1ccccc1-c1[nH]nc(Cl)c1Cl)c1ccc(OCCN2CCCCC2)cc1 ; O=C(Nc1ccccc1-c1cc(Cl)n[nH]1)c1ccc(OCCN2CCCCC2)cc1 ; COc1cc(-c2ccccc2NC(=O)c2ccc(OCCN3CCCCC3)cc2)[nH]n1 ; CC(=O)Oc1cc(-c2ccccc2NC(=O)c2ccc(OCCN3CCCCC3)cc2)[nH]n1]

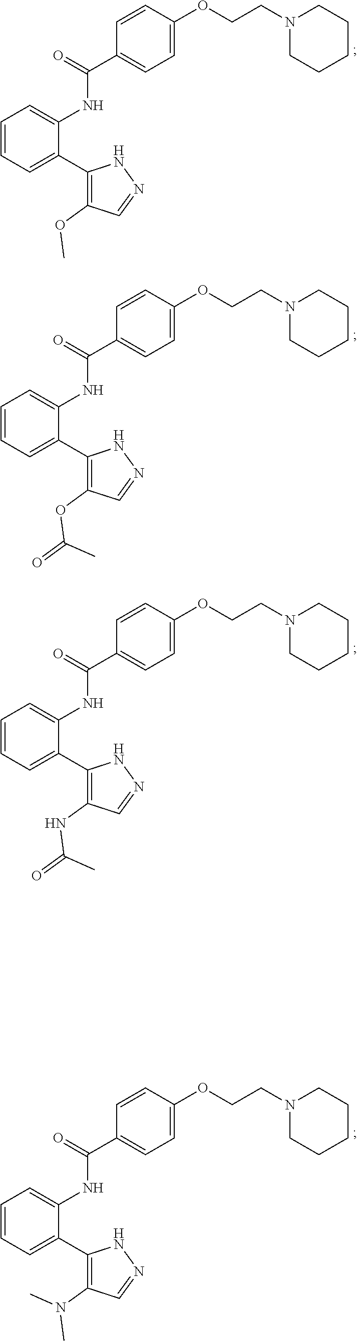 [SMILES: COc1cn[nH]c1-c1ccccc1NC(=O)c1ccc(OCCN2CCCCC2)cc1 ; CC(=O)Oc1cn[nH]c1-c1ccccc1NC(=O)c1ccc(OCCN2CCCCC2)cc1 ; CC(=O)Nc1cn[nH]c1-c1ccccc1NC(=O)c1ccc(OCCN2CCCCC2)cc1 ; CN(C)c1cn[nH]c1-c1ccccc1NC(=O)c1ccc(OCCN2CCCCC2)cc1]

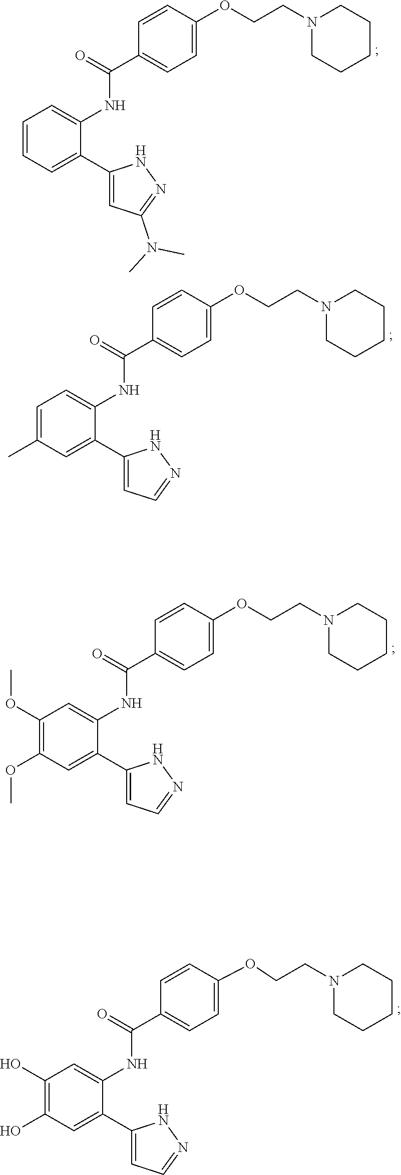

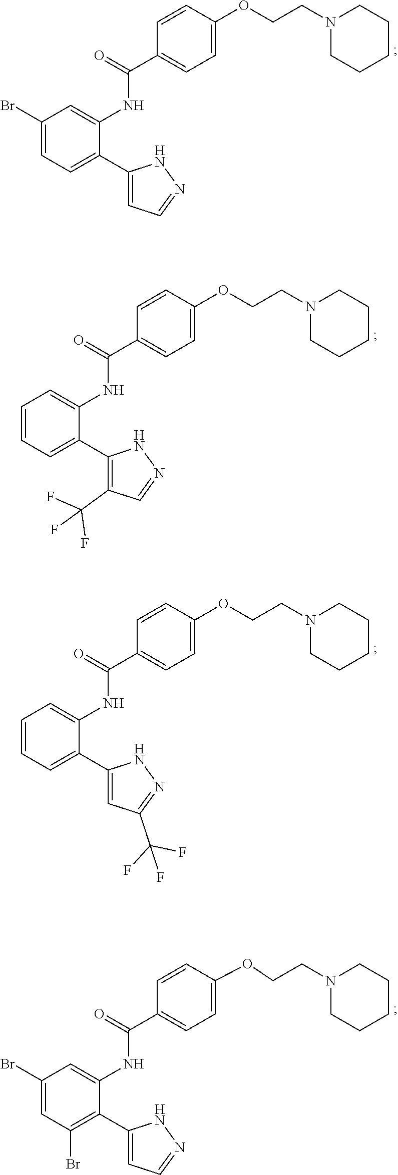 [SMILES: O=C(Nc1cc(Br)ccc1-c1ccn[nH]1)c1ccc(OCCN2CCCCC2)cc1 ; O=C(Nc1ccccc1-c1[nH]ncc1C(F)(F)F)c1ccc(OCCN2CCCCC2)cc1 ; O=C(Nc1ccccc1-c1cc(C(F)(F)F)n[nH]1)c1ccc(OCCN2CCCCC2)cc1 ; O=C(Nc1cc(Br)cc(Br)c1-c1ccn[nH]1)c1ccc(OCCN2CCCCC2)cc1]

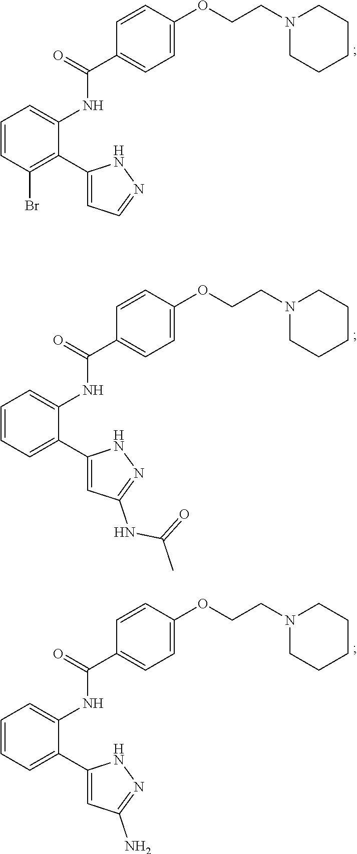

or a pharmaceutically acceptable salt, solvate, hydrate or prodrug thereof.

### Formulae (A) and (F)

In one aspect, provided herein is a compound of Formula (F), or a salt, solvate, hydrate, polymorph, co-crystal, tautomer, stereoisomer, isotopically labeled derivative, or prodrug thereof:

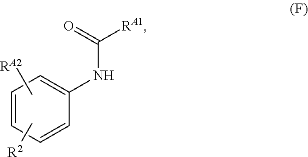

wherein:
R^{A1} is

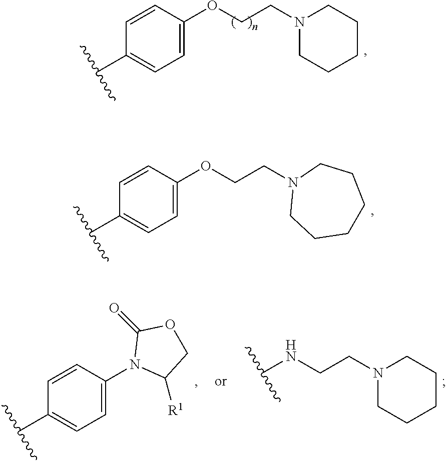

R¹ is optionally substituted alkyl or optionally substituted cycloalkyl;
R^{A2} is H or halogen;
R² is optionally substituted

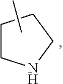

optionally substituted

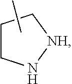

or optionally substituted

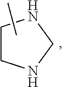

optionally substituted

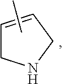

optionally substituted

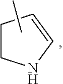

optionally substituted

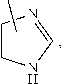

optionally substituted

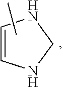

optionally substituted

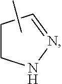

optionally substituted

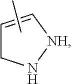

optionally substituted

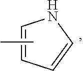

optionally substituted

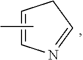

optionally substituted

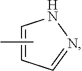

optionally substituted

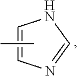

optionally substituted

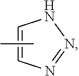

optionally substituted

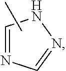

optionally substituted

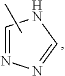

or optionally substituted

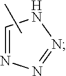

and n is 1 or 2.

In one aspect, provided herein is a compound of Formula (A), or a salt, solvate, hydrate, polymorph, co-crystal, tautomer, stereoisomer, isotopically labeled derivative, or prodrug thereof:

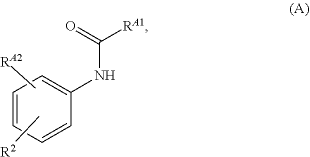

wherein:
R^{A1} is

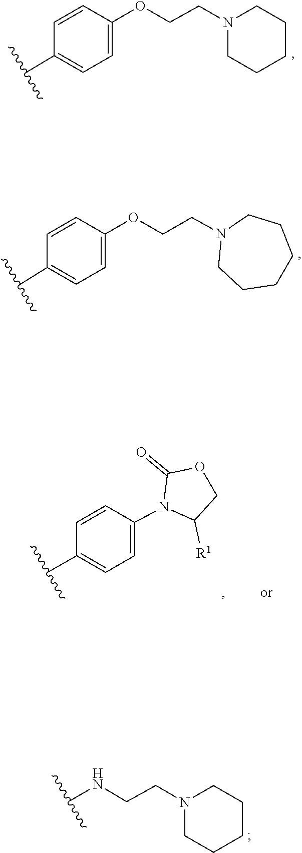

R¹ is optionally substituted alkyl or cycloalkyl;
R^{A2} is H or halogen; and
R² is optionally substituted

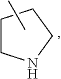

optionally substituted

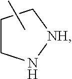

or optionally substituted

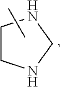

optionally substituted

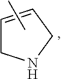

optionally substituted

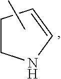

optionally substituted

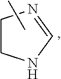

optionally substituted

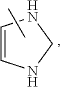

optionally substituted

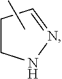

optionally substituted

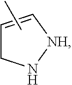

optionally substituted

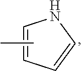

optionally substituted

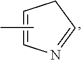

optionally substituted

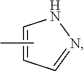

optionally substituted

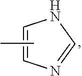

optionally substituted

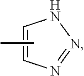

optionally substituted

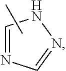

optionally substituted

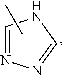

or optionally substituted

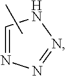

In some embodiments, the compound is of Formula (IV), or a salt, solvate, hydrate, polymorph, co-crystal, tautomer, stereoisomer, isotopically labeled derivative, or prodrug thereof:

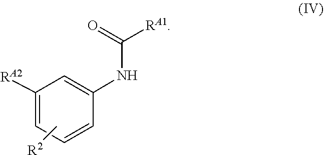

In some embodiments, the compound is of Formula (IV-c), or a salt, solvate, hydrate, polymorph, co-crystal, tautomer, stereoisomer, isotopically labeled derivative, or prodrug thereof:

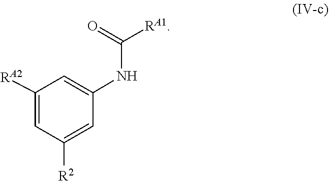

In some embodiments, the compound is of Formula (IV-d), or a salt, solvate, hydrate, polymorph, co-crystal, tautomer, stereoisomer, isotopically labeled derivative, or prodrug thereof:

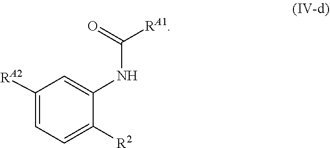

In some embodiments, the compound is of Formula (IV-e), or a salt, solvate, hydrate, polymorph, co-crystal, tautomer, stereoisomer, isotopically labeled derivative, or prodrug thereof:

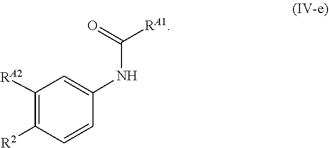

In some embodiments, the compound is of Formula (I), or a salt, solvate, hydrate, polymorph, co-crystal, tautomer, stereoisomer, isotopically labeled derivative, or prodrug thereof:

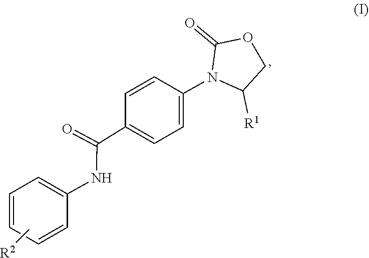

wherein:
R¹ is alkyl or cycloalkyl; and
R² is selected from the group consisting of:

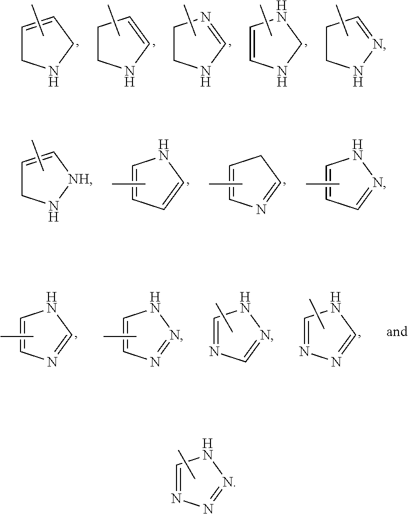

In some embodiments, the compound is of Formula (I-a), or a salt, solvate, hydrate, polymorph, co-crystal, tautomer, stereoisomer, isotopically labeled derivative, or prodrug thereof:

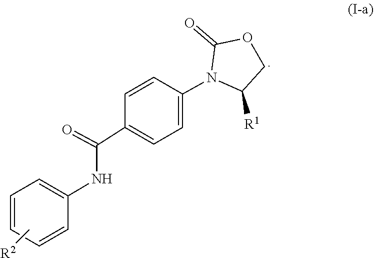

In some embodiments, the compound is of Formula (I-b), or a salt, solvate, hydrate, polymorph, co-crystal, tautomer, stereoisomer, isotopically labeled derivative, or prodrug thereof:

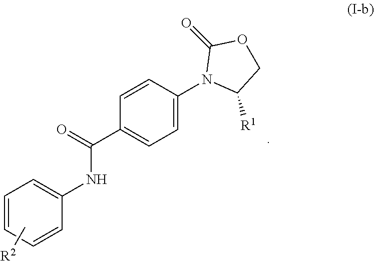

In some embodiments, the compound is of Formula (I-c), or a salt, solvate, hydrate, polymorph, co-crystal, tautomer, stereoisomer, isotopically labeled derivative, or prodrug thereof:

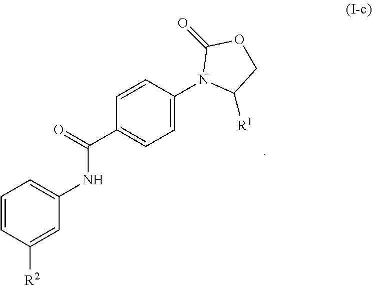

In some embodiments, the compound is of Formula (I-d), or a salt, solvate, hydrate, polymorph, co-crystal, tautomer, stereoisomer, isotopically labeled derivative, or prodrug thereof:

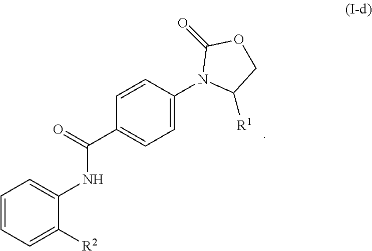

In some embodiments, the compound is of Formula (V), or a salt, solvate, hydrate, polymorph, co-crystal, tautomer, stereoisomer, isotopically labeled derivative, or prodrug thereof:

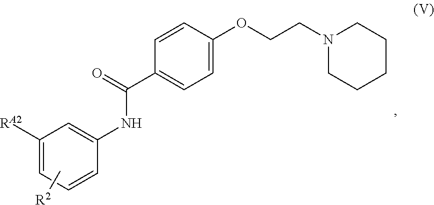

wherein R² is optionally substituted

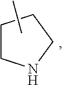

optionally substituted

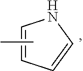

optionally substituted

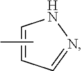

or optionally substituted

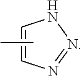

In some embodiments, the compound is of Formula (V-a), or a salt, solvate, hydrate, polymorph, co-crystal, tautomer, stereoisomer, isotopically labeled derivative, or prodrug thereof:

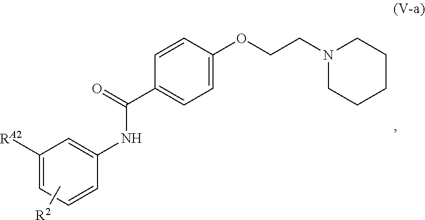

wherein:
R² is

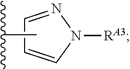

and
R^{A3} is optionally substituted alkyl or optionally substituted carbocyclyl.

In some embodiments, the compound is of Formula (VI), or a salt, solvate, hydrate, polymorph, co-crystal, tautomer, stereoisomer, isotopically labeled derivative, or prodrug thereof:

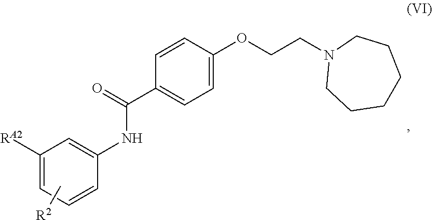

wherein R² is optionally substituted

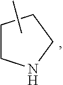

optionally substituted

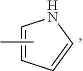

optionally substituted

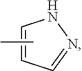

or optionally substituted

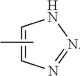

In some embodiments, the compound is of Formula (VII), or a salt, solvate, hydrate, polymorph, co-crystal, tautomer, stereoisomer, isotopically labeled derivative, or prodrug thereof:

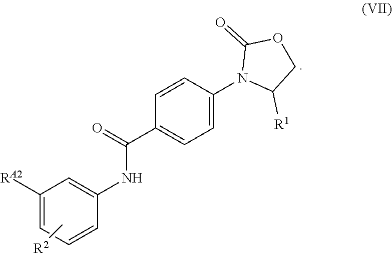

In some embodiments, the compound is of Formula (VIII), or a salt, solvate, hydrate, polymorph, co-crystal, tautomer, stereoisomer, isotopically labeled derivative, or prodrug thereof:

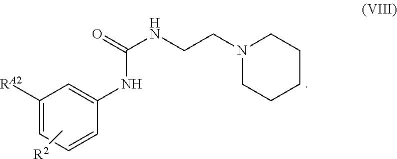

In some embodiments, the compound is of Formula (XVIII), or a salt, solvate, hydrate, polymorph, co-crystal, tautomer, stereoisomer, isotopically labeled derivative, or prodrug thereof:

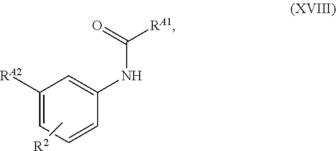

wherein R^{A1} is

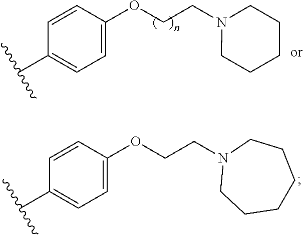

and
R² is

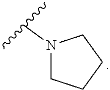 [SMILES: *N1CCCC1]

In some embodiments, the compound is of Formula (XVIII-a), or a salt, solvate, hydrate, polymorph, co-crystal, tautomer, stereoisomer, isotopically labeled derivative, or prodrug thereof:

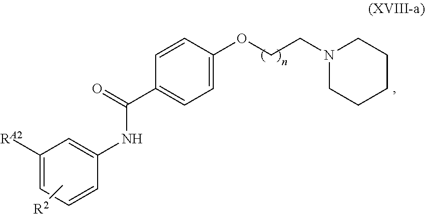

wherein R² is

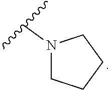 [SMILES: *N1CCCC1]

In some embodiments, the compound is of Formula (XVIII-b), or a salt, solvate, hydrate, polymorph, co-crystal, tautomer, stereoisomer, isotopically labeled derivative, or prodrug thereof:

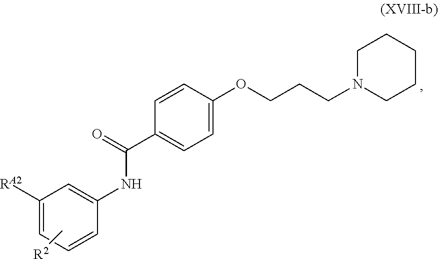

wherein R² is

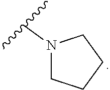 [SMILES: *N1CCCC1]

In some embodiments, the compound is of Formula (XIX), or a salt, solvate, hydrate, polymorph, co-crystal, tautomer, stereoisomer, isotopically labeled derivative, or prodrug thereof:

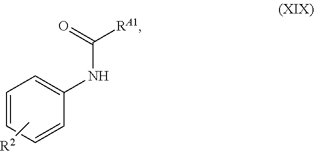

wherein R^{A1} is

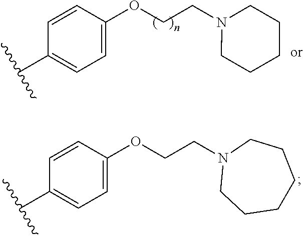

and
R² is

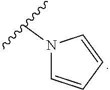 [SMILES: *n1cccc1]

In some embodiments, the compound is of Formula (XIX-a), or a salt, solvate, hydrate, polymorph, co-crystal, tautomer, stereoisomer, isotopically labeled derivative, or prodrug thereof:

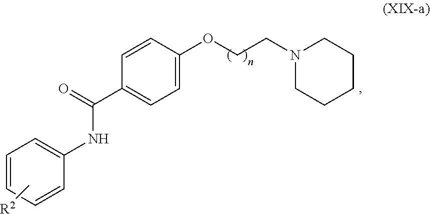

wherein R² is

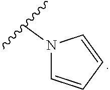 [SMILES: *n1cccc1]

In some embodiments, the compound is of Formula (XIX-b), or a salt, solvate, hydrate, polymorph, co-crystal, tautomer, stereoisomer, isotopically labeled derivative, or prodrug thereof:

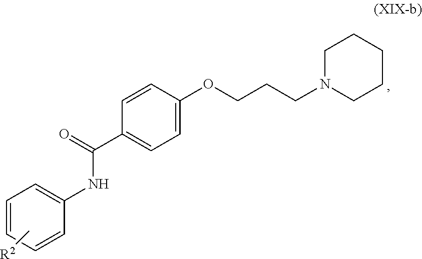

wherein R² is

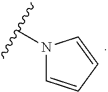 [SMILES: *n1cccc1]

In some embodiments, the compound is of Formula (XX), or a salt, solvate, hydrate, polymorph, co-crystal, tautomer, stereoisomer, isotopically labeled derivative, or prodrug thereof:

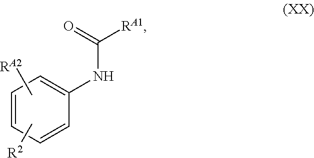

wherein:
R² is

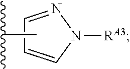

and
R^{A3} is optionally substituted alkyl or optionally substituted carbocyclyl.

In some embodiments, the compound is of Formula (XX-a), or a salt, solvate, hydrate, polymorph, co-crystal, tautomer, stereoisomer, isotopically labeled derivative, or prodrug thereof:

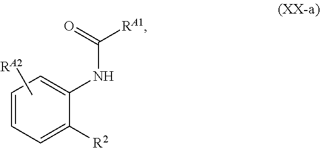

wherein:
R² is

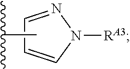

and
R^{A3} is optionally substituted alkyl or optionally substituted carbocyclyl.

In some embodiments, the compound is of Formula (XX-b), or a salt, solvate, hydrate, polymorph, co-crystal, tautomer, stereoisomer, isotopically labeled derivative, or prodrug thereof:

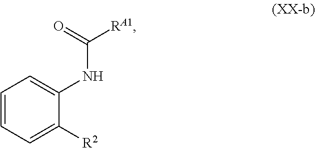

wherein:
R² is

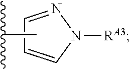

and
R^{A3} is optionally substituted alkyl or optionally substituted carbocyclyl.

In some embodiments, the compound is of Formula (XXI), or a salt, solvate, hydrate, polymorph, co-crystal, tautomer, stereoisomer, isotopically labeled derivative, or prodrug thereof:

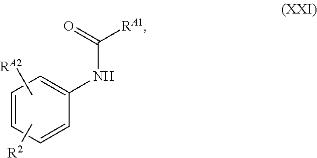

wherein:
R^{A1} is

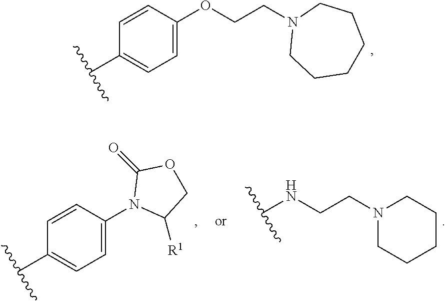

As defined herein, R^{A1} is

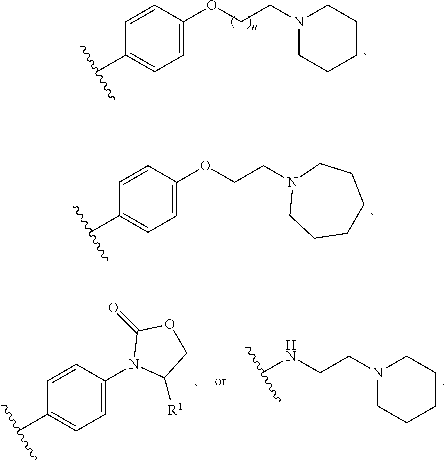

In some embodiments, R^{A1} is

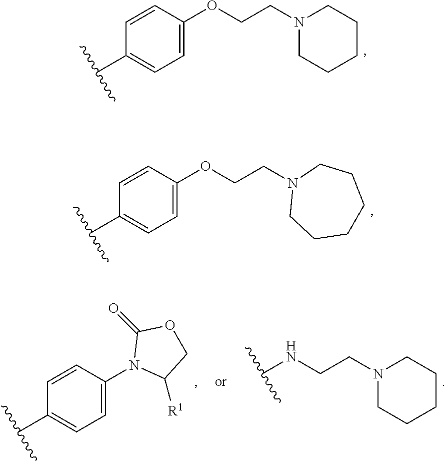

In some embodiments, R^{A1} is

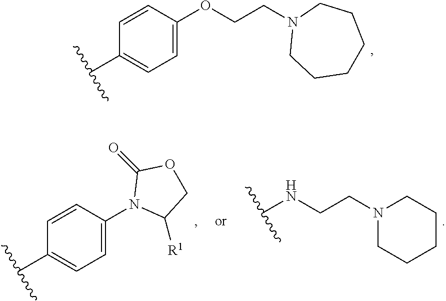

In some embodiments, R^{A1} is

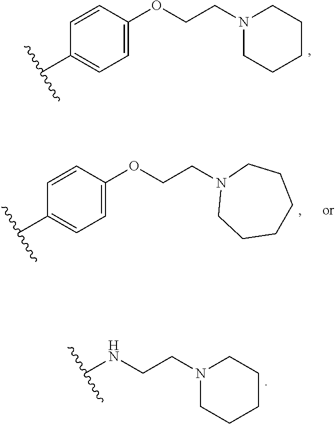 [SMILES: *c1ccc(OCCN2CCCCC2)cc1 ; *c1ccc(OCCN2CCCCCC2)cc1 ; *NCCN1CCCCC1]

In some embodiments, R^{A1} is

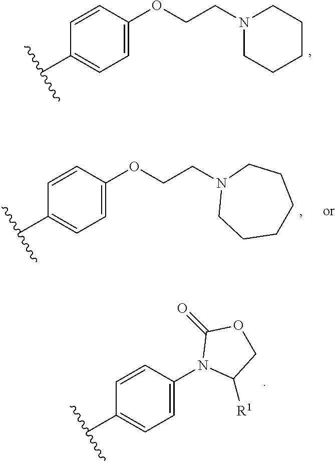

In some embodiments, R^{A1} is

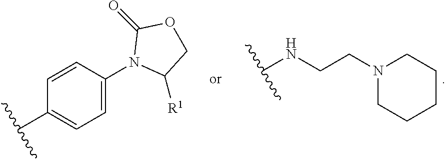

In some embodiments, R^{A1} is

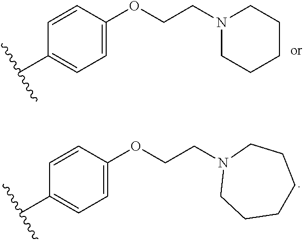 [SMILES: *c1ccc(OCCN2CCCCC2)cc1 ; *c1ccc(OCCN2CCCCCC2)cc1]

In some embodiments, R^{A1} is

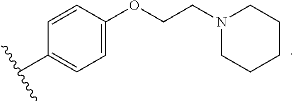 [SMILES: *c1ccc(OCCN2CCCCC2)cc1]

In some embodiments, R^{A1} is

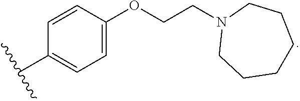 [SMILES: *c1ccc(OCCN2CCCCCC2)cc1]

In some embodiments, R^{A1} is

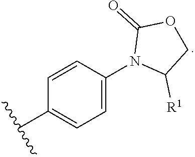

In some embodiments, R^{A1} is

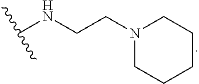 [SMILES: *NCCN1CCCCC1]

In some embodiments, R^{A1} is

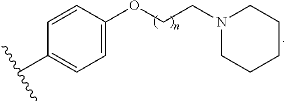

In some embodiments, R^{A1} is

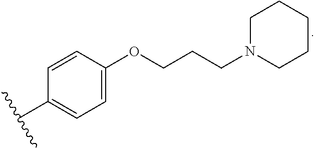 [SMILES: *c1ccc(OCCCN2CCCCC2)cc1]

In some embodiments, R^{A1} is

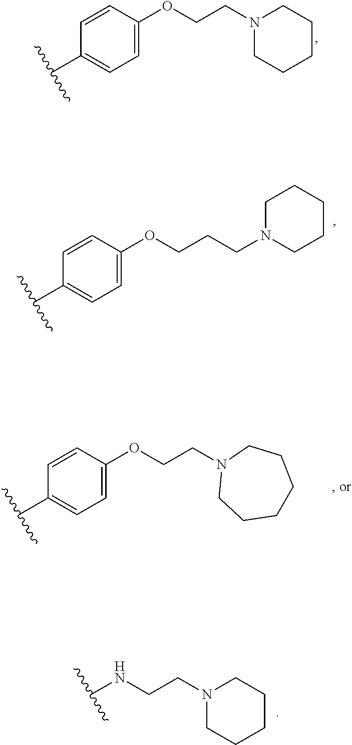 [SMILES: *c1ccc(OCCN2CCCCC2)cc1 ; *c1ccc(OCCCN2CCCCC2)cc1 ; *c1ccc(OCCN2CCCCCC2)cc1 ; *NCCN1CCCCC1]

In some embodiments, R^{A1} is

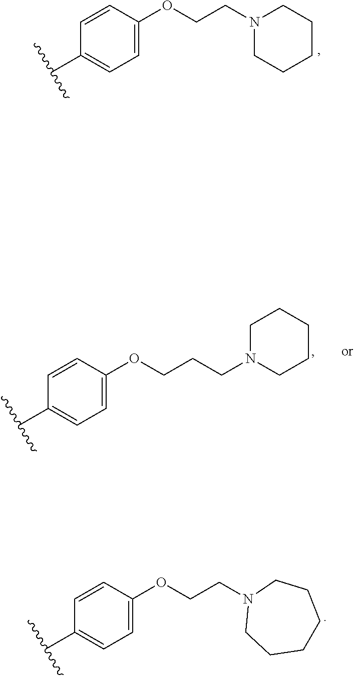 [SMILES: *c1ccc(OCCN2CCCCC2)cc1 ; *c1ccc(OCCCN2CCCCC2)cc1 ; *c1ccc(OCCN2CCCCCC2)cc1]

In some embodiments, R^{A1} is

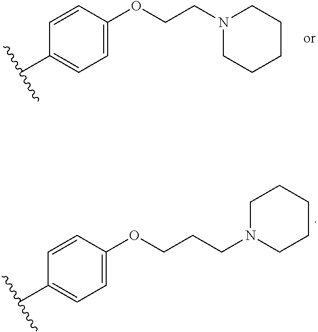 [SMILES: *c1ccc(OCCN2CCCCC2)cc1 ; *c1ccc(OCCCN2CCCCC2)cc1]

In some embodiments, R^{A1} is not

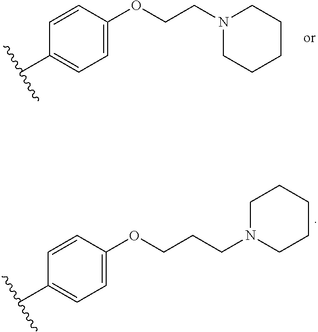 [SMILES: *c1ccc(OCCN2CCCCC2)cc1 ; *c1ccc(OCCCN2CCCCC2)cc1]

In some embodiments, R^{A1} is not

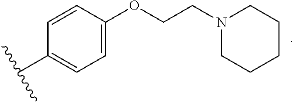 [SMILES: *c1ccc(OCCN2CCCCC2)cc1]

In some embodiments, R^{A1} is not

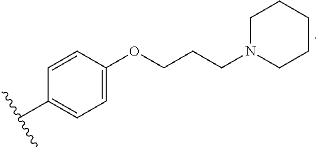 [SMILES: *c1ccc(OCCCN2CCCCC2)cc1]

As defined herein, n is 1 or 2. In some embodiments, n is 1. In some embodiments, n is 2.

As defined herein, R¹ is optionally substituted alkyl or optionally substituted cycloalkyl. In some embodiments, R¹ is optionally substituted alkyl or cycloalkyl. In some embodiments, R¹ is optionally substituted alkyl. In some embodiments, R¹ is alkyl. In some embodiments, R¹ is optionally substituted C₁₋₁₀ alkyl. In some embodiments, R¹ is C₁₋₁₀ alkyl. In some embodiments, R¹ is optionally substituted C₁₋₆ alkyl. In some embodiments, R¹ is C₁₋₆ alkyl. In some embodiments, R¹ is methyl, ethyl, propyl, butyl, pentyl, or hexyl. In some embodiments, R¹ is optionally substituted C₁₋₄ alkyl. In some embodiments, R¹ is C₁₋₄ alkyl. In some embodiments, R¹ is methyl, ethyl, n-propyl, isopropyl, n-butyl, sec-butyl, or tert-butyl. In some embodiments, R¹ is methyl or ethyl. In some embodiments, R¹ is methyl. In some embodiments, R¹ is ethyl. In some embodiments, R¹ is isopropyl. In some embodiments, R¹ is n-propyl. In some embodiments, R¹ is optionally substituted cycloalkyl. In some embodiments, R¹ is unsubstituted cycloalkyl. In some embodiments, R¹ is optionally substituted C₃₋₈ cycloalkyl. In some embodiments, R¹ is C₃₋₈ cycloalkyl. In some embodiments, R¹ is optionally substituted C₃₋₆ cycloalkyl. In some embodiments, R¹ is C₃₋₆ cycloalkyl. In some embodiments, R¹ is optionally substituted C₃₋₄ cycloalkyl. In some embodiments, R¹ is C₃₋₄ cycloalkyl. In some embodiments, R¹ is optionally substituted C₅₋₆ cycloalkyl. In some embodiments, R¹ is C₅₋₆ cycloalkyl. In some embodiments, R¹ is cyclopropyl, cyclobutyl, cyclopentyl, or cyclohexyl. In some embodiments, R¹ is cyclopropyl or cyclobutyl. In some embodiments, R¹ is cyclopentyl or cyclohexyl. In some embodiments, R¹ is cyclopropyl. In some embodiments, R¹ is cyclobutyl. In some embodiments, R¹ is cyclopentyl. In some embodiments, R¹ is cyclohexyl.

As defined herein, R^{A2} is H or halogen. In some embodiments, R^{A2} is H. In some embodiments, R^{A2} is halogen. In some embodiments, R^{A2} is I, Br, Cl, or F. In some embodiments, R^{A2} is Br, Cl, or F. In some embodiments, R^{A2} is I. In some embodiments, R^{A2} is Br. In some embodiments, R^{A2} is Cl. In some embodiments, R^{A2} is F. In some embodiments, R^{A2} is F or H. In some embodiments, R^{A2} is Br or Cl.

As defined herein, R² is optionally substituted

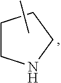

optionally substituted

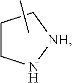

or optionally substituted

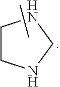

optionally substituted

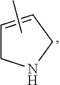

optionally substituted

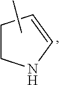

optionally substituted

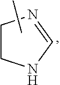

optionally substituted

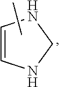

optionally substituted

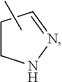

optionally substituted

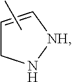

optionally substituted

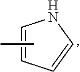

optionally substituted

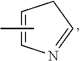

optionally substituted

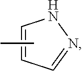

optionally substituted

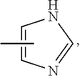

optionally substituted

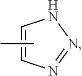

optionally substituted

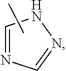

optionally substituted

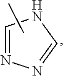

or optionally substituted

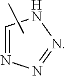

In some embodiments, R² is optionally substituted

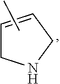

optionally substituted

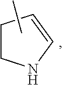

optionally substituted

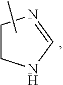

optionally substituted

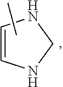

optionally substituted

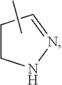

optionally substituted

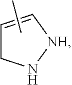

optionally substituted

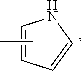

optionally substituted

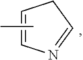

optionally substituted

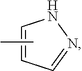

optionally substituted

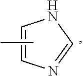

optionally substituted

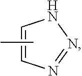

optionally substituted

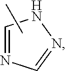

optionally substituted

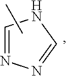

or optionally substituted

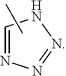

In some embodiments, R² is optionally substituted

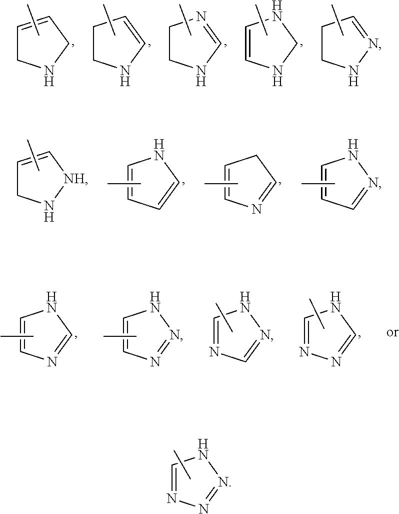

In some embodiments. R² is

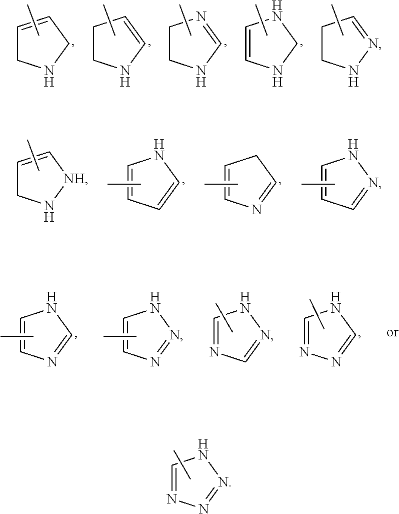

In some embodiments, R² is optionally substituted

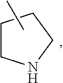

optionally substituted

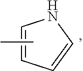

optionally substituted

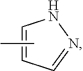

or optionally substituted

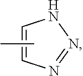

In some embodiments, R² is

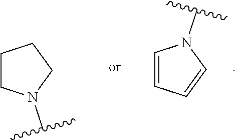 [SMILES: *N1CCCC1 ; *n1cccc1]

In some embodiments, R² is

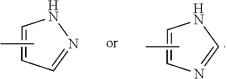

In some embodiments, R² is

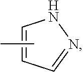

In some embodiments, R² is

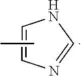

In some embodiments, R² is substituted. In some embodiments, R² is N-substituted. In some embodiments, R² is N-substituted with a nitrogen protecting group, optionally substituted alkyl, or optionally substituted carbocyclyl. In some embodiments, R² is N-substituted with optionally substituted alkyl or optionally substituted carbocyclyl. In some embodiments, R² is substituted with R^{A3}.

In some embodiments, R² is

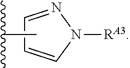

In some embodiments, R² is

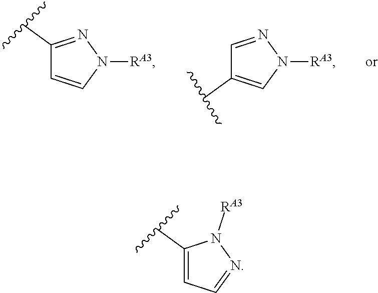

In some embodiments, R² is

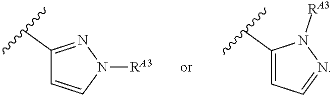

In some embodiments, R² is

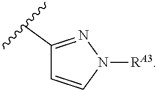

In some embodiments, R² is

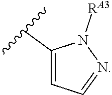

In some embodiments, R² is

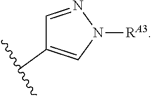

In some embodiments, R² is

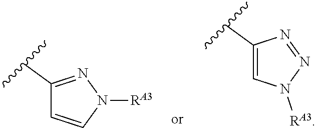

In some embodiments, R² is

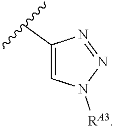

As defined herein, R^{A3} is optionally substituted alkyl or optionally substituted carbocyclyl. In some embodiments, R^{A3} is optionally substituted alkyl or cycloalkyl. In some embodiments, R^{A3} is optionally substituted alkyl. In some embodiments, R^{A3} is alkyl. In some embodiments, R^{A3} is optionally substituted C₁₋₁₀ alkyl. In some embodiments, R^{A3} is C₁₋₁₀ alkyl substituted with halogen, C₃₋₆ carbocyclyl, C₃₋₆ heterocyclyl, C₆₋₁₀ aryl, or C₅₋₁₀ heteroaryl. In some embodiments, R^{A3} is C₁₋₁₀ haloalkyl. In some embodiments, R^{A3} is C₁₋₁₀ alkyl. In some embodiments, R^{A3} is optionally substituted C₁₋₆ alkyl. In some embodiments, R^{A3} is C₁₋₆ alkyl substituted with halogen, C₃₋₆ carbocyclyl, C₃₋₆ heterocyclyl, C₆₋₁₀ aryl, or C₅₋₁₀ heteroaryl. In some embodiments, R^{A3} is C₁₋₆ haloalkyl. In some embodiments, R^{A3} is C₁₋₆ alkyl. In some embodiments, R^{A3} is methyl, ethyl, propyl, butyl, pentyl, or hexyl. In some embodiments, R^{A3} is optionally substituted C₁₋₄ alkyl. In some embodiments, R^{A3} is C₁₋₄ alkyl substituted with halogen, C₃₋₆ carbocyclyl, C₃₋₆ heterocyclyl, C₆₋₁₀ aryl, or C₅₋₁₀ heteroaryl. In some embodiments, R^{A3} is C₁₋₄ haloalkyl. In some embodiments, R^{A3} is C₁₋₄ alkyl. In some embodiments, R^{A3} is methyl, ethyl, n-propyl, isopropyl, n-butyl, sec-butyl, or tert-butyl. In some embodiments, R^{A3} is methyl or ethyl. In some embodiments, R^{A3} is methyl. In some embodiments, R^{A3} is ethyl. In some embodiments, R^{A3} is isopropyl. In some embodiments, R^{A3} is n-propyl. In some embodiments, R^{A3} is

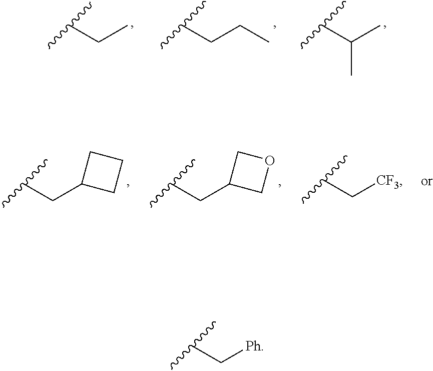

In some embodiments, R^{A3} is

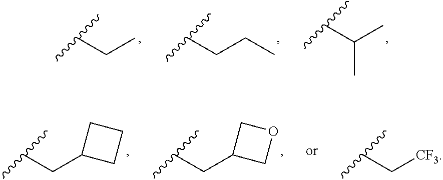

In some embodiments, R^{A3} is optionally substituted cycloalkyl. In some embodiments, R^{A3} is optionally substituted C₃₋₈ cycloalkyl. In some embodiments, R^{A3} is C₃₋₈ cycloalkyl. In some embodiments, R^{A3} is optionally substituted C₃₋₆ cycloalkyl. In some embodiments, R^{A3} is C₃₋₆ cycloalkyl. In some embodiments, R^{A3} is optionally substituted C₃₋₄ cycloalkyl. In some embodiments, R^{A3} is C₃₋₄ cycloalkyl. In some embodiments, R^{A3} is optionally substituted C₅₋₆ cycloalkyl. In some embodiments, R^{A3} is C₅₋₆ cycloalkyl. In some embodiments, R^{A3} is cyclopropyl, cyclobutyl, cyclopentyl, or cyclohexyl. In some embodiments, R^{A3} is cyclopropyl or cyclobutyl. In some embodiments, R^{A3} is cyclopentyl or cyclohexyl. In some embodiments, R^{A3} is cyclopropyl. In some embodiments, R^{A3} is cyclobutyl. In some embodiments, R^{A3} is cyclopentyl. In some embodiments, R^{A3} is cyclohexyl.

In some embodiments, R^{A3} is optionally substituted C₁₋₆ alkyl or optionally substituted C₃₋₈ carbocyclyl. In some embodiments, R^{A3} is

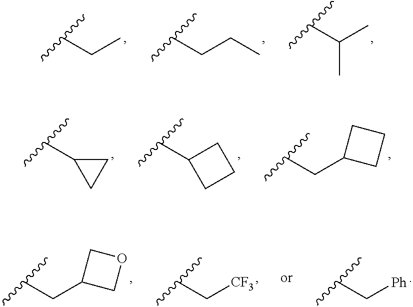

In some embodiments, R^{A3} is

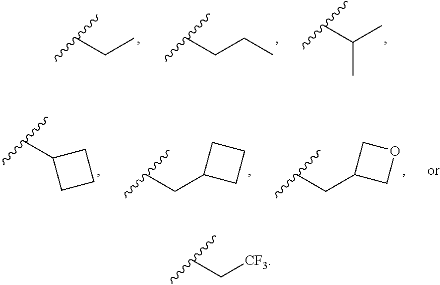

In some embodiments, R² is

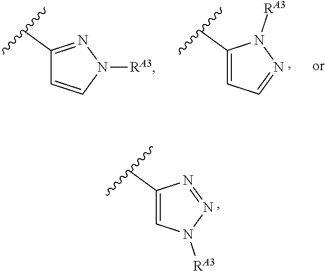

wherein R^{A3} is

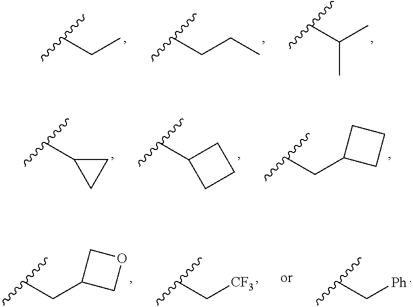

In some embodiments, R² is

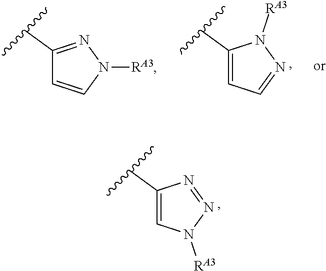

wherein R^{A3} is

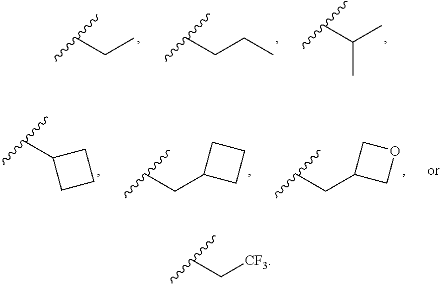

In some embodiments, R² is

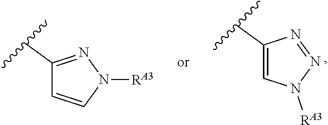

wherein R^{A3} is

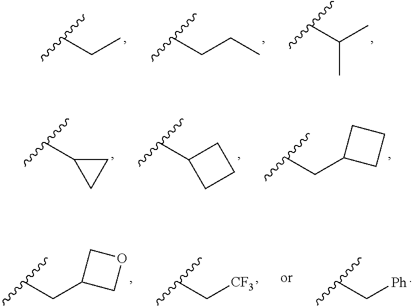

In some embodiments, R² is

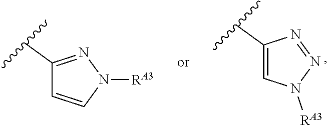

wherein R^{A3} is

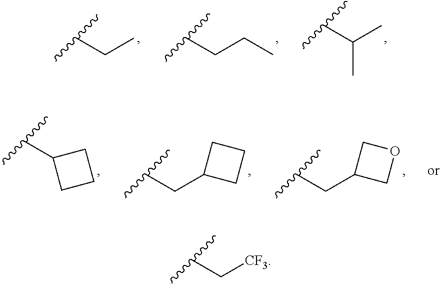

In some embodiments, R² is

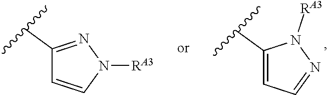

wherein R^{A3} is

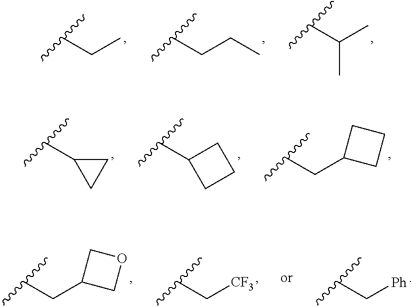

In some embodiments, R² is

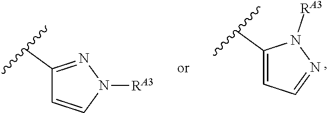

wherein R^{A3} is

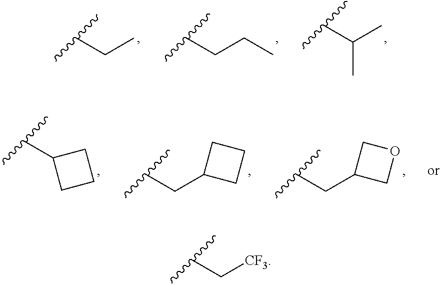

In some embodiments, R² is

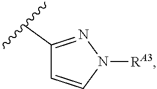

wherein R^{A3} is

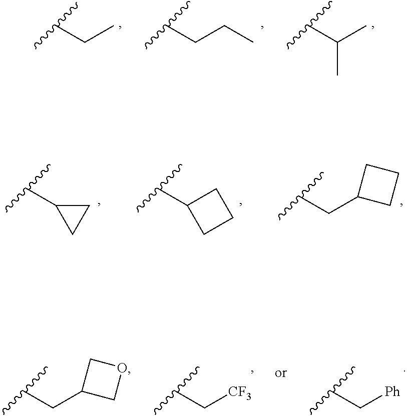

In some embodiments, R² is

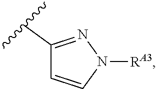

wherein R^{A3} is

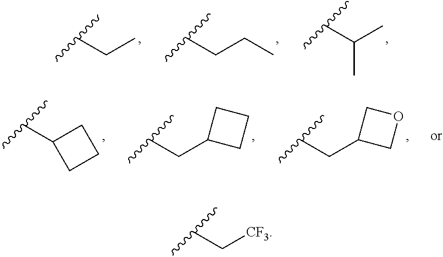

In some embodiments, R² is

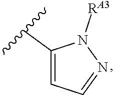

wherein R^{A3} is

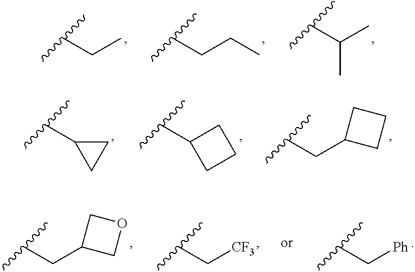

In some embodiments, R² is

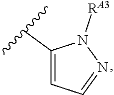

wherein R^{A3} is

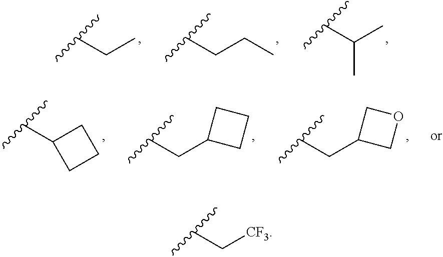

In some embodiments R²

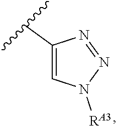

wherein R^{A3} is

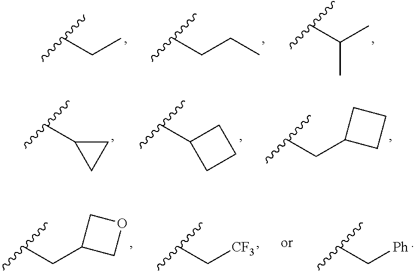

In some embodiments, R² is

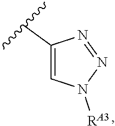

wherein R^{A3} is

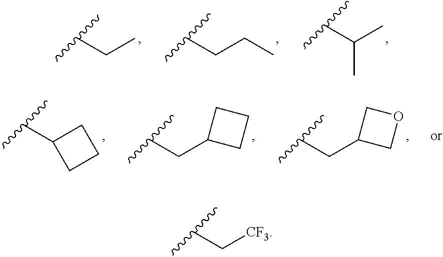

In some embodiments, if R^{A1} is

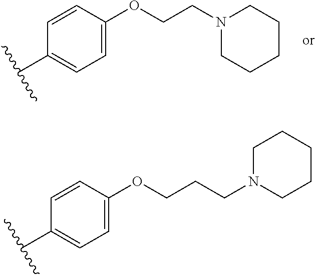 [SMILES: *c1ccc(OCCN2CCCCC2)cc1 ; *c1ccc(OCCCN2CCCCC2)cc1]

and R^{A2} is F, R² is not

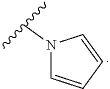 [SMILES: *n1cccc1]

In some embodiments, R^{A1} is

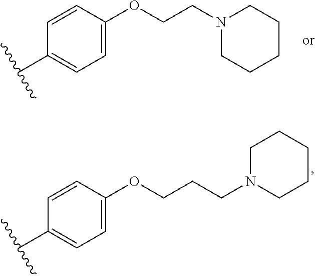 [SMILES: *c1ccc(OCCN2CCCCC2)cc1 ; *c1ccc(OCCCN2CCCCC2)cc1]

### R² is

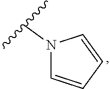 [SMILES: *n1cccc1]

### and R^{A2} is H.

In some embodiments, if R^{A1} is

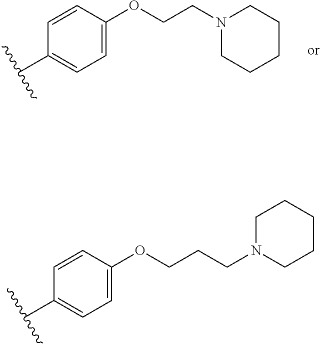 [SMILES: *c1ccc(OCCN2CCCCC2)cc1 ; *c1ccc(OCCCN2CCCCC2)cc1]

and R^{A2} is H, R² is not

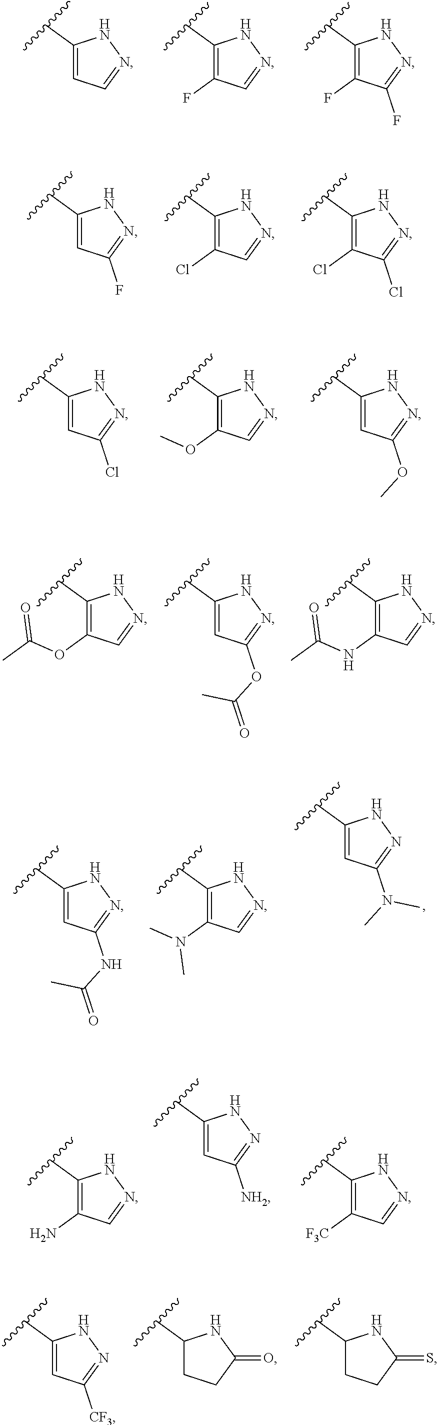

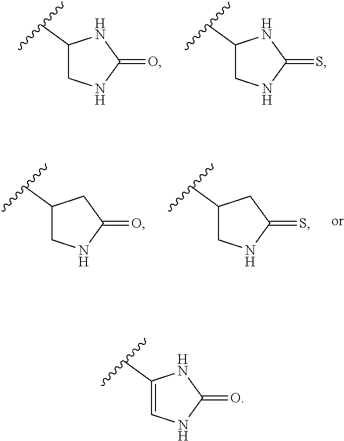 [SMILES: *C1CNC(=O)N1 ; *C1CNC(=S)N1 ; *C1CNC(=O)C1 ; *C1CNC(=S)C1 ; *c1c[nH]c(=O)[nH]1]

In some embodiments, R^{A1} is

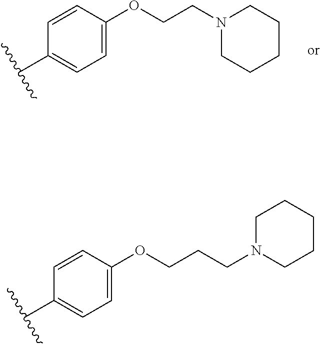 [SMILES: *c1ccc(OCCN2CCCCC2)cc1 ; *c1ccc(OCCCN2CCCCC2)cc1]

### and R² is

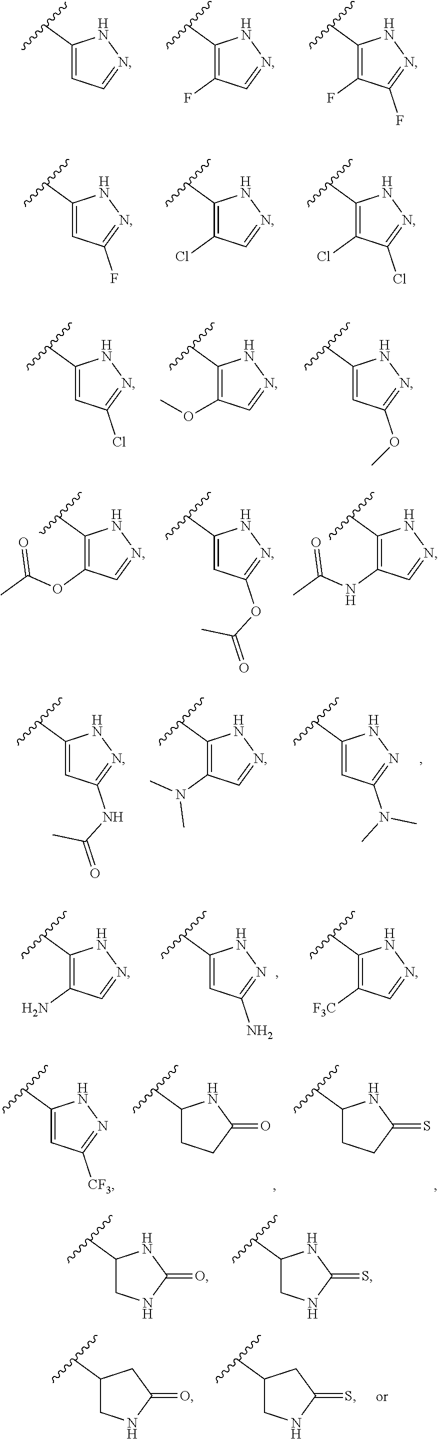

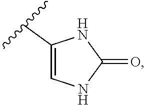 [SMILES: *c1c[nH]c(=O)[nH]1]

and R^{A2} is halogen.

In some embodiments, if R^{A1} is

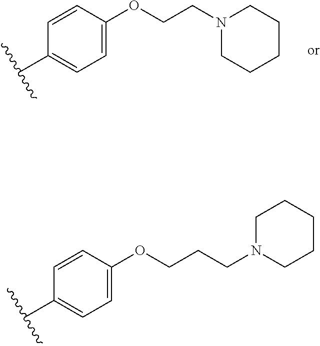 [SMILES: *c1ccc(OCCN2CCCCC2)cc1 ; *c1ccc(OCCCN2CCCCC2)cc1]

and R^{A2} is Br, R² is not

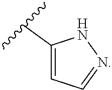 [SMILES: *c1ccn[nH]1]

In some embodiments, R^{A1} is

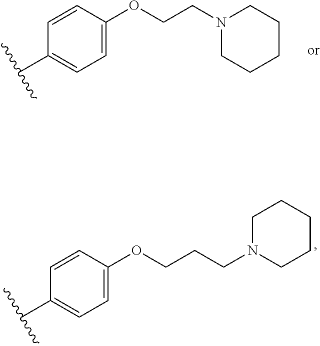 [SMILES: *c1ccc(OCCN2CCCCC2)cc1 ; *c1ccc(OCCCN2CCCCC2)cc1]

### R² is

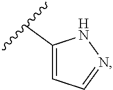 [SMILES: *c1ccn[nH]1]

and R^{A2} is H. In some embodiments, R^{A1} is

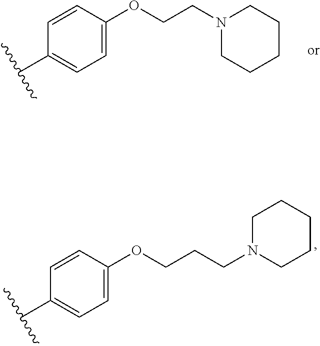 [SMILES: *c1ccc(OCCN2CCCCC2)cc1 ; *c1ccc(OCCCN2CCCCC2)cc1]

### R² is

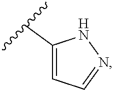 [SMILES: *c1ccn[nH]1]

### and R^{A2} is H, F, or Cl.

In some embodiments, the compound is selected from the group consisting of:

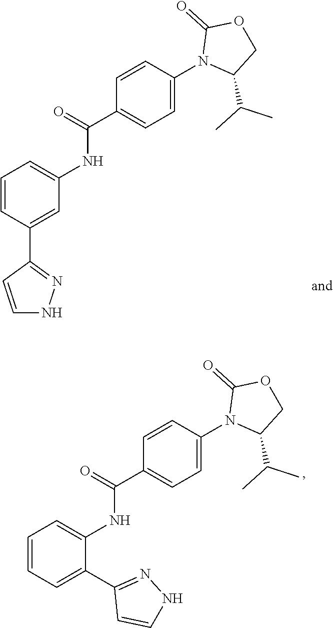 [SMILES: CC(C)[C@H]1COC(=O)N1c1ccc(C(=O)Nc2cccc(-c3cc[nH]n3)c2)cc1 ; CC(C)[C@H]1COC(=O)N1c1ccc(C(=O)Nc2ccccc2-c2cc[nH]n2)cc1]

or a salt, solvate, hydrate, polymorph, co-crystal, tautomer, stereoisomer, isotopically labeled derivative, or prodrug thereof.

In some embodiments, the compound is selected from the group consisting of:

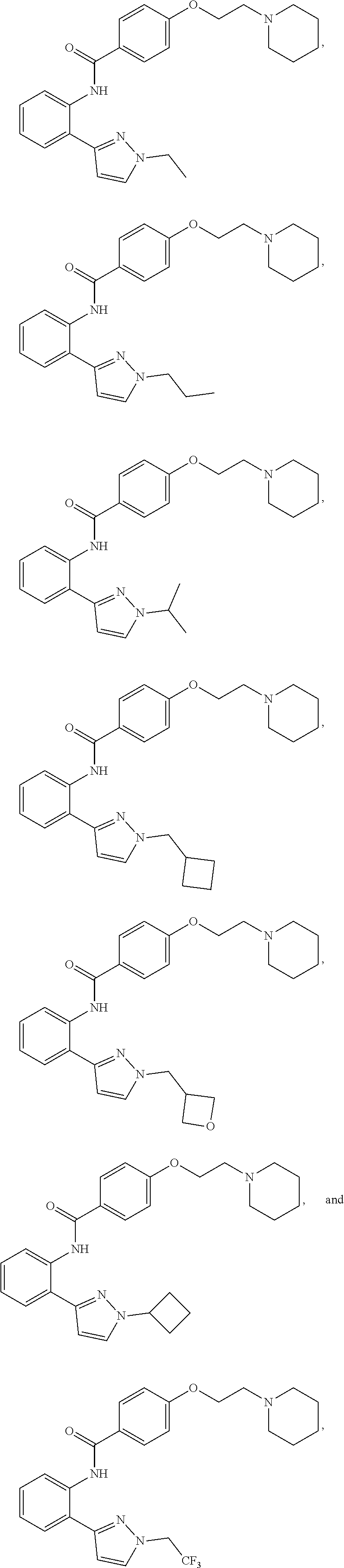

or a salt, solvate, hydrate, polymorph, co-crystal, tautomer, stereoisomer, isotopically labeled derivative, or prodrug thereof.

In some embodiments, the compound is selected from the group consisting of:

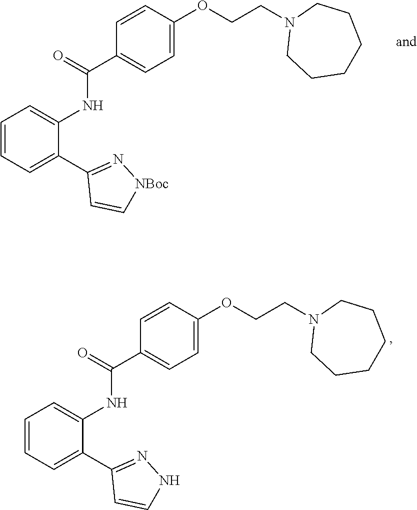 [SMILES: CC(C)(C)OC(=O)n1ccc(-c2ccccc2NC(=O)c2ccc(OCCN3CCCCCC3)cc2)n1 ; O=C(Nc1ccccc1-c1cc[nH]n1)c1ccc(OCCN2CCCCCC2)cc1]

or a salt, solvate, hydrate, polymorph, co-crystal, tautomer, stereoisomer, isotopically labeled derivative, or prodrug thereof.

In some embodiments, the compound is selected from the group consisting of:

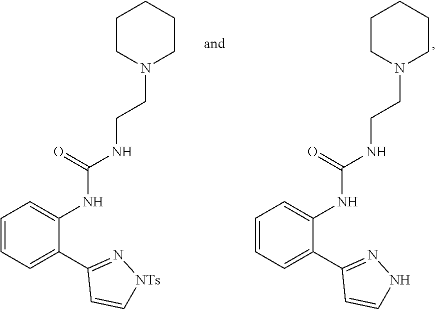 [SMILES: Cc1ccc(S(=O)(=O)n2ccc(-c3ccccc3NC(=O)NCCN3CCCCC3)n2)cc1 ; O=C(NCCN1CCCCC1)Nc1ccccc1-c1cc[nH]n1]

or a salt, solvate, hydrate, polymorph, co-crystal, tautomer, stereoisomer, isotopically labeled derivative, or prodrug thereof.

In some embodiments, the compound is selected from the group consisting of:

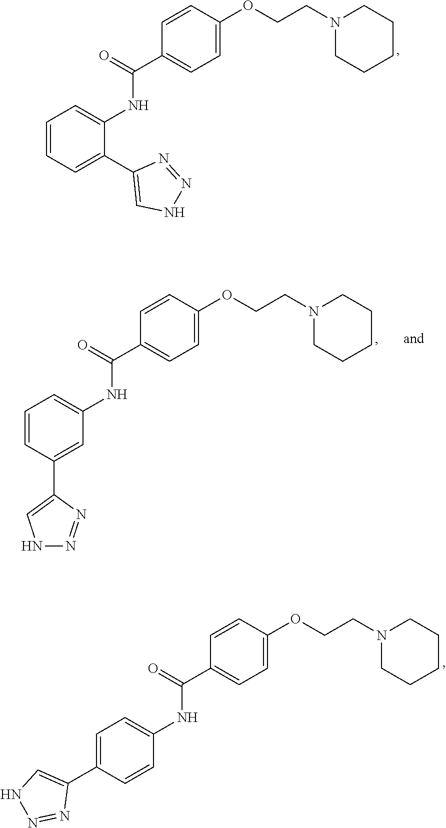 [SMILES: O=C(Nc1ccccc1-c1c[nH]nn1)c1ccc(OCCN2CCCCC2)cc1 ; O=C(Nc1cccc(-c2c[nH]nn2)c1)c1ccc(OCCN2CCCCC2)cc1 ; O=C(Nc1ccc(-c2c[nH]nn2)cc1)c1ccc(OCCN2CCCCC2)cc1]

or a salt, solvate, hydrate, polymorph, co-crystal, tautomer, stereoisomer, isotopically labeled derivative, or prodrug thereof.

In some embodiments, the compound has the structure

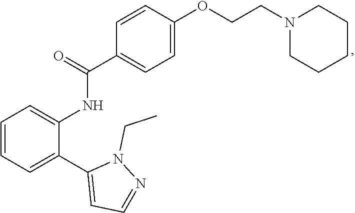 [SMILES: CCn1nccc1-c1ccccc1NC(=O)c1ccc(OCCN2CCCCC2)cc1]

or a salt, solvate, hydrate, polymorph, co-crystal, tautomer, stereoisomer, isotopically labeled derivative, or prodrug thereof.

In some embodiments, the compound is selected from the group consisting of:

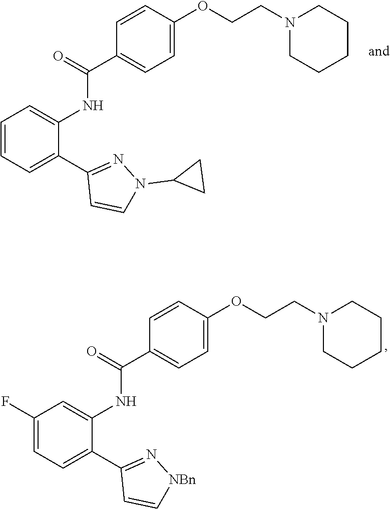 [SMILES: O=C(Nc1ccccc1-c1ccn(C2CC2)n1)c1ccc(OCCN2CCCCC2)cc1 ; O=C(Nc1cc(F)ccc1-c1ccn(Cc2ccccc2)n1)c1ccc(OCCN2CCCCC2)cc1]

or a salt, solvate, hydrate, polymorph, co-crystal, tautomer, stereoisomer, isotopically labeled derivative, or prodrug thereof.

In some embodiments, the compound is selected from the group consisting of:

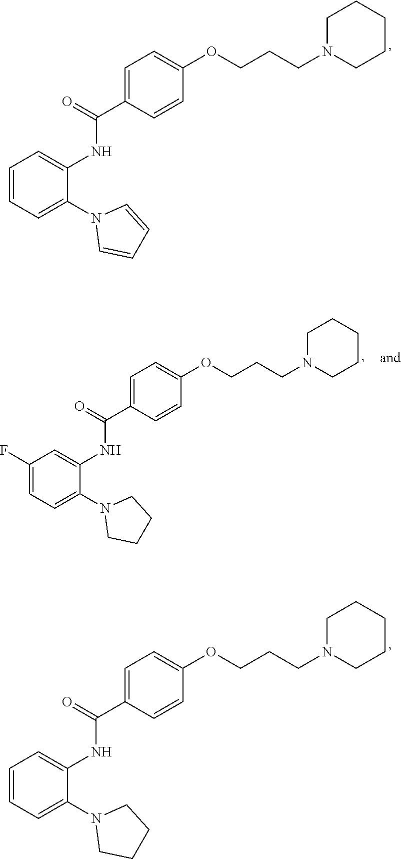 [SMILES: O=C(Nc1ccccc1-n1cccc1)c1ccc(OCCCN2CCCCC2)cc1 ; O=C(Nc1cc(F)ccc1N1CCCC1)c1ccc(OCCCN2CCCCC2)cc1 ; O=C(Nc1ccccc1N1CCCC1)c1ccc(OCCCN2CCCCC2)cc1]

or a salt, solvate, hydrate, polymorph, co-crystal, tautomer, stereoisomer, isotopically labeled derivative, or prodrug thereof.

In some embodiments, a salt of a compound of Formula (A) is an acetate salt. In some embodiments, a solvate of a compound of Formula (A) is an acetic acid solvate.

### Formula (B)

In another aspect, provided herein is a compound of Formula (B), or a salt, solvate, hydrate, polymorph, co-crystal, tautomer, stereoisomer, isotopically labeled derivative, or prodrug thereof:

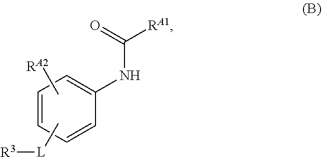

wherein:

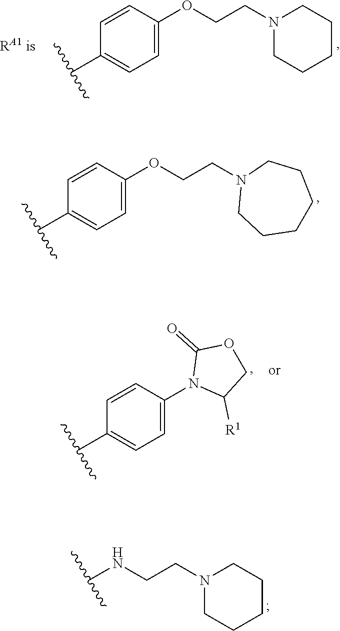

R¹ is optionally substituted alkyl or optionally substituted cycloalkyl;
R^{A2} is H or halogen;
L is a linker selected from the group consisting of —NH(C═O)—, —(C═O)NH—, optionally substituted five-membered N-containing heterocyclylene, optionally substituted five-membered N-containing heteroarylene; and
R³ is a carbohydrate or a carbohydrate substituted with one or more oxygen protecting groups.

In some embodiments, the compound is of Formula (IX), or a salt, solvate, hydrate, polymorph, co-crystal, tautomer, stereoisomer, isotopically labeled derivative, or prodrug thereof:

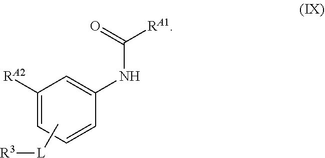

In some embodiments, the compound is of Formula (IX-c), or a salt, solvate, hydrate, polymorph, co-crystal, tautomer, stereoisomer, isotopically labeled derivative, or prodrug thereof:

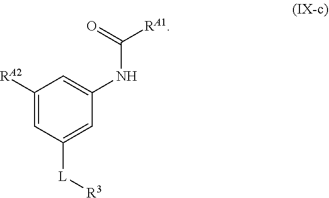

In some embodiments, the compound is of Formula (IX-d), or a salt, solvate, hydrate, polymorph, co-crystal, tautomer, stereoisomer, isotopically labeled derivative, or prodrug thereof:

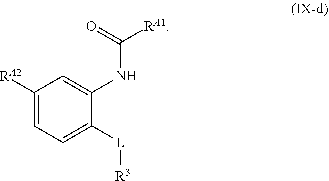

In some embodiments, the compound is of Formula (IX-e), or a salt, solvate, hydrate, polymorph, co-crystal, tautomer, stereoisomer, isotopically labeled derivative, or prodrug thereof:

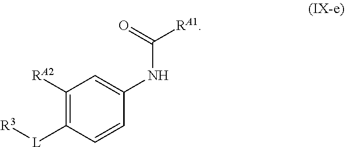

In some embodiments, the compound is of Formula (X-c), (XI-c), (XII-c), or (XIII-c), or a salt, solvate, hydrate, polymorph, co-crystal, tautomer, stereoisomer, isotopically labeled derivative, or prodrug thereof:

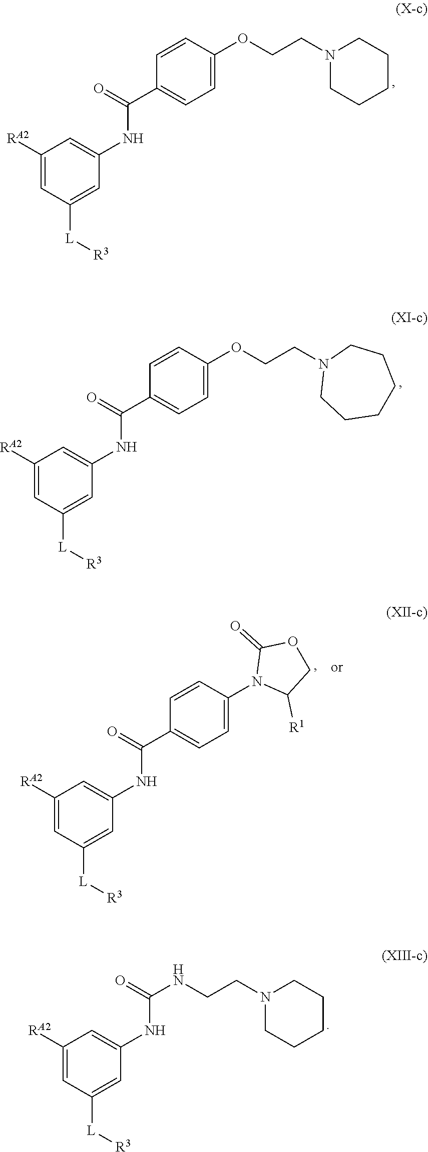

In some embodiments, the compound is of Formula (X-d), (XI-d), (XII-d), or (XIII-d), or a salt, solvate, hydrate, polymorph, co-crystal, tautomer, stereoisomer, isotopically labeled derivative, or prodrug thereof:

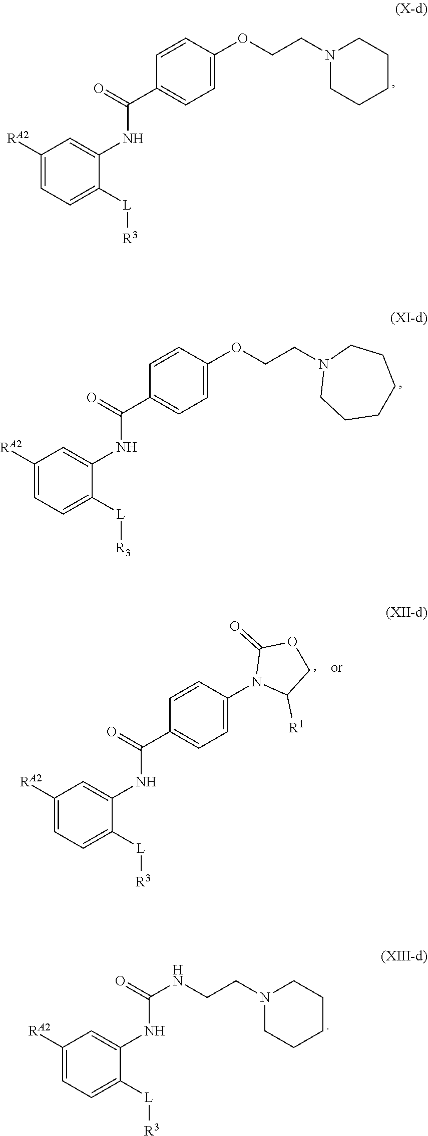

In some embodiments, the compound is of Formula (X-e), (XI-e), (XII-e), or (XIII-e), or a salt, solvate, hydrate, polymorph, co-crystal, tautomer, stereoisomer, isotopically labeled derivative, or prodrug thereof:

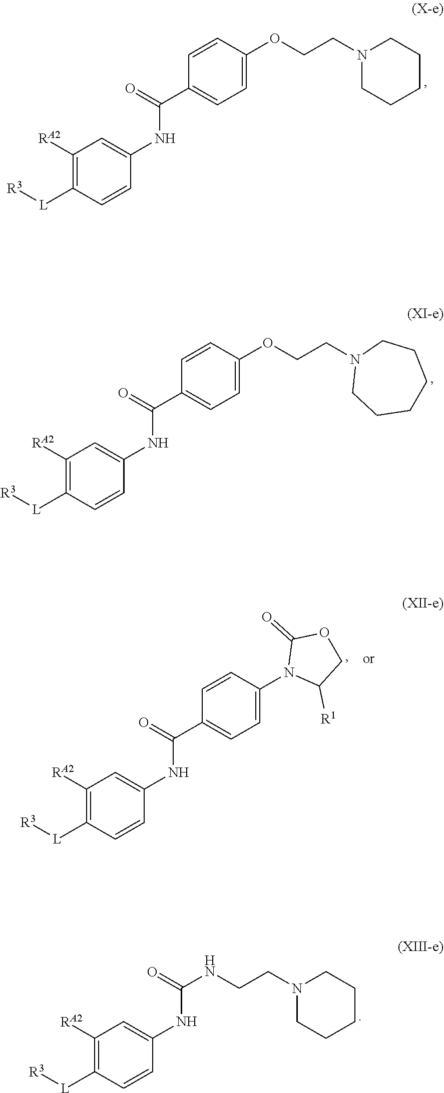

In some embodiments, the compound is of Formula (X-c), (X-d), or (X-e). In some embodiments, the compound is of Formula (XI-c), (XI-d), or (XI-e). In some embodiments, the compound is of Formula (XII-c), (XII-d), or (XII-e). In some embodiments, the compound is of Formula (XIII-c), (XIII-d), or (XIII-e).

In some embodiments, the compound is of Formula (II), or a salt, solvate, hydrate, polymorph, co-crystal, tautomer, stereoisomer, isotopically labeled derivative, or prodrug thereof:

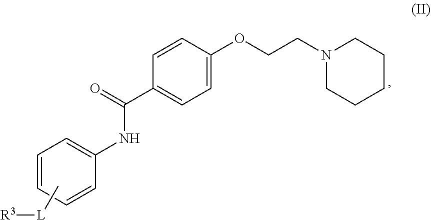

wherein:
L is a linker selected from the group consisting of —NH(C═O)—, —(C═O)NH—,

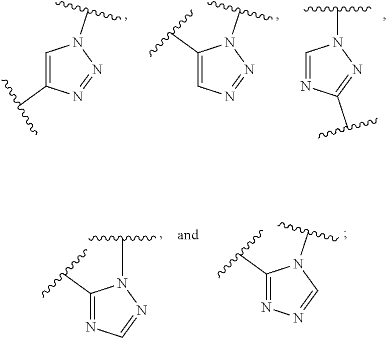 [SMILES: *c1cn(*)nn1 ; *c1cnnn1* ; *c1ncn(*)n1 ; *c1ncnn1* ; *c1nncn1*]

R³ is selected from the group consisting of

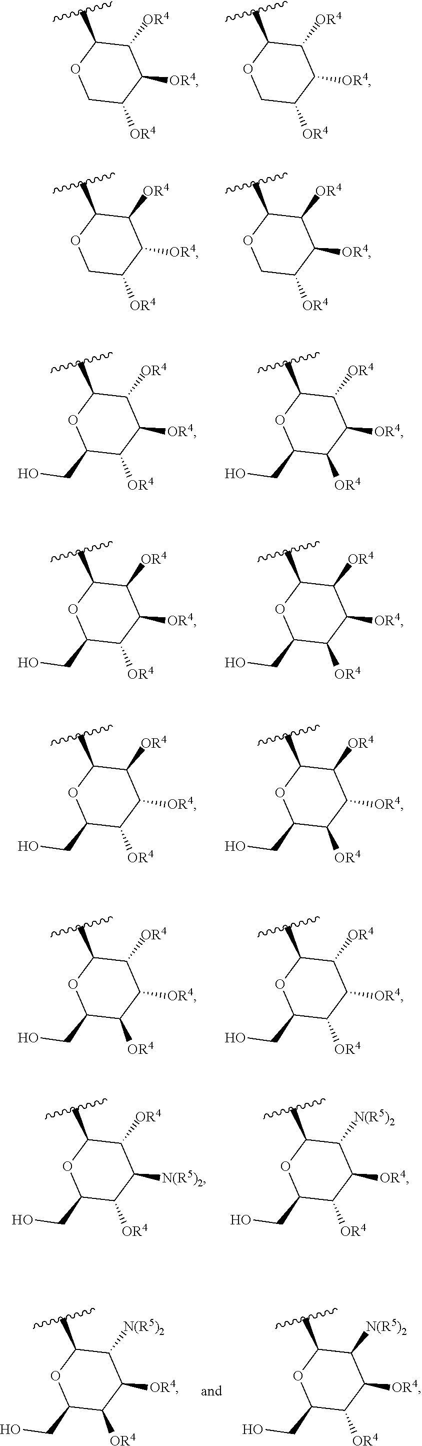

wherein:
each R⁴ is independently hydrogen or an oxygen protecting group; and
each R⁵ is independently hydrogen or a nitrogen protecting group.

In some embodiments, the compound is of Formula (II-a), or a salt, solvate, hydrate, polymorph, co-crystal, tautomer, stereoisomer, isotopically labeled derivative, or prodrug thereof:

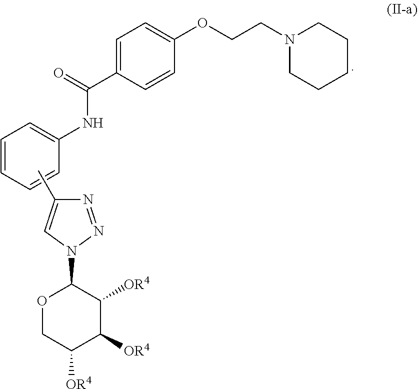

As defined herein, R^{A1} is

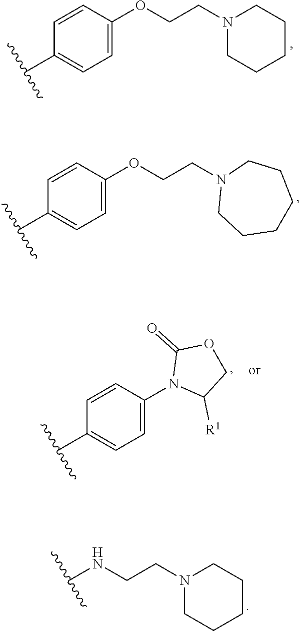

In some embodiments, R^{A1} is

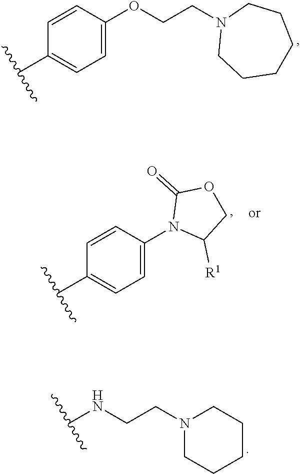

In some embodiments, R^{A1} is

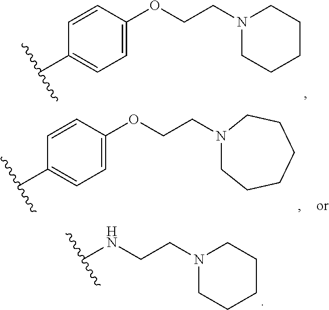 [SMILES: *c1ccc(OCCN2CCCCC2)cc1 ; *c1ccc(OCCN2CCCCCC2)cc1 ; *NCCN1CCCCC1]

In some embodiments, R^{A1} is

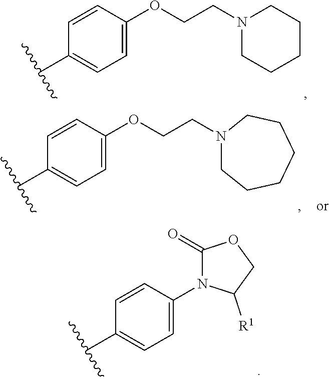

In some embodiments R^{A1} is

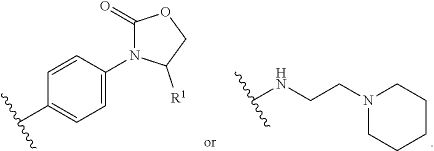

In some embodiments, R^{A1} is

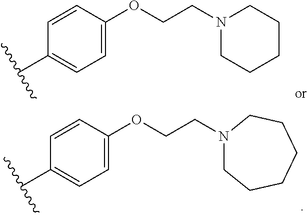

In some embodiments, R^{A1} is

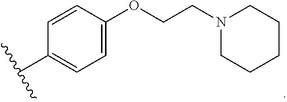 [SMILES: *c1ccc(OCCN2CCCCC2)cc1]

In some embodiments, R^{A1} is

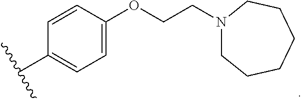 [SMILES: *c1ccc(OCCN2CCCCCC2)cc1]

In some embodiments, R^{A1} is

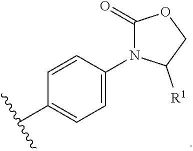

In some embodiments, R^{A1} is

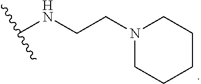 [SMILES: *NCCN1CCCCC1]

As defined herein, R¹ is optionally substituted alkyl or optionally substituted cycloalkyl. In some embodiments, R¹ is optionally substituted alkyl or cycloalkyl. In some embodiments, R¹ is optionally substituted alkyl. In some embodiments, R¹ is alkyl. In some embodiments, R¹ is optionally substituted C₁₋₁₀ alkyl. In some embodiments, R¹ is C₁₋₁₀ alkyl. In some embodiments, R¹ is optionally substituted C₁₋₆ alkyl. In some embodiments, R¹ is C₁₋₆ alkyl. In some embodiments, R¹ is methyl, ethyl, propyl, butyl, pentyl, or hexyl. In some embodiments, R¹ is optionally substituted C₁₋₄ alkyl. In some embodiments, R¹ is C₁₋₄ alkyl. In some embodiments, R¹ is methyl, ethyl, n-propyl, isopropyl, n-butyl, sec-butyl, or tert-butyl. In some embodiments, R¹ is methyl or ethyl. In some embodiments, R¹ is methyl. In some embodiments, R¹ is ethyl. In some embodiments, R¹ is isopropyl. In some embodiments, R¹ is n-propyl. In some embodiments, R¹ is optionally substituted cycloalkyl. In some embodiments, R¹ is unsubstituted cycloalkyl. In some embodiments, R¹ is optionally substituted C₃₋₈ cycloalkyl. In some embodiments, R¹ is C₃₋₈ cycloalkyl. In some embodiments, R¹ is optionally substituted C₃₋₆ cycloalkyl. In some embodiments, R¹ is C₃₋₆ cycloalkyl. In some embodiments, R¹ is optionally substituted C₃₋₄ cycloalkyl. In some embodiments, R¹ is C₃₋₄ cycloalkyl. In some embodiments, R¹ is optionally substituted C₅₋₆ cycloalkyl. In some embodiments, R¹ is C₅₋₆ cycloalkyl. In some embodiments, R¹ is cyclopropyl, cyclobutyl, cyclopentyl, or cyclohexyl. In some embodiments, R¹ is cyclopropyl or cyclobutyl. In some embodiments, R¹ is cyclopentyl or cyclohexyl. In some embodiments, R¹ is cyclopropyl. In some embodiments, R¹ is cyclobutyl. In some embodiments, R¹ is cyclopentyl. In some embodiments, R¹ is cyclohexyl.

As defined herein, R^{A2} is H or halogen. In some embodiments, R^{A2} is H. In some embodiments, R^{A2} is halogen. In some embodiments, R^{A2} is I, Br, Cl, or F. In some embodiments, R^{A2} is Br, Cl, or F. In some embodiments, R^{A2} is I. In some embodiments, R^{A2} is Br. In some embodiments, R^{A2} is Cl. In some embodiments, R^{A2} is F. In some embodiments, R^{A2}is F or H. In some embodiments, R^{A2} is Br or Cl.

As defined herein, L is a linker selected from the group consisting of —NH(C═O)—, —(C═O)NH—, optionally substituted five-membered N-containing heterocyclylene, optionally substituted five-membered N-containing heteroarylene, or optionally substituted five-membered N-containing heteroarylalkylene. In some embodiments, L is a linker selected from the group consisting of —NH(C═O)—, —(C═O)NH—, optionally substituted five-membered N-containing heterocyclylene, and optionally substituted five-membered N-containing heteroarylene.

In some embodiments, L is —NH(C═O)— or —(C═O)NH—. In some embodiments, L is —NH(C═O)—. In some embodiments, L is —(C═O)NH—.

In some embodiments, L is a linker selected from the group consisting of optionally substituted five-membered N-containing heterocyclylene and optionally substituted five-membered N-containing heteroarylene. In some embodiments, L is optionally substituted five-membered N-containing heterocyclylene. In some embodiments, L is optionally substituted five-membered N-containing heteroarylene. In some embodiments, L is optionally substituted pyrrolidinylene, optionally substituted pyrazolidinylene, optionally substituted imadazolidinylene, optionally substituted 3-pyrrolinylene, optionally substituted 2-pyrrolinylene, optionally substituted 2-pyrazolinylene, optionally substituted 2-imidazolinylene, optionally substituted 2H-pyrrolylene, optionally substituted 1H-pyrrolylene, optionally substituted pyrazolylene, optionally substituted imidazolylene, optionally substituted 1,2,4-triazolylene, optionally substituted 1,2,3-triazolylene, or optionally substituted tetrazolylene. In some embodiments, L is optionally substituted pyrazolylene, optionally substituted imidazolylene, optionally substituted 1,2,4-triazolylene, or optionally substituted 1,2,3-triazolylene.

In some embodiments, L is optionally substituted five-membered N-containing heteroarylalkylene. In some embodiments, L is optionally substituted five-membered N-containing heteroaryl(C₁₋₆ alkylene). In some embodiments, L is optionally substituted five-membered N-containing heteroarylmethylene. In some embodiments, the heteroaryl is optionally substituted pyrrolidinyl, optionally substituted pyrazolidinyl, optionally substituted imadazolidinyl, optionally substituted 3-pyrrolinyl, optionally substituted 2-pyrrolinyl, optionally substituted 2-pyrazolinyl, optionally substituted 2-imidazolinyl, optionally substituted 2H-pyrrolyl, optionally substituted 1H-pyrrolyl, optionally substituted pyrazolyl, optionally substituted imidazolyl, optionally substituted 1,2,4-triazolyl, optionally substituted 1,2,3-triazolyl, or optionally substituted tetrazolyl. In some embodiments, the heteroaryl is optionally substituted pyrazolyl, optionally substituted imidazolyl, optionally substituted 1,2,4-triazolyl, or optionally substituted 1,2,3-triazolyl.

In some embodiments, L is

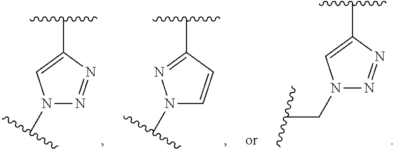 [SMILES: *c1cn(*)nn1 ; *c1ccn(*)n1 ; *Cn1cc(*)nn1]

In some embodiments, L is

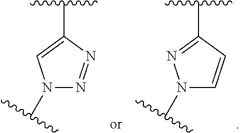 [SMILES: *c1cn(*)nn1 ; *c1ccn(*)n1]

In some embodiments, L is

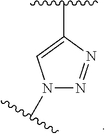 [SMILES: *c1cn(*)nn1]

In some embodiments, L is

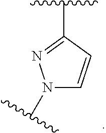 [SMILES: *c1ccn(*)n1]

In some embodiments, L is

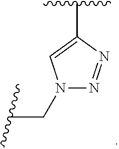 [SMILES: *Cn1cc(*)nn1]

In some embodiments, L is a linker selected from the group consisting of —NH(C═O)—, —(C═O)NH—,

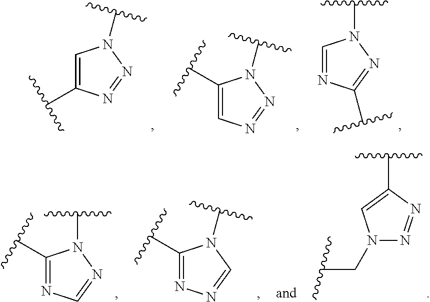

In some embodiments, L is a linker selected from the group consisting of —NH(C═O)—, —(C═O)NH—,

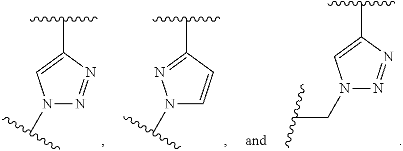 [SMILES: *c1cn(*)nn1 ; *c1ccn(*)n1 ; *Cn1cc(*)nn1]

In some embodiments, L is a linker selected from the group consisting of —NH(C═O)—, —(C═O)NH—,

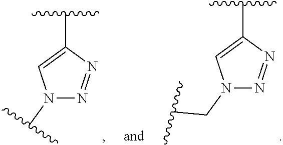 [SMILES: *c1cn(*)nn1 ; *Cn1cc(*)nn1]

In some embodiments, L is a linker selected from the group consisting of —NH(C═O)—, —(C═O)NH—,

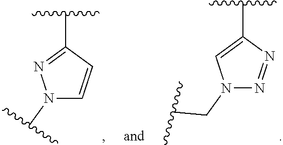 [SMILES: *c1ccn(*)n1 ; *Cn1cc(*)nn1]

In some embodiments, L is a linker selected from the group consisting of —NH(C═O)—, —(C═O)NH—,

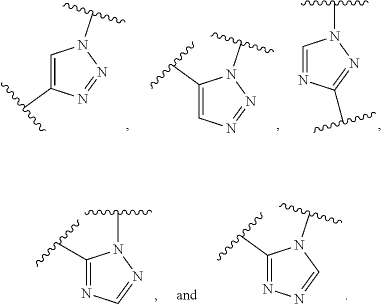 [SMILES: *c1cn(*)nn1 ; *c1cnnn1* ; *c1ncn(*)n1 ; *c1ncnn1* ; *c1nncn1*]

In some embodiments, L is a linker selected from the group consisting of —NH(C═O)—, —(C═O)NH—,

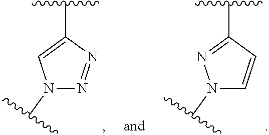 [SMILES: *c1cn(*)nn1 ; *c1ccn(*)n1]

In some embodiments, L is a linker selected from the group consisting of —NH(C═O)—, —(C═O)NH—, and

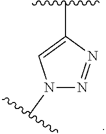 [SMILES: *c1cn(*)nn1]

In some embodiments, L is a linker selected from the group consisting of —NH(C═O)—, —(C═O)NH—, and

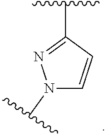 [SMILES: *c1ccn(*)n1]

As defined herein, R³ is a carbohydrate or a carbohydrate substituted with one or more oxygen protecting groups. In some embodiments, R³ is a carbohydrate. In some embodiments, R³ is a carbohydrate substituted with one or more oxygen protecting groups. In some embodiments, the carbohydrate is a monosaccharide. In some embodiments, the carbohydrate is erythrose, threose, ribose, arabinose, xylose, lyxose, ribulose, xylulose, allose, altrose, glucose, mannose, gulose, idose, galactose, telose, galactosamine, N-acetylgalactose, glucosamine, N-acetylglucosamine, sialic acid, talose, psicose, fructose, sorbose, tagatose, fucose, fuculose, rhamonse, sedoheptulose, octose, sulfoquinovose, nonose (neuraminic acid), sucrose, lactulose, lactose, maltose, trehalose, cellobiose, kojibiose, nigerose, isomaltose, β,β-trehalose, α,β-trehalose, sophorose, laminaribiose, gentibiose, turanose, maltulose, palatinose, gentibiulose, mannobiose, melibiose, rutinose, rutinulose, xylobiose, raffinose, melezitose, acarbose, or stachyose. In some embodiments, the carbohydrate is allose, altrose, glucose, mannose, gulose, idose, galactose, or telose.

In some embodiments, R³ is selected from the group consisting of

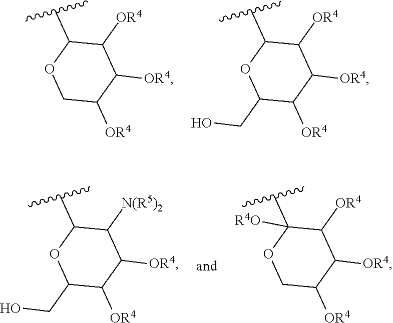

wherein:
each R⁴ is independently hydrogen or an oxygen protecting group or wherein two R⁴ are joined together with the intervening atoms to form an oxygen protecting group; and
each R⁵ is independently hydrogen or a nitrogen protecting group.

In some embodiments, R³ is selected from the group consisting of

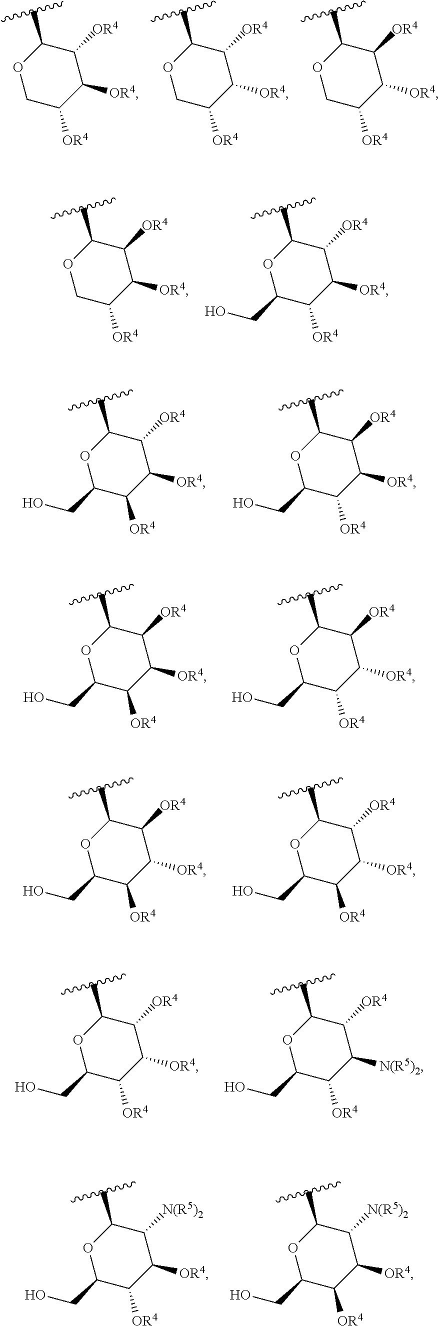

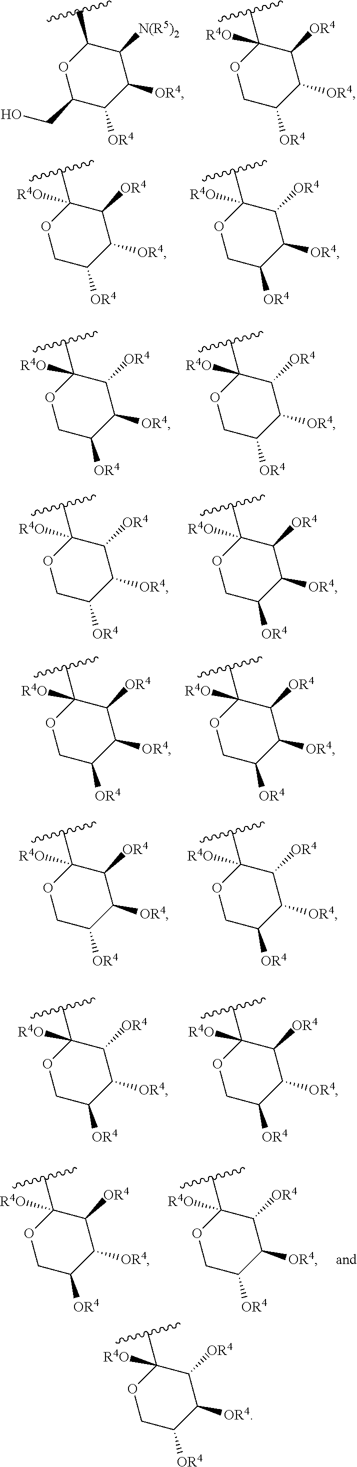

In some embodiments, R³ is

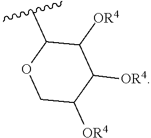

In some embodiments, R³ is

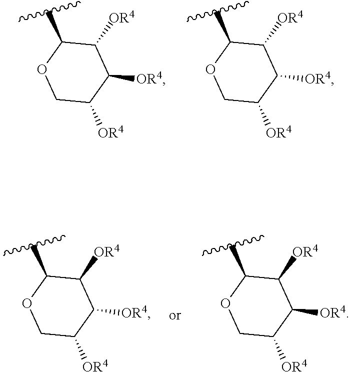

In some embodiments, R³ is

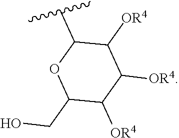

In some embodiments, R³ is

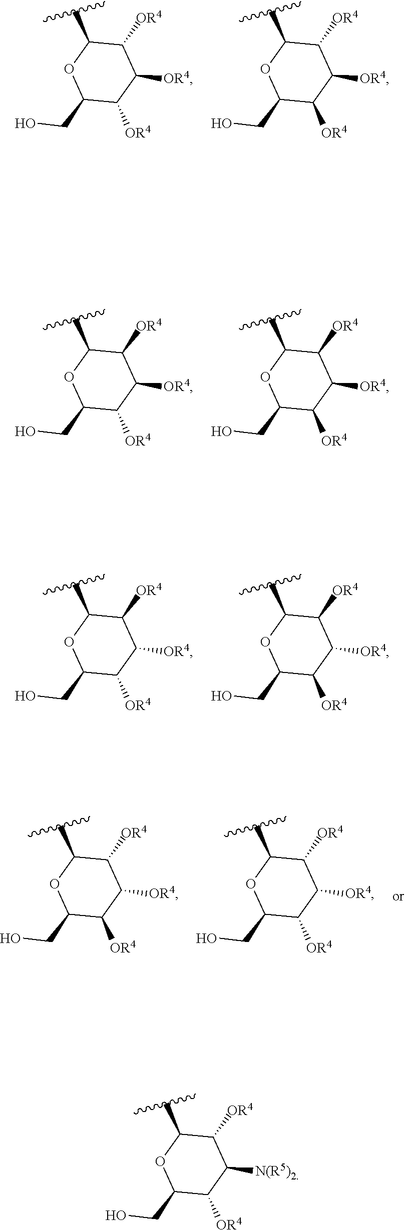

In some embodiments, R³ is

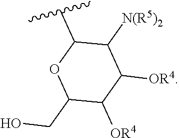

In some embodiments, R³ is

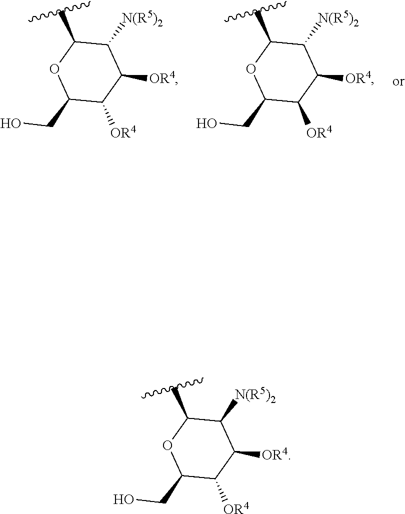

In some embodiments, R³ is

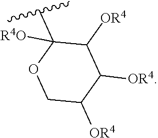

In some embodiments, R³ is

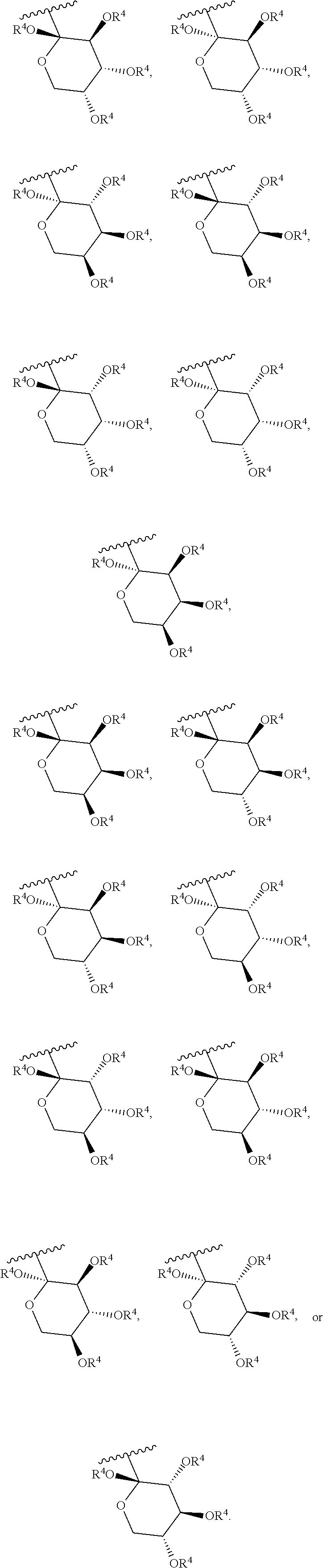

In some embodiments, R³ is selected from the group consisting of

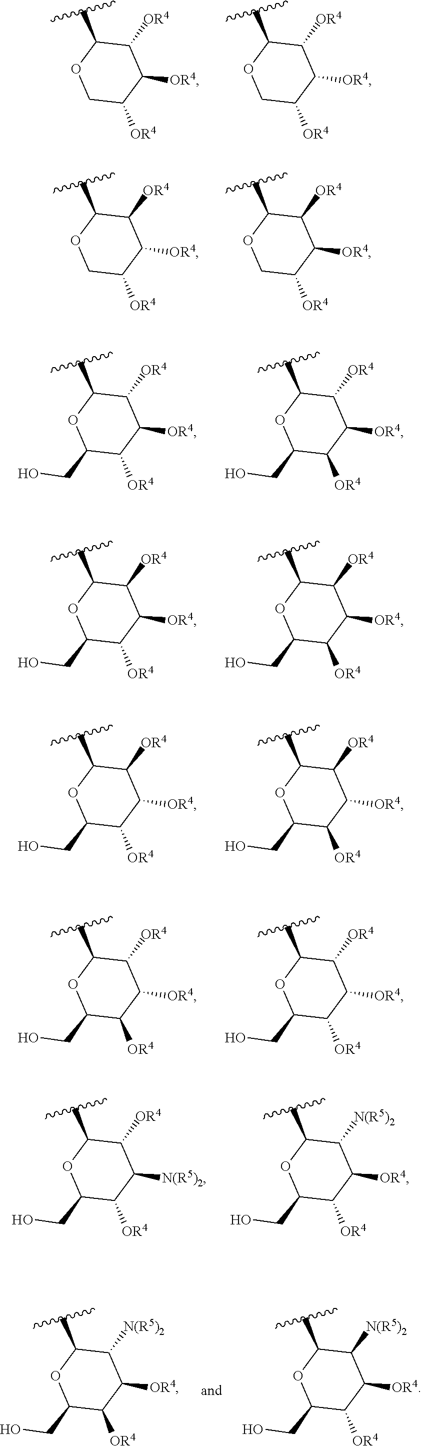

In some embodiments, R³ is

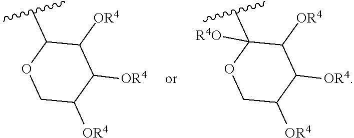

In some embodiments, R³ is

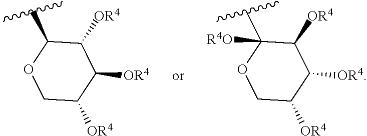

In some embodiments, R³ is

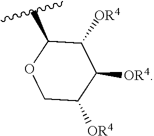

In some embodiments, R³ is

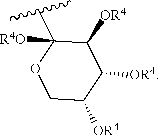

In some embodiments, -L-R³ is

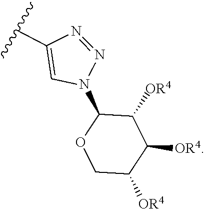

In some embodiments, -L-R³ is

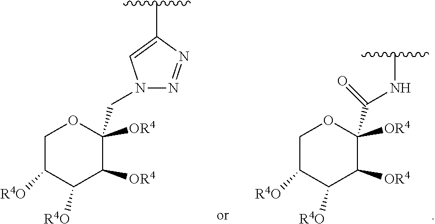

In some embodiments, -L-R³ is

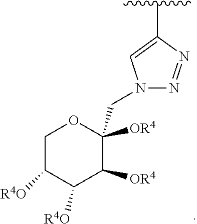

In some embodiments, -L-R³ is

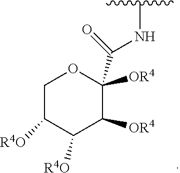

As defined herein, each R⁴ is independently hydrogen or an oxygen protecting group or wherein two R⁴ are joined together with the intervening atoms to form an oxygen protecting group. In some embodiments, each R⁴ is independently hydrogen or an oxygen protecting group. In some embodiments, each R⁴ is independently an oxygen protecting group or wherein two R⁴ are joined together with the intervening atoms to form an oxygen protecting group. In some embodiments, at least one instance of R⁴ is hydrogen. In some embodiments, each R⁴ is hydrogen. In some embodiments, at least one instance of R⁴ is an oxygen protecting group. In some embodiments, each R⁴ is independently an oxygen protecting group. In some embodiments, the oxygen protecting group is silyl, TBDPS, TBDMS, TIPS, TES, TMS, MOM, THP, t-Bu, Bn, allyl, acetyl, pivaloyl, or benzoyl. In some embodiments, the oxygen protecting group is acyl. In some embodiments, the oxygen protecting group is acetyl. In some embodiments, two R⁴ are joined together with the intervening atoms to form an oxygen protecting group. In some embodiments, two R⁴ are joined together with the intervening atoms to form an acetal protecting group.

As defined herein, each R⁵ is independently hydrogen or a nitrogen protecting group. In some embodiments, at least one instance of R⁵ is hydrogen. In some embodiments, each R⁵ is hydrogen. In some embodiments, at least one instance of R⁵ is a nitrogen protecting group. In some embodiments, each R⁵ is independently a nitrogen protecting group. In some embodiments, the nitrogen protecting group is Bn, Boc, Cbz, Fmoc, trifluoroacetyl, triphenylmethyl, acetyl, or Ts.

In some embodiments, the compound is selected from the group consisting of:

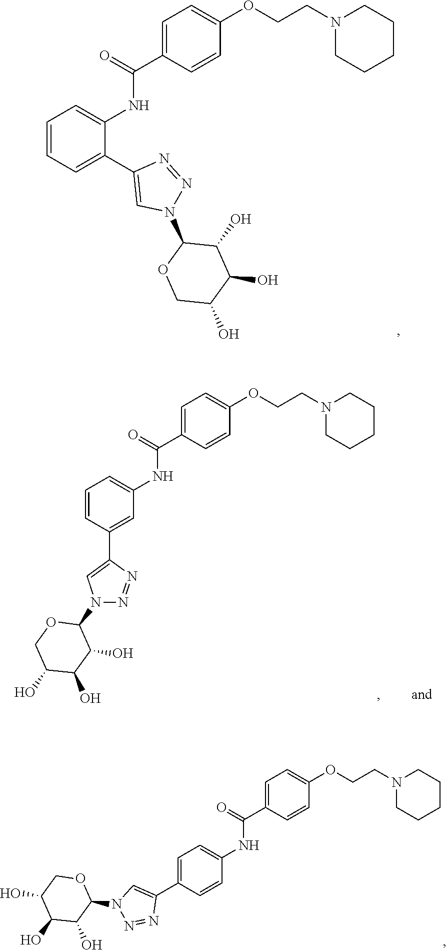 [SMILES: O=C(Nc1ccccc1-c1cn([C@@H]2OC[C@@H](O)[C@H](O)[C@H]2O)nn1)c1ccc(OCCN2CCCCC2)cc1 ; O=C(Nc1cccc(-c2cn([C@@H]3OC[C@@H](O)[C@H](O)[C@H]3O)nn2)c1)c1ccc(OCCN2CCCCC2)cc1 ; O=C(Nc1ccc(-c2cn([C@@H]3OC[C@@H](O)[C@H](O)[C@H]3O)nn2)cc1)c1ccc(OCCN2CCCCC2)cc1]

or a salt, solvate, hydrate, polymorph, co-crystal, tautomer, stereoisomer, isotopically labeled derivative, or prodrug thereof.

In some embodiments, the compound is selected from the group consisting of:

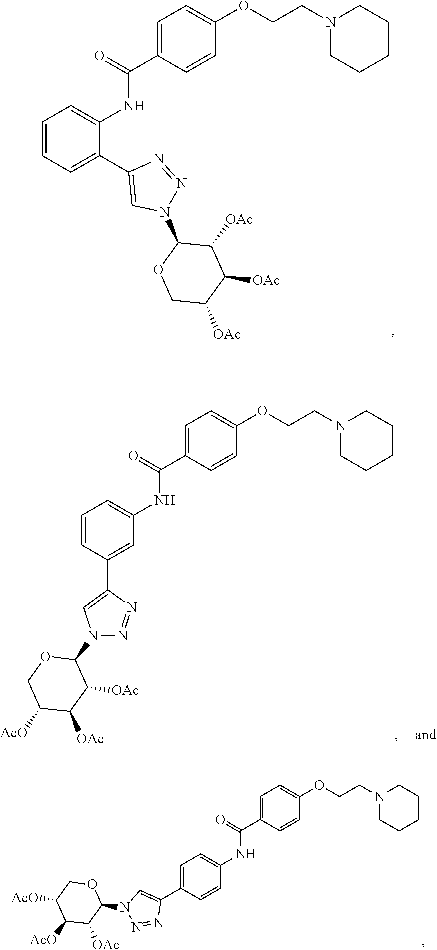 [SMILES: CC(=O)O[C@@H]1[C@@H](OC(C)=O)[C@H](OC(C)=O)CO[C@H]1n1cc(-c2ccccc2NC(=O)c2ccc(OCCN3CCCCC3)cc2)nn1 ; CC(=O)O[C@@H]1[C@@H](OC(C)=O)[C@H](OC(C)=O)CO[C@H]1n1cc(-c2cccc(NC(=O)c3ccc(OCCN4CCCCC4)cc3)c2)nn1 ; CC(=O)O[C@@H]1[C@@H](OC(C)=O)[C@H](OC(C)=O)CO[C@H]1n1cc(-c2ccc(NC(=O)c3ccc(OCCN4CCCCC4)cc3)cc2)nn1]

or a salt, solvate, hydrate, polymorph, co-crystal, tautomer, stereoisomer, isotopically labeled derivative, or prodrug thereof.

In some embodiments, the compound is selected from the group consisting of:

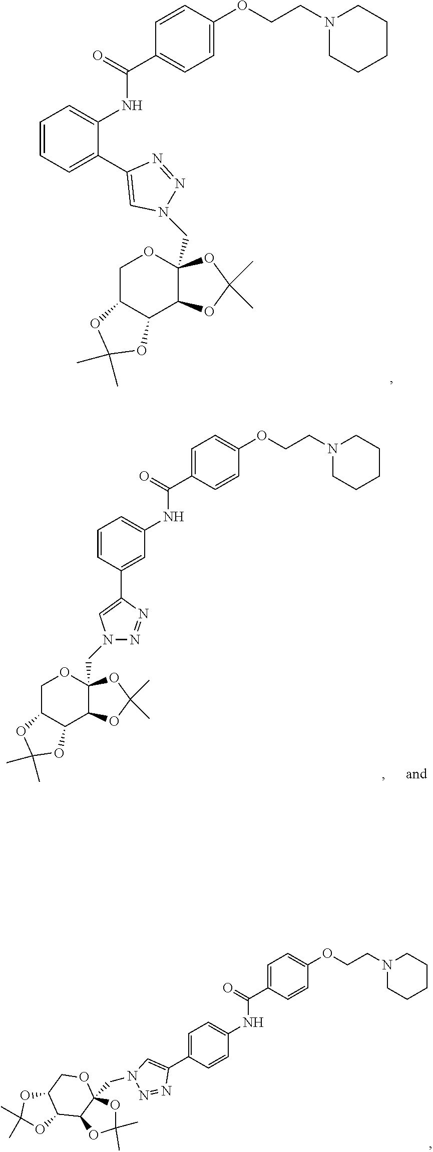 [SMILES: CC1(C)O[C@@H]2[C@@H](CO[C@@]3(Cn4cc(-c5ccccc5NC(=O)c5ccc(OCCN6CCCCC6)cc5)nn4)OC(C)(C)O[C@@H]23)O1 ; CC1(C)O[C@@H]2[C@@H](CO[C@@]3(Cn4cc(-c5cccc(NC(=O)c6ccc(OCCN7CCCCC7)cc6)c5)nn4)OC(C)(C)O[C@@H]23)O1 ; CC1(C)O[C@@H]2[C@@H](CO[C@@]3(Cn4cc(-c5ccc(NC(=O)c6ccc(OCCN7CCCCC7)cc6)cc5)nn4)OC(C)(C)O[C@@H]23)O1]

or a salt, solvate, hydrate, polymorph, co-crystal, tautomer, stereoisomer, isotopically labeled derivative, or prodrug thereof.

In some embodiments, the compound is selected from the group consisting of:

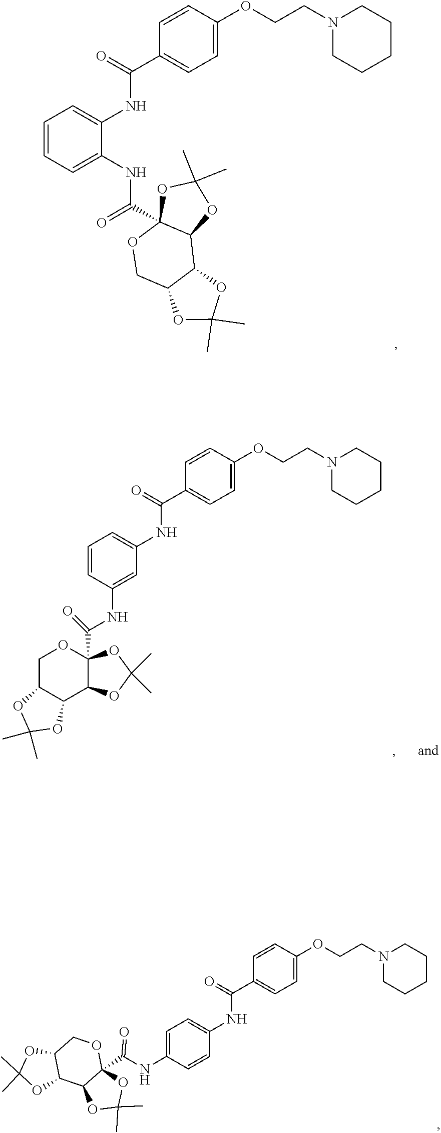 [SMILES: CC1(C)O[C@@H]2[C@@H](CO[C@@]3(C(=O)Nc4ccccc4NC(=O)c4ccc(OCCN5CCCCC5)cc4)OC(C)(C)O[C@@H]23)O1 ; CC1(C)O[C@@H]2[C@@H](CO[C@@]3(C(=O)Nc4cccc(NC(=O)c5ccc(OCCN6CCCCC6)cc5)c4)OC(C)(C)O[C@@H]23)O1 ; CC1(C)O[C@@H]2[C@@H](CO[C@@]3(C(=O)Nc4ccc(NC(=O)c5ccc(OCCN6CCCCC6)cc5)cc4)OC(C)(C)O[C@@H]23)O1]

or a salt, solvate, hydrate, polymorph, co-crystal, tautomer, stereoisomer, isotopically labeled derivative, or prodrug thereof.

In some embodiments, a salt of a compound of Formula (B) is an acetate salt. In some embodiments, a solvate of a compound of Formula (B) is an acetic acid solvate.

### Formula (C)

In another aspect, provided herein is a compound of Formula (C), or a salt, solvate, hydrate, polymorph, co-crystal, tautomer, stereoisomer, isotopically labeled derivative, or prodrug thereof:

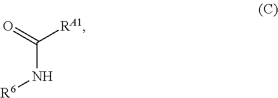

wherein:

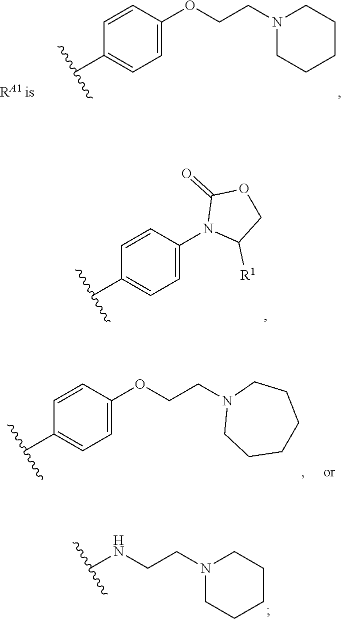

R¹ is optionally substituted alkyl or optionally substituted cycloalkyl;
R⁶ is optionally substituted

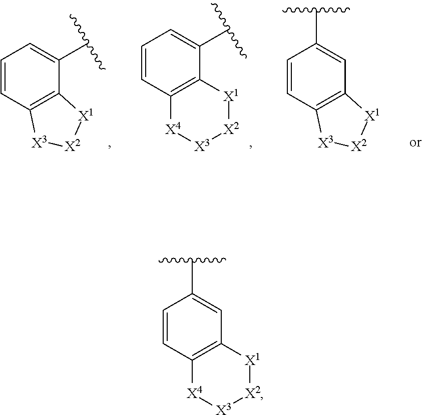

wherein:
X¹, X², X³, and X⁴ are each independently selected from —N(R⁷)—, —O—, —S—, and —C(R⁸)(R⁹)—;
each R⁷ is independently hydrogen, optionally substituted alkyl, or a nitrogen protecting group; and
R⁸ and R⁹ are each independently hydrogen, optionally substituted alkyl, or are taken together with the carbon to which they are attached form a carbonyl.

In some embodiments, the compound is of Formula (III), or a salt, solvate, hydrate, polymorph, co-crystal, tautomer, stereoisomer, isotopically labeled derivative, or prodrug thereof:

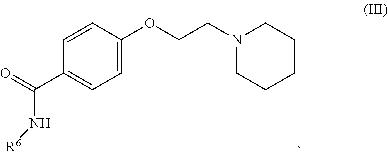

wherein:
R⁶ is

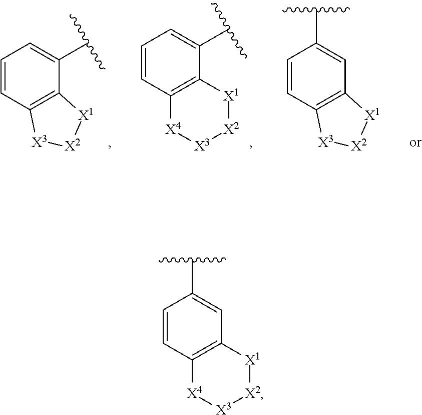

wherein:
X¹, X², X³, and X⁴ are each independently selected from —N(R⁷)—, —O—, —S—, and —C(R⁸)(R⁹)—;
each R⁷ is independently hydrogen, alkyl, or a nitrogen protecting group; and
R⁸ and R⁹ are each independently hydrogen, alkyl, or taken together with the carbon to which they are attached form a carbonyl.

In some embodiments, the compound is of Formula (XIV), or a salt, solvate, hydrate, polymorph, co-crystal, tautomer, stereoisomer, isotopically labeled derivative, or prodrug thereof:

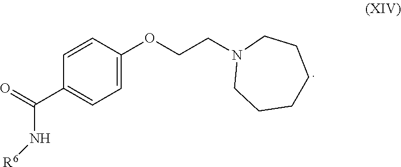

In some embodiments, the compound is of Formula (XV), or a salt, solvate, hydrate, polymorph, co-crystal, tautomer, stereoisomer, isotopically labeled derivative, or prodrug thereof:

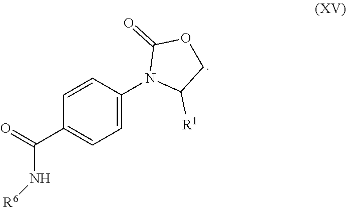

In some embodiments, the compound is of Formula (XVI), or a salt, solvate, hydrate, polymorph, co-crystal, tautomer, stereoisomer, isotopically labeled derivative, or prodrug thereof:

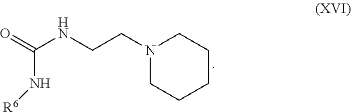

In some embodiments, the compound is of Formula (XVII), or a salt, solvate, hydrate, polymorph, co-crystal, tautomer, stereoisomer, isotopically labeled derivative, or prodrug thereof:

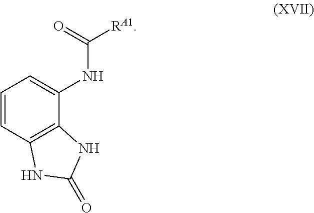

As defined herein, R^{A1} is

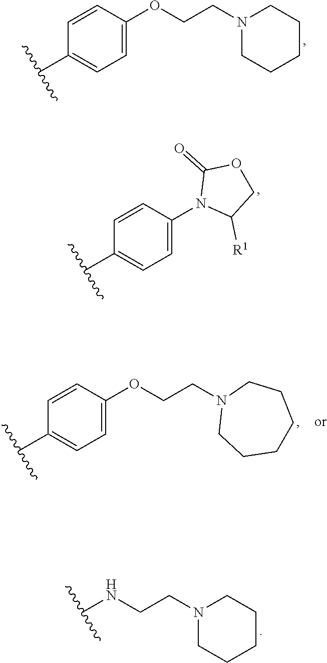

In some embodiments, R^{A1} is

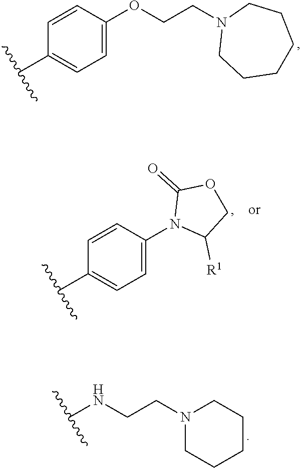

In some embodiments, R^{A1} is

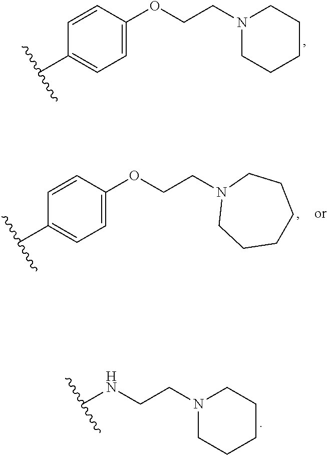 [SMILES: *c1ccc(OCCN2CCCCC2)cc1 ; *c1ccc(OCCN2CCCCCC2)cc1 ; *NCCN1CCCCC1]

In some embodiments, R^{A1} is

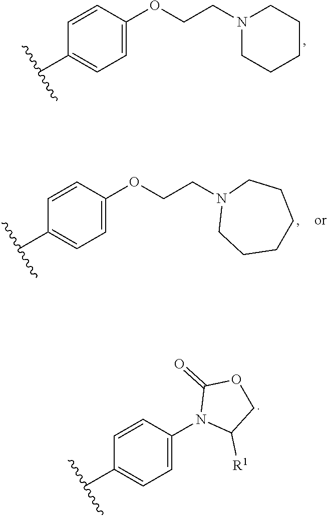

In some embodiments, R^{A1} is

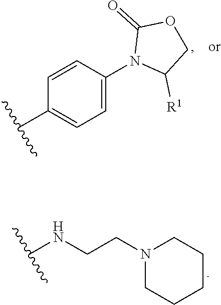

In some embodiments, R^{A1} is

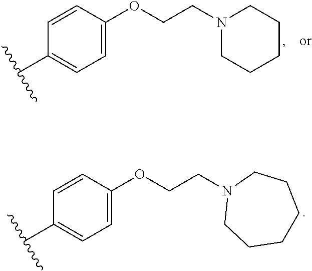 [SMILES: *c1ccc(OCCN2CCCCC2)cc1 ; *c1ccc(OCCN2CCCCCC2)cc1]

In some embodiments, R^{A1} is

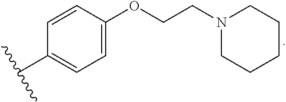 [SMILES: *c1ccc(OCCN2CCCCC2)cc1]

In some embodiments, R^{A1} is

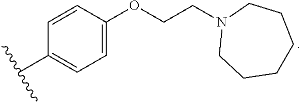 [SMILES: *c1ccc(OCCN2CCCCCC2)cc1]

In some embodiments, R^{A1} is

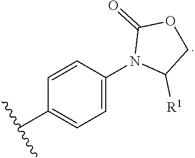

In some embodiments, R^{A1} is

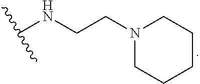 [SMILES: *NCCN1CCCCC1]

As defined herein, R¹ is optionally substituted alkyl or optionally substituted cycloalkyl. In some embodiments, R¹ is optionally substituted alkyl or cycloalkyl. In some embodiments, R¹ is optionally substituted alkyl. In some embodiments, R¹ is alkyl. In some embodiments, R¹ is optionally substituted C₁₋₁₀ alkyl. In some embodiments, R¹ is C₁₋₁₀ alkyl. In some embodiments, R¹ is optionally substituted C₁₋₆ alkyl. In some embodiments, R¹ is C₁₋₆ alkyl. In some embodiments, R¹ is methyl, ethyl, propyl, butyl, pentyl, or hexyl. In some embodiments, R¹ is optionally substituted C₁₋₄ alkyl. In some embodiments, R¹ is C₁₋₄ alkyl. In some embodiments, R¹ is methyl, ethyl, n-propyl, isopropyl, n-butyl, sec-butyl, or tert-butyl. In some embodiments, R¹ is methyl or ethyl. In some embodiments, R¹ is methyl. In some embodiments, R¹ is ethyl. In some embodiments, R¹ is isopropyl. In some embodiments, R¹ is n-propyl. In some embodiments, R¹ is optionally substituted cycloalkyl. In some embodiments, R¹ is unsubstituted cycloalkyl. In some embodiments, R¹ is optionally substituted C₃₋₈ cycloalkyl. In some embodiments, R¹ is C₃₋₈ cycloalkyl. In some embodiments, R¹ is optionally substituted C₃₋₆ cycloalkyl. In some embodiments, R¹ is C₃₋₆ cycloalkyl. In some embodiments, R¹ is optionally substituted C₃₋₄ cycloalkyl. In some embodiments, R¹ is C₃₋₄ cycloalkyl. In some embodiments, R¹ is optionally substituted C₅₋₆ cycloalkyl. In some embodiments, R¹ is C₅₋₆ cycloalkyl. In some embodiments, R¹ is cyclopropyl, cyclobutyl, cyclopentyl, or cyclohexyl. In some embodiments, R¹ is cyclopropyl or cyclobutyl. In some embodiments, R¹ is cyclopentyl or cyclohexyl. In some embodiments, R¹ is cyclopropyl. In some embodiments, R¹ is cyclobutyl. In some embodiments, R¹ is cyclopentyl. In some embodiments, R¹ is cyclohexyl.

As defined herein, R⁶ is optionally substituted

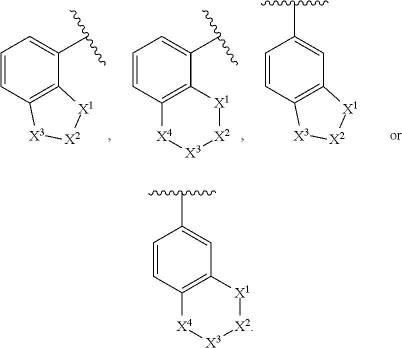

In some embodiments, R⁶ is

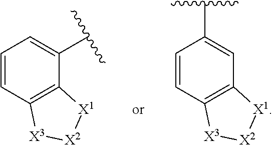

In some embodiments, R⁶ is

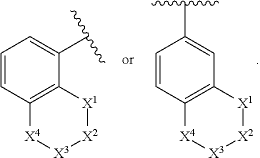

In some embodiments, R⁶ is

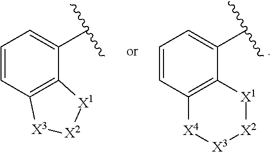

In some embodiments, R⁶ is

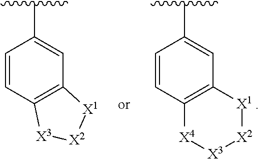

In some embodiments, R⁶ is optionally substituted

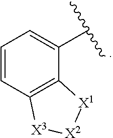

In some embodiments, R⁶ is

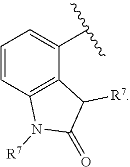

In some embodiments, R⁶ is optionally substituted

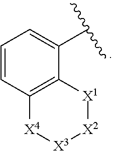

In some embodiments, R⁶ is optionally substituted

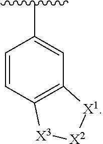

In some embodiments, R⁶ is

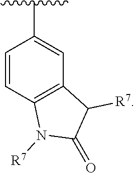

In some embodiments, R⁶ is optionally substituted

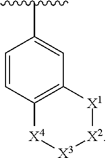

As defined herein, X¹, X², X³, and X⁴ are each independently selected from —N(R⁷)—, —O—, —S—, and —C(R⁸)(R⁹)—. In some embodiments, at least one of X¹, X², X³, and X⁴ is —N(R⁷)—. In some embodiments, one of X¹, X², X³, and X⁴ is —N(R⁷)—. In some embodiments, at least one of X¹, X², X³, and X⁴ is —O—. In some embodiments, one of X¹, X², X³, and X⁴ is —O—. In some embodiments, at least one of X¹, X², X³, and X⁴ is —S—. In some embodiments, one of X¹, X², X³, and X⁴ is —S—. In some embodiments, at least one of X¹, X², X³, and X⁴ is —C(R⁸)(R⁹)—. In some embodiments, at least one of X¹, X², X³, and X⁴ is —C(R⁸)(R⁹)—, and R⁸ and R⁹ taken together with the carbon to which they are attached form a carbonyl.

As defined herein, each R⁷ is independently hydrogen, optionally substituted alkyl, or a nitrogen protecting group. In some embodiments, R⁷ is hydrogen, alkyl, or a nitrogen protecting group. In some embodiments, each R⁷ is independently hydrogen or optionally substituted alkyl. In some embodiments, each R⁷ is independently hydrogen or a nitrogen protecting group. In some embodiments, each R⁷ is independently optionally substituted alkyl or a nitrogen protecting group. In some embodiments, at least one R⁷ is hydrogen. In some embodiments, each R⁷ is hydrogen. In some embodiments, at least one R⁷ is optionally substituted alkyl. In some embodiments, each R⁷ is optionally substituted alkyl. In some embodiments, at least one R⁷ is unsubstituted alkyl. In some embodiments, each R⁷ is unsubstituted alkyl. In some embodiments, at least one R⁷ is optionally substituted C₁₋₆ alkyl. In some embodiments, at least one R⁷ is unsubstituted C₁₋₆ alkyl. In some embodiments, R⁷ is methyl, ethyl, propyl, butyl, pentyl, or hexyl. In some embodiments, R⁷ is optionally substituted C₁₋₄ alkyl. In some embodiments, R⁷ is unsubstituted C₁₋₄ alkyl. In some embodiments, R⁷ is methyl, ethyl, n-propyl, isopropyl, n-butyl, sec-butyl, or tert-butyl. In some embodiments, R⁷ is methyl or ethyl. In some embodiments, R⁷ is methyl. In some embodiments, R⁷ is ethyl. In some embodiments, at least one R⁷ is a nitrogen protecting group. In some embodiments, each R⁷ is a nitrogen protecting group. In some embodiments, the nitrogen protecting group is Bn, Boc, Cbz, Fmoc, trifluoroacetyl, triphenylmethyl, acetyl, or Ts.

As defined herein, R⁸ and R⁹ are each independently hydrogen, optionally substituted alkyl, or are taken together with the carbon to which they are attached form a carbonyl. In some embodiments, R⁸ and R⁹ taken together with the carbon to which they are attached form a carbonyl. In some embodiments, R⁸ is hydrogen or optionally substituted alkyl, and R⁹ is hydrogen. In some embodiments, R⁸ is hydrogen or optionally substituted alkyl, and R⁹ is optionally substituted alkyl. In some embodiments, R⁸ is hydrogen, and R⁹ is hydrogen or optionally substituted alkyl. In some embodiments, R⁸ is optionally substituted alkyl, and R⁹ is hydrogen or optionally substituted alkyl. In some embodiments, R⁸ is hydrogen, and R⁹ is hydrogen. In some embodiments, R⁸ is hydrogen, and R⁹ is optionally substituted alkyl. In some embodiments, R⁸ is optionally substituted alkyl, and R⁹ is hydrogen. In some embodiments, R⁸ is optionally substituted alkyl, and R⁹ is optionally substituted alkyl. In some embodiments, at least one of R⁸ and R⁹ is optionally substituted C₁₋₆ alkyl. In some embodiments, at least one of R⁸ and R⁹ is methyl, ethyl, propyl, butyl, pentyl, or hexyl. In some embodiments, at least one of R⁸ and R⁹ is optionally substituted C₁₋₄ alkyl. In some embodiments, at least one of R⁸ and R⁹ is unsubstituted C₁₋₄ alkyl. In some embodiments, at least one of R⁸ and R⁹ is methyl, ethyl, n-propyl, isopropyl, n-butyl, sec-butyl, or tert-butyl. In some embodiments, at least one of R⁸ and R⁹ is methyl or ethyl. In some embodiments, at least one of R⁸ and R⁹ is methyl. In some embodiments, at least one of R⁸ and R⁹ is ethyl.

In some embodiments, the compound is selected from the group consisting of:

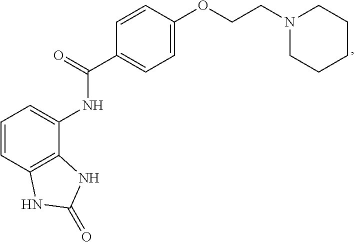 [SMILES: O=C(Nc1cccc2[nH]c(=O)[nH]c12)c1ccc(OCCN2CCCCC2)cc1]

or a salt, solvate, hydrate, polymorph, co-crystal, tautomer, stereoisomer, isotopically labeled derivative, or prodrug thereof.

In some embodiments, the compound is selected from the group consisting of:

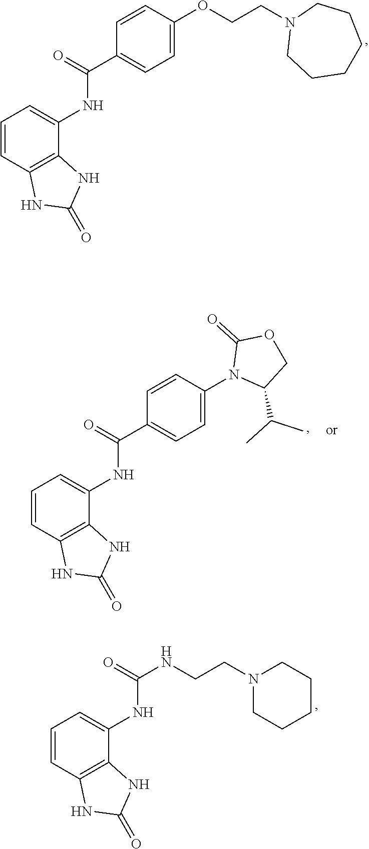 [SMILES: O=C(Nc1cccc2[nH]c(=O)[nH]c12)c1ccc(OCCN2CCCCCC2)cc1 ; CC(C)[C@H]1COC(=O)N1c1ccc(C(=O)Nc2cccc3[nH]c(=O)[nH]c23)cc1 ; O=C(NCCN1CCCCC1)Nc1cccc2[nH]c(=O)[nH]c12]

or a salt, solvate, hydrate, polymorph, co-crystal, tautomer, stereoisomer, isotopically labeled derivative, or prodrug thereof.

In some embodiments, a salt of a compound of Formula (C) is an acetate salt. In some embodiments, a solvate of a compound of Formula (C) is an acetic acid solvate.

### Formula (D)

In another aspect, provided herein is a compound of Formula (D), or a salt, solvate, hydrate, polymorph, co-crystal, tautomer, stereoisomer, isotopically labeled derivative, or prodrug thereof:

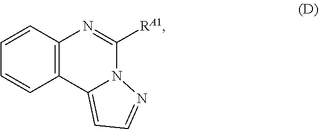

wherein:

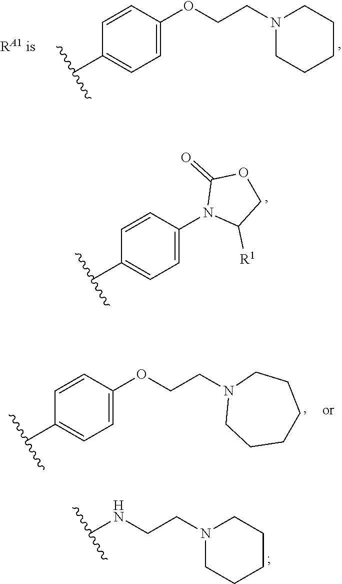

and
R¹ is optionally substituted alkyl or optionally substituted cycloalkyl.

As defined herein, R^{A1} is

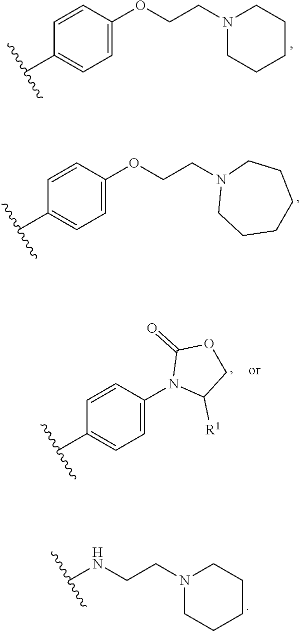

In some embodiments, R^{A1} is

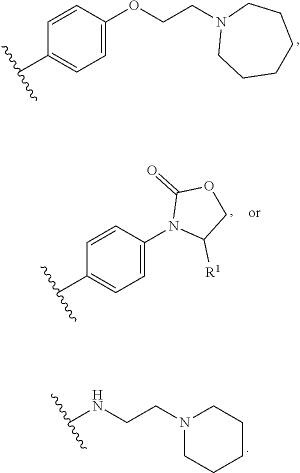

In some embodiments, R^{A1} is

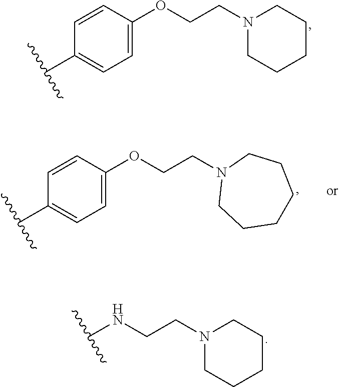 [SMILES: *c1ccc(OCCN2CCCCC2)cc1 ; *c1ccc(OCCN2CCCCCC2)cc1 ; *NCCN1CCCCC1]

In some embodiments, R^{A1} is

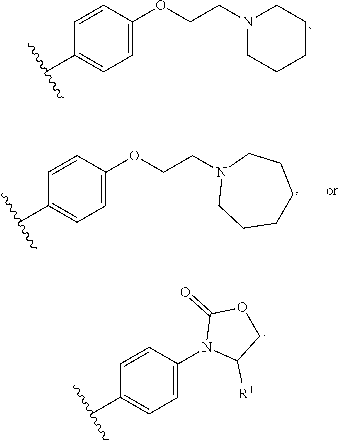

In some embodiments, R^{A1} is

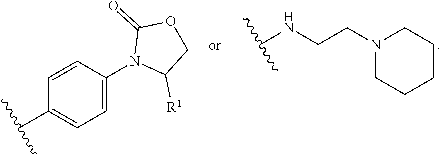

In some embodiments, R^{A1} is

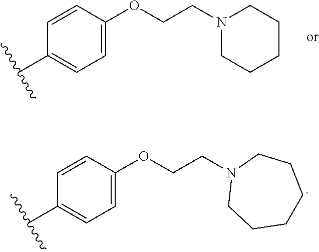 [SMILES: *c1ccc(OCCN2CCCCC2)cc1 ; *c1ccc(OCCN2CCCCCC2)cc1]

In some embodiments, R^{A1} is

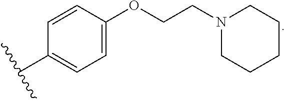 [SMILES: *c1ccc(OCCN2CCCCC2)cc1]

In some embodiments, R^{A1} is

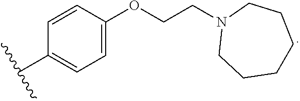 [SMILES: *c1ccc(OCCN2CCCCCC2)cc1]

In some embodiments, R^{A1} is

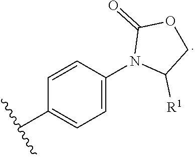

In some embodiments, R^{A1} is

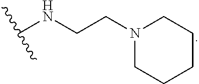 [SMILES: *NCCN1CCCCC1]

As defined herein, R¹ is optionally substituted alkyl or optionally substituted cycloalkyl. In some embodiments, R¹ is optionally substituted alkyl or cycloalkyl. In some embodiments, R¹is optionally substituted alkyl. In some embodiments, R¹ is alkyl. In some embodiments, R¹ is optionally substituted C₁₋₁₀ alkyl. In some embodiments, R¹ is C₁₋₁₀ alkyl. In some embodiments, R¹ is optionally substituted C₁₋₆ alkyl. In some embodiments, R¹ is C₁₋₆ alkyl. In some embodiments, R¹ is methyl, ethyl, propyl, butyl, pentyl, or hexyl. In some embodiments, R¹ is optionally substituted C₁₋₄ alkyl. In some embodiments, R¹ is C₁₋₄ alkyl. In some embodiments, R¹ is methyl, ethyl, n-propyl, isopropyl, n-butyl, sec-butyl, or tert-butyl. In some embodiments, R¹ is methyl or ethyl. In some embodiments, R¹ is methyl. In some embodiments, R¹ is ethyl. In some embodiments, R¹ is isopropyl. In some embodiments, R¹ is n-propyl. In some embodiments, R¹ is optionally substituted cycloalkyl. In some embodiments, R¹ is unsubstituted cycloalkyl. In some embodiments, R¹ is optionally substituted C₃₋₈ cycloalkyl. In some embodiments, R¹ is C₃₋₈ cycloalkyl. In some embodiments, R¹ is optionally substituted C₃₋₆ cycloalkyl. In some embodiments, R¹ is C₃₋₆ cycloalkyl. In some embodiments, R¹ is optionally substituted C₃₋₄ cycloalkyl. In some embodiments, R¹ is C₃₋₄ cycloalkyl. In some embodiments, R¹ is optionally substituted C₅₋₆ cycloalkyl. In some embodiments, R¹ is C₅₋₆ cycloalkyl. In some embodiments, R¹ is cyclopropyl, cyclobutyl, cyclopentyl, or cyclohexyl. In some embodiments, R¹ is cyclopropyl or cyclobutyl. In some embodiments, R¹ is cyclopentyl or cyclohexyl. In some embodiments, R¹ is cyclopropyl. In some embodiments, R¹ is cyclobutyl. In some embodiments, R¹ is cyclopentyl. In some embodiments, R¹ is cyclohexyl.

In some embodiments, the compound has the formula:

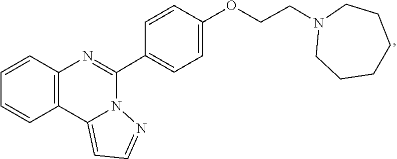 [SMILES: c1ccc2c(c1)nc(-c1ccc(OCCN3CCCCCC3)cc1)n1nccc21]

or a salt, solvate, hydrate, polymorph, co-crystal, tautomer, stereoisomer, isotopically labeled derivative, or prodrug thereof.

In some embodiments, the compound has the formula:

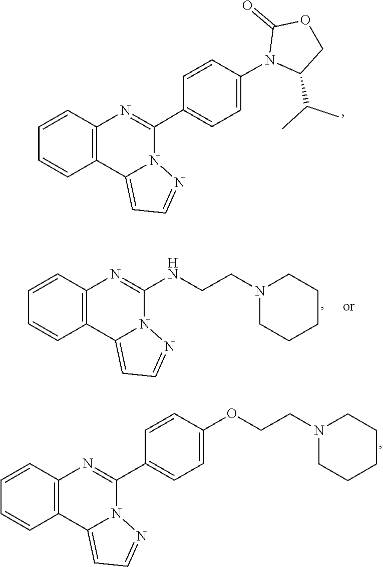

or a salt, solvate, hydrate, polymorph, co-crystal, tautomer, stereoisomer, isotopically labeled derivative, or prodrug thereof.

In some embodiments, a salt of a compound of Formula (D) is an acetate salt. In some embodiments, a solvate of a compound of Formula (D) is an acetic acid solvate.

### Formula (E)

In another aspect, provided herein is a compound of Formula (E), or a salt, solvate, hydrate, polymorph, co-crystal, tautomer, stereoisomer, isotopically labeled derivative, or prodrug thereof:

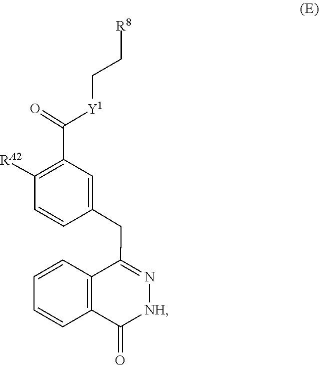

wherein:
R⁸ is

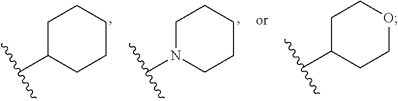

R^{A2} is halogen;
Y¹ is —O— or —N(R⁷)—; and
R⁷ is hydrogen, optionally substituted alkyl, or a nitrogen protecting group.

As defined herein, R⁸ is

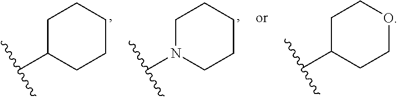

In some embodiments, R⁸ is

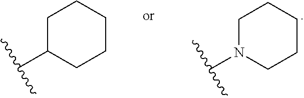 [SMILES: *C1CCCCC1 ; *N1CCCCC1]

In some embodiments, R⁸ is

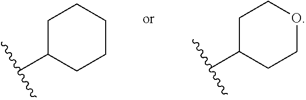 [SMILES: *C1CCCCC1 ; *C1CCOCC1]

In some embodiments, R⁸ is

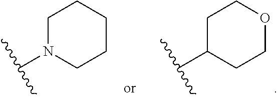 [SMILES: *N1CCCCC1 ; *C1CCOCC1]

In some embodiments, R⁸ is

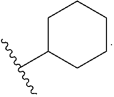 [SMILES: *C1CCCCC1]

In some embodiments, R⁸ is

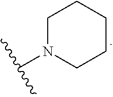 [SMILES: *N1CCCCC1]

In some embodiments, R⁸ is

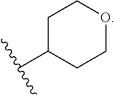 [SMILES: *C1CCOCC1]

As defined herein, R^{A2} is halogen. In some embodiments, R^{A2} is I, Br, Cl, or F. In some embodiments, R^{A2} is Br, Cl, or F. In some embodiments, R^{A2} is F or H. In some embodiments, R^{A2} is Br or Cl. In some embodiments, R^{A2} is I. In some embodiments, R^{A2} is Br. In some embodiments, R^{A2} is Cl.

As defined herein, Y¹ is —O— or —N(R⁷)—. In some embodiments, Y¹ is —O—. In some embodiments, Y¹ is —N(R⁷)—.

In some embodiments

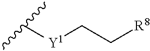

is

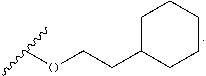 [SMILES: *OCCC1CCCCC1]

In some embodiments,

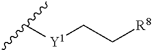

is

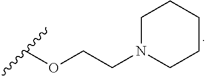 [SMILES: *OCCN1CCCCC1]

In some embodiments,

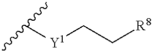

is

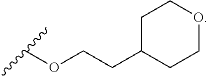 [SMILES: *OCCC1CCOCC1]

In some embodiments,

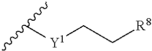

is

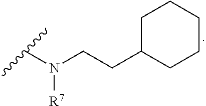

In some embodiments,

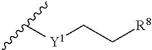

is

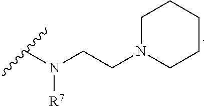

In some embodiments,

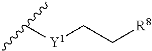

is

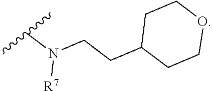

In some embodiments,

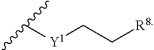

is

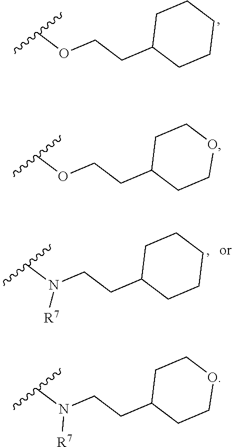

In some embodiments,

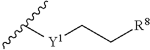

is

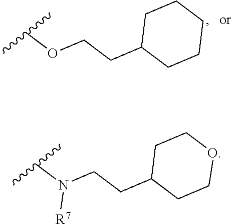

As defined herein, R⁷ is hydrogen, optionally substituted alkyl, or a nitrogen protecting group. In some embodiments, R⁷ is hydrogen, alkyl, or a nitrogen protecting group. In some embodiments, R⁷ is hydrogen or optionally substituted alkyl. In some embodiments, R⁷ is hydrogen or a nitrogen protecting group. In some embodiments, R⁷ is optionally substituted alkyl or a nitrogen protecting group. In some embodiments, R⁷ is hydrogen. In some embodiments, R⁷ is optionally substituted alkyl. In some embodiments, R⁷ is unsubstituted alkyl. In some embodiments, R⁷ is optionally substituted C₁₋₆ alkyl. In some embodiments, R⁷ is unsubstituted C₁₋₆ alkyl. In some embodiments, R⁷ is methyl, ethyl, propyl, butyl, pentyl, or hexyl. In some embodiments, R⁷ is optionally substituted C₁₋₄ alkyl. In some embodiments, R⁷ is unsubstituted C₁₋₄ alkyl. In some embodiments, R⁷ is methyl, ethyl, n-propyl, isopropyl, n-butyl, sec-butyl, or tert-butyl. In some embodiments, R⁷ is methyl or ethyl. In some embodiments, R⁷ is methyl. In some embodiments, R⁷ is ethyl. In some embodiments, R⁷ is a nitrogen protecting group. In some embodiments, the nitrogen protecting group is Bn, Boc, Cbz, Fmoc, trifluoroacetyl, triphenylmethyl, acetyl, or Ts.

In some embodiments, the compound has the formula:

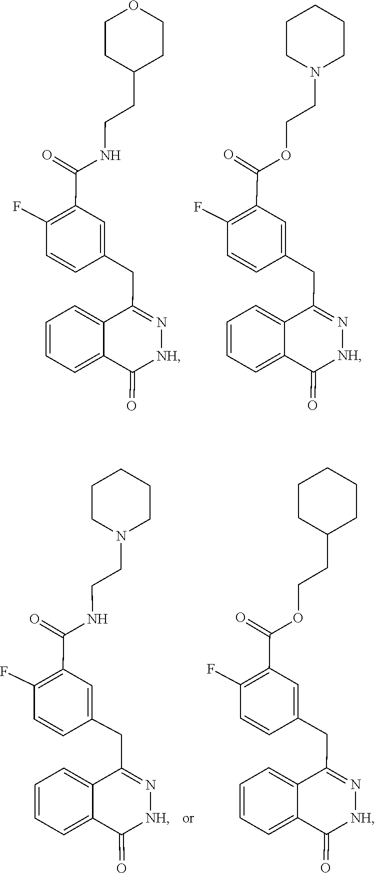 [SMILES: O=C(NCCC1CCOCC1)c1cc(Cc2n[nH]c(=O)c3ccccc23)ccc1F ; O=C(OCCN1CCCCC1)c1cc(Cc2n[nH]c(=O)c3ccccc23)ccc1F ; O=C(NCCN1CCCCC1)c1cc(Cc2n[nH]c(=O)c3ccccc23)ccc1F ; O=C(OCCC1CCCCC1)c1cc(Cc2n[nH]c(=O)c3ccccc23)ccc1F]

or a salt, solvate, hydrate, polymorph, co-crystal, tautomer, stereoisomer, isotopically labeled derivative, or prodrug thereof.

In some embodiments, a salt of a compound of Formula (E) is an acetate salt. In some embodiments, a solvate of a compound of Formula (E) is an acetic acid solvate.

### Pharmaceutical Compositions

In one aspect, provided herein is a pharmaceutical composition comprising a compound provided herein (e.g., a compound of Formula (A), (B), (C), (D), (E), or (F)), or a salt thereof, and a pharmaceutically acceptable carrier. In another aspect, provided herein is a pharmaceutical composition comprising a compound provided herein (e.g., a compound of Formula (A), (B), (C), (D), (E), or (F)), or a salt, solvate, hydrate, polymorph, co-crystal, tautomer, stereoisomer, isotopically labeled derivative, or prodrug thereof, and a pharmaceutically acceptable carrier. In some embodiments, the pharmaceutical composition further comprises an additional therapeutic agent.

Pharmaceutical compositions described herein can be prepared by any method known in the art of pharmacology. In general, such preparatory methods include bringing the compound described herein (i.e., the “active ingredient”) into association (e.g., mixing, blending, combining, extruding) with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj© 45), polyoxyethylene hydrogenated castor oil, polyethoxylated castor oil, polyoxymethylene stearate, and Solutol*), sucrose fatty acid esters, polyethylene glycol fatty acid esters (e.g., Cremophor®), polyoxyethylene ethers, (e.g., polyoxyethylene lauryl ether (Brij© 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

In an embodiment, compound is administered to the subject using a pharmaceutically acceptable formulation, e.g., a pharmaceutically acceptable formulation that provides sustained delivery of the compound to a subject for at least 12 hours, 24 hours, 36 hours, 48 hours, one week, two weeks, three weeks, or four weeks after the pharmaceutically acceptable formulation is administered to the subject.

In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. Actual dosage levels and time course of administration of the active ingredients in the pharmaceutical compositions of this disclosure may be varied so as to obtain an amount of the active ingredient which is effective to achieve the desired therapeutic response for a particular patient, composition, and mode of administration, without being toxic (or unacceptably toxic) to the patient.

A compound or composition, as described herein, can be administered in combination with one or more additional therapeutic agents (e.g., therapeutically and/or prophylactically active agents). In some embodiments, the pharmaceutical composition further comprises an additional therapeutic agent.

The compounds or compositions can be administered in combination with additional therapeutic agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity of a protein kinase in a subject or cell), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects.

In certain embodiments, a pharmaceutical composition described herein including a compound described herein and an additional therapeutic agent shows a synergistic effect that is absent in a pharmaceutical composition including one of the compound and the additional therapeutic agent, but not both. In some embodiments, the additional therapeutic agent achieves a desired effect for the same disorder. In some embodiments, the additional therapeutic agent achieves different effects.

The compound or composition can be administered concurrently with, prior to, or subsequent to one or more additional therapeutic agents, which may be useful as, e.g., combination therapies.

Therapeutic agents include therapeutically active agents. Therapeutic agents also include prophylactically active agents. Therapeutic agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional therapeutic agent is a therapeutic agent useful for treating and/or preventing a disease (e.g., cancer, inflammatory disease, or autoimmune disease). Each additional therapeutic agent may be administered at a dose and/or on a time schedule determined for that therapeutic agent. The additional therapeutic agents may also be administered together with each other and/or with the compound or composition described herein in a single dose or composition or administered separately in different doses or compositions. The particular combination to employ in a regimen will take into account compatibility of the compound described herein with the additional therapeutic agent(s) and/or the desired therapeutic and/or prophylactic effect to be achieved. In general, it is expected that the additional therapeutic agent(s) in combination be utilized at levels that do not exceed the levels at which they are utilized individually. In some embodiments, the levels utilized in combination will be lower than those utilized individually.

The additional therapeutic agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins. In certain embodiments, the additional therapeutic agent is an anti-proliferative agent. In certain embodiments, the additional therapeutic agent is an anti-cancer agent. In certain embodiments, the additional therapeutic agent is an anti-viral agent. In certain embodiments, the additional therapeutic agent is an binder or inhibitor of a protein kinase. In certain embodiments, the additional therapeutic agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy. Additional therapeutic agents include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells.

In some embodiments, the additional therapeutic agent is an anti-cancer agent, antifungal agent, cardiovascular agent, anti-inflammatory agent, chemotherapeutic agent, an anti-angiogenesis agent, cytotoxic agent, an antiproliferation agent, metabolic disease agent, ophthalmologic disease agent, central nervous system (CNS) disease agent, urologic disease agent, or gastrointestinal disease agent. In a further embodiment, the additional therapeutic agent is an anti-cancer agent, chemotherapeutic agent, an anti-angiogenesis agent, cytotoxic agent, or an anti-proliferation agent.

Also encompassed by the disclosure are kits (e.g., pharmaceutical packs).

In one aspect, provided herein is a kit comprising an effective amount of a compound provided herein (e.g., a compound of Formula (A), (B), (C), (D), (E), or (F)), or a salt thereof, or a pharmaceutical composition thereof, and instructions for administering the compound to a subject in need thereof. In another aspect, provided herein is a kit comprising an effective amount of a compound provided herein (e.g., a compound of Formula (A), (B), (C), (D), (E), or (F)), or a salt, solvate, hydrate, polymorph, co-crystal, tautomer, stereoisomer, isotopically labeled derivative, or prodrug thereof, or a pharmaceutical composition thereof, and instructions for administering the compound to a subject in need thereof.

The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., cancer, inflammatory disease, or autoimmune disease) in a subject in need thereof. In certain embodiments, the kits are useful for preventing a disease (e.g., cancer, inflammatory disease, or autoimmune disease) in a subject in need thereof. In certain embodiments, the kits are useful for reducing the risk of developing a disease (e.g., cancer, inflammatory disease, or autoimmune disease) in a subject in need thereof. In certain embodiments, the kits are useful for inhibiting the activity (e.g., aberrant activity, such as increased activity) of IL-6 in a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., cancer, inflammatory disease, or autoimmune disease) in a subject in need thereof. In certain embodiments, the kits and instructions provide for preventing a disease (e.g., cancer, inflammatory disease, or autoimmune disease) in a subject in need thereof. In certain embodiments, the kits and instructions provide for reducing the risk of developing a disease (e.g., cancer, inflammatory disease, or autoimmune disease) in a subject in need thereof. In certain embodiments, the kits and instructions provide for inhibiting the activity (e.g., aberrant activity, such as increased activity) of IL-6 in a subject or cell. A kit described herein may include one or more additional therapeutic agents described herein as a separate composition.

### Methods

Provided herein are methods of using a compound provided herein (e.g., a compound of Formula (A), (B), (C), (D), (E), or (F)), or a salt, solvate, hydrate, polymorph, co-crystal, tautomer, stereoisomer, isotopically labeled derivative, or prodrug thereof, or a pharmaceutical composition thereof. Also provided herein are methods of using a compound provided herein (e.g., a compound of Formula (A), (B), (C), (D), (E), or (F)), or a salt thereof, or a pharmaceutical composition thereof.

In one aspect, provided herein is a method of inhibiting IL-6 signaling comprising administering a compound provided herein (e.g., a compound of Formula (A), (B), (C), (D), (E), or (F)), or a salt thereof, or a pharmaceutical composition thereof.

In another aspect, provided herein is a method of inhibiting IL-6/gp130 comprising administering a compound provided herein (e.g., a compound of Formula (A), (B), (C), (D), (E), or (F)), or a salt thereof, or a pharmaceutical composition thereof.

In some embodiments, the compound is a dual PARP/IL-6 inhibitor.

In some embodiments, the inhibition of IL-6 is in vitro. In some embodiments, the inhibition of IL-6 is in vivo. In some embodiments, any of the methods provided herein further comprise administering the compound to a subject. In some embodiments, the inhibition of IL-6 is in a subject, cell, tissue, or biological sample. In some embodiments, the inhibition of IL-6 is in a subject. In some embodiments, the inhibition of IL-6 is in a cell. In some embodiments, the inhibition of IL-6 is in a tissue. In some embodiments, the inhibition of IL-6 is in a biological sample.

In another aspect, provided herein is a method of treating inflammatory disease in a subject in need thereof, the method comprising administering to the subject a compound provided herein (e.g., a compound of Formula (A), (B), (C), (D), (E), or (F)), or a salt thereof, or a pharmaceutical composition thereof.

In some embodiments, the inflammatory disease is fibrosis. In some embodiments, the fibrosis is liver fibrosis. In some embodiments, the inflammatory disease is nonalcoholic steatohepatitis (NASH). In some embodiments, the method reduces inflammation in the subject.

In another aspect, provided herein is a method of treating cancer in a subject in need thereof, the method comprising administering to the subject a compound provided herein (e.g., a compound of Formula (A), (B), (C), (D), (E), or (F)), or a salt thereof, or a pharmaceutical composition thereof.

In some embodiments, the cancer is breast cancer, pancreatic cancer, bone cancer, or brain cancer. In some embodiments, the cancer is breast cancer or pancreatic cancer. In some embodiments, the cancer is breast cancer. In some embodiments, the breast cancer is triple negative breast cancer. In some embodiments, the cancer is bone cancer or brain cancer. In some embodiments, the cancer is bone cancer. In some embodiments, the cancer is brain cancer.

In some embodiments, the method modulates proliferative activity in the subject. In some embodiments, the method modulates cell proliferation in the subject. In some embodiments, the compound, or a salt thereof, or a pharmaceutical composition thereof, preferentially targets cancer cells over non-transformed cells.

In another aspect, provided herein is a method of treating an autoimmune disease in a subject in need thereof, the method comprising administering to the subject a compound provided herein (e.g., a compound of Formula (A), (B), (C), (D), (E), or (F)), or a salt thereof, or a pharmaceutical composition thereof.

In some embodiments, the autoimmune disease is multiple sclerosis or arthritis. In some embodiments, the autoimmune disease is multiple sclerosis. In some embodiments, the autoimmune disease is arthritis. In some embodiments, the arthritis is rheumatoid arthritis. In some embodiments, the arthritis is giant cell arthritis.

In some embodiments, any of the methods provided herein comprise administering to the subject a therapeutically effective amount of the compound, or a salt thereof, or a pharmaceutical composition thereof.

In some embodiments, the subject is identified as in need of such treatment. Identifying a subject in need of such treatment can be in the judgment of a subject or a healthcare professional and can be subjective (e.g., opinion) or objective (e.g., measurable by a test or diagnostic method). In some embodiments, the subject has such disease or disorder. In some embodiments, the subject is suffering from or susceptible to such disease or disorder. In some embodiments, the subject has been diagnosed with such disease or disorder.

### Examples

### General Procedure for Western Blot Assays

Cells were harvested and lysed in cold radioimmunoprecipitation assay (RIPA) lysis buffer containing proteasome inhibitor cocktail and phosphatase inhibitor cocktail. The protein concentrations were determined using the BCA Protein Assay kit. After adding the loading buffer and boiling at 95° C. for 10 minutes, equivalent amounts of proteins were loaded on and separated by SDS-PAGE, and then were transferred to PVDF membranes. Membranes were probed with primary antibodies (1:1000) against gp130 and GAPDH. Membranes were analyzed using Enhanced Chemiluminescence Plus reagents and either scanned with the Storm Scanner (Amersham Pharmacia Biotech, Inc., Piscataway, NJ) or imaged with ChemiDoc MP imaging system (Bio-Rad, Hercules, CA).

### Drug Affinity Responsive Target Stability (DARTS) Assay

MDA-MB-231 breast cancer cells or MIAPaCa2 pancreatic cancer cells were lysed in cold radioimmunoprecipitation assay (RIPA) lysis buffer containing proteasome inhibitor cocktail and phosphatase inhibitor cocktail. Then lysates were incubated with escalating concentrations (10-1000 μM) of LS-28-3 or bazedoxifene at room temperature for 1 h. Proteolysis was followed by adding protease pronase solution at a ratio of 1 mg of pronase to 1000 mg (or 2000 mg) of lysate protein for 20 min at room temperature. To stop proteolysis, 4 x SDS sample loading buffer was added at 1:3 ratio to each sample and boiled at 95° C. for 10 min. The resulted protein samples were separated by 8% SDS-PAGE gel and analyzed by western blotting.

The DARTS assay is a method for studying the specific protein-ligand binding interactions. It is based on the principle that the target protein structure might be stabilized and become less susceptible to proteolysis by proteases upon drug binding [Lomenick B, Jung G, Wohlschlegel J, et al. Target identification using drug affinity responsive target stability (DARTS). Curr Protoc Chem Biol 2011, 3, 163-180; Lomenick B, Olsen R, Huang J. Identification of direct protein targets of small molecules. ACS Chem Biol. 2011, 6, 34-46]. It can be used to find the targeting protein of a small molecule by running SDS-gel without purifying any specific protein. It can also be used to verify whether a small molecule is targeting a designated protein by western blotting. Recently, this method was successfully used to assess the direct binding of potential inhibitor SC144 to gp130 in human ovarian cells and raloxifene to gp130 in human RH30 sarcoma cells [Li H, Xiao H, Lin L, et al. Drug design targeting protein-protein interactions (PPIs) using multiple ligand simultaneous docking (MLSD) and drug repositioning: discovery of raloxifene and bazedoxifene as novel inhibitors of IL-6/GP130 interface. J Med Chem 2014, 57, 632-641; Xu S, Grande1 F, Garofalo A, et al. Discovery of a novel orally active small-molecule gp130 inhibitor for the treatment of ovarian cancer. Mol Cancer Ther 2013, 12, 937-949].

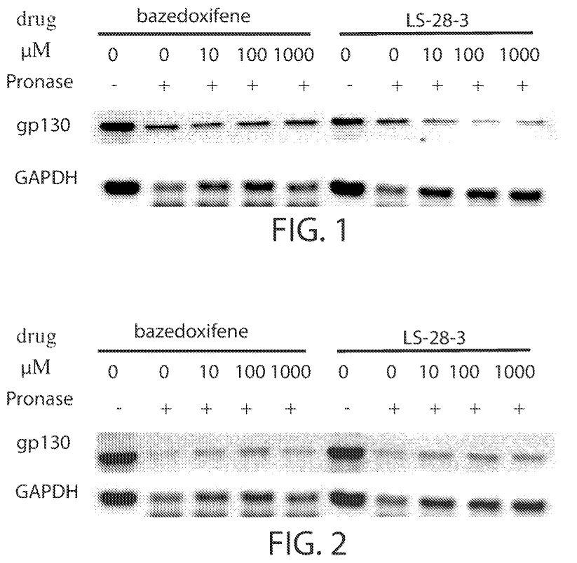

To investigate the binding of LS-28-3 to gp130, DARTS assays were performed using MDA-MB-231 breast cancer cell lysates or MIAPaCa2 pancreatic cancer cell lysates, following the protocol as previously described. In both MDA-MB-231 breast cancer cell lysates (FIG. 1) or MIAPaCA2 pancreatic cancer cell lysates (FIG. 2) using a 1:2000 ratio of pronase:protein with a proteolysis time of 20 minutes, LS-28-3 showed direct binding to gp130, stabilizing its structure and protecting it from pronase proteolysis.

### Cell Viability Assay

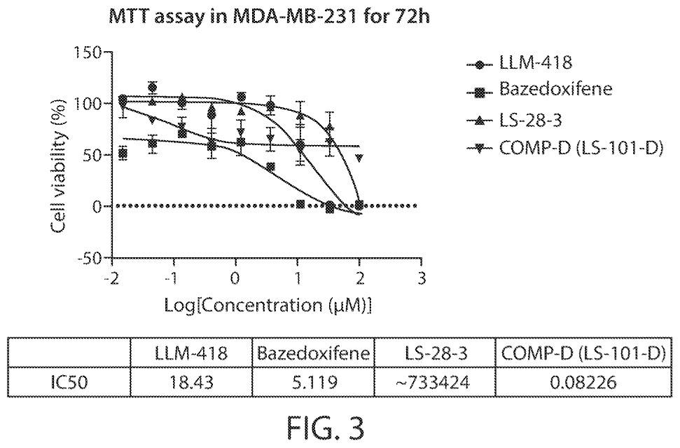

Cell viability of MDA-MB-231 breast cancer cells was evaluated by using the MTT assay in triple replicates in one experiment and repeated for three times (FIG. 3). MDA-MB-231 SUM159, SUM159 gp140ko, MDA-MB-231 breast cancer cells or SUM149 or HCC1937 BRCA mutant breast cancer cells were seeded in 96-well plates at a density of 3,000 cells per well. The cells were incubated at 37° C. for a period of 24 hours. Escalating concentrations of IL-6/gp130 inhibitors (e.g., compounds of Formula (A), (B), (C), (D), (E), or (F) including LLM-418, bazedoxifene, LS-28-3, and COMP-D (LS-101-D)) were added in triplicate to the plates in the presence of 10% FBS. After incubation for 72 hours (unless otherwise noted), a stock solution of MTT (0.5 mg/ml) was then added to each well containing the treated cells, followed by incubation at 37° C. for 3 h. After removal of medium, the MTT dye was dissolved with spectrophotometric grade DMSO and the absorbance was read at 570 nm. The IC₅₀ value is determined by interpolation based on the absorbance value halfway between positive and negative controls.

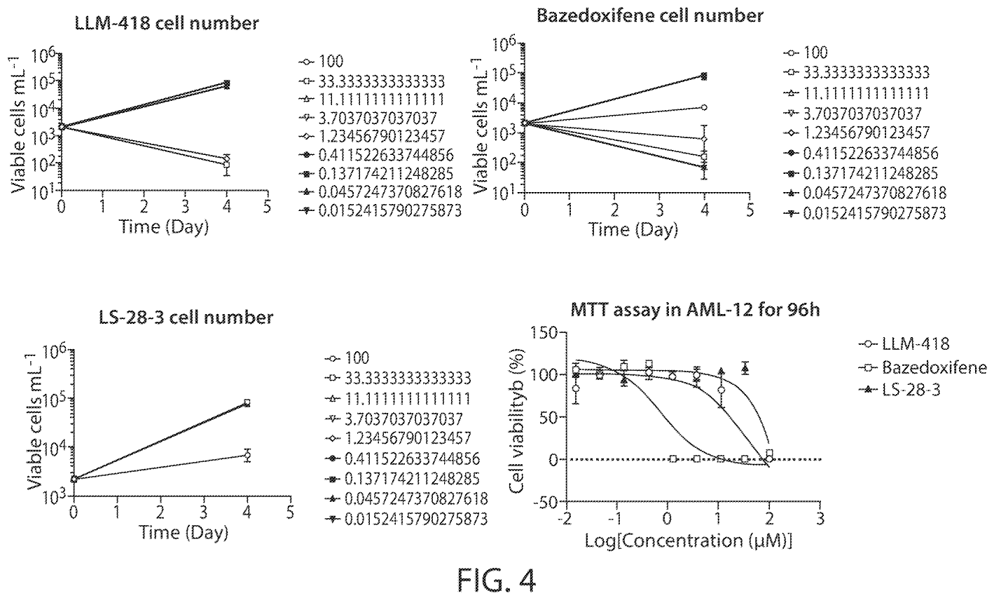

Cell viability of AML-12 hepatocyte cells was evaluated by using the MTT assay in triple replicates in one experiment and repeated for three times (FIG. 4). AML-12 hepatocyte cells were seeded in 96-well plates at a density of 3,000 cells per well. The cells were incubated at 37° C. for a period of 24 hours. Escalating concentrations of IL-6/gp130 inhibitors (e.g., compounds of Formula (A), (B), (C), (D), (E), or (F) including LLM-418, bazedoxifene, and LS-28-3) were added in triplicate to the plates in the presence of 10% FBS. After incubation for 96 hours, a stock solution of MTT (0.5 mg/ml) was then added to each well containing the treated cells, followed by incubation at 37° C. for 3 h. After removal of medium, the MTT dye was dissolved with spectrophotometric grade DMSO and the absorbance was read at 570 nm. The IC₅₀ value is determined by interpolation based on the absorbance value halfway between positive and negative controls. Viable cell numbers (viable cells mL-¹) were determined by measuring the absorbance at 450 nm in a microplate reader.

### MTT Results

TABLE 1
IC₅₀ Values in Breast Cancer Cell Lines (μM)
IC₅₀ Values in Breast Cancer Cell Lines (μM)

| | | | | MDA-MB-231 |
|---|---|---|---|---|
| | MDA-MB- | | SUM159 | Bone |
| Compound | 231 | SUM159 | gp130ko | Metastatic |
| LLM418 | 25.1 | 16.3 | 22.5 | 23.7 |
| LLM418-Et | 17.5 | 13.5 | 18.2 | 16.8 |
| LLM418-Pr | 5.8 | ND | ND | ND |
| LLM418-iPr | 19.0 | 9.1 | 12.1 | ND |
| LLM418-CBu | 49.7 | 5.9 | 16.1 | 11.8 |
| LLM418-MCBu | 18.5 | 10.5 | 13.9 | 7.3 |
| LLM418-MCF₃ | 169 | 16.0 | ND | ND |
| LLM4 | 47.7 | 31.1 | ND | ND |
| LLM4-S1 | 53.1 | 58.7 | ND | ND |
| LLM4-S2 | 47.8 | 24.7 | ND | ND |
| LLM4-S3 | 49.1 | 46.1 | ND | ND |
| LS-TX-2 | Inactive | Inactive | ND | ND |
| LS-TX-3 | Inactive | Inactive | ND | ND |
| LS-TX-4 | Inactive | Inactive | ND | ND |
| LS-TF-4P | 18.4 | 26.1 | 26.7 | ND |
| LS-28-2 | 21%* | 33%* | ND | ND |
| LS-28-3 | 88%*** | Inactive | ND | ND |
| LLM418-SC | 11.3** | ND | ND | ND |

Inactive—Compounds displayed no measurable activity at 20 μM under the conditions used;
ND—Not Determined;
*Percent cells remaining after treatment with 20 μM compound;
**48-hour MTT;
***Percent cells remaining after treatment with 10 μM compound

TABLE 2
MTT IC₅₀ Values in BRCA Mutant Breast Cancer Cell Lines
IC₅₀ Values in BRCA Mutant Breast Cancer Cell Lines (μM)

| Compound | HCC1937 | SUM149 |
|---|---|---|
| LS-O1 | Inactive | Inactive |
| LS-O2 | Inactive | Inactive |
| LS-O3 | 42.0 | 100 |
| LS-O5 | 112 | Inactive |

Inactive—Compounds displayed no activity at 20 μM;
ND—Not Determined;
*Percent cells remaining after treatment with 20 μM compound;
**48-hour MTT

TABLE 3
MTT IC50 Values in Breast Cancer Cell Lines

| | | | MDA-MB-231 |
|---|---|---|---|
| | SUM159 | MDA-MB-231 | Bone Metastatic |
| Compound | IC₅₀ (μM) | IC₅₀ (μM) | IC₅₀ (μM) |
| LLM418 | 16.3 | 28.1 | 23.7 |
| LLM418-SC | ND | 11.3** | ND |
| Bazedoxifene | 7.1 | 12 | ND |
| LMT-28 | 37.8 | ND | 67.8 |
| LS-101-D | ND | ND | ND |
| LLM418-Az | ND | ND | ND |
| LLM418-Et | 17.4 | 17.5 | 16.8 |
| LLM418-Pr | ND | 5.8 | ND |
| LLM418-iPr | 9.1 | 19.0 | 29.7 |
| LLM418-MCBu | 10.5 | 18.5 | 7.3 |
| LLM418-CBu | 15.1 | 30.1 | 21.1 |
| LLM418-MCF3 | 16 | 169 | ND |
| LLM418-MOx | ND | ND | ND |
| LS-U1 | ND | ND | ND |
| LLM418-5NEt | ND | ND | ND |
| LLM418-FBn | ND | ND | ND |
| LLM418-CPr | ND | ND | ND |
| LS-28-2 | 33%* | 21%* | ND |
| LS-28-3 | Inactive | 88%*** | ND |
| LLM4 | 31.1 | 47.7 | ND |
| LLM4-S1 | 58.7 | 53.1 | ND |
| LLM4-S2 | 24.7 | 47.8 | ND |
| LLM4-S3 | 46.1 | 49.1 | ND |
| LS-AF-2P | 103 | 196 | ND |
| LS-AF-3P | 44 | 86 | ND |
| LS-AF-4P | ND | ND | ND |
| LS-TF-2P | 41 | 96 | ND |
| LS-TF-3P | 16 | 67 | ND |
| LS-TF-4P | 26.1 | 18.4 | 26.7 |
| LS-TX-2 | Inactive | Inactive | ND |
| LS-TX-3 | Inactive | Inactive | ND |
| LS-TX-4 | Inactive | Inactive | ND |
| LS-T-2 | ND | ND | ND |
| LS-T-3 | ND | ND | ND |
| LS-T-4 | ND | ND | ND |
| LS-Ac-2 | ND | ND | ND |

Inactive—Compounds displayed no measurable activity at 20 μM under the conditions used;
ND—Not Determined;
*Percent cells remaining after treatment with 20 μM compound;
**48-hour MTT;
***Percent cells remaining after treatment with 10 μM compound; Experiments performed in one set of batch studies are reported in Table 3. These batch studies are independent from another set of batch studies reported in Table 1.

### Selectivity Study

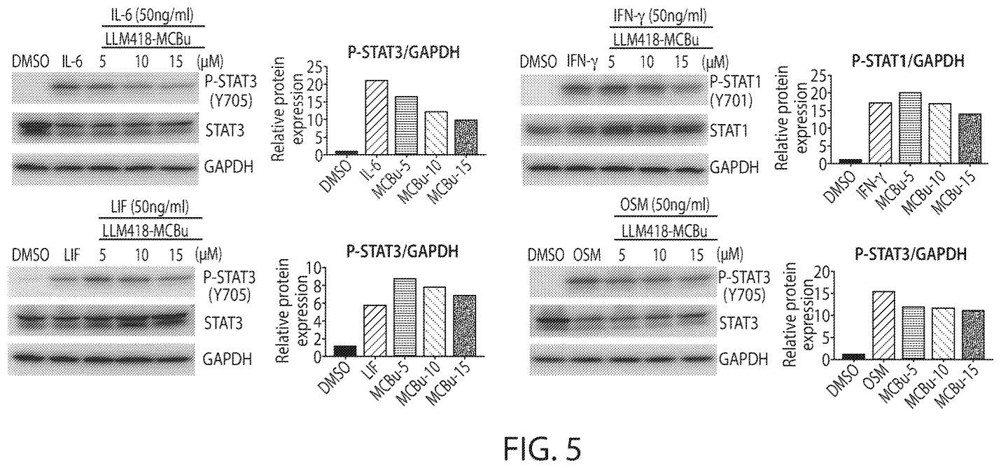

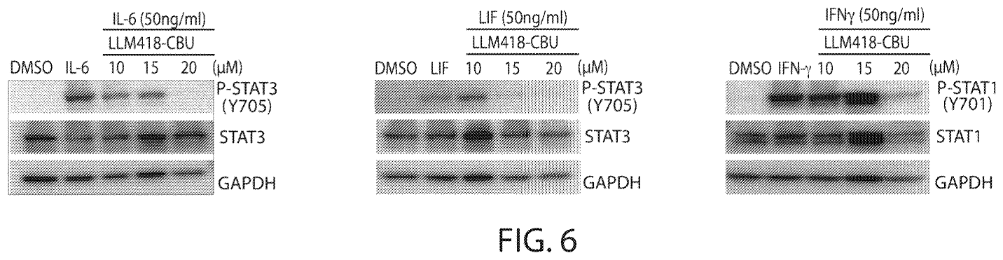

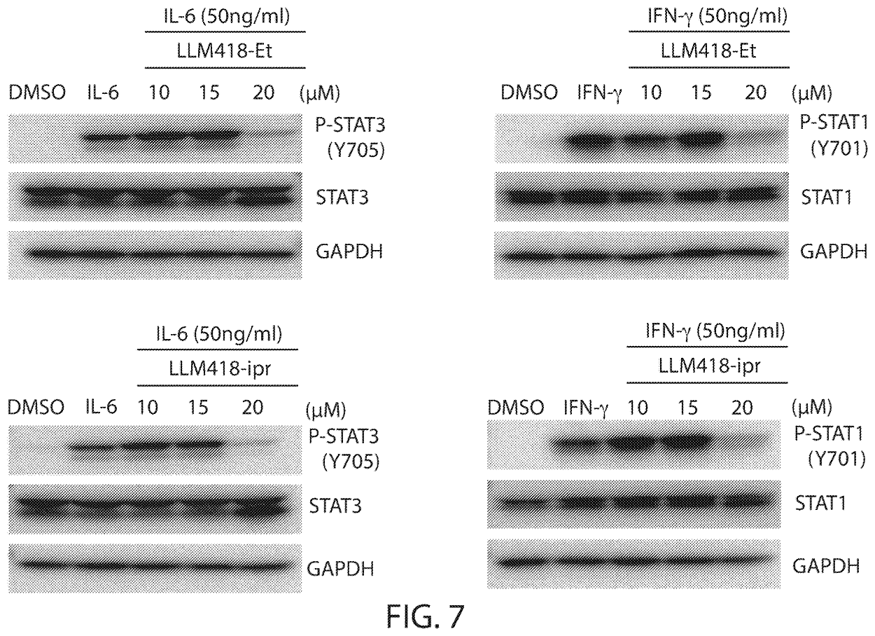

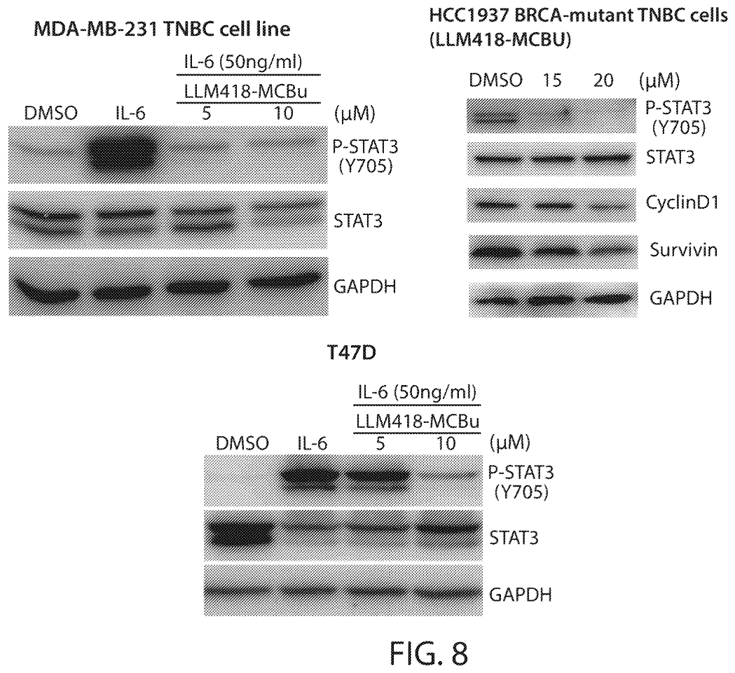

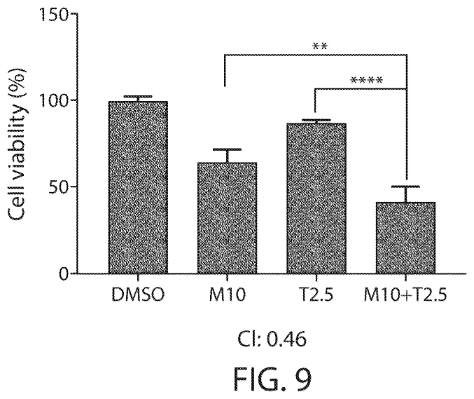

Inhibition of downstream STAT3 phosphorylation was performed. T47D breast cancer cells, which lack endogenous IL-6, were treated with LLM418-MCBu for 2 hours. Then, cytokine was administered for 30 minutes (IL-6, INF, LIF, or OSM). Western blot conducted afterwards to assess dose-dependent inhibition of IL-6 signaling (or lack thereof) (FIG. 5). As anticipated, LLM418-MCBu inhibited IL-6 selectively in a dose-dependent manner.

### DISCUSSION

IL-6 is a pleiotropic cytokine produced by a wide variety of cells in the body and is involved in the regulation of many different cellular processes^{1,2}. Due to this, it has a significant role in the progression of several disease states, including autoimmune diseases, inflammatory diseases, and cancer^{1,2}. Its involvement in these disease states makes it an attractive target.

However, the only currently available drugs are engineered proteins or monoclonal antibodies³, indicating a potential demand for a small molecule alternative. Towards this effort, the natural product Madindoline A (MDL-A) was discovered to be a selective IL-6 inhibitor in 1999⁵. However, later studies indicated that despite its great selectivity, MDL-A had poor activity and a low-yield total synthesis, making analogue development relatively undesirable^{3,6}.

MDL-A was determined to bind to the extracellular domain of glycoprotein 130 (gp130) with a K_{D} of 288 μM⁶. Of the different cytokines that bind to gp130, only IL-6 and IL-11 require two gp130 units and the formation of a hexameric structure for signaling, which is the driving force behind the selectivity of MDL-A^{3,9}. In order to conduct a structure-based drug design approach to developing small molecules with identical selectivity and superior activity, the precise binding mode of MDL-A to gp130 had to be determined. Through the use of docking and molecular dynamics simulations, the binding mode of MDL-A was identified⁹. Three “hot spots” were identified as playing a significant role in the binding of MDL-A to gp130⁹. These include a hydrophobic pocket, which is seen interacting with the aliphatic chain of MDL-A, tyrosine 94, which engages in pi-pi interactions, and asparagine 92, which engages in hydrogen bonding⁹. By maximizing interactions between these hot spots, more effective IL-6 inhibitors can be developed.

Based on the identification of MDL-A as a selective IL-6/gp130 protein-protein interaction inhibitor, a thorough structure-based drug design study has been conducted. With the culmination of several generations of IL-6 signaling inhibitors as well as drug repositioning efforts, a current generation of selective inhibitors has been developed, with lead compounds possessing single-digit micromolar activity. Additionally, the groundwork has been laid for the design and synthetic approach towards future generations, both with a basis on LLM4/LLM418 as well as on indole alkaloid natural products.

### Synthesis

Synthesis of the compounds herein can be performed using standard chemical synthesis methods and reagents, including as delineated in the figures herein and those described in Thomas Sorrell, Organic Chemistry, University Science Books, Sausalito, 1999; Michael B. Smith, March's Advanced Organic Chemistry, 7th Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3rd Edition, Cambridge University Press, Cambridge, 1987, all incorporated by reference.

### Example 1: (S)—N-(3-(JH-pyrazol-3-yl)phenyl)-4-(4-isopropyl-2-oxooxazolidin-3-yl)benzamide (LS-28-3)

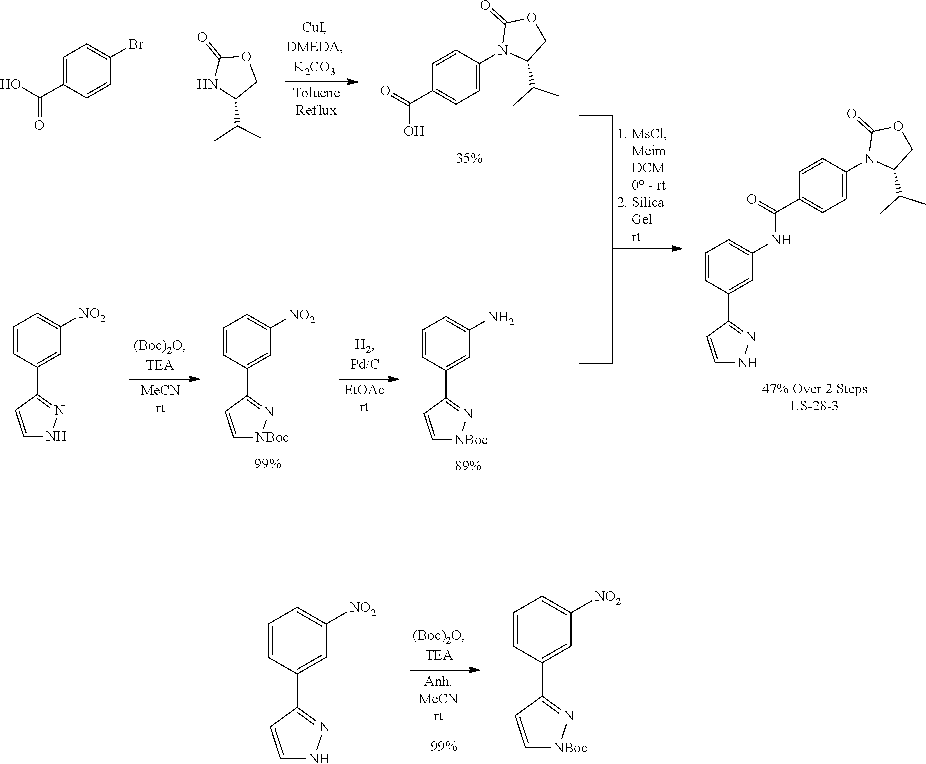

Tert-butyl 3-(3-nitrophenyl)-1H-pyrazole-1-carboxylate: To a 250 mL round-bottom flask was added 3-(3-nitrophenyl)-1H-pyrazole (1.089 g, 5.754 mmol, 1.0 eq), which was then dissolved in 60 mL anhydrous acetonitrile. Di-tert-butyl dicarbonate (1.639 g, 7.512 mmol, 1.3 eq) was then added, followed by triethylamine (1.60 mL, 11.48 mmol, 2.0 eq). The solution was then allowed to stir at room temperature for 44 hours. Upon completion, the solvent was removed under reduced pressure to afford the desired product as a light-yellow solid (1.653 g, 5.714 mmol, 99%). ¹H NMR: (600 MHz, CDCl₃) δ 8.70 (t, J=2.0 Hz, 1H), 8.31-8.27 (m, 1H), 8.23 (ddd, J=8.2, 2.3, 1.1 Hz, 1H), 8.16 (d, J=2.8 Hz, 1H), 7.61 (t, J=8.0 Hz, 1H), 6.79 (d, J=2.8 Hz, 1H), 1.69 (s, 9H). ¹³C NMR: (151 MHz, CDCl₃) δ 153.2, 148.8, 147.5, 134.0, 132.6, 132.3, 129.8, 123.7, 121.5, 106.4, 86.2, 28.1.

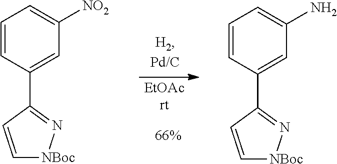 [SMILES: CC(C)(C)OC(=O)n1ccc(-c2cccc([N+](=O)[O-])c2)n1 ; CC(C)(C)OC(=O)n1ccc(-c2cccc(N)c2)n1]

Tert-butyl 3-(3-aminophenyl)-1H-pyrazole-1-carboxylate: To a 100 mL round-bottom flask was added tert-butyl 3-(3-nitrophenyl)-1H-pyrazole-1-carboxylate (503.0 mg, 1.739 mmol, 1.0 eq) and palladium on charcoal (10 wt. %, 56.3 mg, 0.053 mmol Pd, 0.030 eq). Ethyl acetate (45 mL) was added to the flask, and the headspace was evacuated and filled three times with hydrogen. The mixture was then stirred at room temperature under a hydrogen atmosphere for 96 hours, after which the mixture was filtered over celite and evaporated. The crude material was then purified via automated flash column chromatography using a gradient of 0-20% ethyl acetate in hexanes to afford the desired product as a viscous yellow oil (332.9 mg, 1.151 mmol, 66%). ¹H NMR: (600 MHz, CDCl₃) δ 8.08 (d, J=2.8 Hz, 1H), 7.33 (t, J=2.0 Hz, 1H), 7.24 (dt, J=7.7, 1.4 Hz, 1H), 7.20 (t, J=7.7 Hz, 1H), 6.70 (ddd, J=7.8, 2.4, 1.1 Hz, 1H), 6.67 (d, J=2.8 Hz, 1H), 3.78 (s, 2H), 1.67 (s, 9H). ¹³C NMR: (151 MHz, DMSO) δ 155.1, 149.0, 147.1, 132.5, 132.1, 129.2, 114.7, 114.0, 110.9, 106.6, 84.7, 27.5.

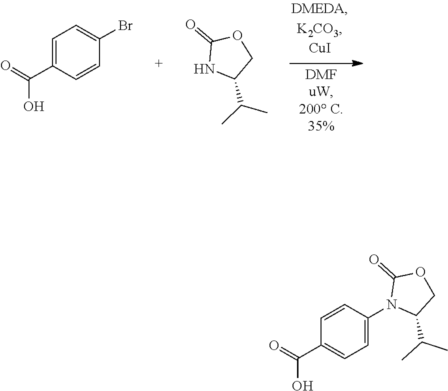

(S)-4-(4-isopropyl-2-oxooxazolidin-3-yl)benzoic acid: To a 20 mL microwave vial was added 4-bromobenzoic acid (561.8 mg, 2.795 mmol, 1.20 eq), (S)-4-isopropyl-2-oxazolidinone (301.4 mg, 2.334 mmol, 1.0 eq), potassium carbonate (1.287 g, 9.309 mmol, 4.0 eq), and copper(I) iodide (288.5 mg, 1.515 mmol, 0.65 eq). Anhydrous N,N-dimethyl formamide (10 mL) was then added, and the mixture was stirred. To the mixture was then added N,N′-dimethylethylenediamine (0.33 mL, 3.066 mmol, 1.3 eq). The microwave vial was sealed and the mixture was heated via microwave at 200° C. for 30 minutes. The mixture was then diluted with 50 mL H₂O and acidified to pH ˜1 with concentrated HCl. The aqueous mixture was then extracted with 3×100 mL portions of DCM. The organic layers were combined, dried over anhydrous Na₂SO₄, and rotovaped. The crude material was then purified via automated flash column chromatography using a gradient of 5-55% EtOAc in hexanes with a constant 1% AcOH additive to afford the desired product as a pale yellow solid (203.9 mg, 0.818 mmol, 35%). ¹H NMR: (600 MHz, CDCl₃) δ 8.15-8.11 (m, 2H), 7.67-7.62 (m, 2H), 4.51 (dt, J=8.9, 3.9 Hz, 1H), 4.44 (t, J=8.8 Hz, 1H), 4.29 (dd, J=8.8, 4.1 Hz, 1H), 2.29-2.18 (m, 1H), 0.96 (d, J=7.0 Hz, 3H), 0.85 (d, J=6.8 Hz, 3H). ¹³C NMR: (151 MHz, CDCl₃) δ 169.6, 155.3, 141.8, 131.4, 124.9, 120.3, 62.4, 59.9, 27.5, 17.8, 14.1.

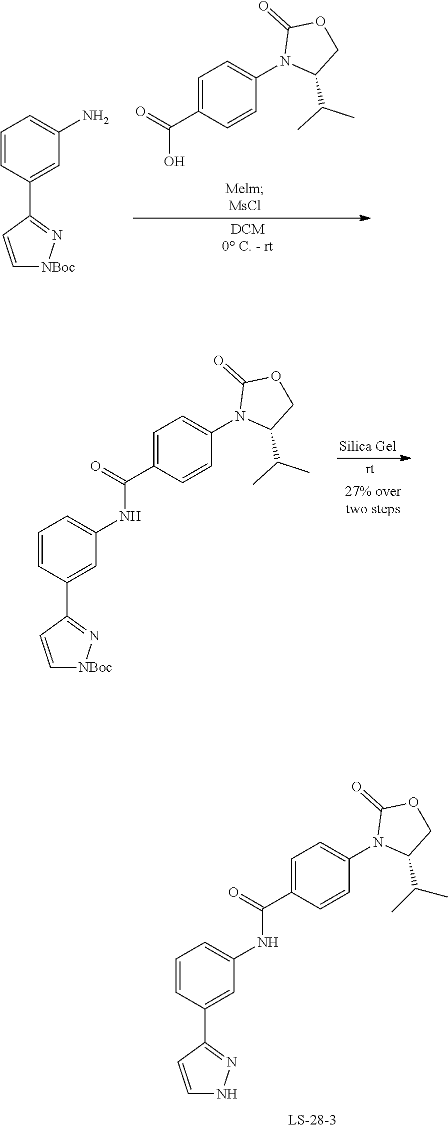 [SMILES: CC(C)(C)OC(=O)n1ccc(-c2cccc(N)c2)n1 ; CC(C)[C@H]1COC(=O)N1c1ccc(C(=O)O)cc1 ; CC(C)[C@H]1COC(=O)N1c1ccc(C(=O)Nc2cccc(-c3ccn(C(=O)OC(C)(C)C)n3)c2)cc1 ; CC(C)[C@H]1COC(=O)N1c1ccc(C(=O)Nc2cccc(-c3cc[nH]n3)c2)cc1]

(S)—N-(3-(1H-pyrazol-3-yl)phenyl)-4-(4-isopropyl-2-oxooxazolidin-3-yl)benzamide: To a vial was added (S)-4-(4-isopropyl-2-oxooxazolidin-3-yl)benzoic acid (76.0 mg, 0.305 mmol, 1.03 eq), which was then dissolved in 4 mL DCM. The solution was cooled to 0° C., and N-methyl imidazole (0.06 mL, 0.753 mmol, 2.5 eq) was added. The solution was stirred at 0° C. for 10 minutes. Then, a solution of methanesulfonyl chloride (0.025 mL, 0.323 mmol, 1.1 eq) in 0.5 mL DCM was added. The solution was stirred at 0° C. for 30 minutes. A solution of Tert-butyl 3-(3-aminophenyl)-1H-pyrazole-1-carboxylate (77.1 mg, 0.297 mmol, 1.0 eq) in 1.5 mL DCM was added. The solution was stirred overnight and allowed to gradually warm to room temperature. After 24 hours, the reaction was quenched with 20 mL ice-cold H₂O and extracted with 2×30 mL portions of DCM. The organic layers were combined, filtered over anhydrous Na₂SO₄, and concentrated. The crude material was then dry loaded onto silica gel and allowed to react over a period of days at room temperature. The crude deprotected product was suspended in DCM/Acetone and separated from silica gel via vacuum filtration. The filtrate was then purified via prep TLC using 1:1 Hex:EtOAc to afford the desired product as a pale yellow solid (30.8 mg, 0.0789 mmol, 27%). ¹H NMR: (600 MHz, CDCl₃) δ 8.07-8.02 (m, 1H), 7.96-7.89 (m, 3H), 7.75-7.69 (m, 1H), 7.67-7.61 (m, 3H), 7.55 (d, J=7.7 Hz, 1H), 7.44 (t, J=7.9 Hz, 1H), 6.67 (d, J=2.3 Hz, 1H), 4.51 (dt, J=8.9, 3.9 Hz, 1H), 4.46 (t, J=8.8 Hz, 1H), 4.30 (dd, J=8.7, 4.2 Hz, 1H), 2.26-2.19 (m, 1H), 0.96 (d, J=7.1 Hz, 3H), 0.86 (d, J=6.8 Hz, 3H).

### Example 2: (S)—N-(2-(JH-pyrazol-3-yl)phenyl)-4-(4-isopropyl-2-oxooxazolidin-3-yl)benzamide (LS-28-2)

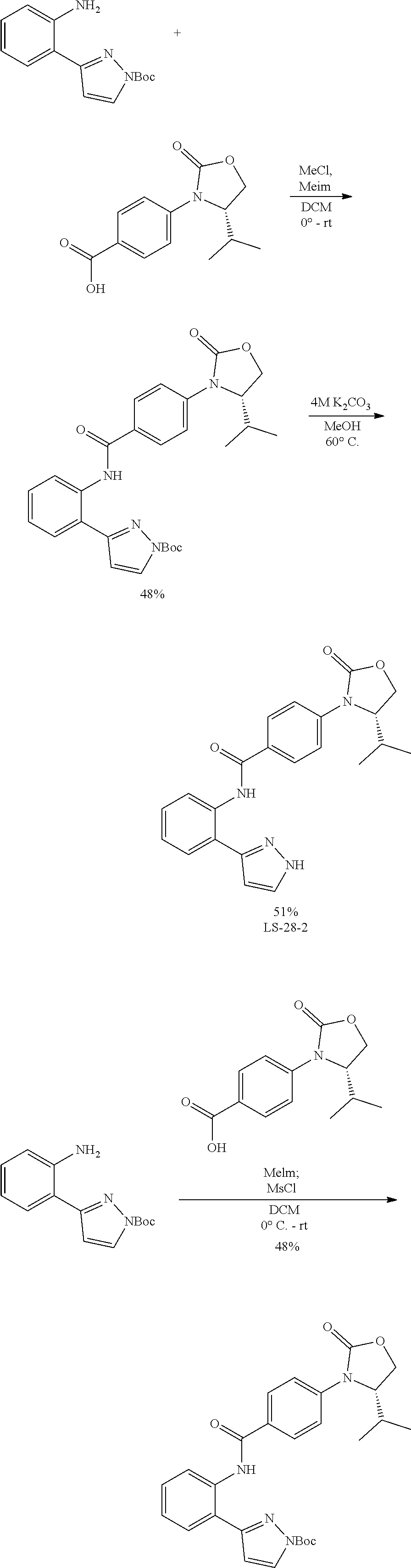

Tert-butyl (S)-3-(2-(4-(4-isopropyl-2-oxooxazolidin-3-yl)benzamido)phenyl)-1H-pyrazole-1-carboxylate: To a vial was added (S)-4-(4-isopropyl-2-oxooxazolidin-3-yl)benzoic acid (75.2 mg, 0.302 mmol, 1.0 eq), which was then dissolved in 4 mL DCM. The solution was then cooled to 0° C. in an ice bath, and N-methyl imidazole (0.06 mL, 0.753 mmol, 2.5 eq) was added. The solution was stirred for 10 minutes, after which a solution of methanesulfonyl chloride (0.025 mL, 0.323 mmol, 1.1 eq) in 0.5 mL DCM was added dropwise. The solution was then stirred for 30 minutes at 0° C. Then, a solution of tert-butyl 3-(2-aminophenyl)-1H-pyrazole-1-carboxylate (78.4 mg, 0.302 mmol, 1.0 eq) in 1 mL DCM was added. The solution was removed from the ice bath and allowed to slowly warm to room temperature as it stirred for 24 hours. After completion of the reaction, it was quenched with 20 mL ice cold H₂O and extracted with 2×30 mL portions of DCM. The organic layers were combined, dried over anhydrous Na₂SO₄, filtered, and concentrated under reduced pressure. The crude material was then purified via automated flash column chromatography using a gradient of 5-60% EtOAc in hexanes to afford the desired product as a pale orange solid (71.3 mg, 0.145 mmol, 48%). ¹H NMR: (600 MHz, CDCl₃) δ 12.22 (s, 1H), 8.95 (dd, J=8.5, 1.2 Hz, 1H), 8.46-8.36 (m, 2H), 8.11 (d, J=2.9 Hz, 1H), 7.72-7.64 (m, 3H), 7.45 (ddd, J=8.6, 7.2, 1.6 Hz, 1H), 7.17 (td, J=7.6, 1.3 Hz, 1H), 6.82 (d, J=2.9 Hz, 1H), 4.52 (dd, J=8.5, 4.5 Hz, 1H), 4.44 (t, J=8.8 Hz, 1H), 4.29 (dd, J=8.8, 4.3 Hz, 1H), 2.31-2.23 (m, 1H), 1.68 (s, 9H), 0.95 (d, J=7.0 Hz, 3H), 0.86 (d, J=6.8 Hz, 3H). ¹³C NMR: (151 MHz, CDCl₃) δ 165.0, 155.5, 155.2, 146.8, 139.9, 137.5, 131.5, 130.9, 130.1, 129.3, 128.4, 123.1, 121.1, 120.8, 118.7, 107.5, 86.0, 62.4, 60.1, 28.0, 27.4, 17.8, 14.1.

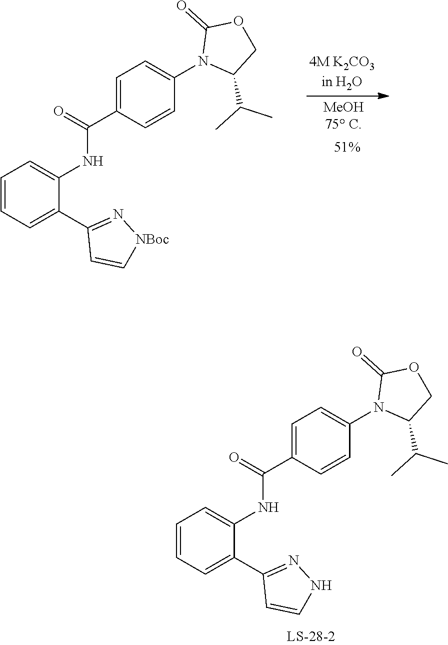 [SMILES: CC(C)[C@H]1COC(=O)N1c1ccc(C(=O)Nc2ccccc2-c2ccn(C(=O)OC(C)(C)C)n2)cc1 ; CC(C)[C@H]1COC(=O)N1c1ccc(C(=O)Nc2ccccc2-c2cc[nH]n2)cc1]

(S)—N-(2-(1H-pyrazol-3-yl)phenyl)-4-(4-isopropyl-2-oxooxazolidin-3-yl)benzamide: To a vial containing Tert-butyl (S)-3-(2-(4-(4-isopropyl-2-oxooxazolidin-3-yl)benzamido)phenyl)-1H-pyrazole-1-carboxylate (176.1 mg, 0.359 mmol) was added 10 mL methanol. The mixture was then stirred and heated to 75° C., after which 0.72 mL of a 4 M K₂CO₃ solution was added. The mixture was then stirred at 75° C. for 45 minutes. Upon completion, the reaction mixture was allowed to cool to room temperature and was then quenched with 10 mL saturated aqueous NH₄Cl, diluted with 25 mL H₂O, and extracted with 3×40 mL portions of DCM. The organic layers were combined, dried over anhydrous Na₂SO₄, filtered, and concentrated under reduced pressure. The crude material was then purified via automated flash column chromatography using a gradient of to afford the desired product as an off-white solid (71.8 mg, 0.184 mmol, 51%). ¹H NMR: (600 MHz, CDCl₃) δ 12.18 (s, 1H), 10.38 (s, 1H), 8.86 (dd, J=8.4, 1.2 Hz, 1H), 8.17-8.10 (m, 2H), 7.72 (dd, J=7.8, 1.6 Hz, 1H), 7.70 (d, J=2.5 Hz, 1H), 7.67-7.61 (m, 2H), 7.39 (ddd, J=8.5, 7.3, 1.6 Hz, 1H), 7.17 (td, J=7.6, 1.2 Hz, 1H), 6.77 (d, J=2.5 Hz, 1H), 4.52 (dt, J=8.9, 4.0 Hz, 1H), 4.46 (t, J=8.8 Hz, 1H), 4.30 (dd, J=8.7, 4.4 Hz, 1H), 2.27-2.19 (m, 1H), 0.95 (d, J=7.1 Hz, 3H), 0.86 (d, J=6.8 Hz, 3H).

### Example 3: 4-(2-(piperidin-1-yl)ethoxy)-N-(2-(1-((2R,3R,4S,5R)-3,4,5-trihydroxytetrahydro-2H-pyran-2-yl)-1H-1,2,3-triazol-4-yl)phenyl)benzamide·acetic acid (LS-TX-2)

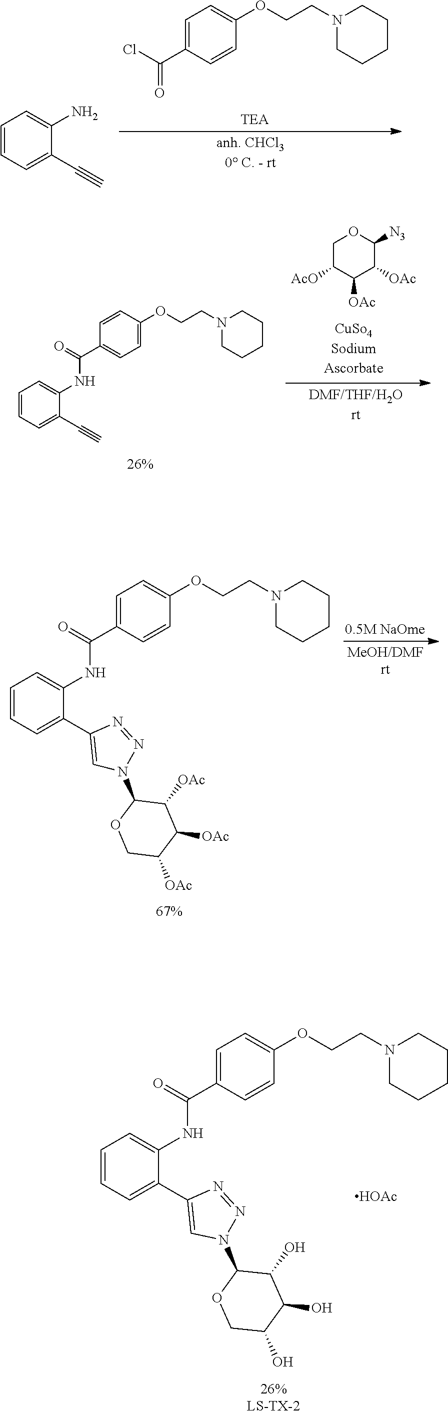

### Example 4: 4-(2-(piperidin-1-yl)ethoxy)-N-(3-(1-((2R,3R,4S,5R)-3,4,5-trihydroxytetrahydro-2H-pyran-2-yl)-1H-1,2,3-triazol-4-yl)phenyl)benzamide·acetic acid (LS-TX-3)

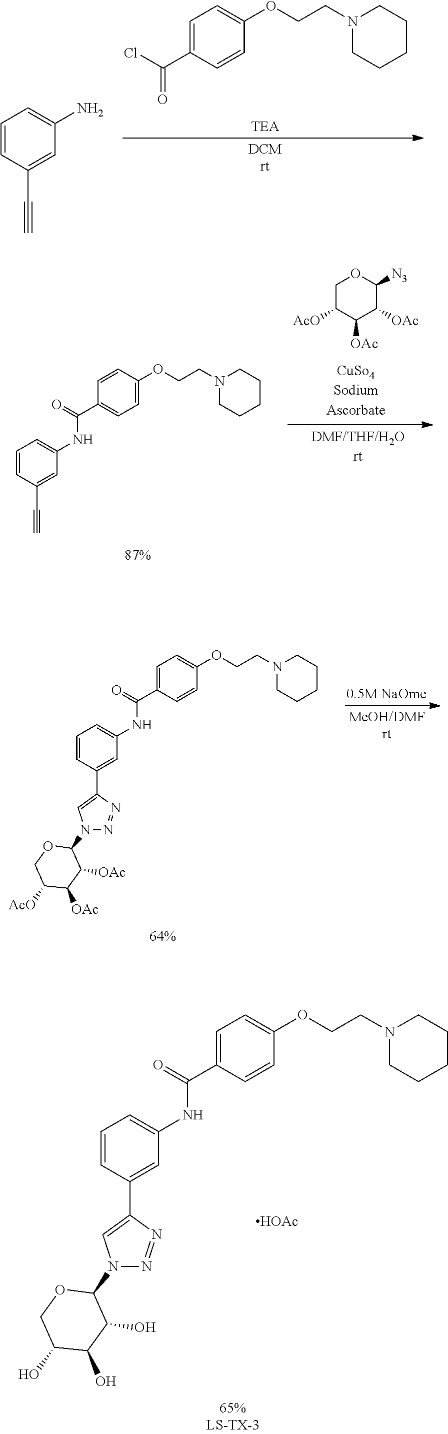

### Example 5: 4-(2-(piperidin-1-yl)ethoxy)-N-(4-(1-((2R,3R,4S,5R)-3,4,5-trihydroxytetrahydro-2H-pyran-2-yl)-1H-1,2,3-triazol-4-yl)phenyl)benzamide·acetic acid (LS-TX-4)

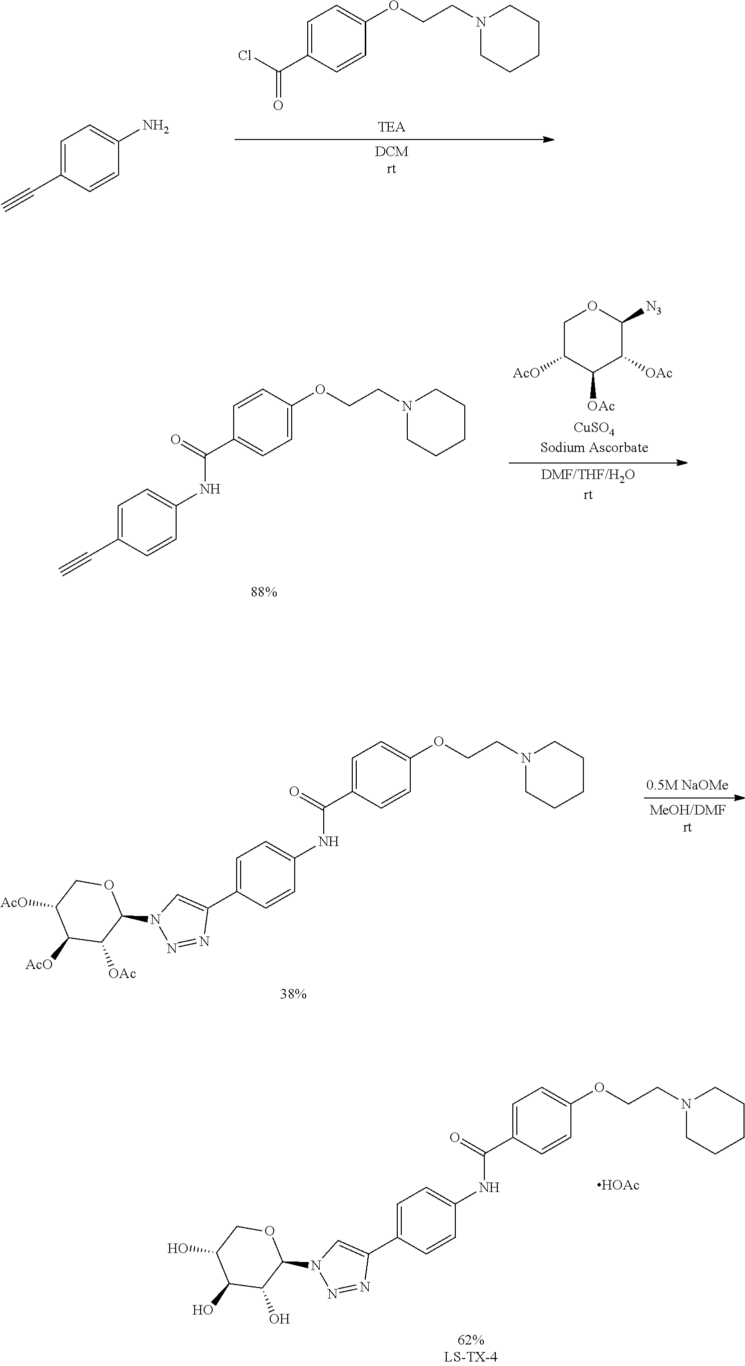

### Example 6: N-(2-oxo-2,3-dihydro-1H-benzo[d]imidazol-4-yl)-4-(2-(piperidin-1-yl)ethoxy)benzamide (LS-101-D)

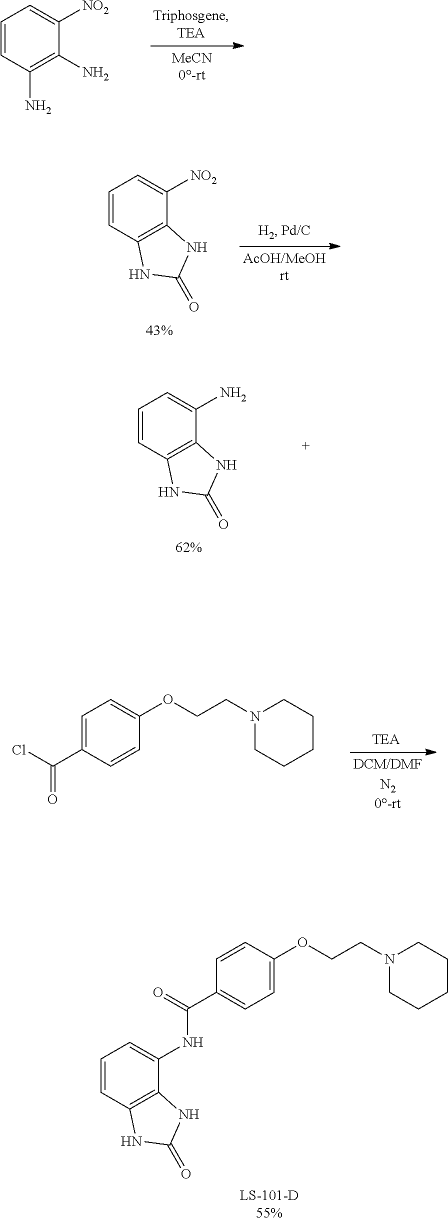 [SMILES: Nc1cccc([N+](=O)[O-])c1N ; O=c1[nH]c2cccc([N+](=O)[O-])c2[nH]1 ; Nc1cccc2[nH]c(=O)[nH]c12 ; O=C(Cl)c1ccc(OCCN2CCCCC2)cc1 ; O=C(Nc1cccc2[nH]c(=O)[nH]c12)c1ccc(OCCN2CCCCC2)cc1]

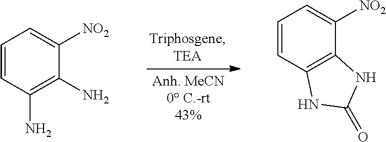 [SMILES: Nc1cccc([N+](=O)[O-])c1N ; O=c1[nH]c2cccc([N+](=O)[O-])c2[nH]1]

4-nitro-1,3-dihydro-2H-benzo[d]imidazol-2-one: To a vial was added 3-nitro-1,2-phenylenediamine (201.1 mg, 1.314 mmol, 1.0 eq), which was then dissolved in 5 mL anhydrous MeCN. The solution was cooled to 0° C., and triethylamine (0.37 mL, 2.655 mmol, 2.0 eq) was added. To the stirring solution was then added a solution of triphosgene (365.3 mg, 1.231 mmol, 0.94 eq) in anhydrous MeCN (3 mL) was added dropwise. The mixture was stirred overnight and allowed to slowly warm to room temperature. After 24 hours, the reaction mixture was filtered and the solid was washed with water. The crude product was then purified via automated flash column chromatography using a gradient of 0-8% MeOH in DCM to afford the desired product as a yellow solid (100.5 mg, 0.561 mmol, 43%). ¹H NMR: (600 MHz, DMSO-d₆) δ 11.55 (s, 1H), 11.36 (s, 1H), 7.75 (d, J=8.6 Hz, 1H), 7.31 (d, J=7.6 Hz, 1H), 7.12 (t, J=8.1 Hz, 1H). ¹³C NMR (151 MHz, DMSO-d₆) δ 155.1, 132.4, 130.1, 125.9, 120.4, 115.3, 114.3.

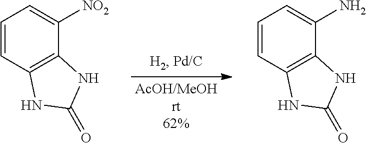 [SMILES: O=c1[nH]c2cccc([N+](=O)[O-])c2[nH]1 ; Nc1cccc2[nH]c(=O)[nH]c12]

4-amino-1,3-dihydro-2H-benzo[d]imidazol-2-one: To a vial was added 4-amino-1,3-dihydro-2H-benzo[d]imidazol-2-one (84.0 mg, 0.469 mmol, 1.0 eq), which was suspended in 10 mL MeOH and 2 mL AcOH. The mixture was stirred and palladium on charcoal (10 wt. %, 23.9 mg, 0.0225 mmol Pd, 0.05 eq) was added. The vial headspace was then evacuated and filled 3x with H₂, and the mixture was stirred at room temperature under an H₂ atmosphere overnight. After 25.25 hours, the mixture was filtered over celite and concentrated. The crude material was then dry loaded onto silica gel and purified via automated flash column chromatography using a gradient of 0-8% MeOH in DCM to afford the desired product as a red-brown solid (43.4 mg, 0.291 mmol, 62%). ¹H NMR: (600 MHz, DMSO-d₆) δ 10.32 (s, 1H), 9.97 (s, 1H), 6.65 (t, J=7.9 Hz, 1H), 6.25 (dd, J=8.0, 0.9 Hz, 1H), 6.21 (d, J=7.6 Hz, 1H), 4.84 (s, 2H). ¹³C NMR: (151 MHz, DMSO-d₆) δ 154.8, 131.4, 130.0, 121.3, 116.0, 106.8, 97.9.

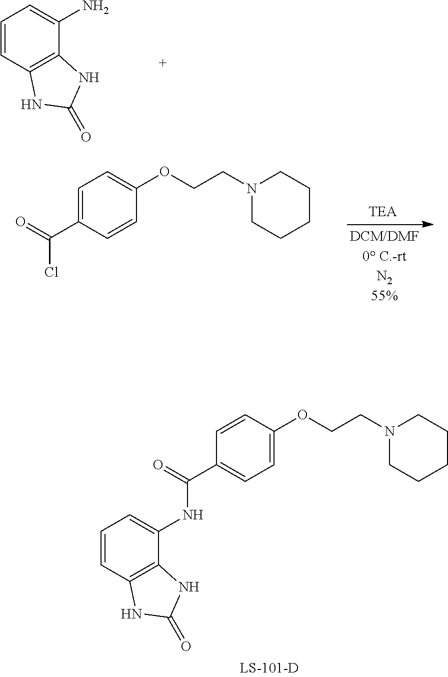

N-(2-oxo-2,3-dihydro-1H-benzo[d]imidazol-4-yl)-4-(2-(piperidin-1-yl)ethoxy)benzamide: To an oven-dried 50 mL round bottom flask was added 4-amino-1,3-dihydro-2H-benzo[d]imidazol-2-one (201.4 mg, 1.350 mmol, 1.0 eq), which was dissolved in 7 mL anhydrous DMF. Triethylamine (0.56 mL, 4.018 mmol, 3.0 eq) was added. The solution was cooled to 0° C. and the flask headspace was evacuated and filled with N₂ three times. To the solution was then added a freshly prepared solution of 4-(2-(piperidin-1-yl)ethoxy)benzoyl chloride (430.8 mg, 1.609 mmol, 1.2 eq) in 4 mL DCM. The solution was then stirred under N₂ overnight and allowed to slowly warm to room temperature. After 17.5 hours, the reaction was quenched with 22 mL saturated aqueous K₂CO₃ and extracted with 110 mL DCM. The organic layer was separated and concentrated. The crude material was purified via automated flash column chromatography using a gradient of 99:0:1 to 89:10:1 EtOAc:MeOH:TEA to afford the desired product as an off-white solid (285.0 mg, 0.749 mmol, 55%). ¹H NMR: (600 MHz, DMSO-d₆) δ 10.65 (s, 1H), 10.36 (s, 1H), 9.74 (s, 1H), 7.96 (d, J=8.4 Hz, 2H), 7.17 (d, J=7.9 Hz, 1H), 7.07 (d, J=8.3 Hz, 2H), 6.92 (t, J=7.9 Hz, 1H), 6.77 (d, J=7.6 Hz, 1H), 4.23-4.10 (m, 2H), 2.74-2.61 (m, 2H), 2.48-2.39 (m, 4H), 1.59-1.44 (m, 4H), 1.44-1.34 (m, 2H). ¹³C NMR: (151 MHz, DMSO) δ 164.7, 161.1, 154.8, 130.5, 129.8, 126.7, 123.2, 120.9, 120.2, 116.0, 114.0, 105.4, 65.7, 57.1, 54.3, 25.4, 23.7.

### Example 7: N-(2-(JH-pyrazol-5-yl)phenyl)-4-(2-(piperidin-1-yl)ethoxy)benzamide (LLM418)

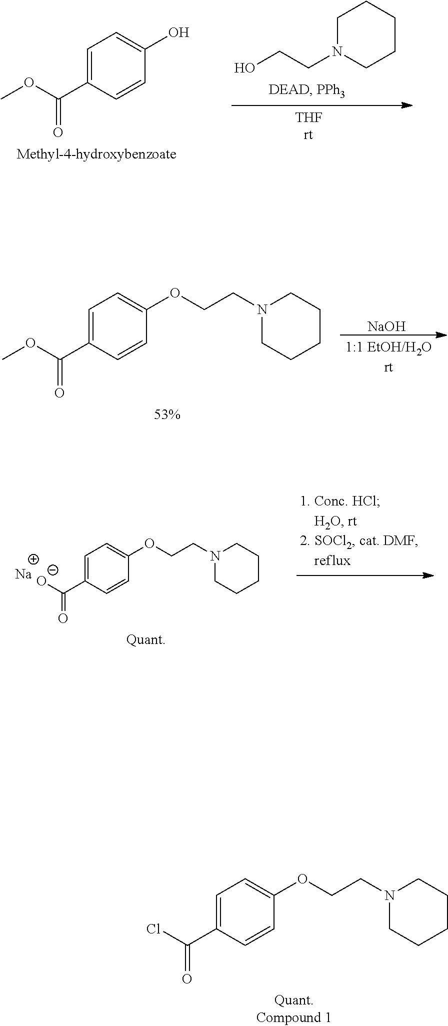

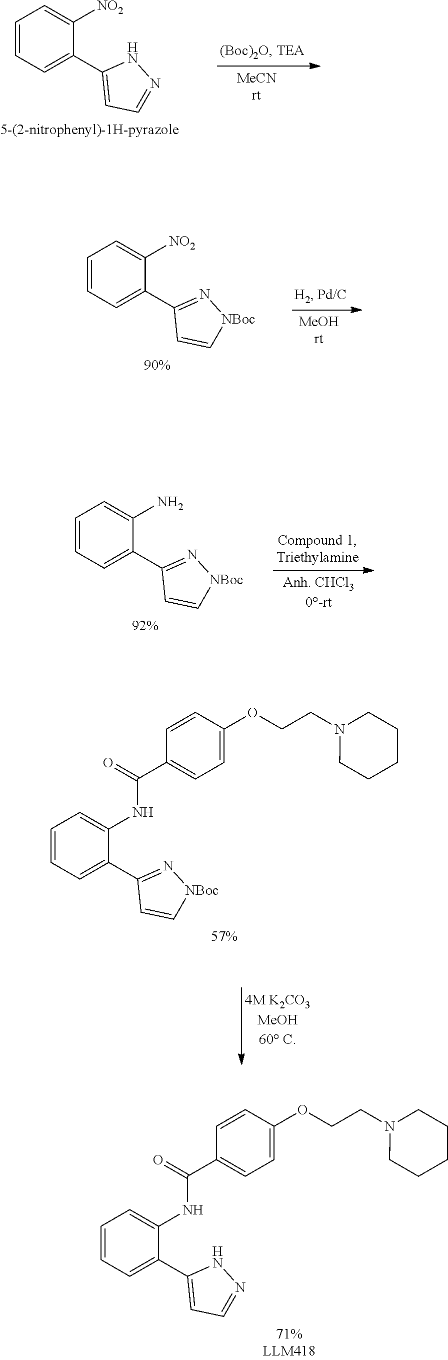 [SMILES: O=[N+]([O-])c1ccccc1-c1ccn[nH]1 ; CC(C)(C)OC(=O)n1ccc(-c2ccccc2[N+](=O)[O-])n1 ; CC(C)(C)OC(=O)n1ccc(-c2ccccc2N)n1 ; CC(C)(C)OC(=O)n1ccc(-c2ccccc2NC(=O)c2ccc(OCCN3CCCCC3)cc2)n1 ; O=C(Nc1ccccc1-c1ccn[nH]1)c1ccc(OCCN2CCCCC2)cc1]

### Example 8: 5-(4-(2-(piperidin-1-yl)ethoxy)phenyl)pyrazolo[1,5-c]quinazoline (LLM418-SC)

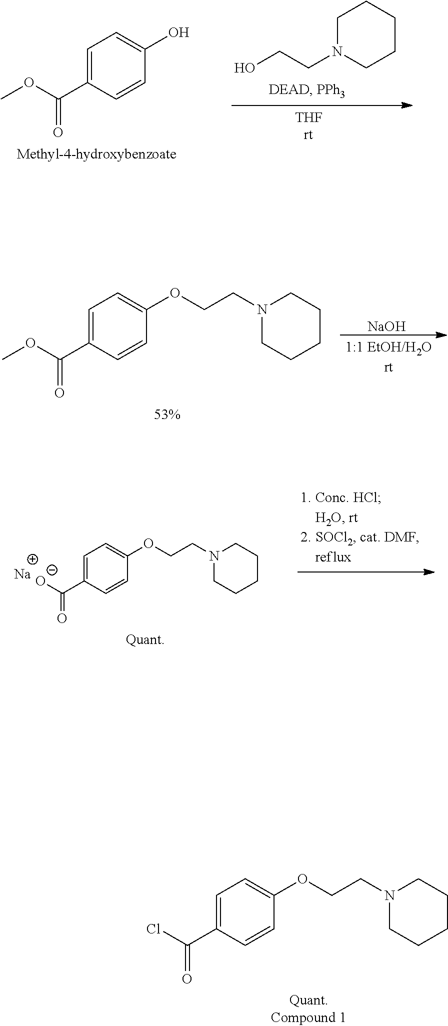

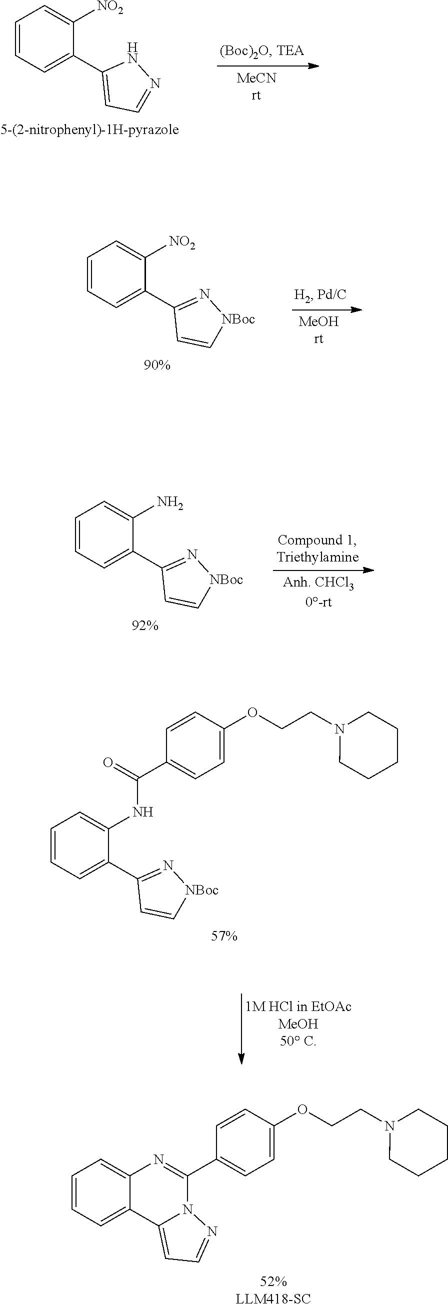 [SMILES: O=[N+]([O-])c1ccccc1-c1ccn[nH]1 ; CC(C)(C)OC(=O)n1ccc(-c2ccccc2[N+](=O)[O-])n1 ; CC(C)(C)OC(=O)n1ccc(-c2ccccc2N)n1 ; CC(C)(C)OC(=O)n1ccc(-c2ccccc2NC(=O)c2ccc(OCCN3CCCCC3)cc2)n1 ; c1ccc2c(c1)nc(-c1ccc(OCCN3CCCCC3)cc1)n1nccc21]

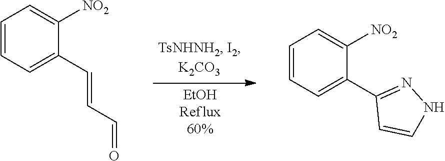 [SMILES: O=C/C=C/c1ccccc1[N+](=O)[O-] ; O=[N+]([O-])c1ccccc1-c1cc[nH]n1]

3-(2-nitrophenyl)-1H-pyrazole: To an oven-dried 250 mL round-bottom flask was added trans-2-nitrocinnamaldehyde (5.004 g, 28.25 mmol, 1.0 eq), para-toluenesulfonyl hydrazide (6.308 g, 33.87 mmol, 1.2 eq), and iodine (173.9 mg, 0.685 mmol, 0.024 eq). 150 mL ethanol was added, and the mixture was refluxed for 10 minutes, after which potassium carbonate (5.861 g, 42.40 mmol, 1.5 eq) was added. The mixture was then refluxed for a further 5 hours and 50 minutes, after which the mixture was filtered and the solvent was removed under reduced pressure. The crude material was then purified via automated flash column chromatography using a gradient of 0-9% ethyl acetate in dichloromethane and was then recrystallized from chloroform to afford the desired product as a brown solid (3.213 g, 16.98 mmol, 60%). ¹H NMR: (600 MHz, CDCl₃) δ 10.48 (s, 1H), 7.74-7.72 (m, 2H), 7.64 (d, J=2.3 Hz, 1H), 7.62-7.59 (m, 1H), 7.49-7.46 (m, 1H), 6.51 (d, J=2.3 Hz). ¹³C NMR: (151 MHz, CDCl₃) δ 149.2, 131.9, 130.9, 128.7, 123.7, 105.0.

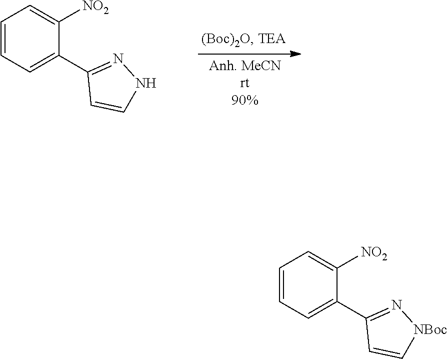 [SMILES: O=[N+]([O-])c1ccccc1-c1cc[nH]n1 ; CC(C)(C)OC(=O)n1ccc(-c2ccccc2[N+](=O)[O-])n1]

Tert-butyl 3-(2-nitrophenyl)-1H-pyrazole-1-carboxylate: To a round-bottom flask was added 5-(2-nitrophenyl)-1H-pyrazole (3.002 g, 15.87 mmol, 1.0 eq), triethylamine (4.5 mL, 32.28 mmol, 2.0 eq), di-tert-butyl dicarbonate (4.511 g, 20.67 mmol, 1.3 eq), and 58 mL anhydrous acetonitrile. The solution was then stirred at room temperature for 96 hours, after which the solvent was removed under reduced pressure. The crude material was then dry loaded onto silica gel and purified via automated flash column chromatography using a gradient of 0-35% ethyl acetate in hexanes to afford the desired product as a red solid (4.149 g, 14.34 mmol, 90%). ¹H NMR: (600 MHz, CDCl₃) δ 8.10 (d, J=2.8 Hz, 1H), 7.85 (dd, J=8.1, 1.3 Hz, 1H), 7.81 (dd, J=7.7, 1.4 Hz, 1H), 7.63 (ddd, J=7.6, 7.6, 1.3 Hz, 1H), 7.53 (ddd, J=8.1, 7.5, 1.4 Hz, 1H), 6.47 (d, J=2.8 Hz, 1H), 1.66 (s, 9H). ¹³C NMR: (150 MHz, CDCl₃) δ 151.7, 149.2, 147.4, 132.5, 131.8, 131.7, 129.7, 127.3, 124.1, 108.6, 85.9, 28.1. HRMS (ESI): calc. for C₁₄H₁₅N₃O₄ [M+Na]⁺: 312.0960, found: 312.0962; [2M+Na]⁺: 601.2023, found: 601.2042.

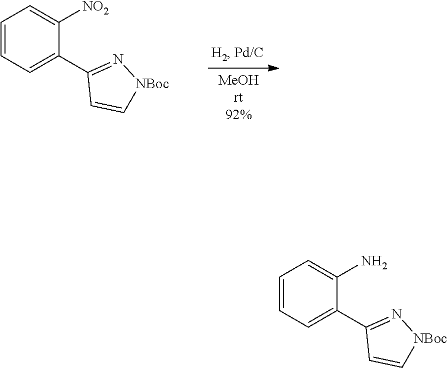 [SMILES: CC(C)(C)OC(=O)n1ccc(-c2ccccc2[N+](=O)[O-])n1 ; CC(C)(C)OC(=O)n1ccc(-c2ccccc2N)n1]

Tert-butyl 3-(2-aminophenyl)-1H-pyrazole-1-carboxylate: Tert-butyl 3-(2-nitrophenyl)-1H-pyrazole-1-carboxylate (4.080 g, 14.10 mmol, 1.0 eq) was dissolved in 100 mL methanol and added to a round-bottom flask. A suspension of palladium over carbon (10 wt. %, 452.0 mg, 0.42 mmol Pd, 0.030 eq) in 20 mL methanol was then added to the flask. While stirring, the flask atmosphere was evacuated and filled with hydrogen three times, after which the mixture was allowed to stir at room temperature under hydrogen for 5.4 hours. Upon completion, the mixture was filtered over celite and rotovaped to afford the desired product as a pale red solid (3.359 g, 12.29 mmol, 92%). ¹H NMR (600 MHz, CDCl₃) δ 8.08 (d, J=2.9 Hz, 1H), 7.53 (dd, J=7.8, 1.4 Hz, 1H), 7.15 (ddd, J=8.5, 7.3, 1.5 Hz, 1H), 6.77-6.72 (m, 3H), 5.74 (s, 2H), 1.66 (s, 9H). ¹³C NMR: (151 MHz, CDCl₃) δ 156.1, 147.7, 146.0, 130.8, 129.8, 128.7, 116.9, 116.6, 114.3, 107.0, 85.1, 28.1. LRMS (ESI): 290 [M+H]⁺.

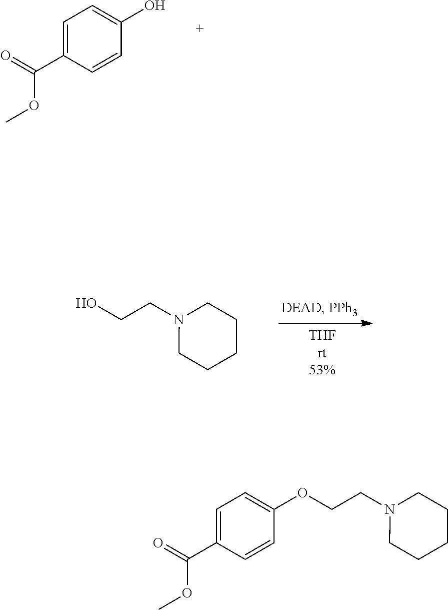 [SMILES: COC(=O)c1ccc(O)cc1 ; OCCN1CCCCC1 ; COC(=O)c1ccc(OCCN2CCCCC2)cc1]

Methyl 4-(2-(piperidin-1-yl)ethoxy)benzoate: To a round-bottom flask containing 145 mL tetrahydrofuran was added methyl-4-hydroxybenzoate (2.001 g, 13.15 mmol, 1.0 eq), triphenylphosphine (3.142 g, 11.98 mmol, 0.91 eq), and 1-(2-hydroxyethyl) piperidine (1.59 mL, 11.97 mmol, 0.91 eq). The solution was then stirred at room temperature while diethylazodicarboxylate (DEAD, 40 wt. % in toluene) (5.21 mL, 11.44 mmol, 0.87 eq) was added dropwise. After 1.5 hours, additional triphenylphosphine (3.145 g, 11.99 mmol, 0.91 eq) and 1-(2-hydroxyethyl) piperidine (1.59 mL, 11.97 mmol, 0.91 eq) were added to the reaction solution, followed by dropwise addition of DEAD (5.21 mL, 11.44 mmol, 0.87 eq). The reaction was then allowed to continue for 7.7 days, after which the solvent was removed under reduced pressure to afford a crude gel. The crude material was then suspended in a 150 mL solution of 2:1 EtOAc:Hexanes and vacuum filtered. The filtrate was then rotovaped to afford a crude yellow oil. The crude oil was then dry loaded onto silica gel and purified via automated flash column chromatography using a gradient of 2-15% ethyl acetate in hexanes with a constant 2% triethylamine additive to afford the desired product as a clear oil (1.829 g, 6.95 mmol, 53%). ¹H NMR: (600 MHz, DMSO-d₆) δ 7.90-7.88 (m, 2H), 7.05-7.03 (m, 2H), 4.13 (t, J=5.9, 2H), 3.80 (s, 3H), 2.65 (t, J=5.9 Hz, 2H), 2.46-2.36 (m, 4H), 1.48 (p, J=5.6 Hz, 4H), 1.40-1.32 (m, 2H). ¹³C NMR: (151 MHz, DMSO-d₆) δ 165.9, 162.4, 131.2, 121.7, 114.5, 66.0, 57.2, 54.4, 51.8, 25.6, 23.9. LRMS (ESI): 265 [M+H]⁺.

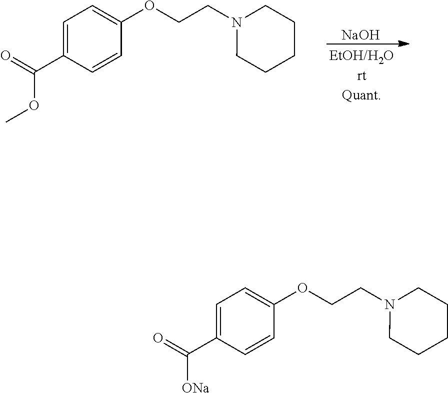

Sodium 4-(2-(piperidin-1-yl)ethoxy)benzoate: Methyl 4-(2-(piperidin-1-yl)ethoxy)benzoate (1.734 g, 6.59 mmol, 1.0 eq) was added to a flask and dissolved in 15 mL ethanol. Then, 15 mL 0.9 M aqueous NaOH solution (13.5 mmol, 2.0 eq) was added. The solution was then stirred at room temperature for 43 hours. Upon completion, the solvent was removed via rotovap, and the salt was dried overnight under vacuum while heating to 60° C. This afforded the desired product as a white solid (2.014 g, quantitative yield). ¹H NMR: (600 MHz, DMSO-d₆) δ 7.81-7.79 (m, 2H), 6.80-6.78 (m, 2H), 4.04 (t, J=5.9 Hz, 2H), 2.63 (t, J=5.9 Hz, 2H), 2.46-2.38 (m, 4H), 1.49 (p, J=5.6 Hz 4H), 1.40-1.33 (m, 2H). ¹³C NMR: (150 MHz, DMSO-d₆) δ 169.9, 159.1, 133.0, 130.6, 112.7, 65.5, 57.5, 54.4, 25.6, 24.0. LRMS (ESI): 250 [M+H]⁻, 248 [M−H]⁻.

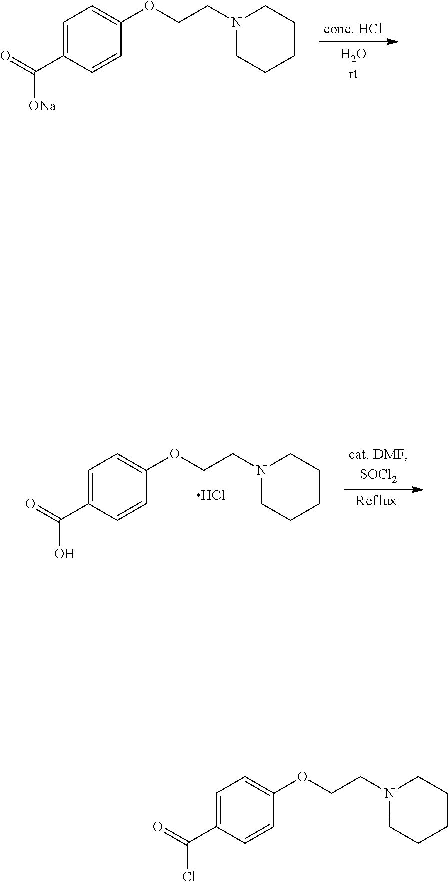

4-(2-(piperidin-1-yl)ethoxy)benzoyl chloride: Sodium 4-(2-(piperidin-1-yl)ethoxy)benzoate (504.7 mg, 1.86 mmol) was dissolved in 10 mL H₂O, and the solution was then acidified to pH 1 by addition of concentrated HCl. The acidic solution was then cooled in an ice bath. The crystallized solid was then dried under vacuum overnight while heating to 60° C. This afforded a 4-(2-(piperidin-1-yl)ethoxy)benzoic acid hydrochloride as a white solid in quantitative yield. The hydrogen chloride salt was then suspended in 10 mL thionyl chloride, with 4 drops anhydrous DMF added to the mixture. The mixture was then refluxed at 80° C. for 2 hours, after which the volatiles were removed via rotovap to afford the desired product as a pale yellow solid. The crude product was taken directly to the next step without purification.

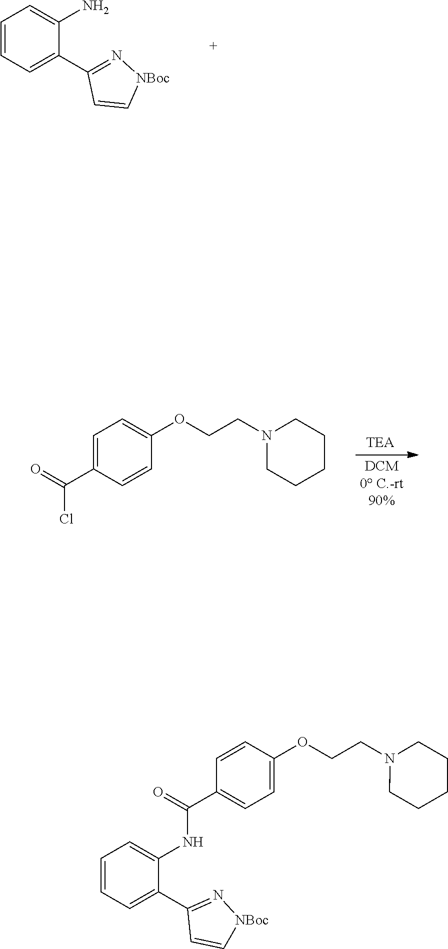 [SMILES: CC(C)(C)OC(=O)n1ccc(-c2ccccc2N)n1 ; O=C(Cl)c1ccc(OCCN2CCCCC2)cc1 ; CC(C)(C)OC(=O)n1ccc(-c2ccccc2NC(=O)c2ccc(OCCN3CCCCC3)cc2)n1]

Tert-butyl 3-(2-(4-(2-(piperidin-1-yl)ethoxy)benzamido)phenyl)-1H-pyrazole-1-carboxylate: To an oven-dried vial was added tert-butyl 3-(2-aminophenyl)-1H-pyrazole-1-carboxylate (351.5 mg, 1.356 mmol, 1.0 eq), which was dissolved in 6 mL DCM. The solution was cooled to 0° C., and triethylamine (0.47 mL, 3.372 mmol, 2.5 eq) was added. To the stirring solution was then added a freshly prepared solution of 4-(2-(piperidin-1-yl)ethoxy)benzoyl chloride (722.9 mg, 2.700 mmol, 2.0 eq) in 6 mL DCM. The solution was stirred overnight and allowed to slowly warm to room temperature. After 73 hours, the reaction was quenched with 15 mL saturated aqueous K₂CO₃ and extracted with 60 mL DCM. The organic layer was separated and concentrated. The crude material was then purified via automated flash column chromatography using a gradient of 85:13:2 to 45:53:2 Hex:EtOAc:TEA to afford the desired product as an off-white solid (602.1 mg, 1.227 mmol, 90%). ¹H NMR: (600 MHz, CDCl₃) δ 12.10 (s, 1H), 8.96 (dd, J=8.4, 1.2 Hz, 1H), 8.34-8.32 (m, 2H), 8.10 (d, J=2.9 Hz, 1H), 7.69 (dd, J=7.9, 1.6 Hz, 1H), 7.43 (ddd, J=8.6, 7.3, 1.6 Hz, 1H), 7.15 (td, J=7.6, 1.3 Hz, 1H), 7.05-7.02 (m, 2H), 6.82 (d, J=2.9 Hz, 1H), 4.20 (t, J=6.1 Hz, 2H), 2.81 (t, J=6.1 Hz, 2H), 2.60-2.46 (m, 4H), 1.69 (s, 9H), 1.62 (p, J=5.7 Hz, 4H), 1.50-1.42 (m, 2H). ¹³C NMR: (151 MHz, CDCl₃) δ 165.4, 161.7, 155.3, 146.7, 137.7, 131.4, 130.1, 130.0, 128.3, 127.2, 122.8, 121.1, 118.5, 114.5, 107.4, 85.7, 66.1, 57.9, 55.1, 28.0, 26.0, 24.2.

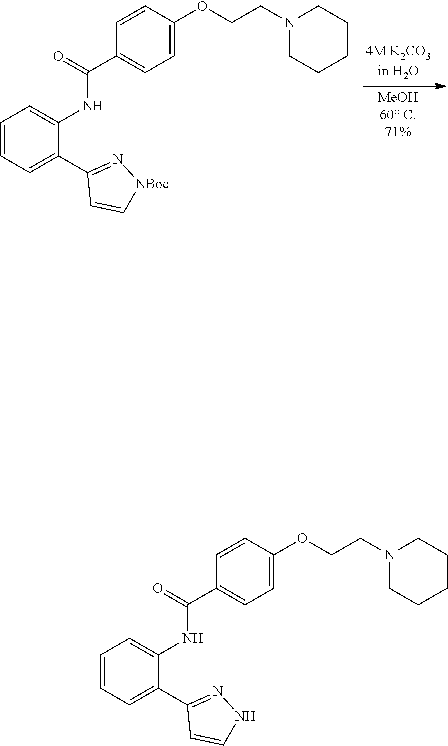 [SMILES: CC(C)(C)OC(=O)n1ccc(-c2ccccc2NC(=O)c2ccc(OCCN3CCCCC3)cc2)n1 ; O=C(Nc1ccccc1-c1cc[nH]n1)c1ccc(OCCN2CCCCC2)cc1]

N-(2-(1H-pyrazol-3-yl)phenyl)-4-(2-(piperidin-1-yl)ethoxy)benzamide (LLM418): To a vial containing tert-butyl 3-(2-(4-(2-(piperidin-1-yl)ethoxy)benzamido)phenyl)-1H-pyrazole-1-carboxylate (347.2 mg, 0.708 mmol, 1.0 eq) was added 10 mL methanol. The solution was stirred, and 1.42 mL 4 M K₂CO₃ (5.68 mmol, 8.0 eq) was added dropwise. The solution was then heated to 60° C. After 3 hours, the reaction was quenched with 15 mL saturated NH₄Cl. The mixture was diluted with 50 mL H₂O, and the organics were extracted with 3×300 mL portions of DCM. The organic layers were then combined, dried over anhydrous Na₂SO₄, filtered, and rotovaped. The compound was dry loaded onto silica gel and purified via automated flash column chromatography using a gradient of 50-100% EtOAc in hexanes, followed by 0-10% methanol in EtOAc, with a constant 2% triethylamine additive. The relevant fractions were combined and rotovaped to afford a yellow crystalline solid (197.5 mg, 0.506 mmol, 71%). ¹H NMR: (600 MHz, DMSO-d₆) δ 13.40 (s, 1H), 12.47 (s, 1H), 8.72-8.70 (m, 1H), 8.05-8.02 (m, 2H), 7.98-7.97 (m, 1H), 7.87 (m, 1H), 7.35-7.32 (m, 1H), 7.17-7.15 (m, 1H), 7.11-7.08 (m, 2H), 6.94-6.93 (m, 1H), 4.17 (t, J=5.9 Hz, 2H), 2.70 (t, J=5.6 Hz, 2H), 2.48-2.41 (m, 4H), 1.51 (p, J=5.6 Hz, 4H), 1.41-1.35 (m, 2H). ¹³C NMR: (150 MHz, DMSO-d₆) δ 164.3, 161.3, 150.2, 136.3, 130.1, 129.2, 128.0, 127.8, 127.1, 123.1, 120.4, 120.0, 114.6, 103.4, 65.9, 57.2, 54.4, 25.6, 23.9. LRMS (ESI): 391 [M+H].

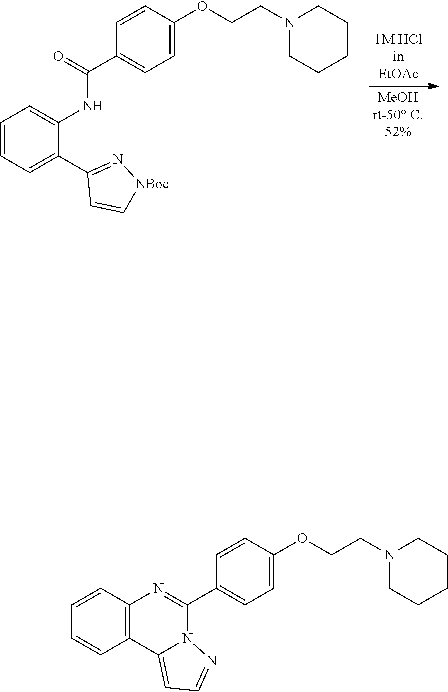 [SMILES: CC(C)(C)OC(=O)n1ccc(-c2ccccc2NC(=O)c2ccc(OCCN3CCCCC3)cc2)n1 ; c1ccc2c(c1)nc(-c1ccc(OCCN3CCCCC3)cc1)n1nccc21]

5-(4-(2-(piperidin-1-yl)ethoxy)phenyl)pyrazolo[1,5-c]quinazoline (LLM418-SC): Tert-butyl 3-(2-(4-(2-(piperidin-1-yl)ethoxy)benzamido)phenyl)-1H-pyrazole-1-carboxylate (148.6 mg, 0.30 mmol, 1.0 eq) was added to a vial and dissolved in 5 mL methanol. To the solution was added 5 mL 1M HCl in EtOAc (5.0 mmol, 16.7 eq). The solution was then stirred at room temperature for 1.5 hours, after which the solution was heated to 50° C. overnight. Upon completion, the reaction was quenched with 10 mL saturated aqueous NaHCO₃and extracted with 3×100 mL portions of DCM. The organic layers were then combined and rotovaped. The crude product was then dry loaded onto silica gel and twice purified via automated flash column chromatography using an EtOAc/Hexanes solvent system with 2% triethylamine. The relevant fractions were combined and the solvents were removed under reduced pressure to afford LLM418-SC as a viscous white oil (58.5 mg, 0.157 mmol, 52%). ¹H NMR (600 MHz, acetone-d₆) δ 8.65-8.62 (m, 2H), 8.26 (dd, J=8.0, 1.4 Hz, 1H), 8.19 (d, J=2.0 Hz, 1H), 7.98-7.95 (m, 1H), 7.72 (ddd, J=8.4, 7.1, 1.4 Hz, 1H), 7.64 (ddd, J=8.2, 7.1, 1.1 Hz, 1H), 7.32 (d, J=2.0 Hz, 1H), 7.16-7.14 (m, 2H), 4.25 (t, J=6.0 Hz, 2H), 2.77 (t, J=6.0 Hz, 2H), 2.56-2.47 (m, 4H), 1.57 (p, J=5.6 Hz, 4H), 1.46-1.39 (m, 2H). ¹³C NMR: (150 MHz, acetone-d₆) δ 162.3, 147.5, 143.9, 141.5, 140.7, 133.4, 130.6, 129.2, 128.3, 125.9, 124.1, 120.4, 114.6, 99.2, 67.3, 58.6, 55.8, 26.9, 25.1. LRMS (ESI): 373 [M+H]⁺.

### Example 9: LLM418-Et, LLM418-Pr, LLM418-iPr, LLM418-MCBu, LLM418-CBu, LLM418-CPr, LLM418-MCF3, and LLM418-MOx

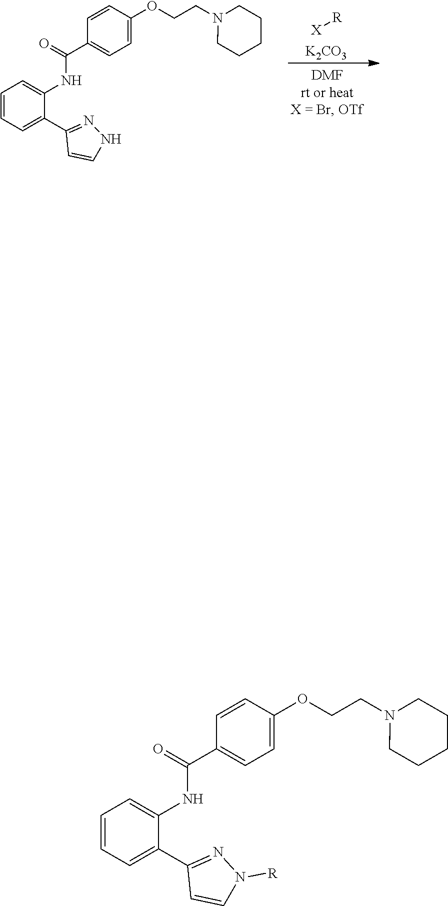

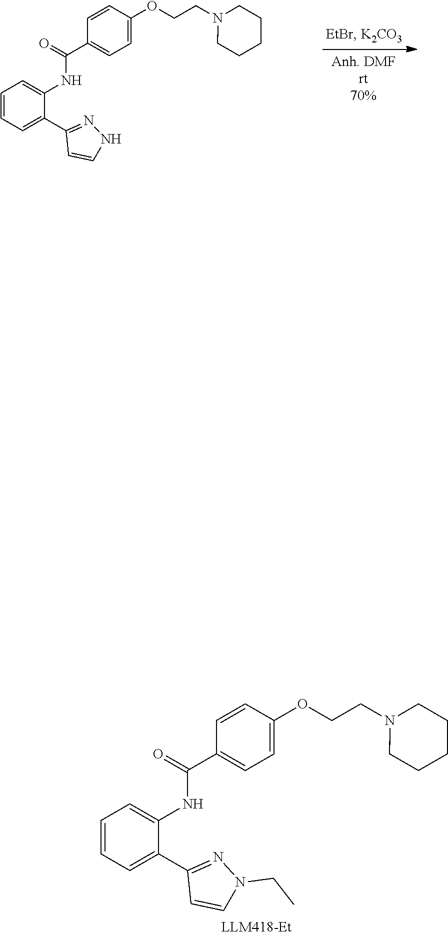 [SMILES: O=C(Nc1ccccc1-c1cc[nH]n1)c1ccc(OCCN2CCCCC2)cc1 ; CCn1ccc(-c2ccccc2NC(=O)c2ccc(OCCN3CCCCC3)cc2)n1]

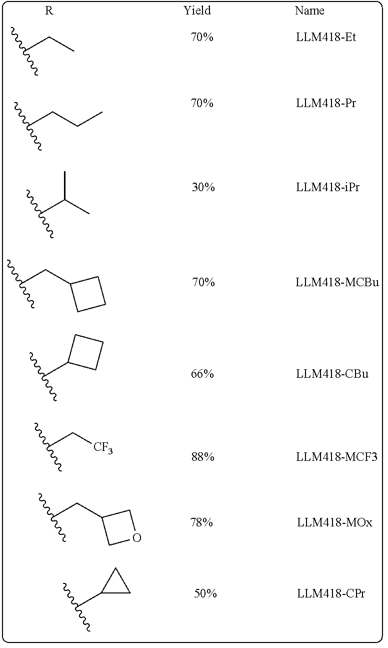

N-(2-(1-ethyl-1H-pyrazol-3-yl)phenyl)-4-(2-(piperidin-1-yl)ethoxy)benzamide: To a vial was added LLM418 (35.0 mg, 0.0896 mmol, 1.0 eq) and K₂CO₃ (37.9 mg, 0.274 mmol, 3.1 eq). Anhydrous DMF (3 mL) was added, and the mixture was stirred at room temperature for 30 minutes. To the mixture was then added slowly 0.5 mL of a freshly prepared solution of 0.2 M bromoethane in anhydrous DMF (0.10 mmol, 1.1 eq). The mixture was then stirred at room temperature overnight. After approximately 48 hours, the reaction mixture was diluted with 10 mL DCM and washed with 10 mL brine. The organic layer was separated and concentrated. The crude material was then purified via prep TLC using a solvent system of 1:1:0.04 Hex:EtOAc:TEA to afford the desired product as a pale brown solid (26.4 mg, 0.0631 mmol, 70%). ¹H NMR: (600 MHz, CDCl₃) δ 12.15 (s, 1H), 8.86 (dd, J=8.4, 1.2 Hz, 1H), 8.11-8.07 (m, 2H), 7.67 (dd, J=7.8, 1.5 Hz, 1H), 7.47 (d, J=2.3 Hz, 1H), 7.35 (ddd, J=8.6, 7.3, 1.6 Hz, 1H), 7.12 (td, J=7.5, 1.3 Hz, 1H), 7.01-6.97 (m, 2H), 6.65 (d, J=2.4 Hz, 1H), 4.27 (q, J=7.3 Hz, 2H), 4.19 (t, J=6.1 Hz, 2H), 2.81 (t, J=6.1 Hz, 2H), 2.59-2.46 (m, 4H), 1.63 (quint, J=5.7 Hz, 4H), 1.56 (t, J=7.3 Hz, 2H), 1.51-1.42 (m, 2H). ¹³C NMR (151 MHz, CDCl₃) δ 165.5, 161.5, 151.2, 136.8, 129.5, 129.4, 128.5, 128.5, 127.4, 123.0, 120.6, 120.3, 114.2, 104.0, 66.1, 57.8, 55.1, 47.2, 25.9, 24.2, 15.6.

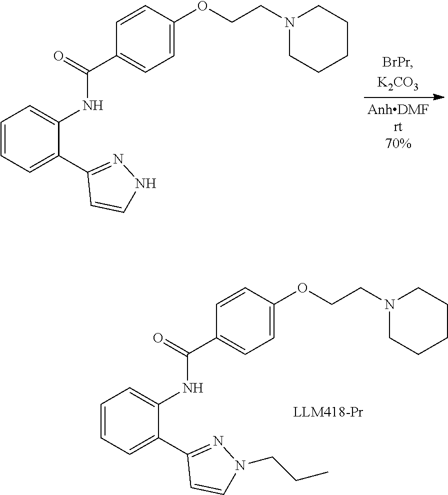 [SMILES: O=C(Nc1ccccc1-c1cc[nH]n1)c1ccc(OCCN2CCCCC2)cc1 ; CCCn1ccc(-c2ccccc2NC(=O)c2ccc(OCCN3CCCCC3)cc2)n1]

N-(2-(1-propyl-1H-pyrazol-3-yl)phenyl)-4-(2-(piperidin-1-yl)ethoxy)benzamide: To a vial was added LLM418 (40.1 mg, 0.103 mmol, 1.0 eq) and K₂CO₃ (44.3 mg, 0.321 mmol, 3.1 eq). Anhydrous DMF (3 mL) was added, and the mixture was stirred at room temperature for 30 minutes. To the mixture was then added dropwise 0.57 mL of a freshly prepared solution of 0.2 M 1-bromopropane (0.114 mmol, 1.1 eq) in anhydrous DMF. The mixture was then stirred at room temperature overnight. After 4.8 days, the reaction mixture was diluted with 15 mL DCM and washed with 15 mL brine. The organic layer was then separated and concentrated. The crude material was then purified via automated flash column chromatography using a gradient of 48:50:0:2 to 0:98:0:2 to 0:93:5:2 Hex:EtOAc:MeOH:TEA to afford the desired product as a light red solid (31.3 mg, 0.0724 mmol, 70%). ¹H NMR: (600 MHz, CDCl₃) δ 12.18 (s, 1H), 8.87 (dd, J=8.4, 1.2 Hz, 1H), 8.11-8.06 (m, 2H), 7.67 (dd, J=7.8, 1.6 Hz, 1H), 7.45 (d, J=2.4 Hz, 1H), 7.35 (ddd, J=8.6, 7.2, 1.6 Hz, 1H), 7.12 (td, J=7.5, 1.2 Hz, 1H), 7.00-6.96 (m, 2H), 6.65 (d, J=2.4 Hz, 1H), 4.22-4.13 (m, 4H), 2.81 (t, J=6.1 Hz, 2H), 2.58-2.47 (m, 4H), 1.95 (sextet, J=7.3 Hz, 2H), 1.63 (quint, J=5.6 Hz, 4H), 1.50-1.42 (m, 2H), 0.95 (t, J=7.4 Hz, 3H). ¹³C NMR: (151 MHz, CDCl₃) δ 165.5, 161.5, 151.3, 136.8, 130.3, 129.4, 128.5, 128.5, 127.4, 123.0, 120.7, 120.2, 114.2, 103.9, 66.2, 57.8, 55.1, 54.2, 25.9, 24.2, 23.8, 11.2.

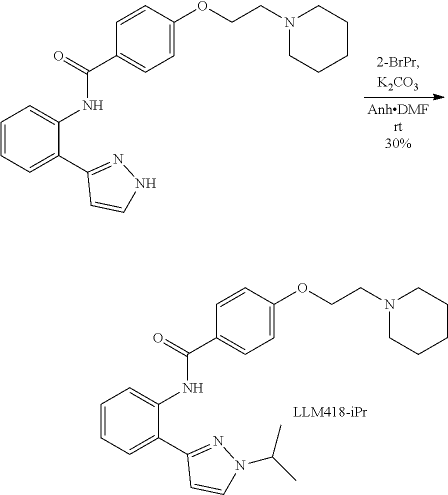 [SMILES: O=C(Nc1ccccc1-c1cc[nH]n1)c1ccc(OCCN2CCCCC2)cc1 ; CC(C)n1ccc(-c2ccccc2NC(=O)c2ccc(OCCN3CCCCC3)cc2)n1]

N-(2-(1-isopropyl-1H-pyrazol-3-yl)phenyl)-4-(2-(piperidin-1-yl)ethoxy)benzamide: To an oven-dried vial was added LLM418 (40.4 mg, 0.103 mmol, 1.0 eq) and K₂CO₃ (43.4 mg, 0.314 mmol, 3.0 eq). Anhydrous DMF (2.5 mL) was added, and the mixture was stirred at room temperature for 30 minutes. To the mixture was then added slowly 0.58 mL of a freshly prepared solution of 0.2 M 2-bromopropane (0.116 mmol, 1.1 eq) in anhydrous DMF. The mixture was then stirred at room temperature overnight. After 3.8 days, the reaction mixture was diluted with 10 mL DCM and washed with 10 mL brine. The organic layer was then separated and concentrated. The crude material was then purified via prep TLC using a solvent system of 66:31:3 Hex:EtOAc:TEA to afford the desired product as an off-white residue (13.3 mg, 0.0307 mmol, 30%). ¹H NMR: (600 MHz, Chloroform-d) δ 12.10 (s, 1H), 8.84 (dd, J=8.5, 1.3 Hz, 1H), 8.11-8.05 (m, 2H), 7.67 (dd, J=7.8, 1.5 Hz, 1H), 7.50 (d, J=2.4 Hz, 1H), 7.35 (ddd, J=8.6, 7.3, 1.6 Hz, 1H), 7.12 (td, J=7.6, 1.3 Hz, 1H), 7.01-6.95 (m, 2H), 6.65 (d, J=2.4 Hz, 1H), 4.58 (quint, J=6.7 Hz, 1H), 4.31-4.20 (m, 2H), 2.97-2.84 (m, 2H), 2.78-2.48 (m, 4H), 1.76-1.67 (m, 4H), 1.57 (d, J=6.7 Hz, 6H), 1.53-1.47 (m, 2H).

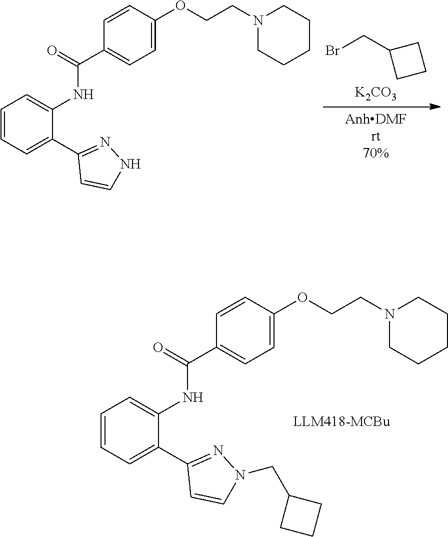

N-(2-(1-(cyclobutylmethyl)-1H-pyrazol-3-yl)phenyl)-4-(2-(piperidin-1-yl)ethoxy)benzamide: To an oven-dried vial was added LLM418 (40.3 mg, 0.103 mmol, 1.0 eq), which was dissolved in 2.5 mL anhydrous DMF. To the solution was then added K₂CO₃ (42.4 mg, 0.307 mmol, 3.0 eq), and the mixture was stirred at room temperature for 40 minutes. To the mixture was then added slowly 0.58 mL of a freshly prepared solution of 0.2 M bromomethyl cyclobutane (0.116 mmol, 1.1 eq) in anhydrous DMF. The mixture was then stirred at room temperature overnight. After 75 hours, the reaction mixture was diluted with 10 mL DCM and washed with 10 mL brine. The organic layer was then separated and concentrated. The crude material was then purified via automated flash column chromatography using a gradient of 78:20:2 to 16:82:2 Hex:EtOAc:TEA to afford the desired product as a pale orange solid (33.0 mg, 0.0720 mmol, 70%). ¹H NMR: (600 MHz, CDCl₃) δ 12.18 (s, 1H), 8.85 (dd, J=8.4, 1.3 Hz, 1H), 8.12-8.05 (m, 2H), 7.67 (dd, J=7.8, 1.6 Hz, 1H), 7.43 (d, J=2.4 Hz, 1H), 7.35 (ddd, J=8.5, 7.2, 1.6 Hz, 1H), 7.12 (td, J=7.5, 1.3 Hz, 1H), 7.02-6.96 (m, 2H), 6.64 (d, J=2.4 Hz, 1H), 4.21 (d, J=7.3 Hz, 2H), 4.19 (t, J=6.0 Hz, 2H), 2.87 (quint, J=7.7 Hz, 1H), 2.81 (t, J=6.1 Hz, 2H), 2.60-2.46 (m, 4H), 2.10-2.02 (m, 2H), 1.95-1.83 (m, 2H), 1.83-1.75 (m, 2H), 1.62 (quint, J=5.6 Hz, 4H), 1.50-1.42 (m, 2H).

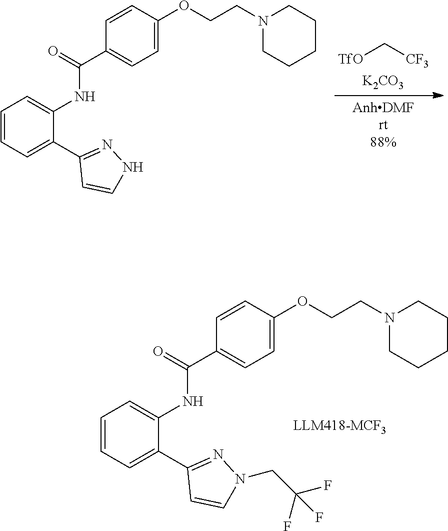

4-(2-(piperidin-1-yl)ethoxy)-N-(2-(1-(2,2,2-trifluoroethyl)-1H-pyrazol-3-yl)phenyl)benzamide: To an oven-dried vial was added LLM418 (41.0 mg, 0.105 mmol, 1.0 eq), which was dissolved in 2.5 mL anhydrous DMF. To the solution was then added K₂CO₃ (44.5 mg, 0.322 mmol, 3.1 eq), and the mixture was stirred at room temperature for 30 minutes. To the mixture was then added slowly 0.58 mL of a freshly prepared solution of 0.2 M 2,2,2-trifluoroethyl trifluoromethanesulfonate (0.116 mmol, 1.1 eq) in anhydrous DMF. The mixture was then stirred at room temperature overnight. After 22 hours, the reaction mixture was diluted with 10 mL DCM and washed with 10 mL brine. The organic layer was then separated and concentrated. The crude material was then purified via automated flash column chromatography using a gradient of 83:15:2 to 18:80:2 Hex:EtOAc:TEA to afford the desired product as an off-white solid (43.6 mg, 0.0923 mmol, 88%). ¹H NMR: (600 MHz, CDCl₃) δ 11.74 (s, 1H), 8.87 (dd, J=8.5, 1.3 Hz, 1H), 8.04-8.00 (m, 2H), 7.67 (dd, J=7.8, 1.6 Hz, 1H), 7.60 (d, J=2.5 Hz, 1H), 7.39 (ddd, J=8.6, 7.2, 1.6 Hz, 1H), 7.14 (td, J=7.5, 1.2 Hz, 1H), 7.01-6.96 (m, 2H), 6.80 (d, J=2.5 Hz, 1H), 4.75 (q, J=8.3 Hz, 2H), 4.18 (t, J=6.1 Hz, 2H), 2.81 (t, J=6.1 Hz, 2H), 2.60-2.42 (m, 4H), 1.63 (quint, J=5.7 Hz, 4H), 1.50-1.42 (m, 2H).

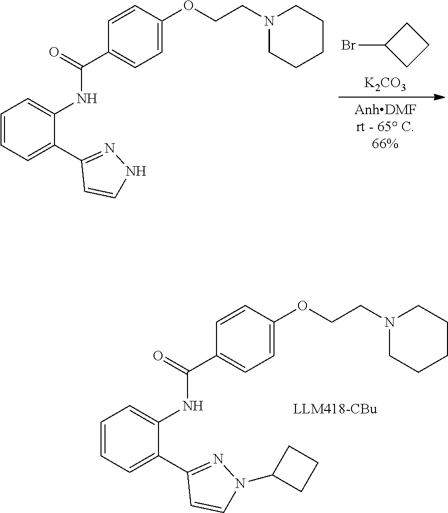

N-(2-(1-cyclobutyl-1H-pyrazol-3-yl)phenyl)-4-(2-(piperidin-1-yl)ethoxy)benzamide: To an oven-dried vial was added LLM418 (41.0 mg, 0.105 mmol, 1.0 eq), which was dissolved in 2.5 mL anhydrous DMF. To the solution was then added K₂CO₃ (43.4 mg, 0.314 mmol, 3.0 eq), and the mixture was stirred at room temperature for 30 minutes. To the mixture was then added slowly 0.58 mL of a freshly prepared solution of 0.2 M bromocyclobutane (0.116 mmol, 1.1 eq) in anhydrous DMF. The mixture was then heated to 65° C. and stirred overnight, during which an additional 0.03 mL bromocyclobutane was added (0.319 mmol, 3.0 eq). After 19 hours, the reaction mixture was quenched with 10 mL aqueous K₂CO₃ and extracted with 20 mL DCM. The organic layer was then separated and concentrated. The crude material was then purified via automated flash column chromatography using a gradient of 48:50:2 to 0:98:2 Hex:EtOAc:TEA to afford the desired product as an off-white residue (30.8 mg, 0.0693 mmol, 66%). ¹H NMR: (600 MHz, CDCl₃) δ 12.02 (s, 1H), 8.83 (dd, J=8.4, 1.2 Hz, 1H), 8.09-8.03 (m, 2H), 7.66 (dd, J=7.8, 1.6 Hz, 1H), 7.50 (d, J=2.4 Hz, 1H), 7.35 (ddd, J=8.6, 7.2, 1.6 Hz, 1H), 7.12 (td, J=7.5, 1.2 Hz, 1H), 7.02-6.97 (m, 2H), 6.64 (d, J=2.4 Hz, 1H), 4.85-4.77 (m, 1H), 4.18 (t, J=6.1 Hz, 2H), 2.81 (t, J=6.1 Hz, 2H), 2.61-2.46 (m, 8H), 1.94-1.86 (m, 2H), 1.63 (quint, J=5.6 Hz, 5H), 1.50-1.42 (m, 2H). ¹³C NMR: (151 MHz, CDCl₃) δ 165.7, 161.5, 151.3, 136.7, 129.4, 128.7, 128.5, 128.5, 127.6, 123.0, 120.9, 120.5, 114.2, 103.9, 66.2, 57.8, 55.8, 55.1, 30.5, 25.9, 24.2, 14.9.

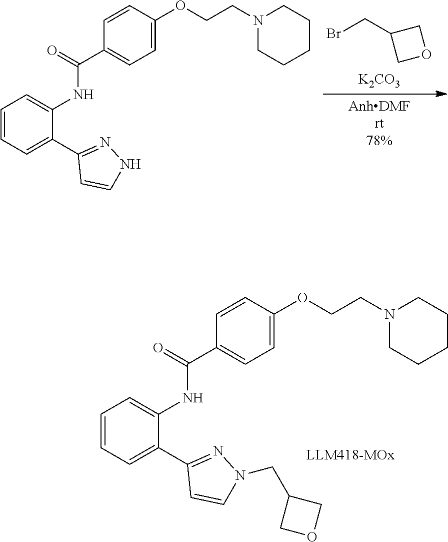

N-(2-(1-(oxetan-3-ylmethyl)-1H-pyrazol-3-yl)phenyl)-4-(2-(piperidin-1-yl)ethoxy)benzamide: To an oven-dried vial was added LLM418 (40.7 mg, 0.104 mmol, 1.0 eq), which was dissolved in 2.5 mL anhydrous DMF. To the solution was then added K₂CO₃ (43.3 mg, 0.313 mmol, 3.0 eq), and the mixture was stirred at room temperature for 30 minutes. To the mixture was then added a solution of 3-bromomethyl oxetane (62.9 mg, 0.417 mmol, 4.0 eq) in 0.5 mL anhydrous DMF. The mixture was then stirred overnight at room temperature. After 6 days, the reaction mixture was quenched with 10 mL aqueous K₂CO₃ and extracted with 2×20 mL portions of DCM. The organic layers were combined and concentrated. The crude material was then purified via automated flash column chromatography using a gradient of 0-10% MeOH in DCM to afford the desired product as yellow solid (37.5 mg, 0.0814 mmol, 78%). ¹H NMR: (600 MHz, CDCl₃) δ 11.91 (s, 1H), 8.81 (dd, J=8.4, 1.2 Hz, 1H), 8.06-8.01 (m, 2H), 7.65 (dd, J=7.8, 1.6 Hz, 1H), 7.47 (d, J=2.4 Hz, 1H), 7.36 (ddd, J=8.5, 7.3, 1.6 Hz, 1H), 7.13 (td, J=7.6, 1.3 Hz, 1H), 7.03-6.99 (m, 2H), 6.66 (d, J=2.4 Hz, 1H), 4.79-4.73 (m, 2H), 4.52 (d, J=7.6 Hz, 2H), 4.47-4.42 (m, 2H), 4.34-4.26 (m, 2H), 3.54-3.46 (m, 1H), 3.02-2.91 (m, 2H), 2.81-2.58 (m, 4H), 1.78-1.69 (m, 4H), 1.55-1.47 (m, 2H). ¹³C NMR: (151 MHz, CDCl₃) δ 165.5, 161.1, 151.9, 136.7, 130.4, 129.4, 128.8, 128.8, 127.6, 123.1, 120.8, 120.1, 114.3, 104.6, 74.5, 65.4, 57.4, 54.9, 54.8, 35.6, 25.1, 23.6.

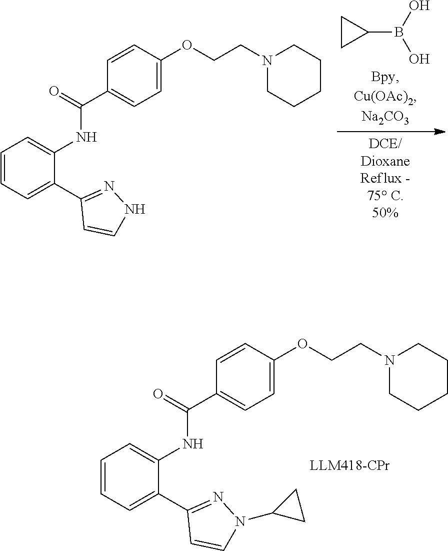

N-(2-(1-cyclopropyl-1H-pyrazol-3-yl)phenyl)-4-(2-(piperidin-1-yl)ethoxy)benzamide: To a vial was added copper diacetate (20.8 mg, 0.115 mmol, 1.06 eq) and 2,2′-bipyridine (21.0 mg, 0.134 mmol, 1.2 eq), which were then suspended in 2 mL anhydrous 1,2-dichloroethane. The mixture was then refluxed for 15 minutes. To a separate vial was added LLM418 (42.4 mg, 0.109 mmol, 1.0 eq), cyclopropyl boronic acid (19.9 mg, 0.232 mmol, 2.1 eq), and Na₂CO₃ (23.3 mg, 0.220 mmol, 2.0 eq), and the reagents were then suspended in 3 mL anhydrous 1,2-dichloroethane and 2 mL anhydrous 1,4-dioxane. To this mixture was added the dark blue-green Cu(OAc)₂/bpy solution. The mixture was stirred and heated to 75° C. for 3.75 hours, after which the mixture was cooled to room temperature and quenched with 10 ml AQ K₂CO₃. The aqueous layer was then extracted with 2×25 mL portions of DCM. The organic layers were combined, dried over anhydrous Na₂SO₄, filtered, and concentrated. The crude material was purified via automated flash column chromatography using a gradient of 0-8% MeOH in DCM to afford the desired product as a red-orange solid (23.5 mg, 0.0546 mmol, 50%). ¹H NMR: (600 MHz, CDCl₃) δ 12.23 (s, 1H), 8.88 (dd, J=8.4, 1.2 Hz, 1H), 8.20-8.14 (m, 2H), 7.66 (dd, J=7.8, 1.6 Hz, 1H), 7.53 (d, J=2.5 Hz, 1H), 7.35 (ddd, J=8.6, 7.3, 1.6 Hz, 1H), 7.12 (td, J=7.6, 1.2 Hz, 1H), 7.03-6.98 (m, 2H), 6.63 (d, J=2.4 Hz, 1H), 4.40-4.16 (m, 2H), 3.73-3.64 (m, 1H), 3.03-2.83 (m, 2H), 2.83-2.47 (m, 4H), 1.77-1.66 (m, 4H), 1.56-1.46 (m, 2H), 1.20-1.16 (m, 2H), 1.16-1.10 (m, 2H).

### Example 10: LLM418-Az

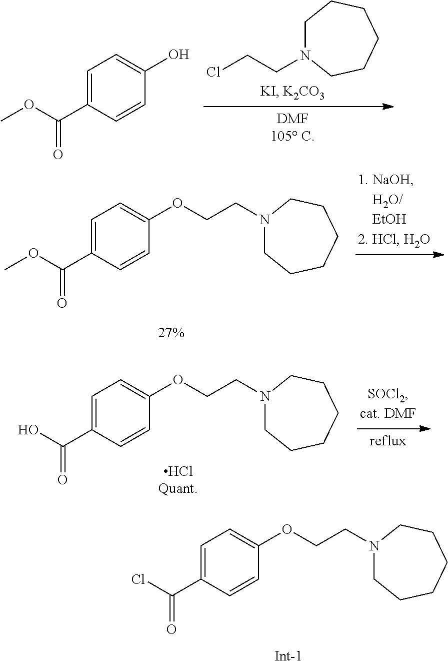 [SMILES: COC(=O)c1ccc(O)cc1 ; ClCCN1CCCCCC1 ; COC(=O)c1ccc(OCCN2CCCCCC2)cc1 ; O=C(O)c1ccc(OCCN2CCCCCC2)cc1 ; O=C(Cl)c1ccc(OCCN2CCCCCC2)cc1]

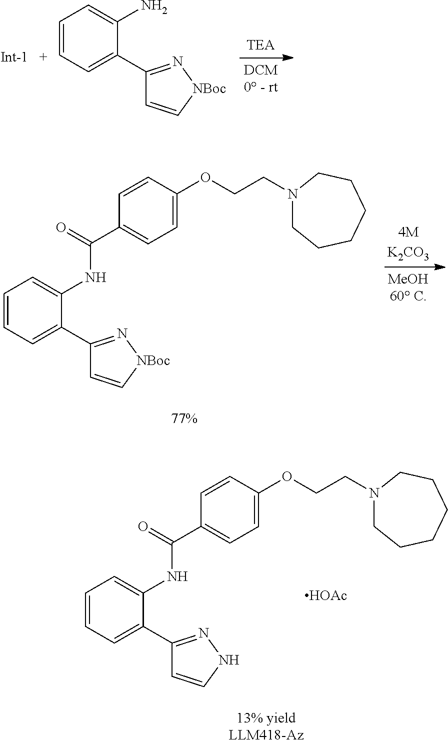 [SMILES: CC(C)(C)OC(=O)n1ccc(C2=C[C+]=CC=C2N)n1 ; CC(C)(C)OC(=O)n1ccc(-c2ccccc2NC(=O)c2ccc(OCCN3CCCCCC3)cc2)n1 ; O=C(Nc1ccccc1-c1cc[nH]n1)c1ccc(OCCN2CCCCCC2)cc1 ; "·HOAc"]

### Example 11: 1-(2-(JH-pyrazol-3-yl)phenyl)-3-(2-(piperidin-1-yl)ethyl)urea (LS-U1)

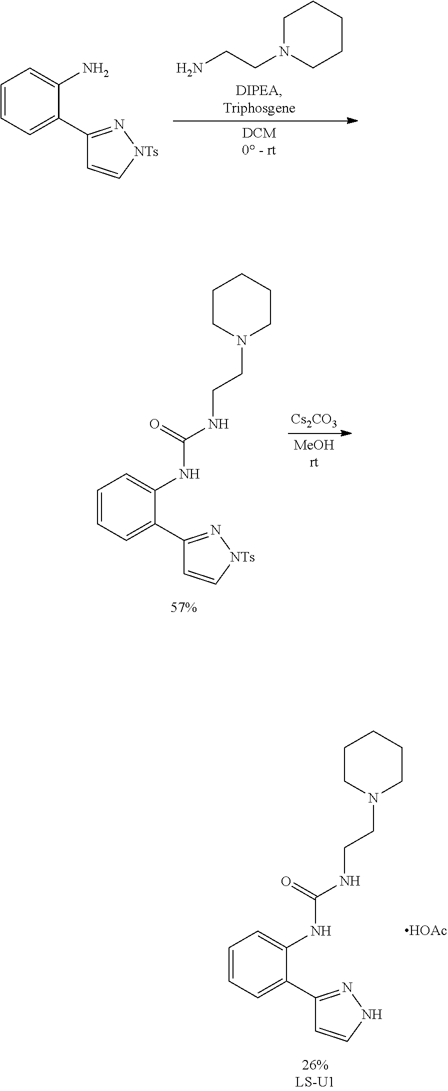

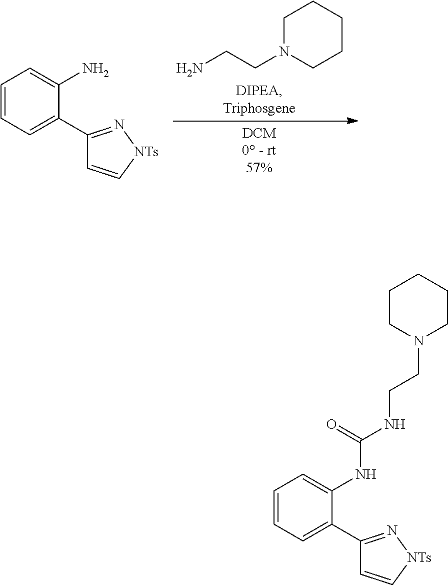 [SMILES: NCCN1CCCCC1 ; Cc1ccc(S(=O)(=O)n2ccc(-c3ccccc3N)n2)cc1 ; Cc1ccc(S(=O)(=O)n2ccc(-c3ccccc3NC(=O)NCCN3CCCCC3)n2)cc1]

1-(2-(piperidin-1-yl)ethyl)-3-(2-(1-tosyl-1H-pyrazol-3-yl)phenyl)urea: To an oven-dried vial was added triphosgene (55.6 mg, 0.187 mmol, 0.54 eq), which was then dissolved in 3 mL DCM. DIPEA (0.15 mL, 0.86 mmol, 2.5 eq) was added. The solution was cooled to 0° C. To the solution was then added a solution of 2-(1-tosyl-1H-pyrazol-3-yl)aniline (109.1 mg, 0.348 mmol, 1.0 eq) in 2 mL DCM. The solution was stirred at 0° C. for 10 minutes. Then, a solution of 1-(2-aminoethyl)piperidine (0.08 mL, 0.459 mmol, 1.3 eq) in 1 mL DCM was added. The solution was stirred overnight and allowed to slowly warm to room temperature. After 23 h, the reaction was quenched with 10 mL AQ K₂CO₃ and extracted with 50 mL DCM. The organic layer was separated and concentrated. The crude material was purified via automated flash column chromatography using a gradient of 88:10:2 to 18:80:2 Hex:EtOAc:TEA to afford the desired product as a pale yellow residue (91.9 mg, 0.197 mmol, 57%).

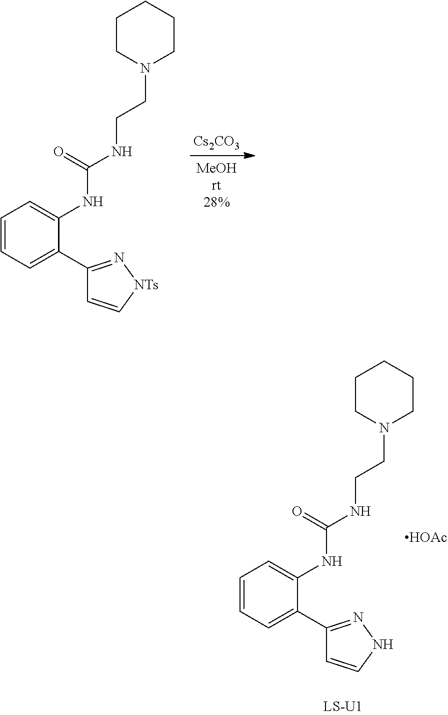

1-(2-(1H-pyrazol-3-yl)phenyl)-3-(2-(piperidin-1-yl)ethyl)urea: To a vial was added 1-(2-(piperidin-1-yl)ethyl)-3-(2-(1-tosyl-1H-pyrazol-3-yl)phenyl)urea (69.9 mg, 0.149 mmol, 1.0 eq), which was dissolved in 5 mL MeOH. Cs₂CO₃ (392.2 mg, 1.204 mmol, 8.1 eq) was added, and the mixture was stirred at room temperature for 1.5 hours. Upon completion of the reaction, it was diluted with 5 mL H₂O and extracted with 3×50 mL portions of DCM. The AQ layer was then diluted with 5 mL sat. AQ NH₄Cl and extracted with 50 mL DCM. The organic layers were combined and concentrated. The crude material was then purified via preparative HPLC using an H₂O:MeCN:AcOH solvent system to afford the acetate salt of the desired product as a translucent white residue (15.4 mg, 0.0412 mmol, 28%).

### Example 12: LS-TF Compounds (LS-TF-2P, LS-TF-3P, LS-TF-4P)

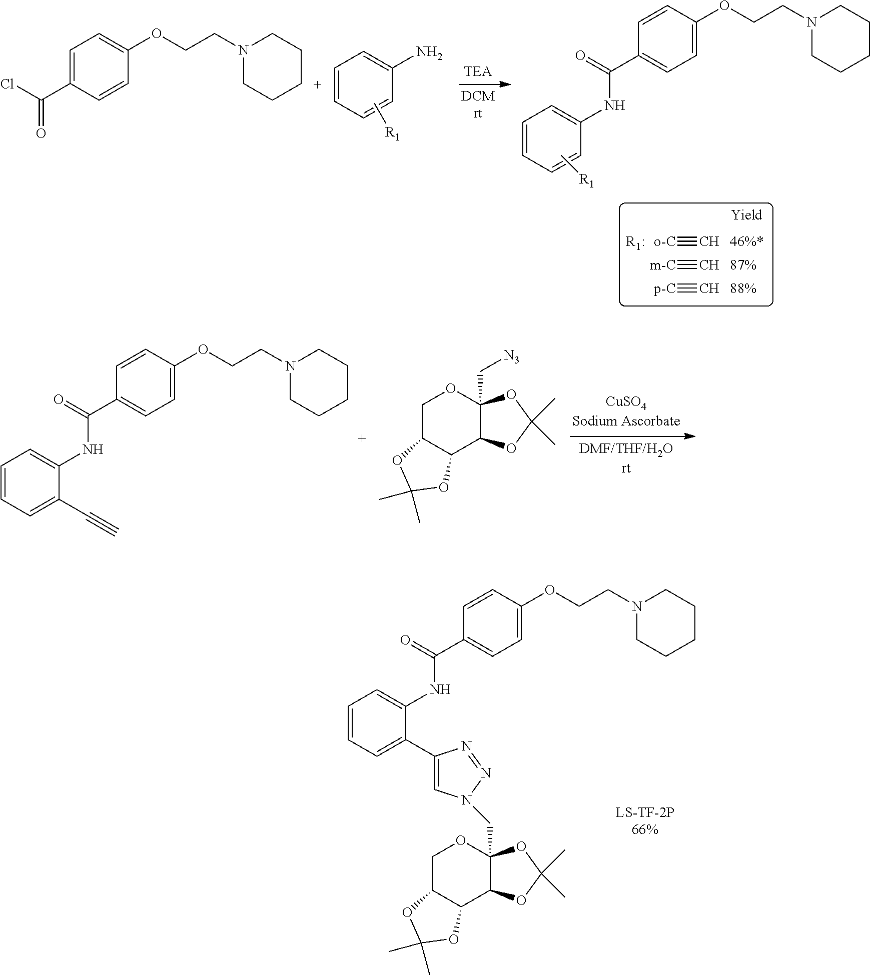

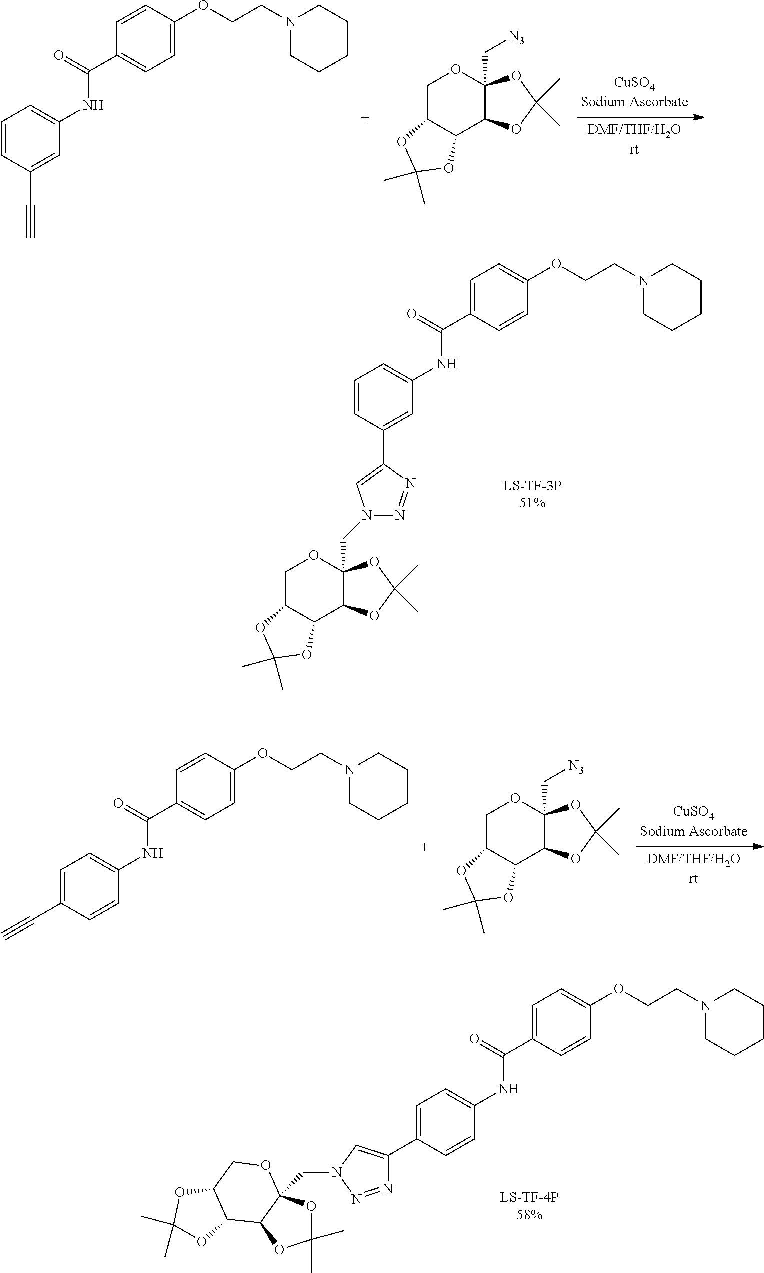

### Example 13: LS-AF Compounds (LS-AF-2P, LS-AF-3P, and LS-AF-4P,)

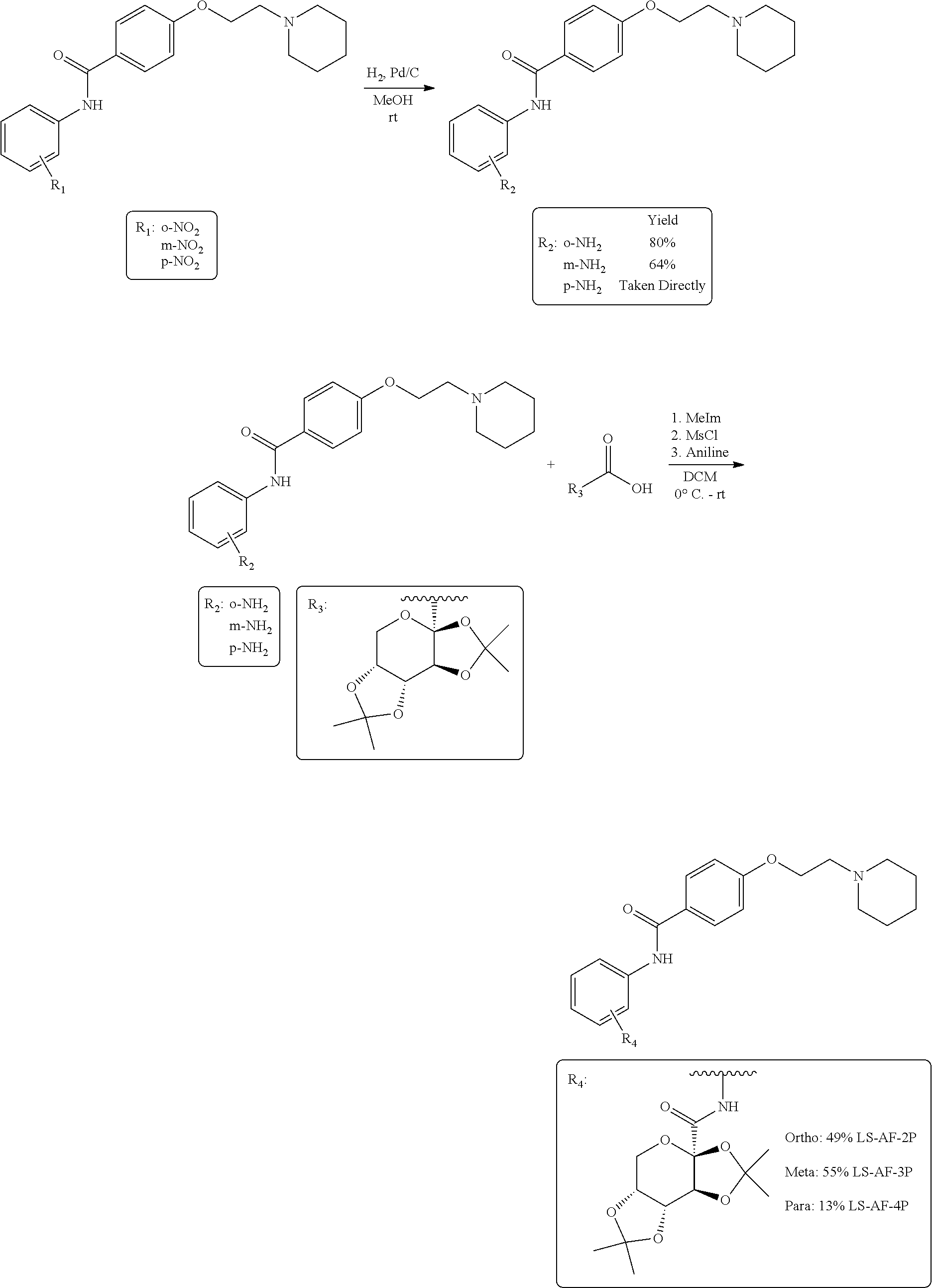

### Example 14: LS-T Compounds (LS-T-2, LS-T-3, and LS-T-4)

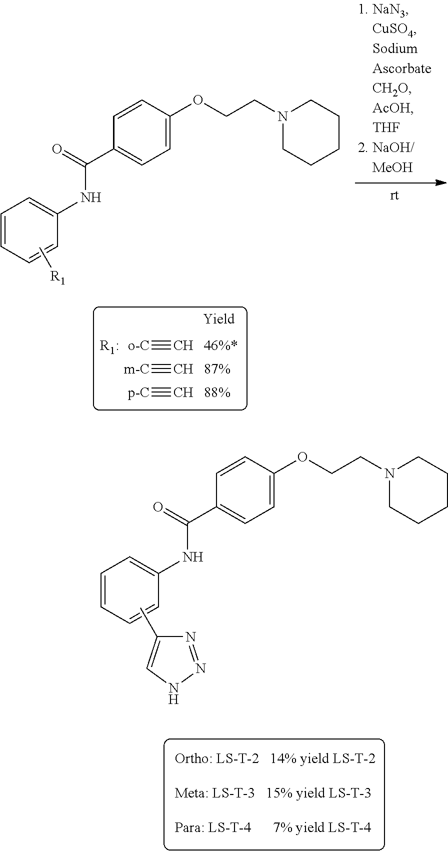

### Example 15: LS-O Compounds (LS-01, LS-02, LS-03, and LS-05)

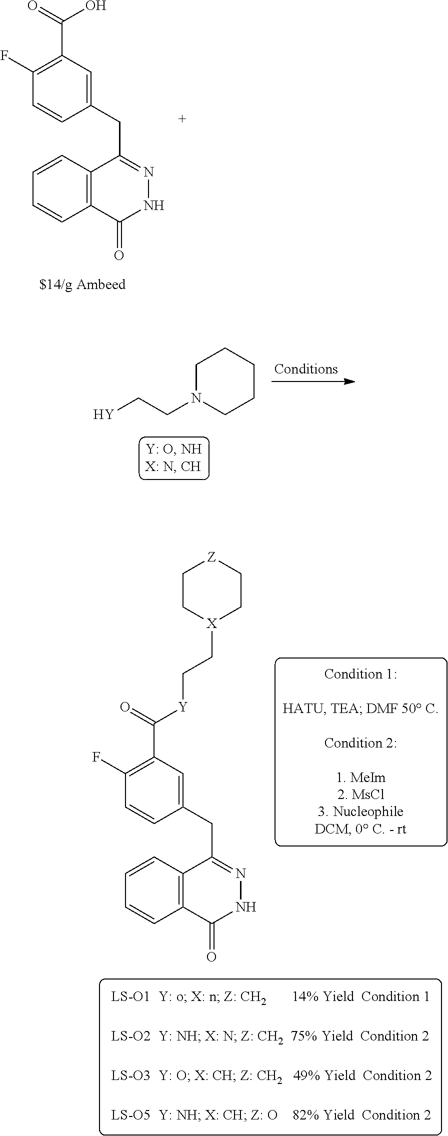

### Example 16: LLM4 Compounds (LLM4, LLM4-S1, LLM4-S2, and LLM4-S3)

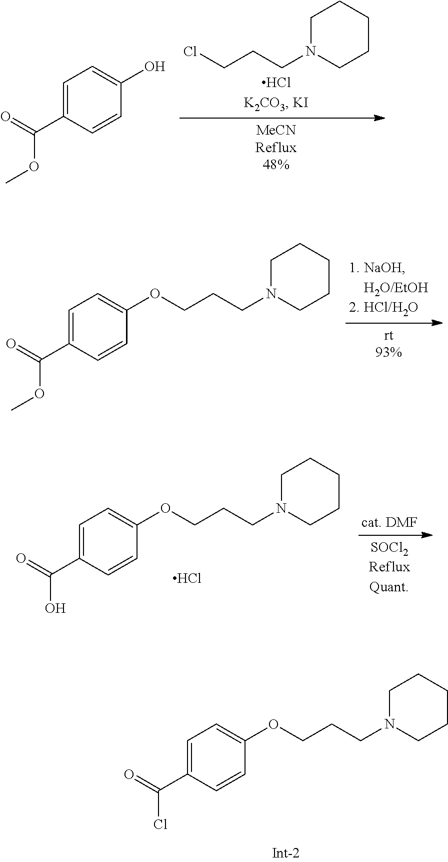 [SMILES: COC(=O)c1ccc(O)cc1 ; ClCCCN1CCCCC1 ; COC(=O)c1ccc(OCCCN2CCCCC2)cc1 ; O=C(O)c1ccc(OCCCN2CCCCC2)cc1 ; Cl ; O=C(Cl)c1ccc(OCCCN2CCCCC2)cc1]

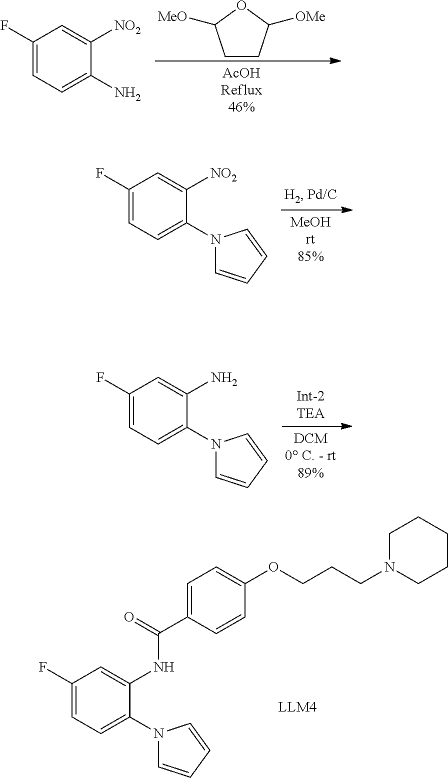 [SMILES: Nc1ccc(F)cc1[N+](=O)[O-] ; COC1CCC(OC)O1 ; O=[N+]([O-])c1cc(F)ccc1-n1cccc1 ; Nc1cc(F)ccc1-n1cccc1 ; O=C(Nc1cc(F)ccc1-n1cccc1)c1ccc(OCCCN2CCCCC2)cc1]

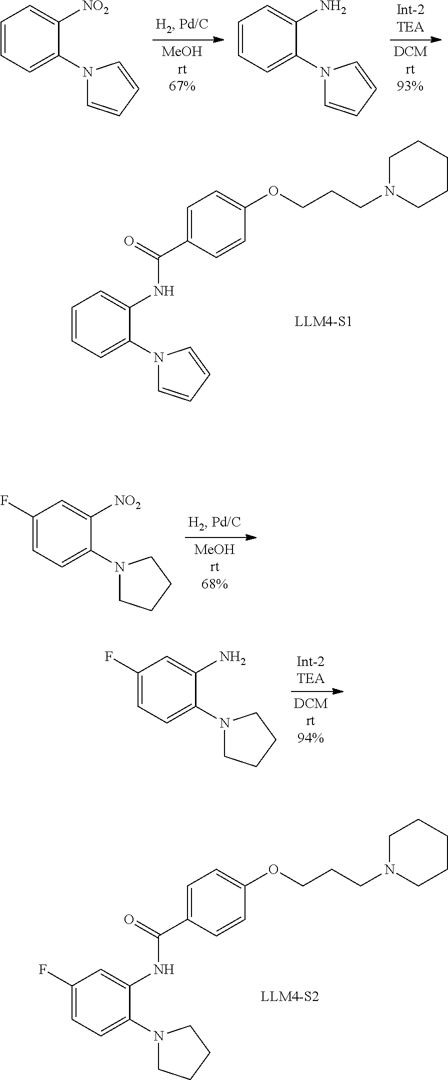 [SMILES: O=[N+]([O-])c1ccccc1-n1cccc1 ; Nc1ccccc1-n1cccc1 ; O=C(Nc1ccccc1-n1cccc1)c1ccc(OCCCN2CCCCC2)cc1 ; O=[N+]([O-])c1cc(F)ccc1N1CCCC1 ; Nc1cc(F)ccc1N1CCCC1 ; O=C(Nc1cc(F)ccc1N1CCCC1)c1ccc(OCCCN2CCCCC2)cc1]

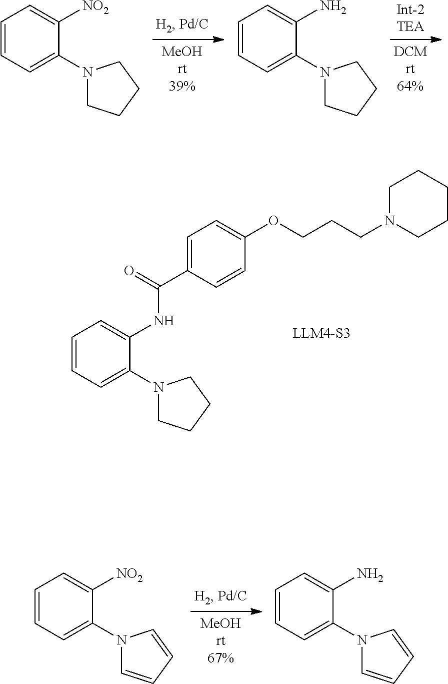 [SMILES: O=[N+]([O-])c1ccccc1N1CCCC1 ; Nc1ccccc1N1CCCC1 ; O=C(Nc1ccccc1N1CCCC1)c1ccc(OCCCN2CCCCC2)cc1 ; O=[N+]([O-])c1ccccc1-n1cccc1 ; Nc1ccccc1-n1cccc1]

2-(1H-pyrrol-1-yl)aniline. To a 50 mL round bottom flask was added 1-(2-nitrophenyl)pyrrole (301.4 mg, 1.602 mmol, 1.0 eq), which was then dissolved in 13 mL methanol. To the solution was then added palladium on carbon (10 wt. %, 59.9 mg, 56.3 μmol Pd, 0.035 eq). The flask headspace was then evacuated and filled with H₂ three times, and the reaction mixture was stirred at room temperature under H₂ for 28.75 hours. Upon completion of the reaction, the mixture was diluted with 15 mL MeOH, filtered over celite, and concentrated. The crude material was then purified via automated flash column chromatography using a gradient of 100:0 to 96:4 Hex:EtOAc to afford the desired product as a white solid (170.1 mg, 1.075 mmol, 67% yield). ¹H NMR: (600 MHz, CDCl₃) δ 7.20-7.12 (m, 2H), 6.84 (dd, J=2.1, 2.1 Hz, 2H), 6.82-6.76 (m, 2H), 6.34 (dd, J=2.1, 2.1 Hz, 2H), 3.71 (s, 2H). ¹³C NMR: (151 MHz, CDCl₃) δ 142.1, 128.5, 127.5, 127.1, 121.7, 118.4, 116.1, 109.4.

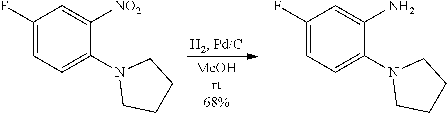

5-fluoro-2-(pyrrolidin-1-yl)aniline. To a 50 mL round bottom flask was added 1-(4-fluoro-2-nitrophenyl)pyrrolidine (302.3 mg, 1.438 mmol, 1.0 eq), which was then dissolved in 13 mL methanol. To the solution was then added palladium on carbon (10 wt. %, 53.6 mg, 50.4 μmol Pd, 0.035 eq). The flask headspace was then evacuated and filled three times with H₂. The mixture was then stirred at room temperature under H₂ for 27.58 hours. Upon completion of the reaction, the mixture was diluted with 13 mL methanol, filtered over celite, and concentrated. The crude material was then purified via automated flash column chromatography using a gradient of 100:0 to 92:8 Hex:EtOAc to afford the desired product as a dark red oil (177.6 mg, 0.985 mmol, 68% yield). ¹H NMR: (600 MHz, CDCl₃) δ 6.92 (dd, J=8.7, 5.7 Hz, 1H), 6.43 (dd, J=10.2, 2.9 Hz, 1H), 6.39 (ddd, J=8.5, 8.5, 2.9 Hz, 1H), 4.03 (s, 2H), 3.10-2.88 (m, 4H), 1.99-1.85 (m, 4H). ¹³C NMR: (151 MHz, CDCl₃) δ 159.8 (d, J_{C-F}=239 Hz), 143.3 (d, J_{C-F}=10.9 Hz), 133.4 (d, J_{C-F}=2.4 Hz), 119.9 (d, J_{C-F}=9.9 Hz), 104.1 (d, J_{C-F}=22.1 Hz), 101.9 (d, J_{C-F}=25.4 Hz), 51.5, 24.1.

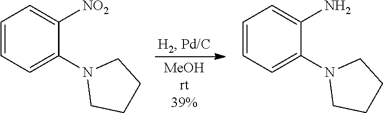

2-(pyrrolidin-1-yl)aniline. To a 50 mL round bottom flask was added 2-nitro-1-pyrrolidinobenzene (304.3 mg, 1.583 mmol, 1.0 eq), which was then dissolved in 13 mL methanol. To the solution was then added palladium on carbon (10 wt. %, 59.9 mg, 56.3 μmol Pd, 0.035 eq). The flask headspace was then evacuated and filled three times with H₂, and the mixture was then stirred under H₂ at room temperature for 18 hours. Upon completion of the reaction, the mixture was then diluted with 13 mL methanol, filtered over celite, and concentrated. The crude material was then purified via automated flash column chromatography using a gradient of 100:0 to 93:7 Hex:EtOAc to afford the desired product as a golden yellow oil (99.4 mg, 0.613 mmol, 39% yield). ¹H NMR: (600 MHz, CDCl₃) δ 7.02-6.98 (m, 1H), 6.91-6.86 (m, 1H), 6.76-6.71 (m, 2H), 3.12-2.99 (m, 4H), 2.06-1.86 (m, 4H). ¹³C NMR: (151 MHz, CDCl₃) δ 141.3, 137.7, 123.4, 118.6, 118.5, 115.4, 50.9, 24.1.

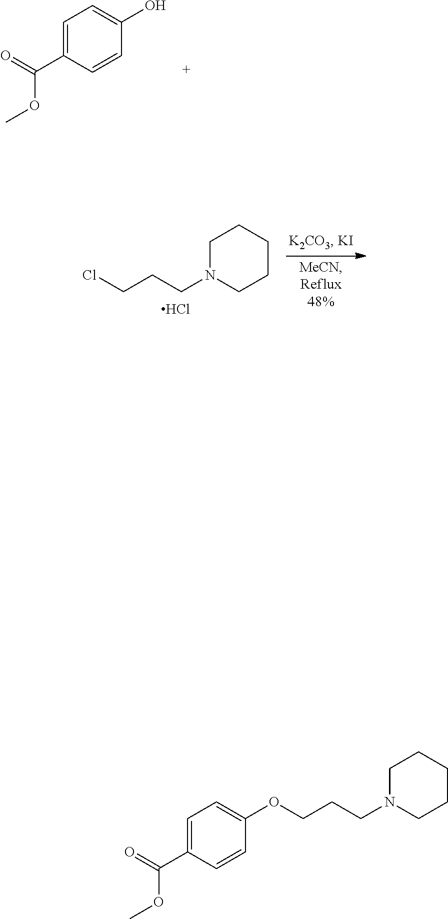 [SMILES: COC(=O)c1ccc(O)cc1 ; ClCCCN1CCCCC1 ; Cl ; COC(=O)c1ccc(OCCCN2CCCCC2)cc1]

Methyl 4-(3-(piperidin-1-yl)propoxy)benzoate. To an oven-dried 50 mL round bottom flask was added methyl 4-hydroxybenzoate (3.002 g, 19.73 mmol, 1.0 eq), 1-(3-chloropropyl)piperidine monohydrochloride (5.865 g, 29.60 mmol, 1.5 eq), potassium carbonate (8.182 g, 59.20 mmol, 3.0 eq), and potassium iodide (329.8 mg, 1.987 mmol, 0.10 eq). Acetonitrile (45 mL) was added, and the mixture was then refluxed for 19.5 hours. Upon completion, the mixture was immediately filtered, washing with 100 mL DCM. The organic layer was then washed with 100 mL AQ K₂CO₃. The organic layer was separated, and the aqueous layer was extracted with 2×100 mL portions of DCM. The organic layers were combined, dried over anhydrous Na₂SO₄, filtered, and concentrated. The crude material was then purified via automated flash column chromatography using a gradient of 95:5 to 92:8 DCM:MeOH to afford the desired product as a viscous orange oil (2.615 g, 9.427 mmol, 48% yield). ¹H NMR: (600 MHz, DMSO-d₆) δ 7.91-7.88 (m, 2H), 7.04-7.01 (m, 2H), 4.07 (t, J=6.4 Hz, 2H), 3.80 (s, 3H), 2.41-2.35 (m, 6H), 1.90-1.85 (m, 2H), 1.51-1.47 (m, 4H), 1.39-1.36 (m, 2H). ¹³C NMR: (151 MHz, DMSO-d₆) δ 165.9, 162.5, 131.2, 121.7, 114.4, 66.3, 54.9, 54.0, 51.8, 26.0, 25.5, 24.0.

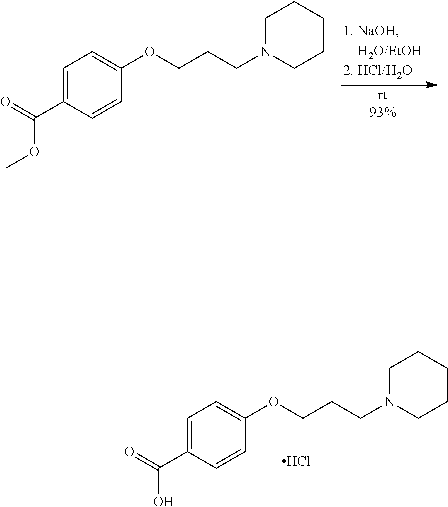 [SMILES: COC(=O)c1ccc(OCCCN2CCCCC2)cc1 ; O=C(O)c1ccc(OCCCN2CCCCC2)cc1 ; Cl]

4-(3-(piperidin-1-yl)propoxy)benzoic acid hydrochloride. To a 100 mL double-necked round bottom flask was added a solution of methyl 4-(3-(piperidin-1-yl)propoxy)benzoate (2.503 g, 9.024 mmol, 1.0 eq) in 30 mL ethanol. To the stirring solution was then added 22 mL 0.91 M NaOH (20.02 mmol, 2.2 eq). The solution was then stirred at room temperature for 69.5 hours. Upon completion, the solvent was removed under reduced pressure at 60° C., and the resultant sodium salt was dried at 60° C. overnight. The sodium salt was then dissolved in 35 mL H₂O, and the solution was slowly acidified to pH ˜1 using concentrated HCl. A pale yellow solid precipitated, which was then isolated via vacuum filtration and dried at 60° C. overnight. The filtrate was then concentrated and filtered twice more, with the resultant solid being dried at 60° C. overnight. This afforded the desired product as a pale yellow solid (2.518 g, 8.398 mmol, 93% yield). ¹H NMR: (600 MHz, DMSO-d₆) δ 10.74 (s, 1H), 7.90-7.87 (m, 2H), 7.03-7.01 (m, 2H), 4.13 (t, J=6.1 Hz, 2H), 3.43-3.41 (m, 2H), 3.16-3.12 (m, 2H), 2.89-2.83 (m, 2H), 2.25-2.20 (m, 2H), 1.84 (qt, J=13.4, 3.6 Hz, 2H), 1.78-1.74 (m, 2H), 1.71-1.67 (m, 1H), 1.38 (qt, J=12.7, 4.0 Hz, 1H). ¹³C NMR: (151 MHz, DMSO) δ 166.9, 161.8, 131.4, 123.2, 114.3, 65.4, 53.2, 52.0, 23.1, 22.3, 21.4.

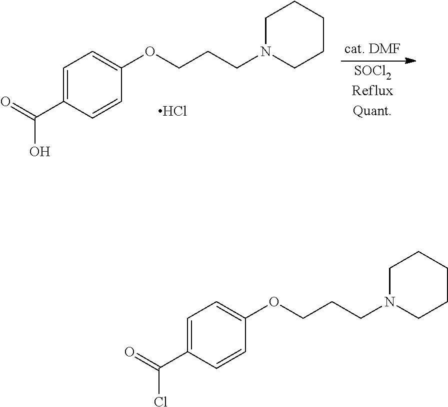 [SMILES: O=C(O)c1ccc(OCCCN2CCCCC2)cc1 ; Cl ; O=C(Cl)c1ccc(OCCCN2CCCCC2)cc1]

4-(3-(piperidin-1-yl)propoxy)benzoyl chloride. To an oven-dried 50 mL pear-shaped flask was added 4-(3-(piperidin-1-yl)propoxy)benzoic acid hydrochloride (1.555 g, 5.187 mmol, 1.0 eq), which was then suspended in thionyl chloride (5.5 mL, 75.40 mmol, 14.5 eq). A catalytic amount of N,N-dimethylformamide (4 drops) was added, and the mixture was then refluxed for 2.5 hours. Upon completion of the reaction, the mixture was evaporated to dryness to afford the desired product as a yellow solid (quantitative yield), which was taken directly to the next step.

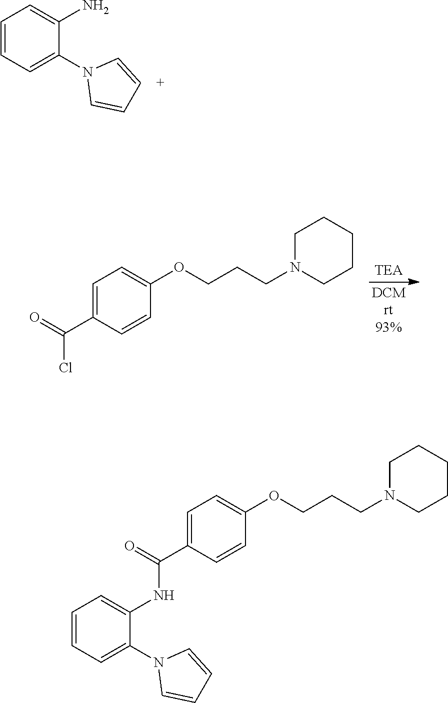 [SMILES: Nc1ccccc1-n1cccc1 ; O=C(Cl)c1ccc(OCCCN2CCCCC2)cc1 ; O=C(Nc1ccccc1-n1cccc1)c1ccc(OCCCN2CCCCC2)cc1]

N-(2-(1H-pyrrol-1-yl)phenyl)-4-(3-(piperidin-1-yl)propoxy)benzamide. To an oven-dried vial was added 2-(1H-pyrrol-1-yl)aniline (79.7 mg, 0.504 mmol, 1.0 eq), which was dissolved in 4 mL dichloromethane. To the solution was then added triethylamine (0.26 mL, 1.87 mmol, 3.7 eq), followed by a solution of 4-(3-(piperidin-1-yl)propoxy)benzoyl chloride in DCM (0.692 mM; 1 mL, 0.692 mmol, 1.4 eq). The solution was then stirred at room temperature overnight for 23 hours. Upon completion, the reaction was quenched with 10 mL AQ K₂CO₃ and extracted with 50 mL DCM. The organic layer was separated and concentrated. The crude material was then purified via automated flash column chromatography using a gradient of 100:0 to 90:10 DCM:MeOH to afford the desired product as a yellow foam (189.5 mg, 0.470 mmol, 93% yield).¹H NMR: (600 MHz, CDCl₃) δ 8.61 (dd, J=8.3, 1.4 Hz, 1H), 7.68 (s, 1H), 7.57-7.55 (m, 2H), 7.45 (td, J=7.9, 1.6 Hz, 1H), 7.36 (dd, J=7.8, 1.6 Hz, 1H), 7.19 (td, J=7.6, 1.4 Hz, 1H), 6.89-6.87 (m, 2H), 6.85 (t, J=2.1 Hz, 2H), 6.46 (t, J=2.1 Hz, 2H), 4.08 (t, J=6.0 Hz, 2H), 2.96-2.58 (m, 6H), 2.31-2.17 (m, 2H), 1.95-1.73 (m, 4H), 1.63-1.50 (m, 2H). ¹³C NMR: (151 MHz, CDCl₃) δ 164.5, 161.6, 134.5, 130.6, 129.1, 128.9, 126.8, 126.7, 123.9, 122.2, 120.9, 114.4, 110.6, 77.2, 77.0, 76.8, 65.9, 55.5, 54.1, 25.2, 24.2, 23.3.

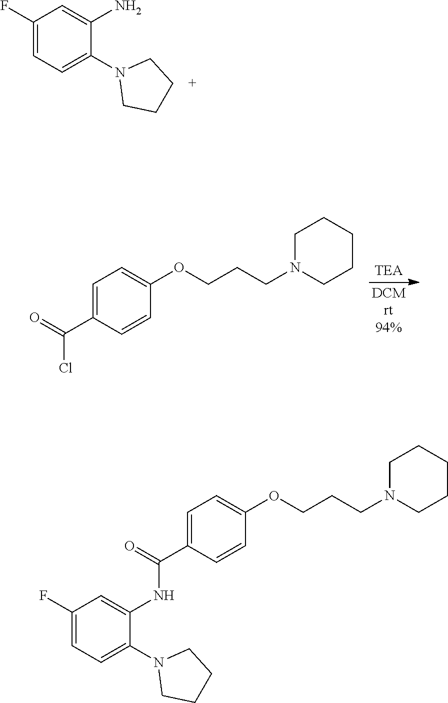 [SMILES: Nc1cc(F)ccc1N1CCCC1 ; O=C(Cl)c1ccc(OCCCN2CCCCC2)cc1 ; O=C(Nc1cc(F)ccc1N1CCCC1)c1ccc(OCCCN2CCCCC2)cc1]

N-(5-fluoro-2-(pyrrolidin-1-yl)phenyl)-4-(3-(piperidin-1-yl)propoxy)benzamide. To an oven-dried vial was added 5-fluoro-2-(pyrrolidine-1-yl)aniline (89.5 mg, 0.497 mmol, 1.0 eq), which was then dissolved in 4 mL DCM. To the solution was then added triethylamine (0.26 mL, 1.87 mmol, 3.8 eq), followed by a solution of 4-(3-(piperidin-1-yl)propoxy)benzoyl chloride in DCM (0.692 mM; 1 mL, 0.692 mmol, 1.4 eq). The solution was then stirred at room temperature for 61.75 hours. Upon completion, the reaction was quenched with 10 mL AQ K₂CO₃ and extracted with 50 mL DCM. The organic layer was separated and concentrated. The crude material was purified via automated flash column chromatography using a gradient of 100:0 to 90:10 DCM:MeOH to afford the desired product as a red solid (199.0 mg, 0.468 mmol, 94% yield). ¹H NMR: (600 MHz, CDCl₃) δ 9.38 (s, 1H), 8.30 (dd, J=10.9, 3.0 Hz, 1H), 7.84-7.82 (m, 2H), 7.16 (dd, J=8.8, 5.5 Hz, 1H), 6.99-6.96 (m, 2H), 6.75 (ddd, J=8.4, 8.4, 3.0 Hz, 1H), 4.13 (t, J=6.0 Hz, 2H), 3.03-3.00 (m, 4H), 2.96-2.63 (m, 6H), 2.33-2.20 (m, 2H), 2.02-1.98 (m, 4H), 1.95-1.76 (m, 4H), 1.65-1.50 (m, 2H).

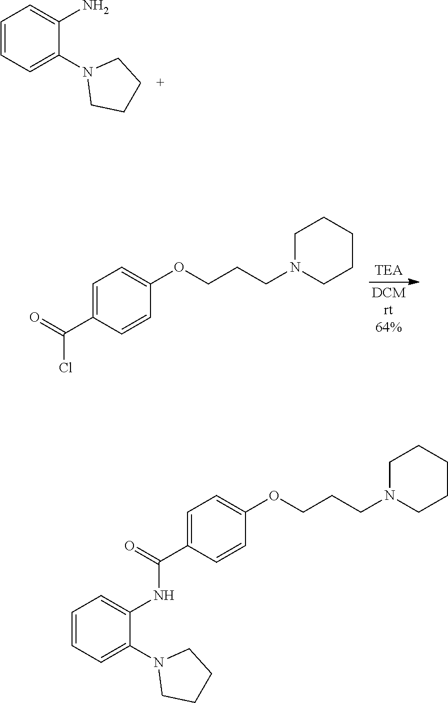 [SMILES: Nc1ccccc1N1CCCC1 ; O=C(Cl)c1ccc(OCCCN2CCCCC2)cc1 ; O=C(Nc1ccccc1N1CCCC1)c1ccc(OCCCN2CCCCC2)cc1]

4-(3-(piperidin-1-yl)propoxy)-N-(2-(pyrrolidin-1-yl)phenyl)benzamide. To an oven-dried vial was added 2-(pyrrolidine-1-yl)aniline (80.9 mg, 0.499 mmol, 1.0 eq), which was then dissolved in 4 mL DCM. To the solution was added triethylamine (0.26 mL, 1.87 mmol, 3.7 eq), followed by a solution of 4-(3-(piperidin-1-yl)propoxy)benzoyl chloride in DCM (0.692 mM; 1 mL, 0.692 mmol, 1.4 eq). The solution was then stirred at room temperature for 61.75 hours. Upon completion, the reaction was quenched with 10 mL AQ K2CO3 and extracted with 50 mL DCM. The organic layer was separated and concentrated. The crude material was then purified via automated flash column chromatography using a gradient of 100:0 to 90:10 DCM:MeOH to afford the desired product as a red foam (130.3 mg, 0.320 mmol, 64% yield). ¹H NMR: (600 MHz, CDCl₃) δ 9.06 (s, 1H), 8.39 (d, J=7.7 Hz, 1H), 7.86-7.84 (m, 2H), 7.19 (dd, J=7.8, 1.5 Hz, 1H), 7.13 (td, J=7.7, 1.5 Hz, 1H), 7.08 (td, J=7.6, 1.5 Hz, 1H), 6.98-6.95 (m, 2H), 4.14 (t, J=5.8 Hz, 2H), 3.08-3.05 (m, 4H), 3.04-2.73 (m, 6H), 2.40-2.30 (m, 2H), 2.02-1.86 (m, 8H), 1.70-1.51 (m, 2H). ¹³C NMR: (151 MHz, CDCl₃) δ 164.3, 161.2, 140.3, 133.4, 128.8, 128.0, 124.4, 124.0, 120.3, 119.5, 114.4, 77.2, 77.0, 76.8, 65.7, 55.5, 53.9, 52.6, 24.7, 24.5, 23.6, 22.9.

### Example 17. N-(2-(1-ethyl-1H-pyrazol-5-yl)phenyl)-4-(2-(piperidin-1-yl)ethoxy)benzamide acetic acid salt (LLM418-5NEt)

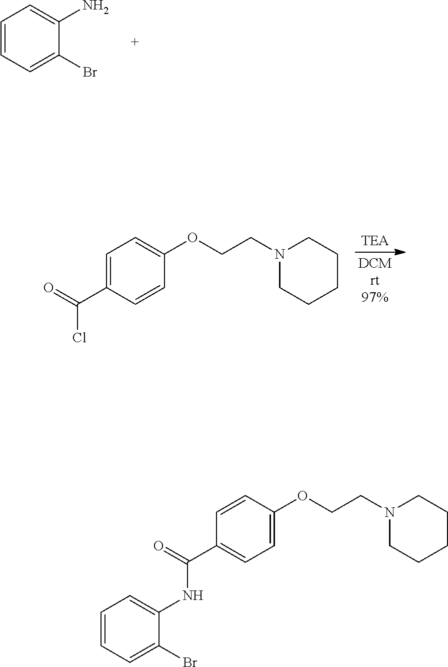 [SMILES: Nc1ccccc1Br ; O=C(Cl)c1ccc(OCCN2CCCCC2)cc1 ; O=C(Nc1ccccc1Br)c1ccc(OCCN2CCCCC2)cc1]

N-(2-bromophenyl)-4-(2-(piperidin-1-yl)ethoxy)benzamide: To an oven-dried 100 mL round bottom flask was added a solution of 2-bromoaniline (690.2 mg, 4.012 mmol, 1.0 eq) in 15 mL DCM. Triethylamine (1.51 mL, 10.833 mmol, 2.7 eq) was added to the solution, followed by a freshly prepared solution of 4-(2-(piperidin-1-yl)ethoxy)benzoyl chloride (1.446 g, 5.402 mmol, 1.35 eq) in 15 mL DCM. The solution was stirred at room temperature overnight. After 50 h, the reaction was quenched with 20 mL AQ K₂CO₃ and extracted with 100 mL DCM. The organic layer was separated and concentrated. The crude material was then purified via automated flash column chromatography using a gradient of 84:15:1 to 52:47:1 Hex:EtOAc:TEA to afford the desired product as a cream-colored solid (1.5699 g, 3.892 mmol, 97%).

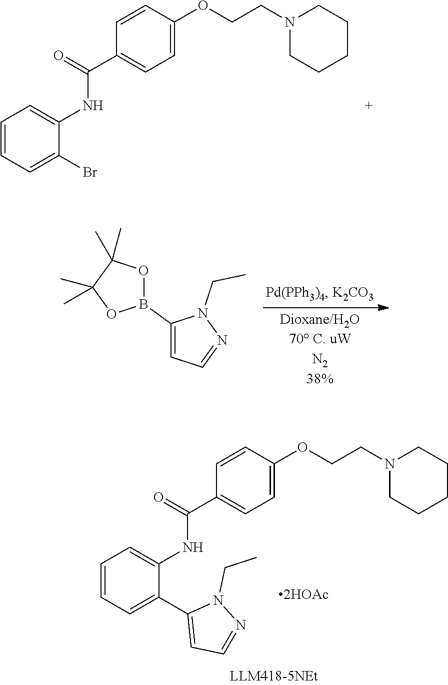 [SMILES: O=C(Nc1ccccc1Br)c1ccc(OCCN2CCCCC2)cc1 ; CCn1nccc1B1OC(C)(C)C(C)(C)O1 ; CCn1nccc1-c1ccccc1NC(=O)c1ccc(OCCN2CCCCC2)cc1]

N-(2-(1-ethyl-1H-pyrazol-5-yl)phenyl)-4-(2-(piperidin-1-yl)ethoxy)benzamide acetic acid salt: To a 5 mL W vial was added N-(2-bromophenyl)-4-(2-(piperidin-1-yl)ethoxy)benzamide (101.8 mg, 0.252 mmol, 1.0 eq), 1-ethyl-5-(4,4,5,5-tetramethyl-1,3,2-dioxaborolane-2-yl)-1H-pyrazole (73.2 mg, 0.330 mmol, 1.33 eq), K₂CO₃ (104.6 mg, 0.757 mmol, 3.0 eq), and Pd(PPh₃)₄(14.8 mg, 0.0128 mmol, 0.05 eq). 3 mL degassed 5:1 1,4-dioxane:H₂O was then added. The mixture was degassed with N₂. The vial was then sealed and the mixture was heated to 70° C. via microwave for 2 hours. Upon completion of the reaction, 5 mL saturated aqueous NaHCO₃was added. The mixture was then extracted with 3×15 mL portions of DCM. The organic layers were combined and concentrated. The crude material was then purified via preparative HPLC using H₂O:MeCN:AcOH to afford the diacetate salt of the desired product as a red-gold oil (50.9 mg, 0.0945 mmol, 38%).

### Example 18: N-(2-(1-benzyl-1H-pyrazol-3-yl)-5-fluorophenyl)-4-(2-(piperidin-1-yl)ethoxy)benzamide (LLM418-FBn)

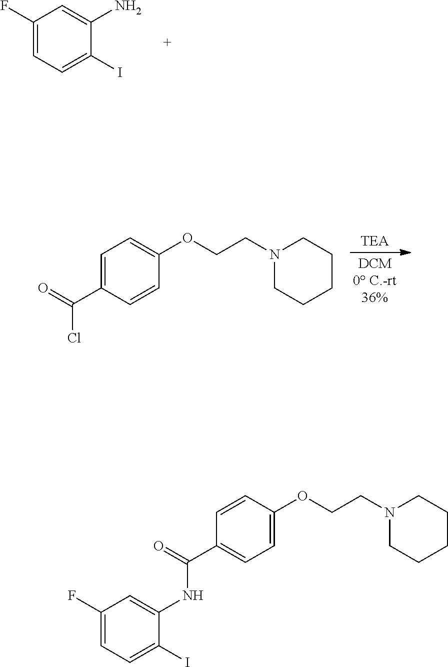 [SMILES: Nc1cc(F)ccc1I ; O=C(Cl)c1ccc(OCCN2CCCCC2)cc1 ; O=C(Nc1cc(F)ccc1I)c1ccc(OCCN2CCCCC2)cc1]

N-(5-fluoro-2-iodophenyl)-4-(2-(piperidin-1-yl)ethoxy)benzamide: To an oven-dried vial was added 5-fluoro-2-iodoaniline (202.4 mg, 0.854 mmol, 1.0 eq), which was dissolved in 6 mL DCM. The solution was cooled to 0° C., and TEA (0.30 mL, 2.15 mmol, 2.5 eq) was added. To the solution was then added 3.25 mL of a freshly prepared 0.391M solution of 4-(2-(piperidin-1-yl)ethoxy)benzoyl chloride in DCM (1.27 mmol, 1.5 eq). The solution was stirred overnight and allowed to gradually warm to room temperature. After 15 hours, the reaction was quenched with 10 mL AQ K₂CO₃ and extracted with 2×25 mL portions of DCM. The organic layers were combined, dried over anhydrous Na₂SO₄, and concentrated. The crude material was then purified via automated flash column chromatography using a gradient of 83:15:2 to 28:70:2 Hex:EtOAc:TEA to afford the desired product as an off-white solid (145.4 mg, 0.310 mmol, 36%).

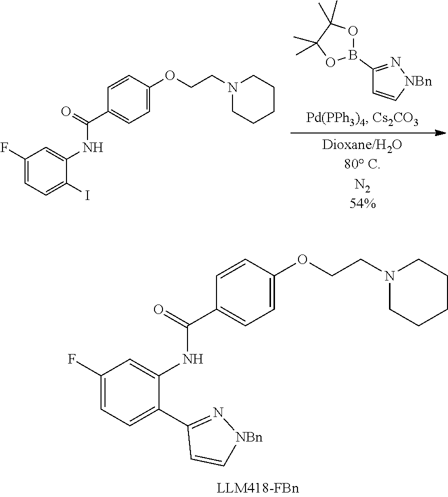 [SMILES: O=C(Nc1cc(F)ccc1I)c1ccc(OCCN2CCCCC2)cc1 ; CC1(C)OB(c2ccn(Cc3ccccc3)n2)OC1(C)C ; O=C(Nc1cc(F)ccc1-c1ccn(Cc2ccccc2)n1)c1ccc(OCCN2CCCCC2)cc1]

N-(2-(1-benzyl-1H-pyrazol-3-yl)-5-fluorophenyl)-4-(2-(piperidin-1-yl)ethoxy)benzamide: To a vial was added 1-benzyl-3-(4,4,5,5-tetramethyl-1,3,2-dioxaborolan-2-yl)-1H-pyrazole (116.7 mg, 0.411 mmol, 1.5 eq), followed by N-(5-fluoro-2-iodophenyl)-4-(2-(piperidin-1-yl)ethoxy)benzamide (124.8 mg, 0.266 mmol, 1.0 eq). The reagents were suspended in 3 mL degassed 10:1 1,4-dioxane:H₂O. To the suspension was then added Cs₂CO₃ (265.1 mg, 0.814 mmol, 3.1 eq) and Pd(PPh₃)₄(31.2 mg, 0.027 mmol, 0.1 eq). The mixture was degassed with N₂, then heated to 80° C. under N₂ for 42 hours. Upon completion of the reaction, the mixture was diluted with EtOAc, filtered over celite, and concentrated. The crude material was purified via automated flash column chromatography using a gradient of 0-10% MeOH in DCM to afford the desired product as an orange-yellow residue (71.1 mg, 0.143 mmol, 54%).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### REFERENCES

1. Yao, X.; Huang, J.; Zhong, H.; Shen, N.; Faggioni, R.; Fung, M.; Yao, Y. Pharmacology & Therapeutics. 2014, 141, 125.
2. Stein, B.; Sutherland, M. S. K. Drug Discovery Today. 1998, 3 (5), 202.
3. Guqin, Shi. Ph.D. Dissertation. The Ohio State University, Columbus, O H, 2017.
4. Mao, Liguang. Ph.D. Dissertation. The Ohio State University, Columbus, O H, 2017.
5. Omura, S.; Hayashi, M.; Tomoda, H. Pure Appl. Chem. 1999, 71, 1673.
6. Saleh, A. Z. M.; Greenman, K. L.; Billings, S.; Van Vranken, D. L.; Krolewski, J. J. Biochemistry. 2005, 44, 10822.
7. Li, H.; Xiao, H.; Lin, L.; Jou, D.; Kumari, V.; Lin, J.; Li, C. J. Med. Chem. 2014, 57, 632.
8. Hong, S.; Choi, J.; Lee, S.; et al. J. Immunol. 2015, 195, 237.
9. Boulanger, M. J.; Chow, D.; Brevnova, E. E.; Garcia, K. C. Science 2003, 300, 2101.

### Embodiments

1. A compound of Formula (F), or a salt thereof:

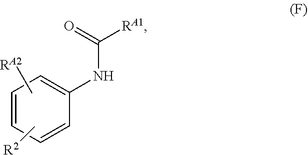

wherein:

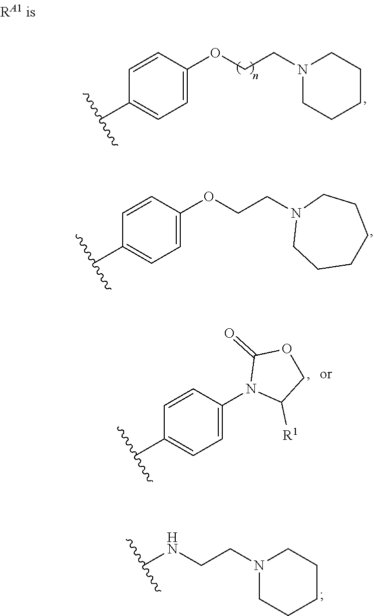

R¹ is optionally substituted alkyl or optionally substituted cycloalkyl;
R^{A2} is H or halogen;
R² is optionally substituted

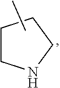

optionally substituted

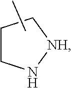

or optionally substituted

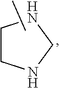

optionally substituted

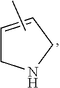

optionally substituted

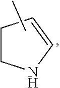

optionally substituted

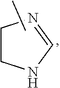

optionally substituted

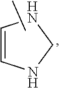

optionally substituted

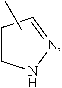

optionally substituted

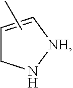

optionally substituted

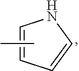

optionally substituted

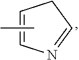

optionally substituted

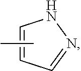

optionally substituted

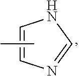

optionally substituted

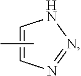

optionally substituted

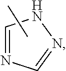

optionally substituted

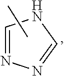

or optionally substituted

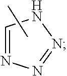

and
n is 1 or 2.

2. The compound of embodiment 1, or a salt thereof, wherein the compound is of Formula (A):

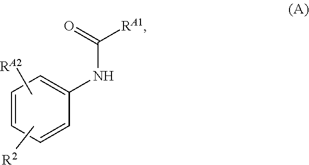

wherein:
R^{A1} is

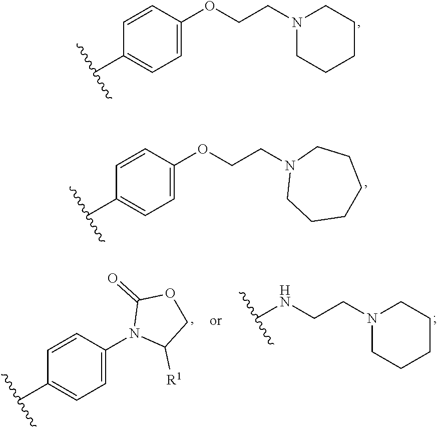

R¹ is optionally substituted alkyl or cycloalkyl;
R^{A2} is H or halogen; and
R² is optionally substituted

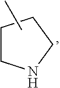

optionally substituted

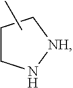

or optionally substituted

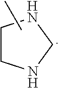

optionally substituted

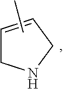

optionally substituted

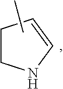

optionally substituted

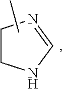

optionally substituted

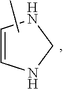

optionally substituted

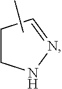

optionally substituted

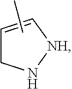

optionally substituted

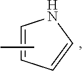

optionally substituted

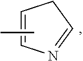

optionally substituted

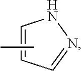

optionally substituted

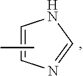

optionally substituted

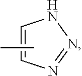

optionally substituted

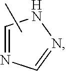

optionally substituted

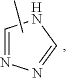

or optionally substituted

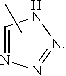

3. The compound of embodiment 1 or 2, wherein the compound is of Formula (IV), or a salt thereof:

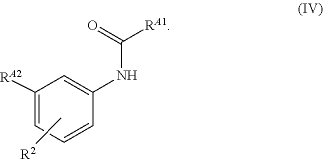

4. The compound of embodiment 3, wherein the compound is of Formula (IV-c), or a salt thereof:

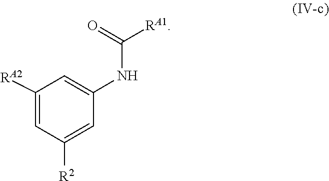

5. The compound of embodiment 3, wherein the compound is of Formula (IV-d), or a salt thereof:

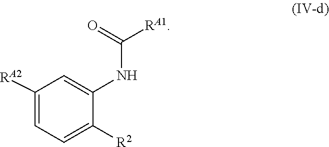

6. The compound of embodiment 3, wherein the compound is of Formula (IV-e), or a salt thereof:

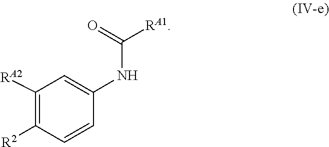

7. The compound of embodiment 1 or 2, wherein compound is of Formula (I), or a salt thereof:

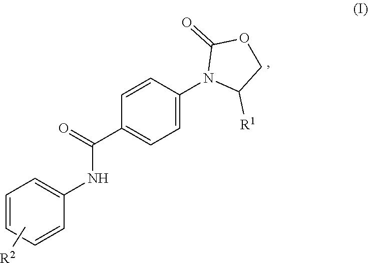

wherein:
R¹ is alkyl or cycloalkyl; and
R² is selected from the group consisting of:

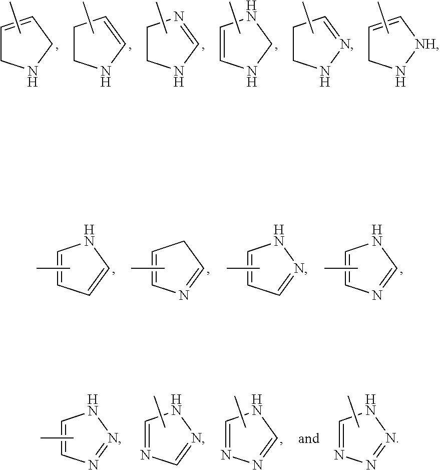

8. The compound of embodiment 7, or a salt thereof, according to Formula (I-a):

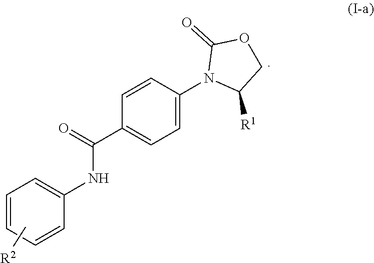

9. The compound of embodiment 7, or a salt thereof, according to Formula (I-b):

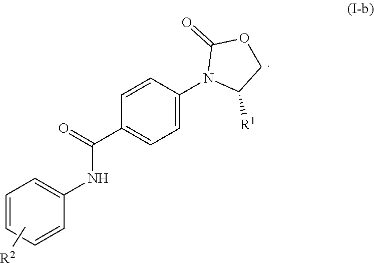

10. The compound of any one of embodiments 7-9, or a salt thereof, according to Formula (I-c):

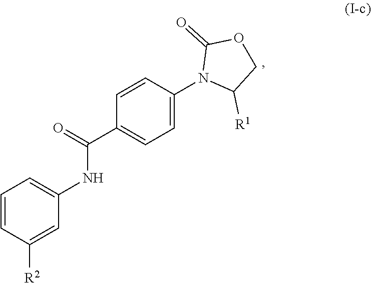

11. The compound of any one of embodiments 7-9, or a salt thereof, according to Formula (I-d):

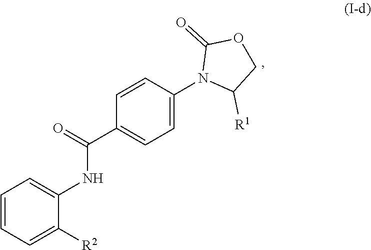

12. The compound of embodiment 1 or 2, wherein the compound is of Formula (V) or (VI), or a salt thereof:

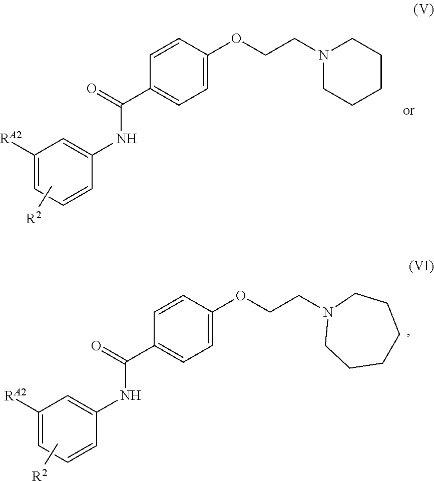

wherein R² is optionally substituted

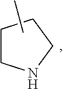

optionally substituted

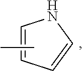

optionally substituted

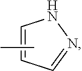

or optionally substituted

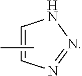

13. The compound of embodiment 12, wherein the compound is of Formula (V-A), or a salt thereof:

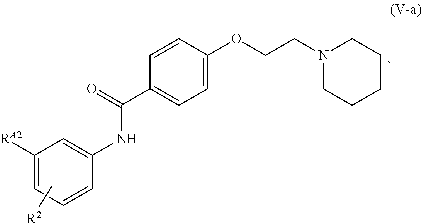

wherein:
R² is

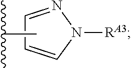

and
R^{A3} is optionally substituted alkyl or optionally substituted carbocyclyl.

14. The compound of embodiment 1 or 2, wherein the compound is of Formula (VII) or (VIII), or a salt thereof:

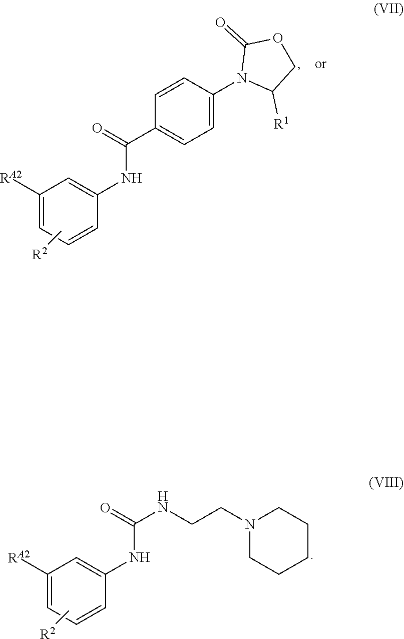

15. The compound of any one of embodiments 1-6, 12, or 14, or a salt thereof, wherein R² is optionally substituted

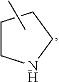

optionally substituted

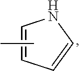

optionally substituted

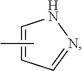

or optionally substituted

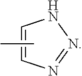

16. The compound of any one of embodiments 1-6, 12, or 14, or a salt thereof, wherein R² is

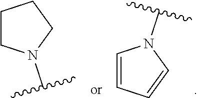 [SMILES: *N1CCCC1 ; *n1cccc1]

17. The compound of any one of embodiments 1-6, 12, or 14, or a salt thereof, wherein R² is

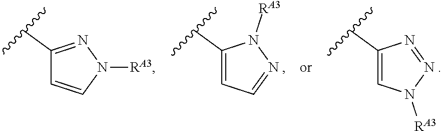

18. The compound of embodiment 13 or 17, wherein R^{A3} is optionally substituted C₁₋₆ alkyl or optionally substituted C₃₋₈ carbocyclyl.

19. The compound of embodiment 13, 17, or 18, wherein R^{A3} is

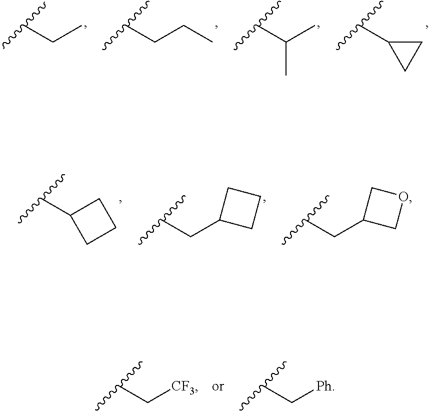

20. The compound of any one of embodiments 1-6, 12, or 14, or a salt thereof, wherein R² is

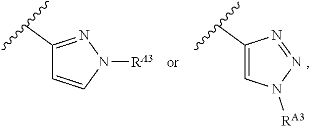

wherein R^{A3} is

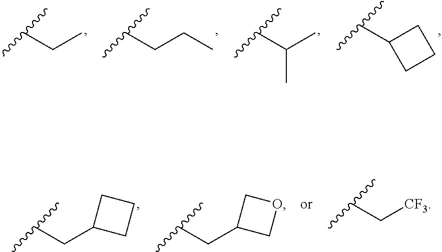

21. The compound of any one of embodiments 1-15, 17, or 20, or a salt thereof, wherein R² is

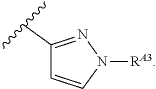

22. The compound of any one of embodiments 1-11, or a salt thereof, wherein R² is

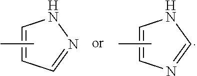

23. The compound of embodiment 22, or a salt thereof, wherein R² is

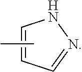

24. The compound of any one of the preceding embodiments, or a salt thereof, wherein R¹ is C₁₋₆ alkyl.

25. The compound of embodiment 24, or a salt thereof, wherein R¹ is isopropyl.

26. The compound of any one of embodiments 1-23, or a salt thereof, wherein R¹ is C₁₋₈ cycloalkyl.

27. The compound of embodiment 26, or a salt thereof, wherein R¹ is cyclopropyl or cyclobutyl.

28. The compound of any one of the preceding embodiments, or a salt thereof, wherein R^{A2} is hydrogen.

29. The compound of any one of the preceding embodiments, or a salt thereof, wherein R^{A2} is halogen.

30. The compound of embodiment 29, or a salt thereof, wherein R^{A2} is fluorine.

31. The compound of embodiment 1 or 2, wherein the compound is selected from the group consisting of:

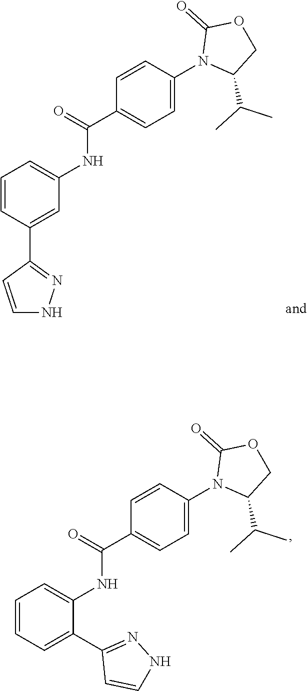 [SMILES: CC(C)[C@H]1COC(=O)N1c1ccc(C(=O)Nc2cccc(-c3cc[nH]n3)c2)cc1 ; CC(C)[C@H]1COC(=O)N1c1ccc(C(=O)Nc2ccccc2-c2cc[nH]n2)cc1]

or a salt thereof.

32. The compound of embodiment 1 or 2, wherein the compound is selected from the group consisting of

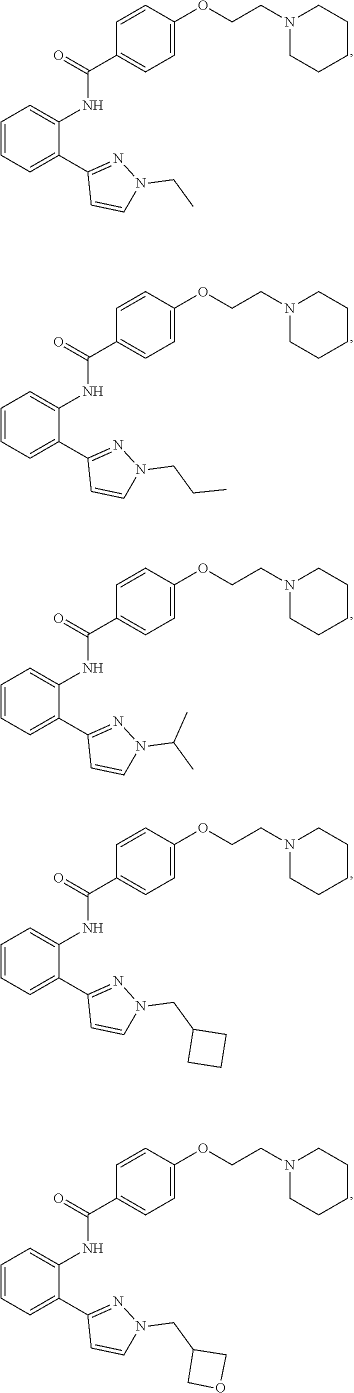 [SMILES: CCn1ccc(-c2ccccc2NC(=O)c2ccc(OCCN3CCCCC3)cc2)n1 ; CCCn1ccc(-c2ccccc2NC(=O)c2ccc(OCCN3CCCCC3)cc2)n1 ; CC(C)n1ccc(-c2ccccc2NC(=O)c2ccc(OCCN3CCCCC3)cc2)n1 ; O=C(Nc1ccccc1-c1ccn(CC2CCC2)n1)c1ccc(OCCN2CCCCC2)cc1 ; O=C(Nc1ccccc1-c1ccn(CC2COC2)n1)c1ccc(OCCN2CCCCC2)cc1]

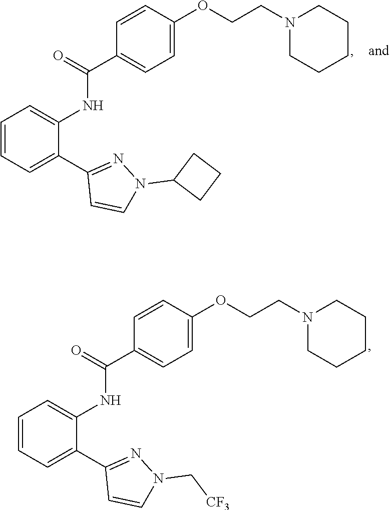 [SMILES: O=C(Nc1ccccc1-c1ccn(C2CCC2)n1)c1ccc(OCCN2CCCCC2)cc1 ; O=C(Nc1ccccc1-c1ccn(CC(F)(F)F)n1)c1ccc(OCCN2CCCCC2)cc1]

or a salt thereof.

33. The compound of embodiment 1 or 2, wherein the compound is selected from the group consisting of

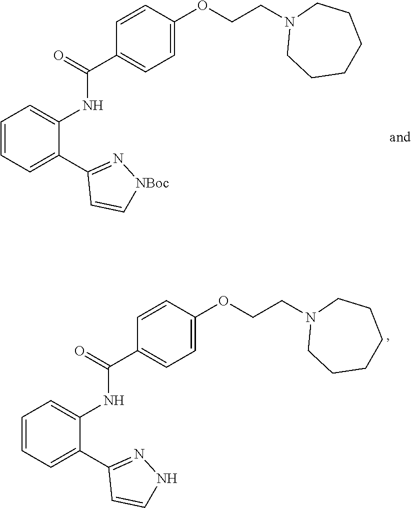 [SMILES: CC(C)(C)OC(=O)n1ccc(-c2ccccc2NC(=O)c2ccc(OCCN3CCCCCC3)cc2)n1 ; O=C(Nc1ccccc1-c1cc[nH]n1)c1ccc(OCCN2CCCCCC2)cc1]

or a salt thereof.

34. The compound of embodiment 1 or 2, wherein the compound is selected from the group consisting of

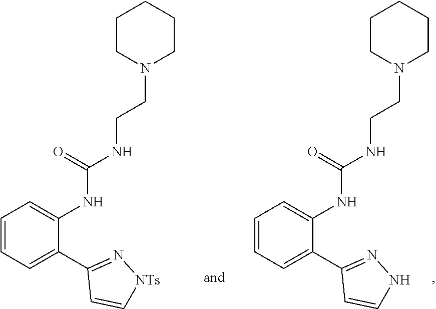 [SMILES: Cc1ccc(S(=O)(=O)n2ccc(-c3ccccc3NC(=O)NCCN3CCCCC3)n2)cc1 ; O=C(NCCN1CCCCC1)Nc1ccccc1-c1cc[nH]n1]

or a salt thereof.

35. The compound of embodiment 1 or 2, wherein the compound is selected from the group consisting of

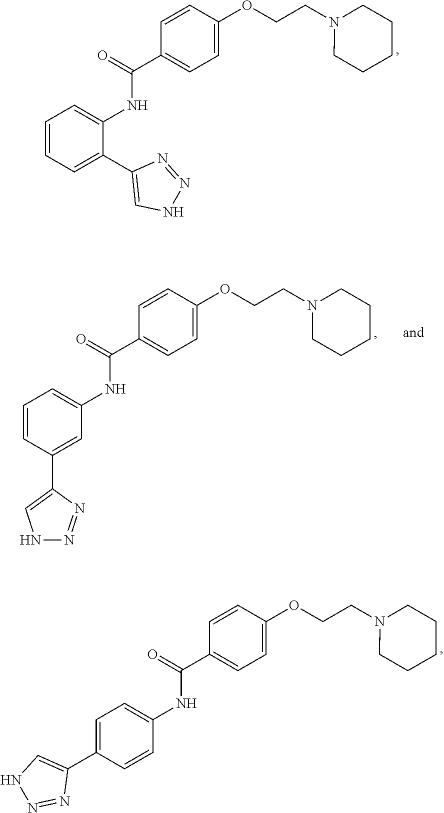 [SMILES: O=C(Nc1ccccc1-c1c[nH]nn1)c1ccc(OCCN2CCCCC2)cc1 ; O=C(Nc1cccc(-c2c[nH]nn2)c1)c1ccc(OCCN2CCCCC2)cc1 ; O=C(Nc1ccc(-c2c[nH]nn2)cc1)c1ccc(OCCN2CCCCC2)cc1]

or a salt thereof.

36. The compound of embodiment 1 or 2, wherein the compound has the structure

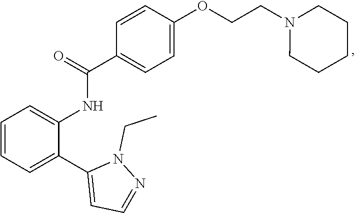 [SMILES: CCn1nccc1-c1ccccc1NC(=O)c1ccc(OCCN2CCCCC2)cc1]

or a salt thereof.

37. The compound of embodiment 1 or 2, wherein the compound is selected from the group consisting of

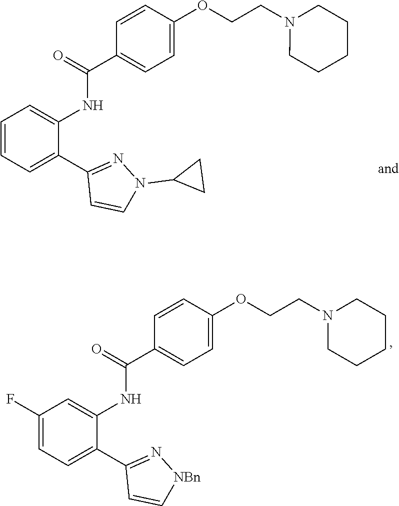 [SMILES: O=C(Nc1ccccc1-c1ccn(C2CC2)n1)c1ccc(OCCN2CCCCC2)cc1 ; O=C(Nc1cc(F)ccc1-c1ccn(Cc2ccccc2)n1)c1ccc(OCCN2CCCCC2)cc1]

or a salt thereof.

38. The compound of embodiment 1, wherein the compound is selected from the group consisting of

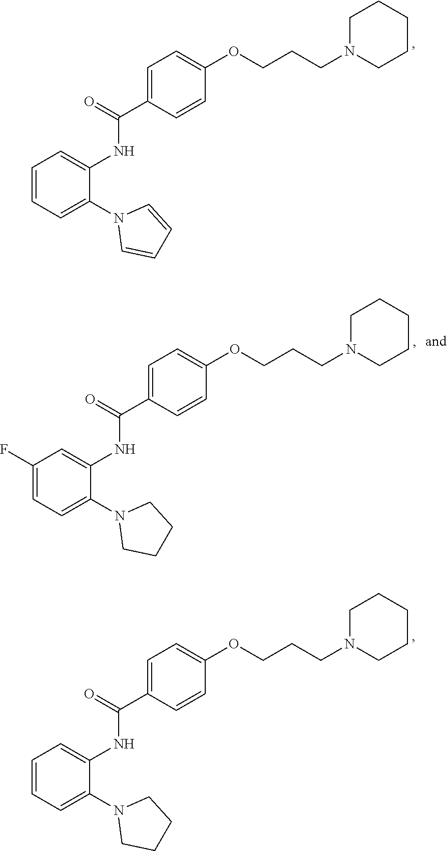 [SMILES: O=C(Nc1ccccc1-n1cccc1)c1ccc(OCCCN2CCCCC2)cc1 ; O=C(Nc1cc(F)ccc1N1CCCC1)c1ccc(OCCCN2CCCCC2)cc1 ; O=C(Nc1ccccc1N1CCCC1)c1ccc(OCCCN2CCCCC2)cc1]

or a salt thereof.

39. A compound of Formula (B), or a salt thereof:

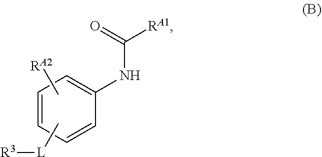

wherein:

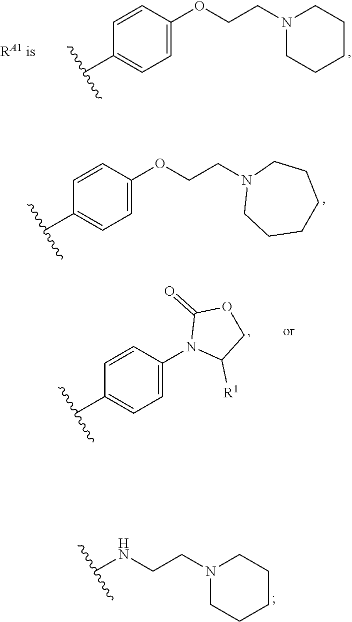

R¹ is optionally substituted alkyl or optionally substituted cycloalkyl;
R^{A2} is H or halogen;
L is a linker selected from the group consisting of —NH(C═O)—, —(C═O)NH—, optionally substituted five-membered N-containing heterocyclylene, optionally substituted five-membered N-containing heteroarylene, or optionally substituted heteroarylalkylene; and
R³ is a carbohydrate or carbohydrate substituted with one or more oxygen protecting groups.

40. The compound of embodiment 39, wherein the compound is of Formula (IX), or a salt thereof:

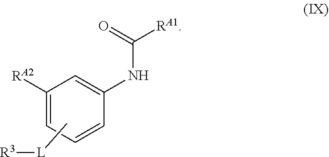

41. The compound of embodiment 40, wherein the compound is of Formula (IX-c), or a salt thereof:

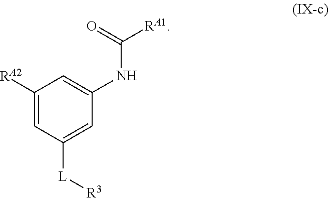

42. The compound of embodiment 40, wherein the compound is of Formula (IX-d), or a salt thereof:

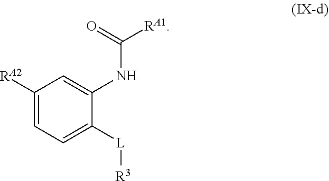

43. The compound of embodiment 40, wherein the compound is of Formula (IX-e), or a salt thereof:

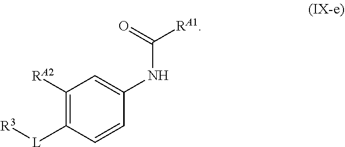

44. The compound of embodiment 40, wherein the compound is of Formula (X), (XI), (XII), or (XIII), or a salt thereof:

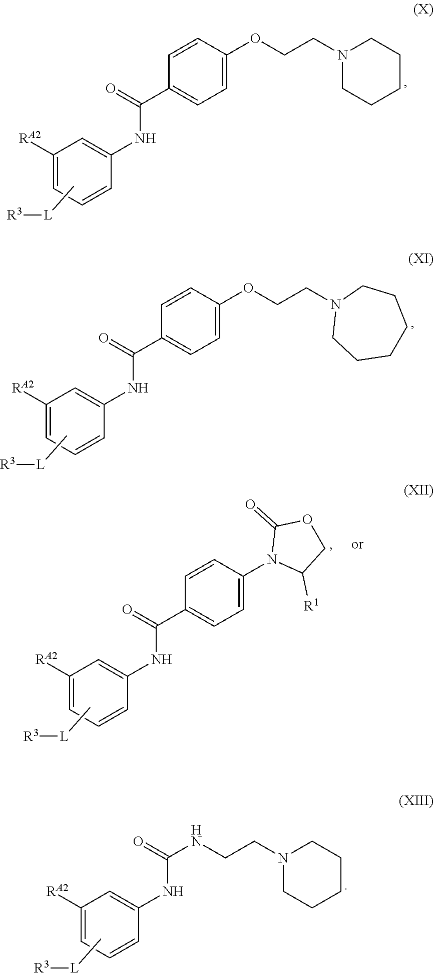

45. The compound of embodiment 44, wherein the compound is of Formula (X-c), (XI-c), (XII-c), or (XIII-c), or a salt thereof:

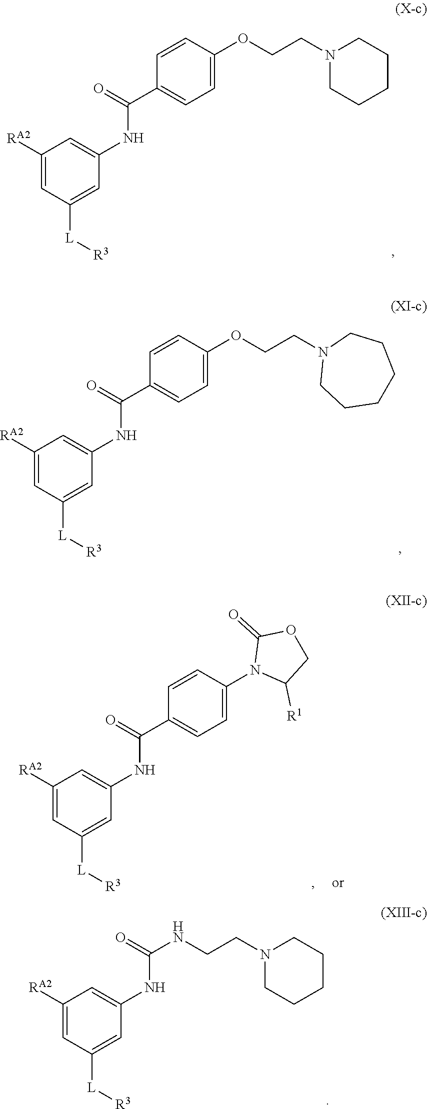

46. The compound of embodiment 44, wherein the compound is of Formula (X-d), (XI-d), (XII-d), or (XIII-d), or a salt thereof:

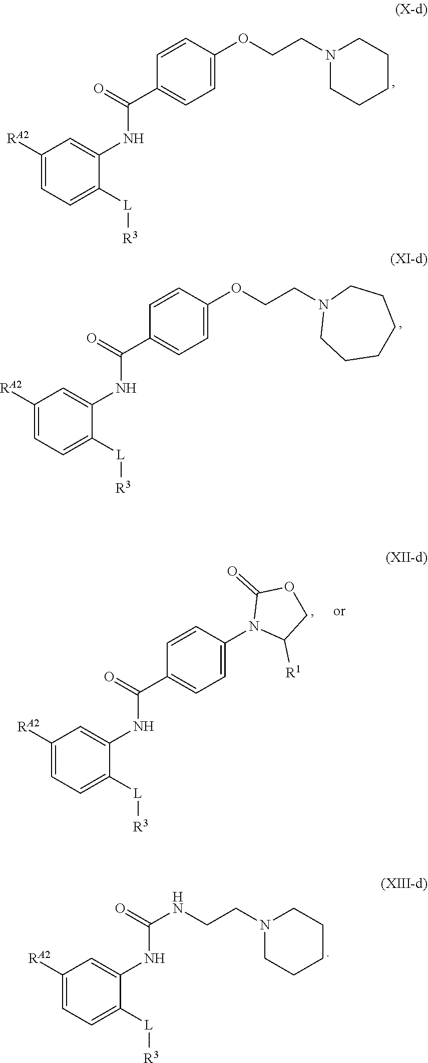

47. The compound of embodiment 44, wherein the compound is of Formula (X-e), (XI-e), (XII-e), or (XIII-e), or a salt thereof:

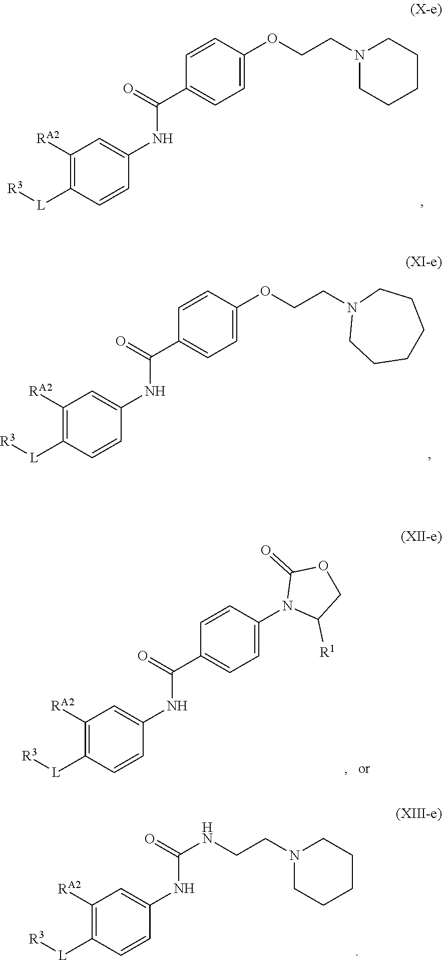

48. The compound of any one of embodiments 39-47, or a salt thereof, wherein L is —NH(C═O)— or —(C═O)NH—.

49. The compound of any one of embodiments 39-47, wherein L is a linker selected from the group consisting of optionally substituted five-membered N-containing heterocyclylene and optionally substituted five-membered N-containing heteroarylene.

50. The compound of embodiment 49, or a salt thereof, wherein L is optionally substituted pyrrolidinylene, optionally substituted pyrazolidinylene, optionally substituted imadazolidinylene, optionally substituted 3-pyrrolinylene, optionally substituted 2-pyrrolinylene, optionally substituted 2-pyrazolinylene, optionally substituted 2-imidazolinylene, optionally substituted 2H-pyrrolylene, optionally substituted 1H-pyrrolylene, optionally substituted pyrazolylene, optionally substituted imidazolylene, optionally substituted 1,2,4-triazolylene, optionally substituted 1,2,3-triazolylene, or optionally substituted tetrazolylene.

51. The compound of embodiment 50, or a salt thereof, wherein L is optionally substituted pyrazolylene, optionally substituted imidazolylene, optionally substituted 1,2,4-triazolylene, or optionally substituted 1,2,3-triazolylene.

52. The compound of embodiment 51, or a salt thereof, wherein L is

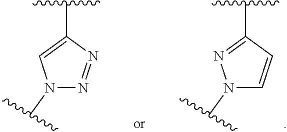 [SMILES: *c1cn(*)nn1 ; *c1ccn(*)n1]

53. The compound of embodiment 52, or a salt thereof, wherein L is

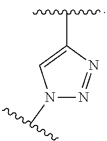 [SMILES: *c1cn(*)nn1]

54. The compound of any one of embodiments 39-53, wherein R³ is selected from the group consisting of

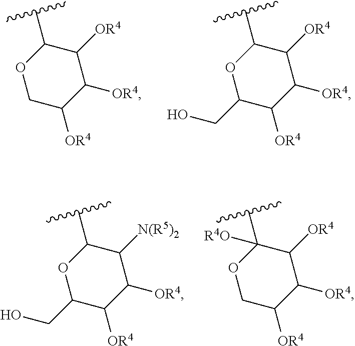

wherein:
each R⁴ is independently hydrogen or an oxygen protecting group or wherein two R⁴ are joined together with the intervening atoms to form an oxygen protecting group; and
each R⁵ is independently hydrogen or a nitrogen protecting group.

55. The compound of embodiment 54, wherein R³ is selected from the group consisting of

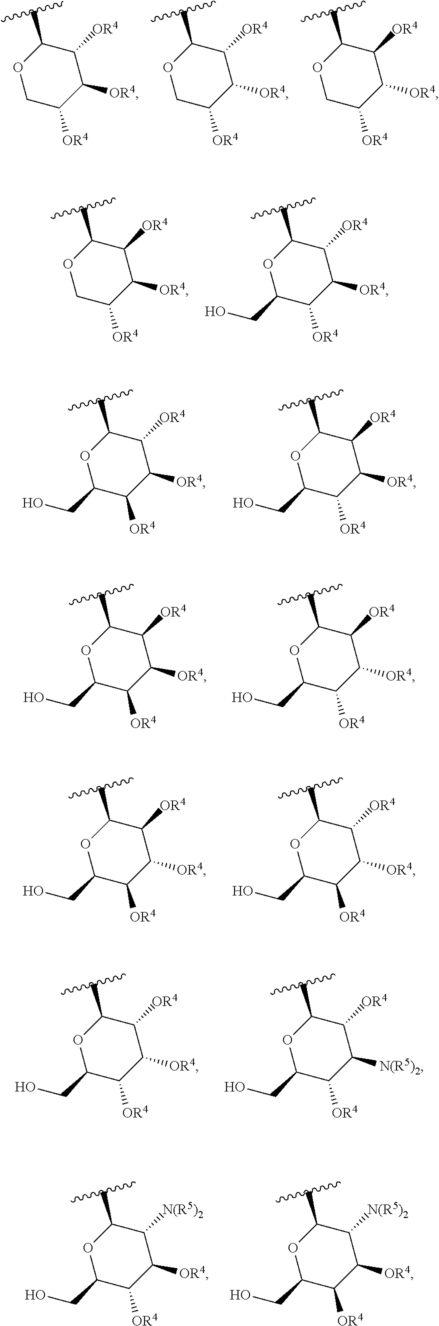

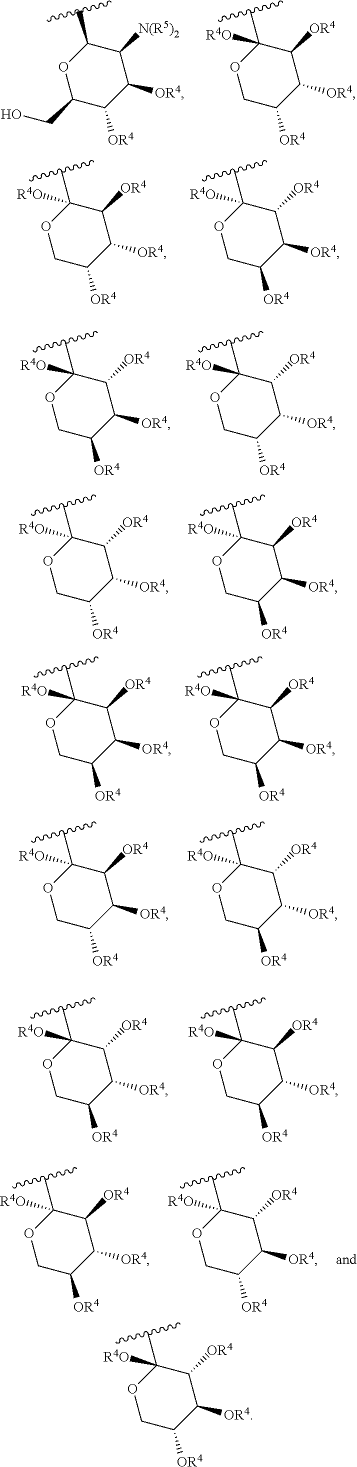

56. The compound of embodiment 39, wherein the compound is of Formula (II), or a salt thereof:

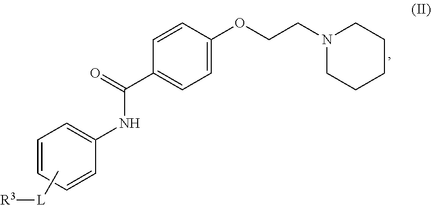

wherein:
L is a linker selected from the group consisting of —NH(C═O)—, —(C═O)NH—,

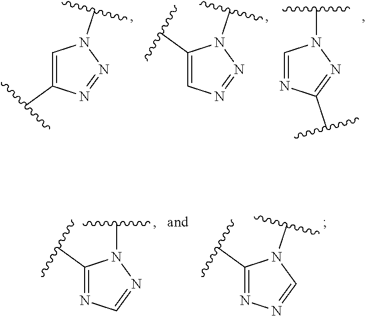 [SMILES: *c1cn(*)nn1 ; *c1cnnn1* ; *c1ncn(*)n1 ; *c1ncnn1* ; *c1nncn1*]

R³ is selected from the group consisting of

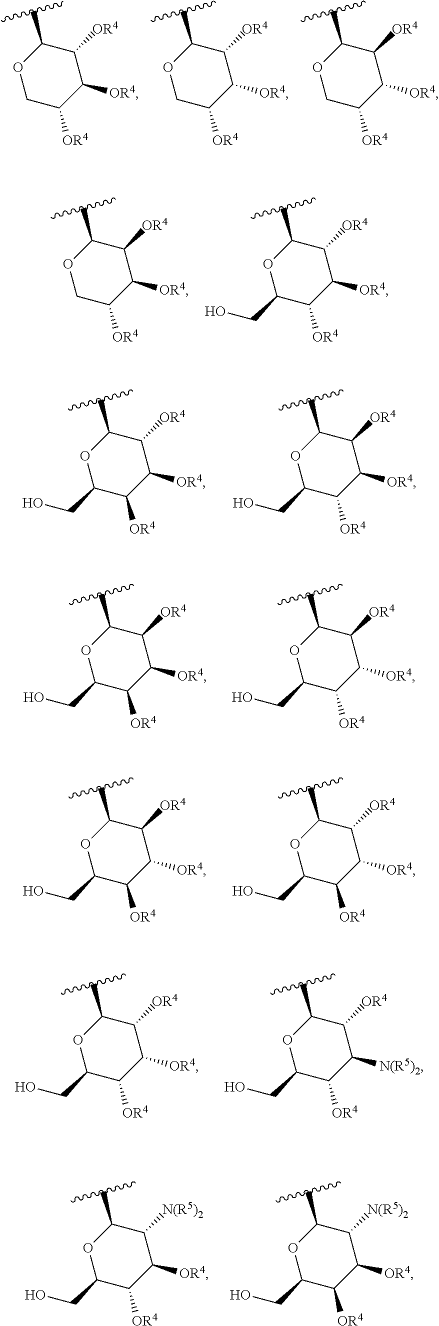

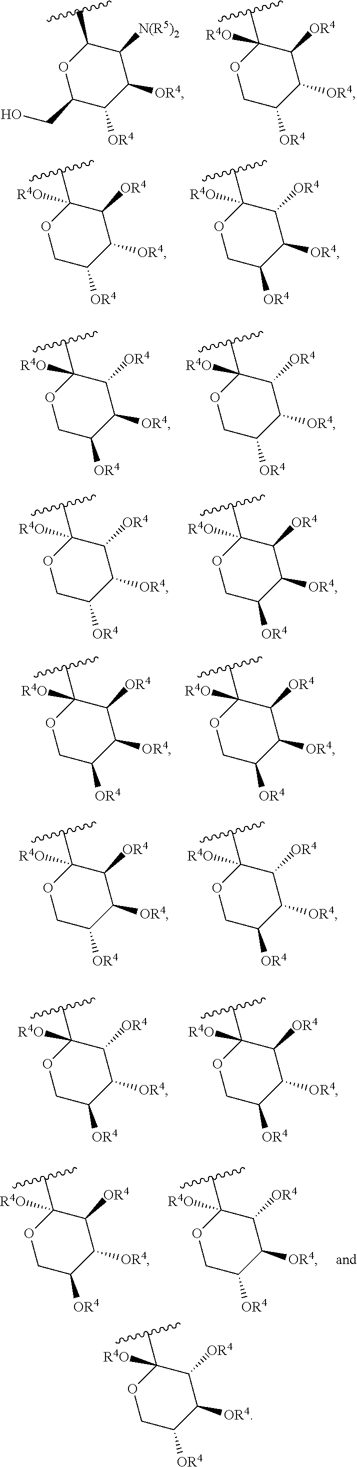

wherein:
each R⁴ is independently hydrogen or an oxygen protecting group; and
each R⁵ is independently hydrogen or a nitrogen protecting group.

57. The compound of 44, or a salt thereof, wherein R³ is

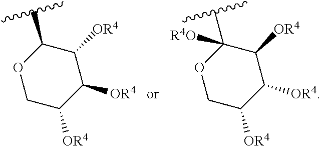

58. The compound of embodiment 57, or a salt thereof, wherein R³ is

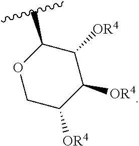

59. The compound any one of embodiments 39-47 or 49-58, or a salt thereof, wherein -L-R³ is

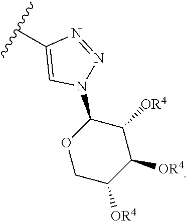

60. The compound of any one of embodiments 39-55 or 57, or a salt thereof, wherein -L-R³ is

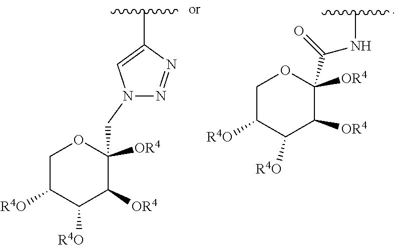

61. The compound of embodiment 56, or a salt thereof, according to Formula (II-a):

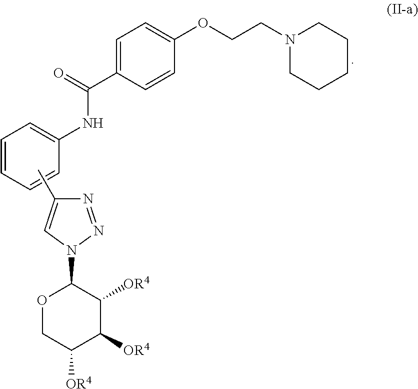

62. The compound of any one of embodiments 54-61, or a salt thereof, wherein R⁴ is H.

63. The compound of any one of embodiments 54-61, or a salt thereof, wherein R⁴ is an oxygen protecting group.

64. The compound of embodiment 63, or a salt thereof, wherein the oxygen protecting group is acyl.

65. The compound of any one of embodiments 54-61, or a salt thereof, wherein two R⁴ are joined together with the intervening atoms to form an oxygen protecting group.

66. The compound of embodiment 65, or a salt thereof, wherein two R⁴ are joined together with the intervening atoms to form an acetal protecting group.

67. The compound of any one of embodiments 39-66, or a salt thereof, wherein R^{A2} is hydrogen.

68. The compound of any one of embodiments 39-66, or a salt thereof, wherein R^{A2} is halogen.

69. The compound of embodiment 68, or a salt thereof, wherein R^{A2} is fluorine.

70. The compound of embodiment 39, wherein the compound is selected from the group consisting of:

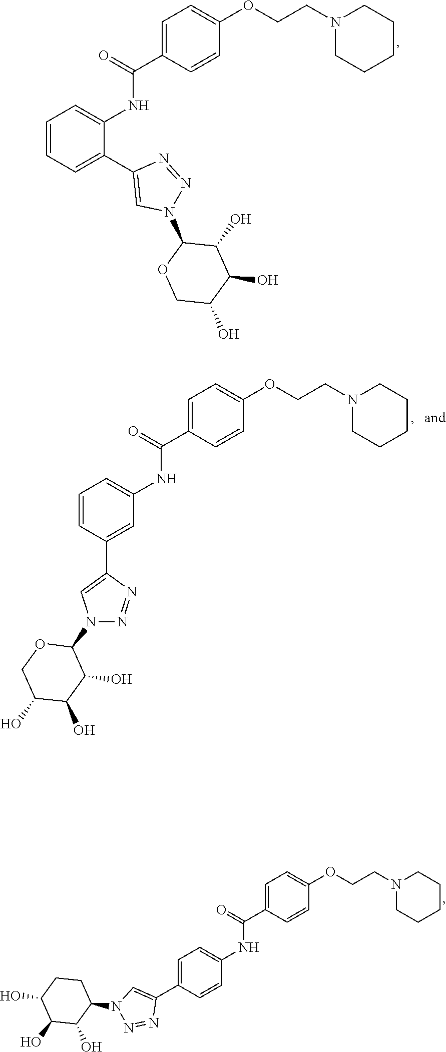 [SMILES: O=C(Nc1ccccc1-c1cn([C@@H]2OC[C@@H](O)[C@H](O)[C@H]2O)nn1)c1ccc(OCCN2CCCCC2)cc1 ; O=C(Nc1cccc(-c2cn([C@@H]3OC[C@@H](O)[C@H](O)[C@H]3O)nn2)c1)c1ccc(OCCN2CCCCC2)cc1 ; O=C(Nc1ccc(-c2cn([C@@H]3CC[C@@H](O)[C@H](O)[C@H]3O)nn2)cc1)c1ccc(OCCN2CCCCC2)cc1]

or a salt thereof.

71. The compound of embodiment 39, wherein the compound is selected from the group consisting of:

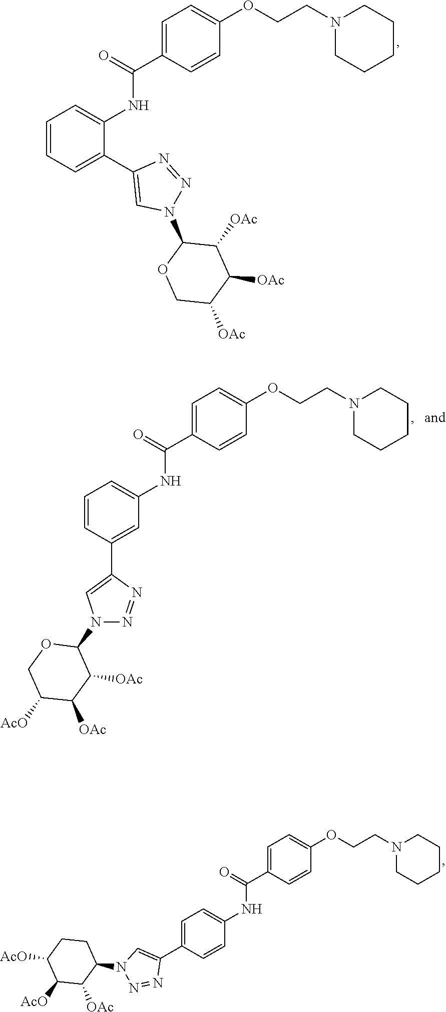 [SMILES: CC(=O)O[C@@H]1[C@@H](OC(C)=O)[C@H](OC(C)=O)CO[C@H]1n1cc(-c2ccccc2NC(=O)c2ccc(OCCN3CCCCC3)cc2)nn1 ; CC(=O)O[C@@H]1[C@@H](OC(C)=O)[C@H](OC(C)=O)CO[C@H]1n1cc(-c2cccc(NC(=O)c3ccc(OCCN4CCCCC4)cc3)c2)nn1 ; CC(=O)O[C@@H]1[C@@H](OC(C)=O)[C@H](OC(C)=O)CC[C@H]1n1cc(-c2ccc(NC(=O)c3ccc(OCCN4CCCCC4)cc3)cc2)nn1]

or a salt thereof.

72. The compound of embodiment 39, wherein the compound is selected from the group consisting of:

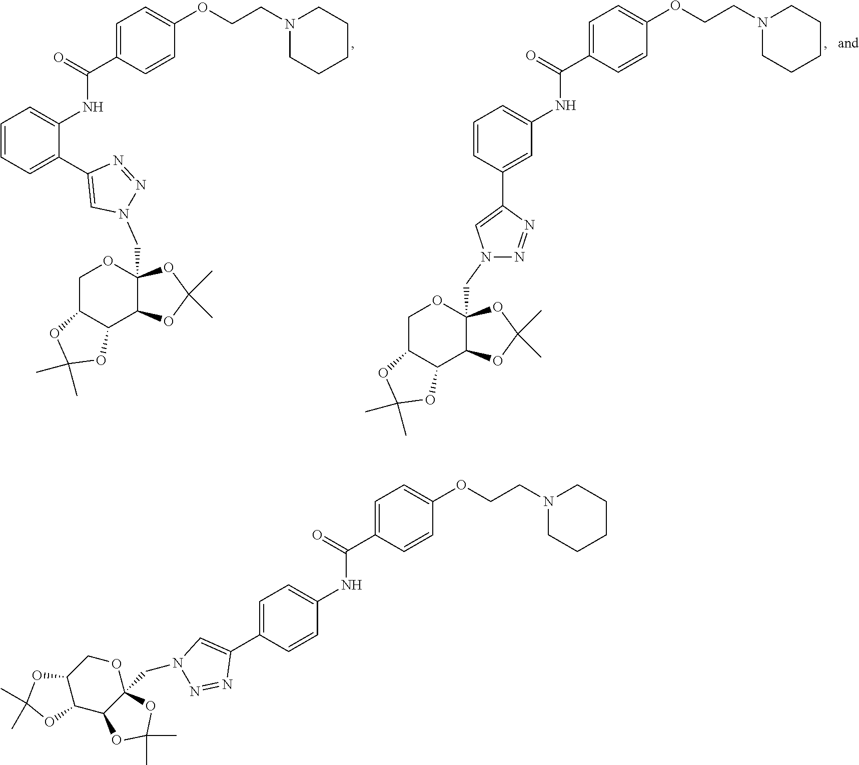 [SMILES: CC1(C)O[C@@H]2[C@@H](CO[C@@]3(Cn4cc(-c5ccccc5NC(=O)c5ccc(OCCN6CCCCC6)cc5)nn4)OC(C)(C)O[C@@H]23)O1 ; CC1(C)O[C@@H]2[C@@H](CO[C@@]3(Cn4cc(-c5cccc(NC(=O)c6ccc(OCCN7CCCCC7)cc6)c5)nn4)OC(C)(C)O[C@@H]23)O1 ; CC1(C)O[C@@H]2[C@@H](CO[C@@]3(Cn4cc(-c5ccc(NC(=O)c6ccc(OCCN7CCCCC7)cc6)cc5)nn4)OC(C)(C)O[C@@H]23)O1]

or a salt thereof.

73. The compound of embodiment 39, wherein the compound is selected from the group consisting of:

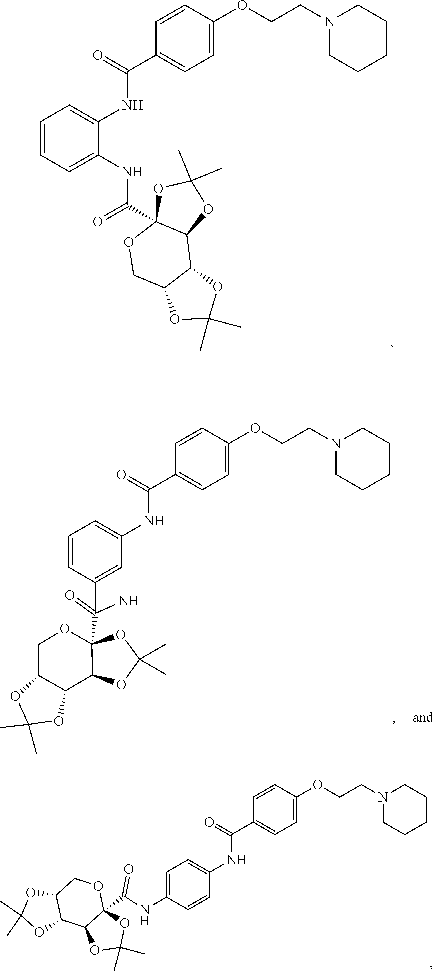

or a salt thereof.

74. A compound of Formula (C), or a salt thereof:

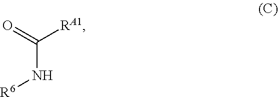

wherein:
R^{A1} is

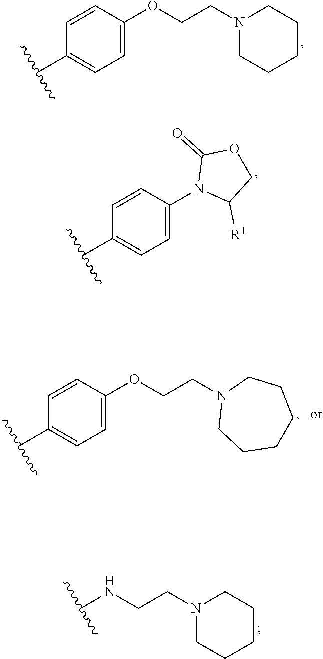

R¹ is optionally substituted alkyl or optionally substituted cycloalkyl;
R⁶ is optionally substituted

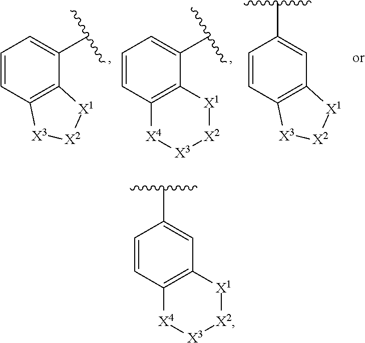

wherein:
X¹, X², X³, and X⁴ are each independently selected from —N(R⁷)—, —O—, —S—, and —C(R⁸)(R⁹)—;
each R⁷ is independently hydrogen, optionally substituted alkyl, or a nitrogen protecting group; and
R⁸ and R⁹ are each independently hydrogen, optionally substituted alkyl, or are taken together with the carbon to which they are attached form a carbonyl.

75. The compound of embodiment 74, wherein the compound is of Formula (III), or a salt thereof:

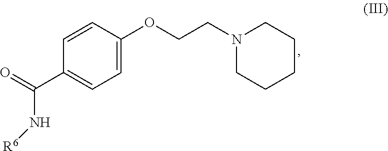

wherein:
R⁶ is

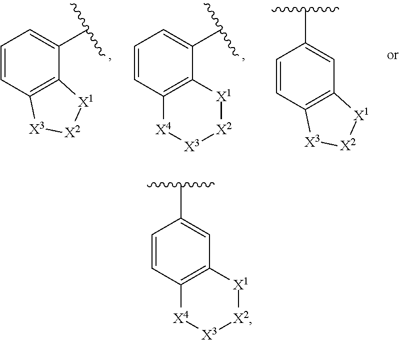

wherein:
X¹, X², X³, and X⁴ are each independently selected from —N(R⁷)—, —O—, —S—, and —C(R⁸)(R⁹)—;
each R⁷ is independently hydrogen, alkyl, or a nitrogen protecting group; and
R⁸ and R⁹ are each independently hydrogen, alkyl, or taken together with the carbon to which they are attached form a carbonyl.

76. The compound of embodiment 74, wherein the compound is of Formula (XIV), (XV), or (XVI), or a salt thereof:

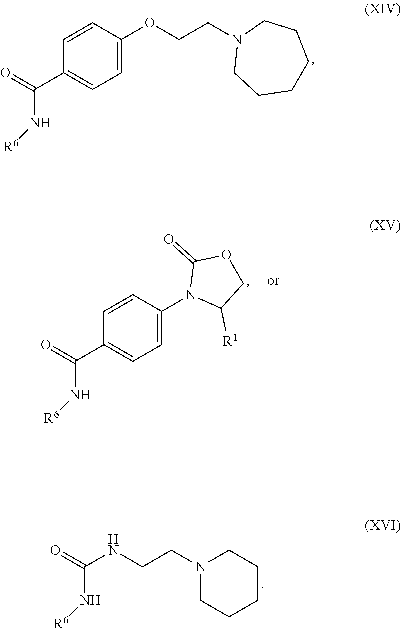

77. The compound of any one of embodiments 74-76, or a salt thereof, wherein R⁶ is

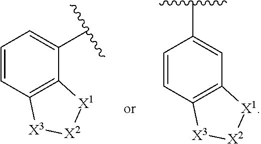

78. The compound of any one of embodiments 74-76, or a salt thereof, wherein R⁶ is

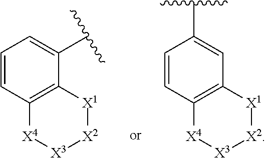

79. The compound of any one of embodiments 74-76, or a salt thereof, wherein R⁶ is

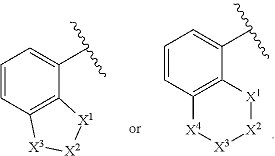

80. The compound of any one of embodiments 74-76, or a salt thereof, wherein R⁶ is

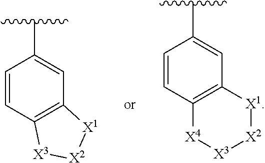

81. The compound of any one of embodiments 74-80, or a salt thereof, wherein at least one of X¹, X², X³, and X⁴ is —N(R⁷)—.

82. The compound of embodiment 81, or a salt thereof, wherein R⁷ is hydrogen.

83. The compound of any one of embodiments 74-82, or a salt thereof, wherein at least one of X¹, X², X³, and X⁴ is —C(R⁸)(R⁹)— and R⁸ and R⁹ taken together with the carbon to which they are attached form a carbonyl.

84. The compound of embodiment 74, wherein the compound is of Formula (XVII), or a salt thereof:

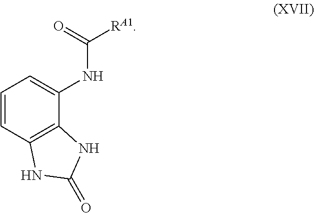

85. The compound of embodiment 76, or a salt thereof, wherein R¹ is C₁₋₆ alkyl.

86. The compound of embodiment 85, or a salt thereof, wherein R¹ is isopropyl.

87. The compound of embodiment 74, wherein the compound has the formula:

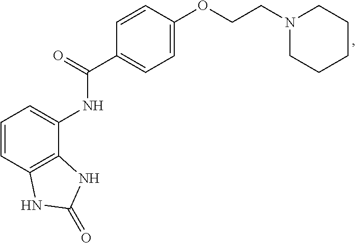 [SMILES: O=C(Nc1cccc2[nH]c(=O)[nH]c12)c1ccc(OCCN2CCCCC2)cc1]

or a salt thereof.

88. The compound of embodiment 74, wherein the compound has the formula:

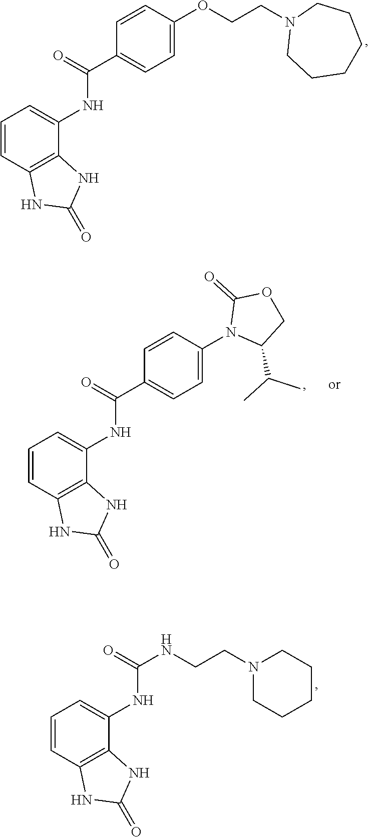 [SMILES: O=C(Nc1cccc2[nH]c(=O)[nH]c12)c1ccc(OCCN2CCCCCC2)cc1 ; CC(C)[C@H]1COC(=O)N1c1ccc(C(=O)Nc2cccc3[nH]c(=O)[nH]c23)cc1 ; O=C(NCCN1CCCCC1)Nc1cccc2[nH]c(=O)[nH]c12]

or a salt thereof.

89. A compound of Formula (D), or a salt thereof:

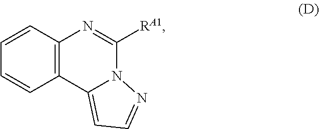

wherein:

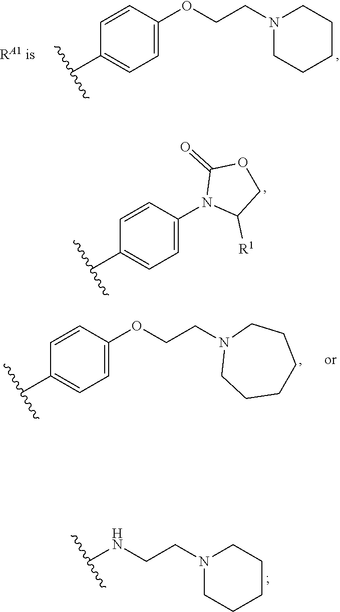

and
R¹ is optionally substituted alkyl or optionally substituted cycloalkyl.

90. The compound of embodiment 89, or a salt thereof, wherein R¹ is C₁₋₆ alkyl.

91. The compound of embodiment 90, or a salt thereof, wherein R¹ is isopropyl.

92. The compound of embodiment 89, wherein the compound has the formula:

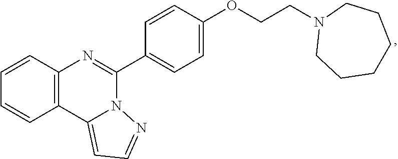 [SMILES: c1ccc2c(c1)nc(-c1ccc(OCCN3CCCCCC3)cc1)n1nccc21]

or a salt thereof.

93. The compound of embodiment 89, wherein the compound has the formula:

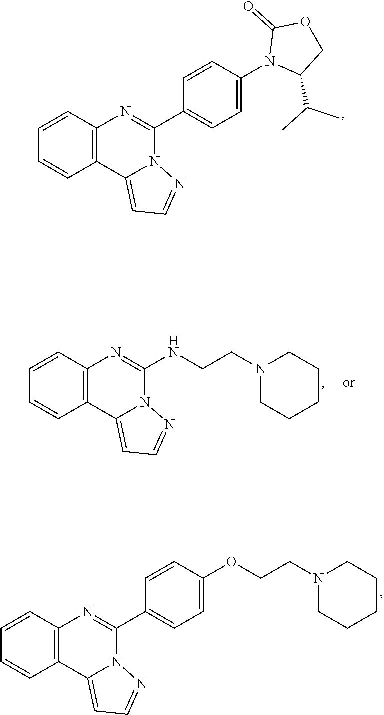 [SMILES: CC(C)[C@H]1COC(=O)N1c1ccc(-c2nc3ccccc3c3ccnn23)cc1 ; c1ccc2c(c1)nc(NCCN1CCCCC1)n1nccc21 ; c1ccc2c(c1)nc(-c1ccc(OCCN3CCCCC3)cc1)n1nccc21]

or a salt thereof.

94. A compound of Formula (E), or a salt thereof:

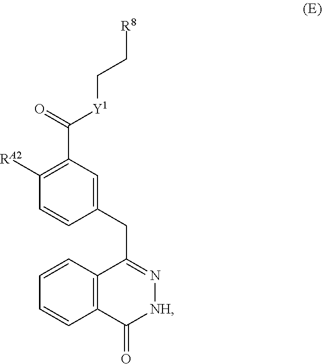

wherein:
R⁸ is

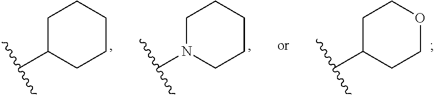 [SMILES: *C1CCCCC1 ; *N1CCCCC1 ; *C1CCOCC1]

R^{A2} is halogen;
Y¹ is —O— or —N(R⁷)—; and
R⁷ is hydrogen, alkyl, or a nitrogen protecting group.

95. The compound of embodiment 94, or a salt thereof, wherein R^{A2} is fluorine.

96. The compound of embodiment 94 or 95, or a salt thereof, wherein Y¹ is —O— or —NH—.

97. The compound of embodiment 94, wherein the compound has the formula:

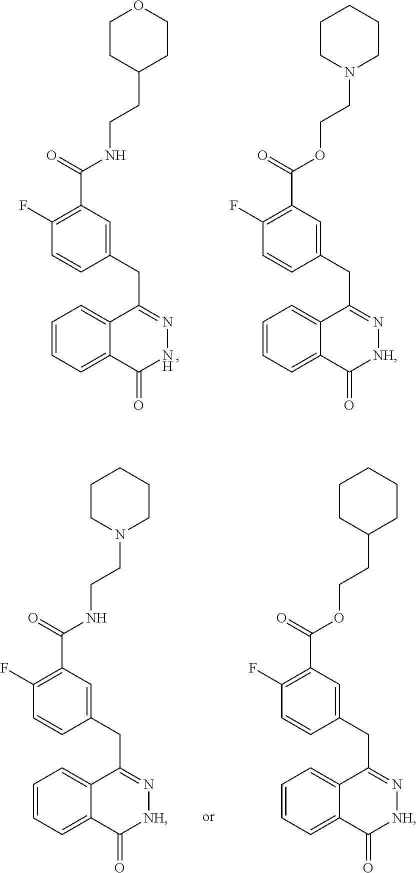 [SMILES: O=C(NCCC1CCOCC1)c1cc(Cc2n[nH]c(=O)c3ccccc23)ccc1F ; O=C(OCCN1CCCCC1)c1cc(Cc2n[nH]c(=O)c3ccccc23)ccc1F ; O=C(NCCN1CCCCC1)c1cc(Cc2n[nH]c(=O)c3ccccc23)ccc1F ; "or" ; O=C(OCCC1CCCCC1)c1cc(Cc2n[nH]c(=O)c3ccccc23)ccc1F]

or a salt thereof.

98. A pharmaceutical composition comprising a compound of any one of the preceding embodiments, or a salt thereof, and a pharmaceutically acceptable carrier.

99. The pharmaceutical composition of embodiment 98, further comprising an additional therapeutic agent.

100. A method of inhibiting IL-6 signaling comprising administering a compound of any one of embodiments 1-97, or a salt thereof, or a pharmaceutical composition of any one of embodiments 98 or 99.

101. The method of embodiment 100, wherein the compound is a dual PARP/IL-6 inhibitor.

102. The method of embodiment 100 or 101, wherein the inhibition of IL-6 is in vitro.

103. The method of embodiment 100 or 101, wherein the inhibition of IL-6 is in vivo.

104. The method of any one of embodiments 100-103, further comprising administering the compound to a subject.

105. A method of inhibiting IL-6/gp130 comprising administering a compound of any one of embodiments 1-97, or a salt thereof, or a pharmaceutical composition of any one of embodiments 98 or 99.

106. The method of embodiment 105, wherein the compound is a dual PARP/IL-6 inhibitor.

107. The method of embodiment 105 or 106, wherein the inhibition is in vitro.

108. The method of embodiment 105 or 106, wherein the inhibition is in vivo.

109. The method of any one of embodiments 105-108, further comprising administering the compound to a subject.

110. A method of treating inflammatory disease in a subject in need thereof, the method comprising administering to the subject a compound of any one of embodiments 1-97, or a salt thereof, or a pharmaceutical composition of any one of embodiments 98 or 99.

111. The method of embodiment 110, wherein the inflammatory disease is fibrosis.

112. The method of embodiment 111, wherein the fibrosis is liver fibrosis.

113. The method of embodiment 110, wherein the inflammatory disease is nonalcoholic steatohepatitis (NASH).

114. A method of treating cancer in a subject in need thereof, the method comprising administering to the subject a compound of any one of embodiments 1-97, or a salt thereof, or a pharmaceutical composition of any one of embodiments 98 or 99.

115. The method of embodiment 114, wherein the cancer is breast cancer or pancreatic cancer.

116. The method of embodiment 115, wherein the breast cancer is triple negative breast cancer.

117. The method of embodiment 114, wherein the cancer is bone cancer or brain cancer.

118. A method of treating an autoimmune disease in a subject in need thereof, the method comprising administering to the subject a compound of any one of embodiments 1-97, or a salt thereof, or a pharmaceutical composition of any one of embodiments 98 or 99.

119. The method of any one of embodiments 100-118, comprising administering to the subject a therapeutically effective amount of a compound of any one of embodiments 1-97, or a salt thereof, or a pharmaceutical composition of any one of embodiments 98 or 99.

120. The method of any one of embodiments 100-119, wherein the subject is identified as in need of such treatment.

121. The method of any one of embodiments 100-120, wherein the subject has such disease or disorder.

What is claimed is:

1. A compound of Formula (F), or a salt thereof: 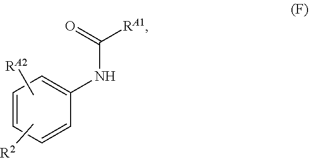
wherein:
R^{A1} is 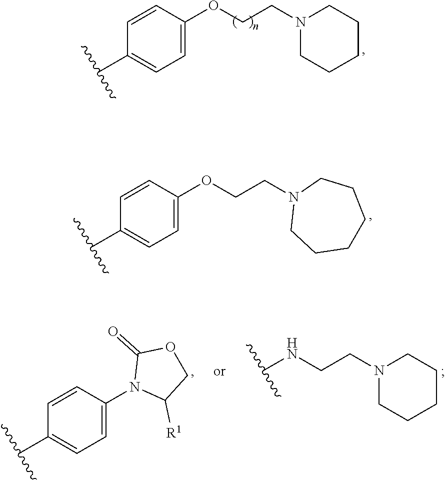
R¹ is optionally substituted alkyl or optionally substituted cycloalkyl;
R^{A2} is H or halogen;
R² is optionally substituted 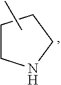
optionally substituted 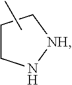
or optionally substituted 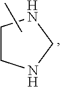
optionally substituted 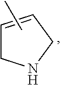
optionally substituted 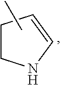
optionally substituted 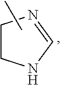
optionally substituted 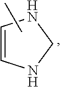
optionally substituted 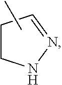
optionally substituted 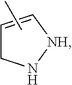
optionally substituted 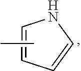
optionally substituted 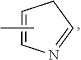
optionally substituted 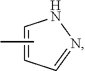
optionally substituted 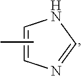
optionally substituted 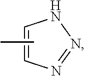
optionally substituted 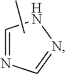
optionally substituted 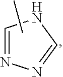
or optionally substituted 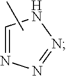
and
n is 1 or 2.

2. The compound of claim 1, or a salt thereof, wherein the compound is of Formula (A): 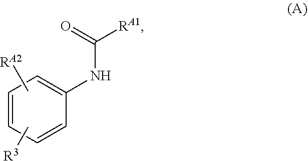
wherein:
R^{A1} is 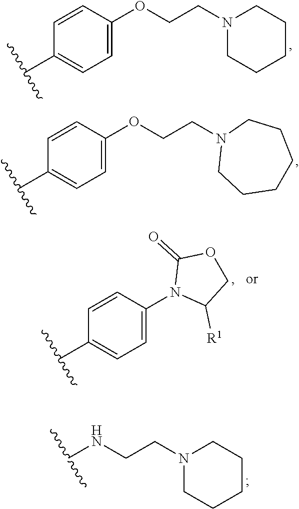
R¹ is optionally substituted alkyl or cycloalkyl;
R^{A2} is H or halogen; and
R² is optionally substituted 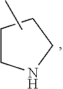
optionally substituted 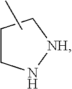
or optionally substituted 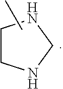
optionally substituted 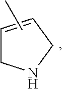
optionally substituted 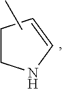
optionally substituted 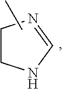
optionally substituted 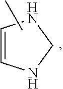
optionally substituted 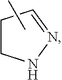
optionally substituted 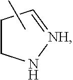
optionally substituted 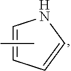
optionally substituted 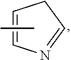
optionally substituted 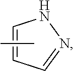
optionally substituted 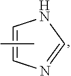
optionally substituted 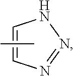
optionally substituted 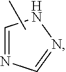
optionally substituted 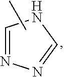
or optionally substituted

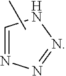

3. The compound of claim 1 or 2, wherein the compound is of Formula (IV), or a salt thereof:

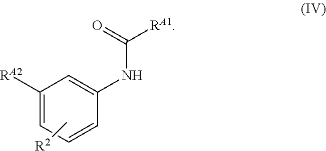

4. The compound of claim 3, wherein the compound is of Formula (IV-c), or a salt thereof:

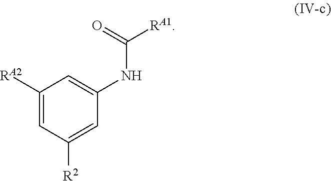

5. The compound of claim 3, wherein the compound is of Formula (IV-d), or a salt thereof:

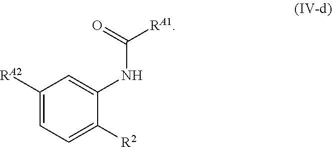

6. The compound of claim 3, wherein the compound is of Formula (IV-e), or a salt thereof:

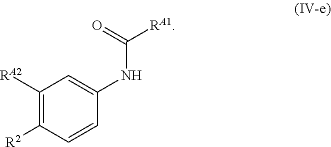

7. The compound of claim 1 or 2, wherein compound is of Formula (I), or a salt thereof: 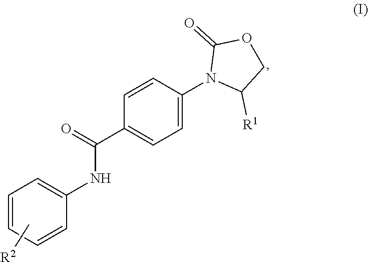
wherein:
R¹ is alkyl or cycloalkyl; and
R² is selected from the group consisting of:

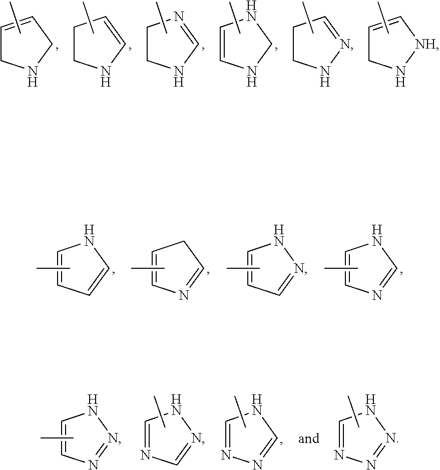

8. The compound of claim 7, or a salt thereof, according to Formula (I-a):

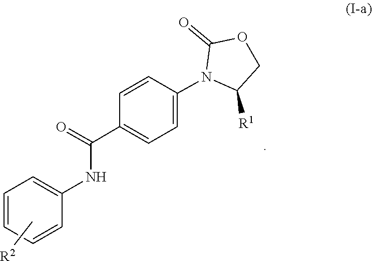

9. The compound of claim 7, or a salt thereof, according to Formula (I-b):

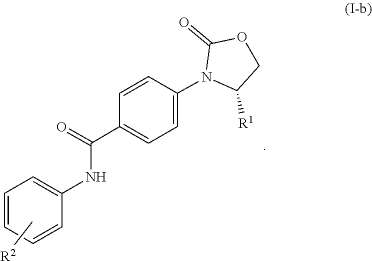

10. The compound of any one of claims 7-9, or a salt thereof, according to Formula (I-c):

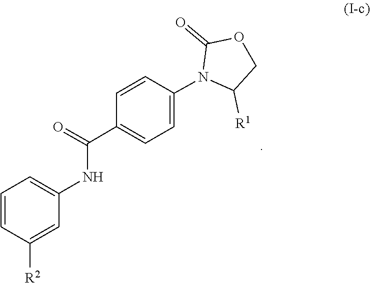

11. The compound of any one of claims 7-9, or a salt thereof, according to Formula (I-d):

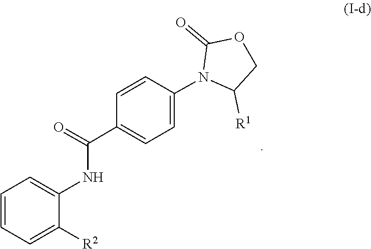

12. The compound of claim 1 or 2, wherein the compound is of Formula (V) or (VI), or a salt 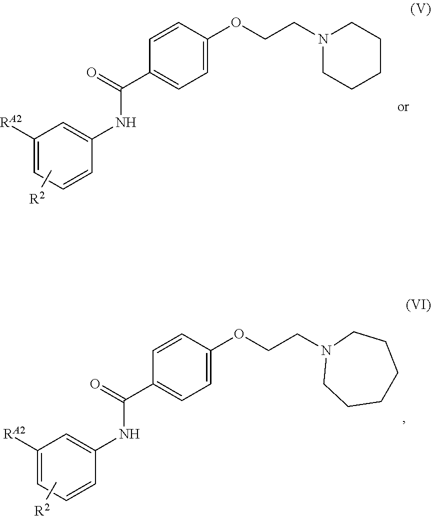
wherein R² is optionally substituted 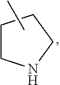
optionally substituted 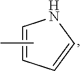
optionally substituted 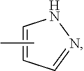
or optionally substituted

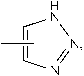

13. The compound of claim 12, wherein the compound is of Formula (V-A), or a salt thereof: 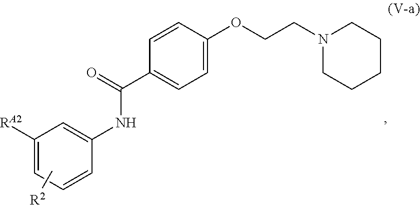
wherein:
R² is 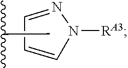
and
R^{A3} is optionally substituted alkyl or optionally substituted carbocyclyl.

14. The compound of claim 1 or 2, wherein the compound is of Formula (VII) or (VIII), or a salt thereof:

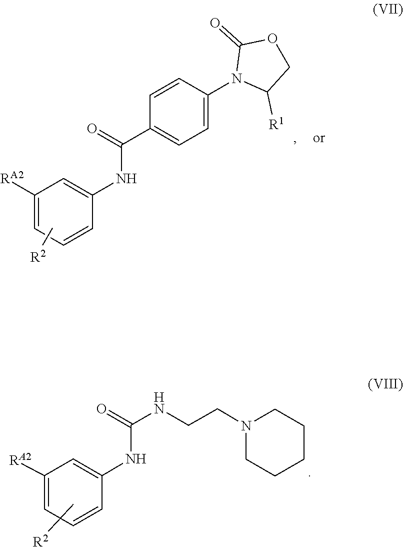

15. The compound of any one of claims 1-6, 12, or 14, or a salt thereof, wherein R² is optionally substituted 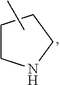
optionally substituted 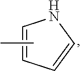
optionally substituted 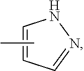
or optionally substituted

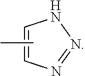

16. The compound of any one of claims 1-6, 12, or 14, or a salt thereof, wherein R² is

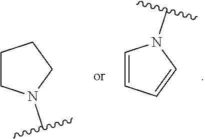 [SMILES: *N1CCCC1 ; *n1cccc1]

17. The compound of any one of claims 1-6, 12, or 14, or a salt thereof, wherein R² is

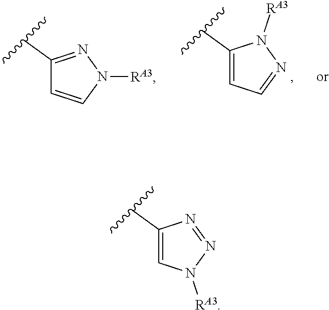

18. The compound of claim 13 or 17, wherein R^{A3} is optionally substituted C₁₋₆ alkyl or optionally substituted C₃₋₈ carbocyclyl.

19. The compound of claim 13, 17, or 18, wherein R^{A3} is

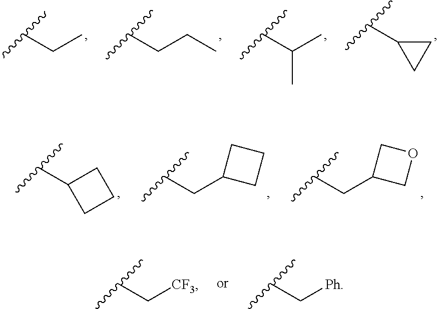

20. The compound of any one of claims 1-6, 12, or 14, or a salt thereof, wherein R² is 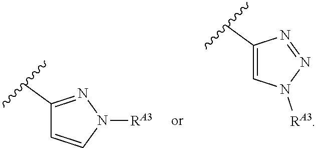
wherein R^{A3} is

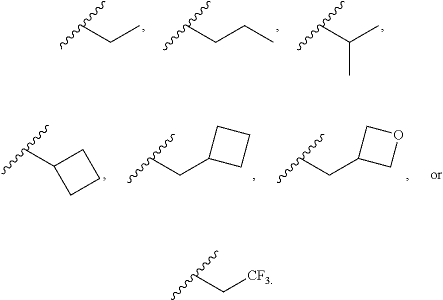

21. The compound of any one of claims 1-15, 17, or 20, or a salt thereof, wherein R² is

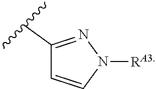

22. The compound of any one of claims 1-11, or a salt thereof, wherein R² is

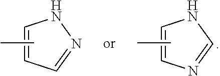

23. The compound of claim 22, or a salt thereof, wherein R² is

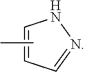

24. The compound of any one of the preceding claims, or a salt thereof, wherein R¹ is C₁₋₆ alkyl.

25. The compound of claim 24, or a salt thereof, wherein R¹ is isopropyl.

26. The compound of any one of claims 1-23, or a salt thereof, wherein R¹ is C₁₋₈ cycloalkyl.

27. The compound of claim 26, or a salt thereof, wherein R¹ is cyclopropyl or cyclobutyl.

28. The compound of any one of the preceding claims, or a salt thereof, wherein R^{A2} is hydrogen.

29. The compound of any one of the preceding claims, or a salt thereof, wherein R^{A2} is halogen.

30. The compound of claim 29, or a salt thereof, wherein R^{A2} is fluorine.

31. The compound of claim 1 or 2, wherein the compound is selected from the group consisting of: 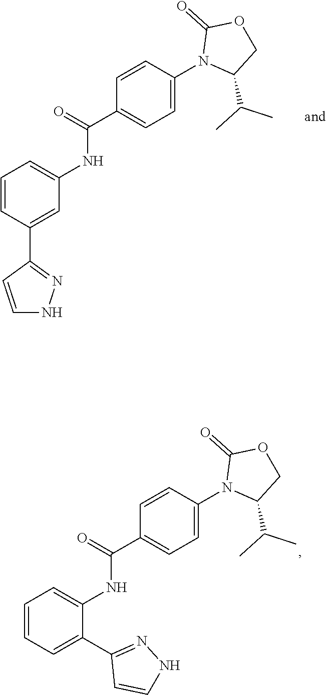 [SMILES: CC(C)[C@H]1COC(=O)N1c1ccc(C(=O)Nc2cccc(-c3cc[nH]n3)c2)cc1 ; CC(C)[C@H]1COC(=O)N1c1ccc(C(=O)Nc2ccccc2-c2cc[nH]n2)cc1]
or a salt thereof.

32. The compound of claim 1 or 2, wherein the compound is selected from the group consisting of 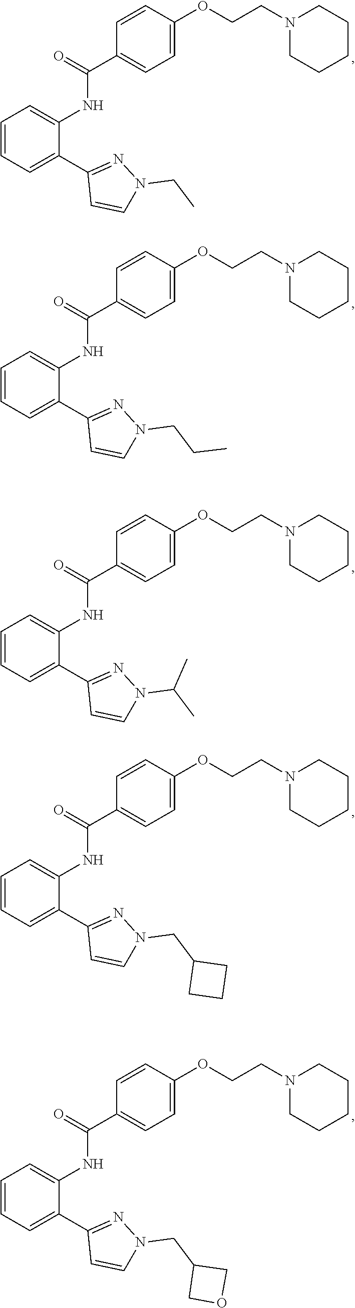 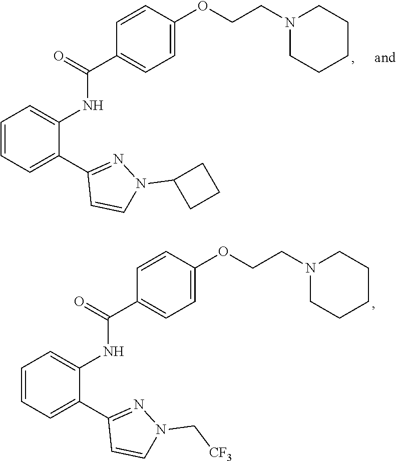 [SMILES: O=C(Nc1ccccc1-c1ccn(C2CCC2)n1)c1ccc(OCCN2CCCCC2)cc1 ; O=C(Nc1ccccc1-c1ccn(CC(F)(F)F)n1)c1ccc(OCCN2CCCCC2)cc1]
or a salt thereof.

33. The compound of claim 1 or 2, wherein the compound is selected from the group consisting of 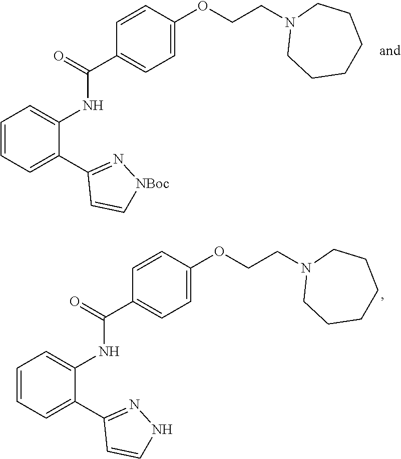 [SMILES: CC(C)(C)OC(=O)n1ccc(-c2ccccc2NC(=O)c2ccc(OCCN3CCCCCC3)cc2)n1 ; O=C(Nc1ccccc1-c1cc[nH]n1)c1ccc(OCCN2CCCCCC2)cc1]
or a salt thereof.

34. The compound of claim 1 or 2, wherein the compound is selected from the group consisting of 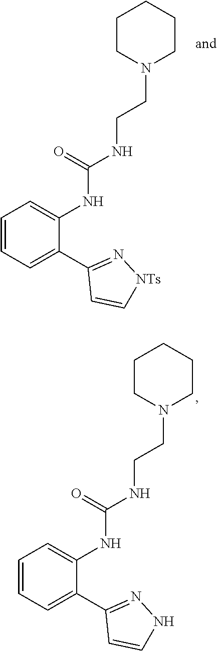 [SMILES: Cc1ccc(S(=O)(=O)n2ccc(-c3ccccc3NC(=O)NCCN3CCCCC3)n2)cc1 ; O=C(NCCN1CCCCC1)Nc1ccccc1-c1cc[nH]n1]
or a salt thereof.

35. The compound of claim 1 or 2, wherein the compound is selected from the group consisting of 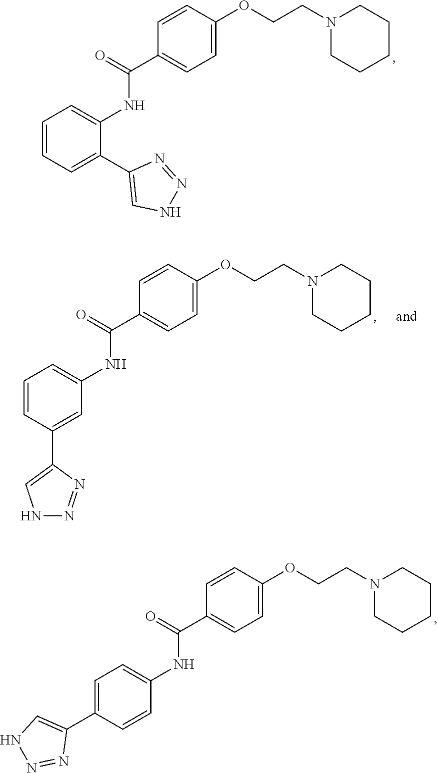 [SMILES: O=C(Nc1ccccc1-c1c[nH]nn1)c1ccc(OCCN2CCCCC2)cc1 ; O=C(Nc1cccc(-c2c[nH]nn2)c1)c1ccc(OCCN2CCCCC2)cc1 ; O=C(Nc1ccc(-c2c[nH]nn2)cc1)c1ccc(OCCN2CCCCC2)cc1]
or a salt thereof.

36. The compound of claim 1 or 2, wherein the compound has the structure 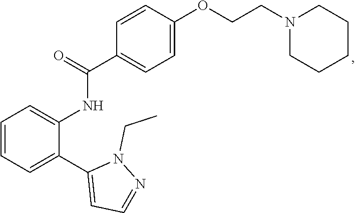 [SMILES: CCn1nccc1-c1ccccc1NC(=O)c1ccc(OCCN2CCCCC2)cc1]
or a salt thereof.

37. The compound of claim 1 or 2, wherein the compound is selected from the group consisting of 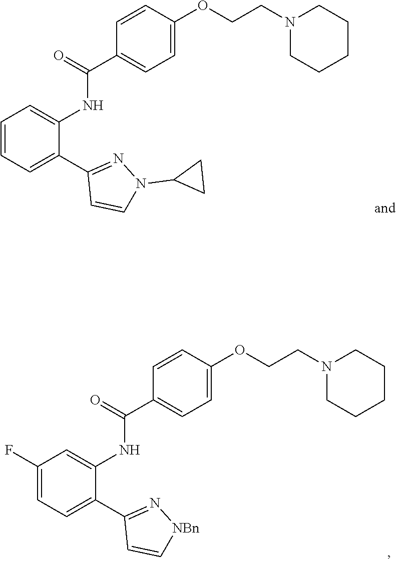 [SMILES: O=C(Nc1ccccc1-c1ccn(C2CC2)n1)c1ccc(OCCN2CCCCC2)cc1 ; O=C(Nc1cc(F)ccc1-c1ccn(Cc2ccccc2)n1)c1ccc(OCCN2CCCCC2)cc1]
or a salt thereof.

38. The compound of claim 1, wherein the compound is selected from the group consisting of 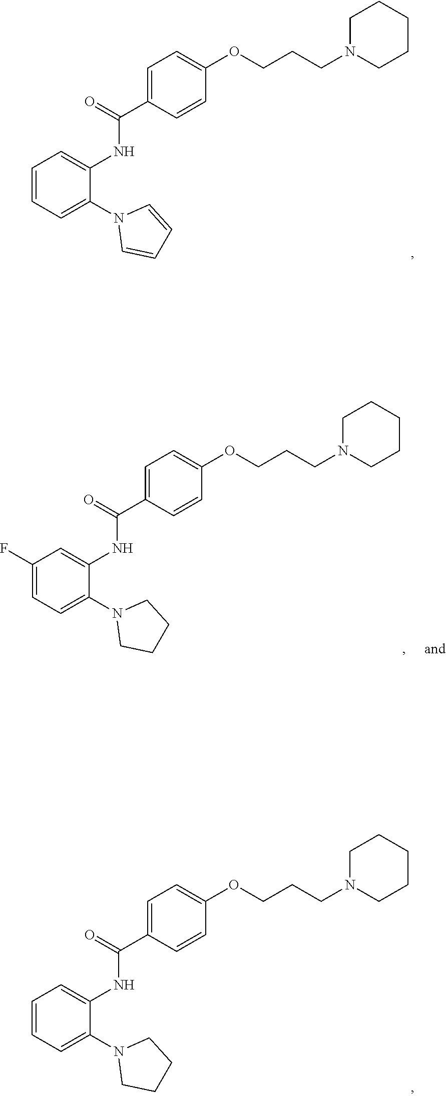 [SMILES: O=C(Nc1ccccc1-n1cccc1)c1ccc(OCCCN2CCCCC2)cc1 ; O=C(Nc1cc(F)ccc1N1CCCC1)c1ccc(OCCCN2CCCCC2)cc1 ; O=C(Nc1ccccc1N1CCCC1)c1ccc(OCCCN2CCCCC2)cc1]
or a salt thereof.

39. A compound of Formula (B), or a salt thereof: 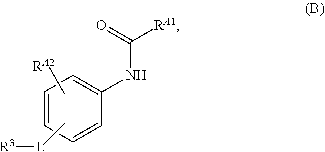
wherein:
R^{A1} is 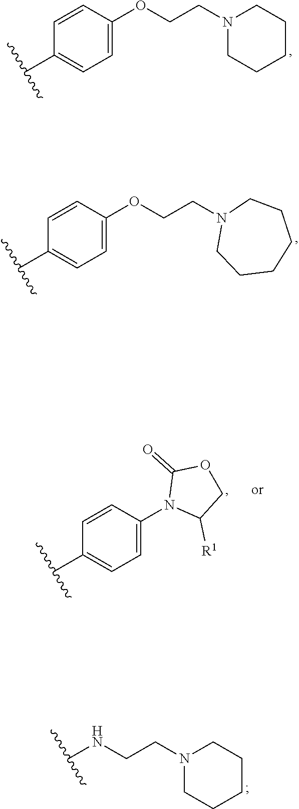
R¹ is optionally substituted alkyl or optionally substituted cycloalkyl;
R^{A2} is H or halogen;
L is a linker selected from the group consisting of —NH(C═O)—, —(C═O)NH—, optionally substituted five-membered N-containing heterocyclylene, optionally substituted five-membered N-containing heteroarylene, or optionally substituted heteroarylalkylene; and
R³ is a carbohydrate or carbohydrate substituted with one or more oxygen protecting groups.

40. The compound of claim 39, wherein the compound is of Formula (IX), or a salt thereof:

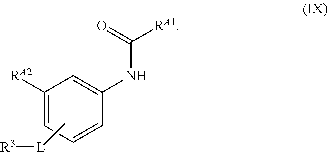

41. The compound of claim 40, wherein the compound is of Formula (IX-c), or a salt thereof:

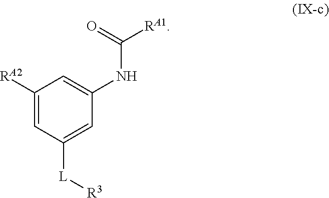

42. The compound of claim 40, wherein the compound is of Formula (IX-d), or a salt thereof:

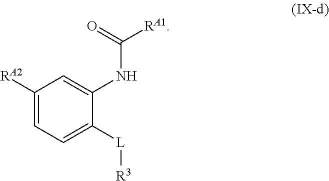

43. The compound of claim 40, wherein the compound is of Formula (IX-e), or a salt thereof:

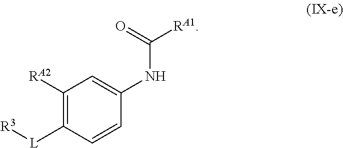

44. The compound of claim 40, wherein the compound is of Formula (X), (XI), (XII), or (XIII), or a salt thereof:

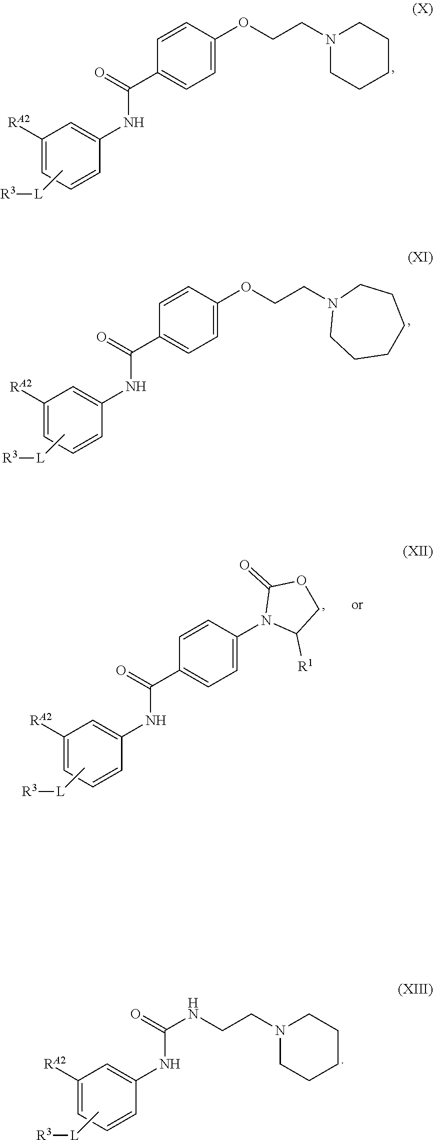

45. The compound of claim 44, wherein the compound is of Formula (X-c), (XI-c), (XII-c), or (XIII-c), or a salt thereof:

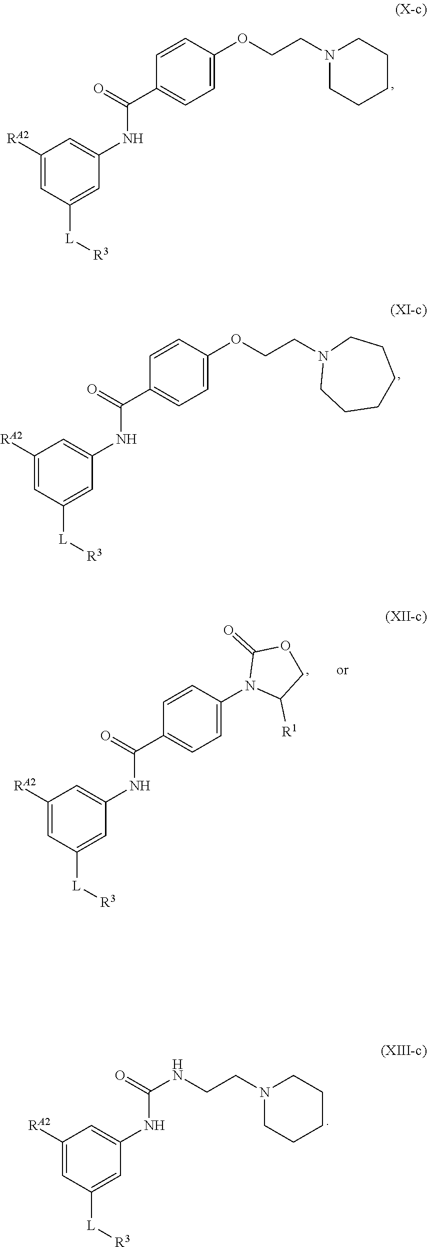

46. The compound of claim 44, wherein the compound is of Formula (X-d), (XI-d), (XII-d), or (XIII-d), or a salt thereof:

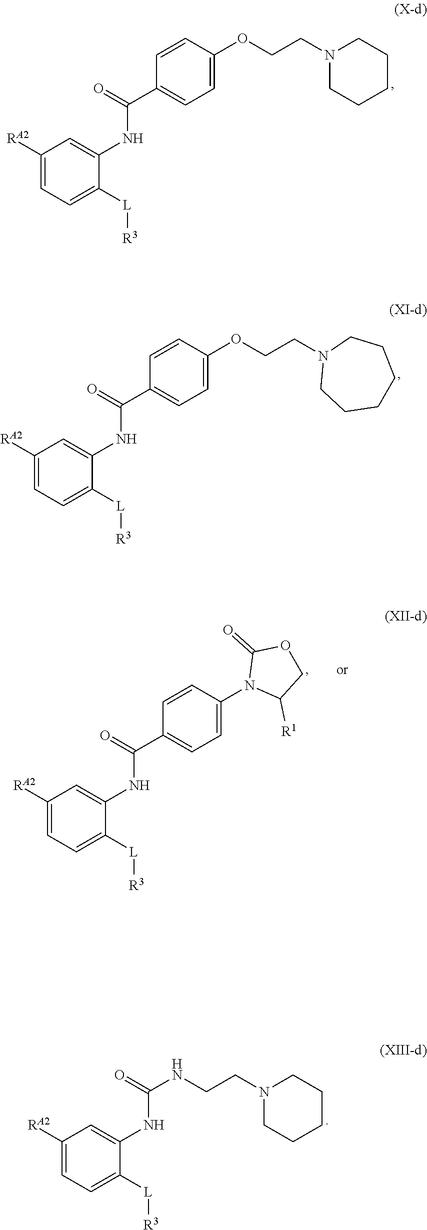

47. The compound of claim 44, wherein the compound is of Formula (X-e), (XI-e), (XII-e), or (XIII-e), or a salt thereof:

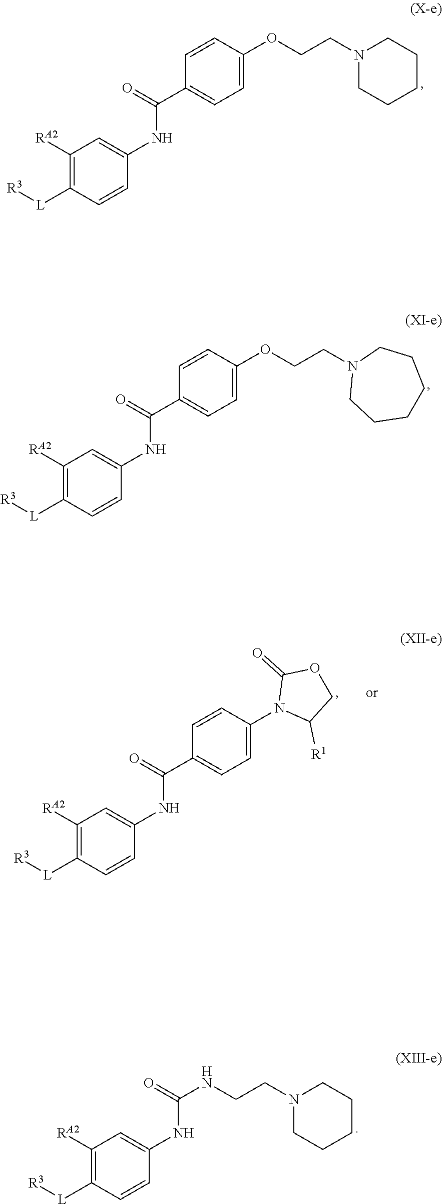

48. The compound of any one of claims 39-47, or a salt thereof, wherein L is —NH(C═O)— or —(C═O)NH—.

49. The compound of any one of claims 39-47, wherein L is a linker selected from the group consisting of optionally substituted five-membered N-containing heterocyclylene and optionally substituted five-membered N-containing heteroarylene.

50. The compound of claim 49, or a salt thereof, wherein L is optionally substituted pyrrolidinylene, optionally substituted pyrazolidinylene, optionally substituted imadazolidinylene, optionally substituted 3-pyrrolinylene, optionally substituted 2-pyrrolinylene, optionally substituted 2-pyrazolinylene, optionally substituted 2-imidazolinylene, optionally substituted 2H-pyrrolylene, optionally substituted 1H-pyrrolylene, optionally substituted pyrazolylene, optionally substituted imidazolylene, optionally substituted 1,2,4-triazolylene, optionally substituted 1,2,3-triazolylene, or optionally substituted tetrazolylene.

51. The compound of claim 50, or a salt thereof, wherein L is optionally substituted pyrazolylene, optionally substituted imidazolylene, optionally substituted 1,2,4-triazolylene, or optionally substituted 1,2,3-triazolylene.

52. The compound of claim 51, or a salt thereof, wherein L is

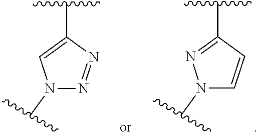 [SMILES: *c1cn(*)nn1 ; *c1ccn(*)n1]

53. The compound of claim 52, or a salt thereof, wherein L is

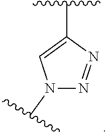 [SMILES: *c1cn(*)nn1]

54. The compound of any one of claims 39-53, wherein R³ is selected from the group consisting of 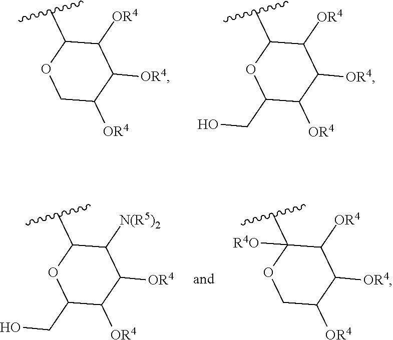
wherein:
each R⁴ is independently hydrogen or an oxygen protecting group or wherein two R⁴ are joined together with the intervening atoms to form an oxygen protecting group; and
each R⁵ is independently hydrogen or a nitrogen protecting group.

55. The compound of claim 54, wherein R³ is selected from the group consisting of

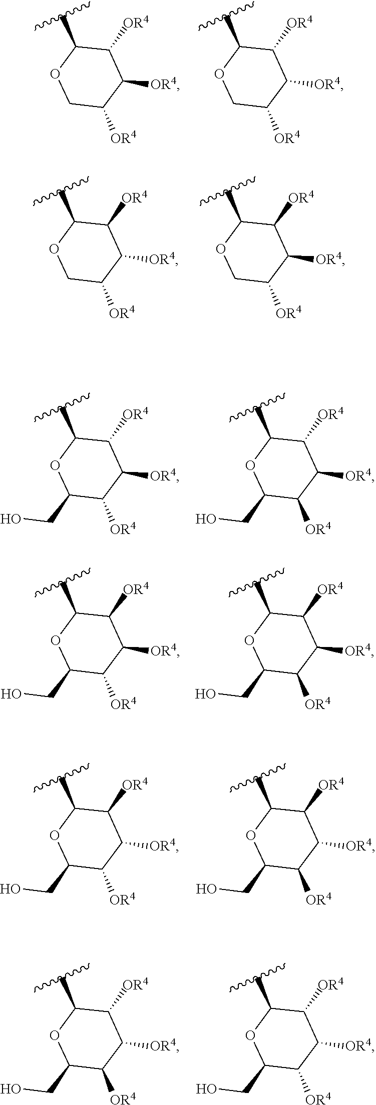

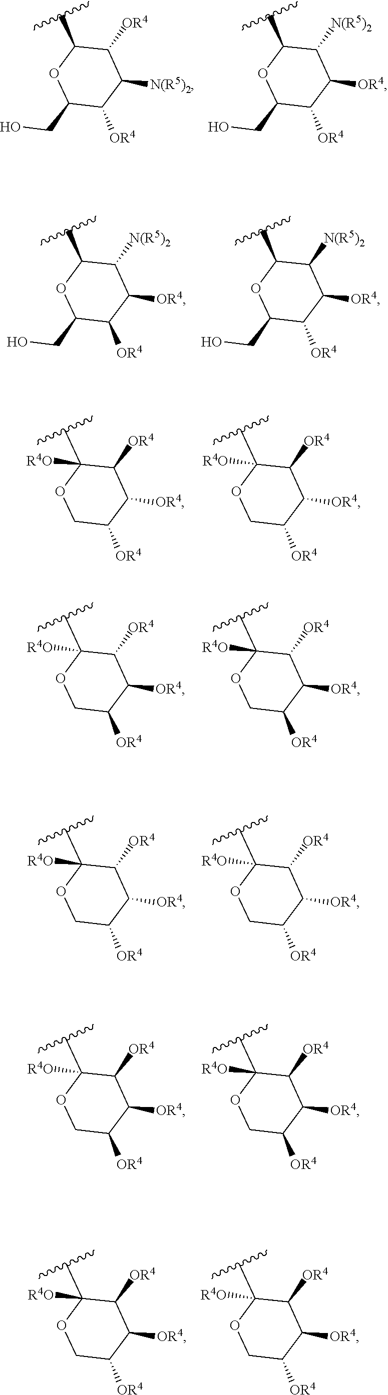

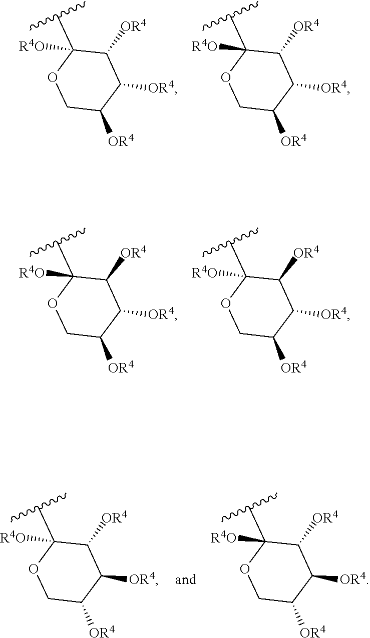

56. The compound of claim 39, wherein the compound is of Formula (II), or a salt thereof: 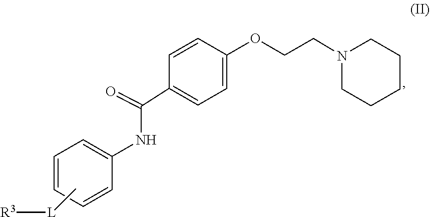
wherein:
L is a linker selected from the group consisting of —NH(C═O)—, —(C═O)NH—, 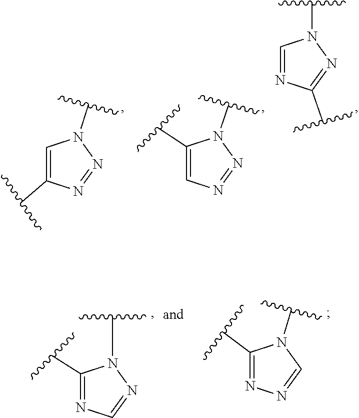 [SMILES: *c1cn(*)nn1 ; *c1cnnn1* ; *c1ncn(*)n1 ; *c1ncnn1* ; *c1nncn1*]
R³ is selected from the group consisting of 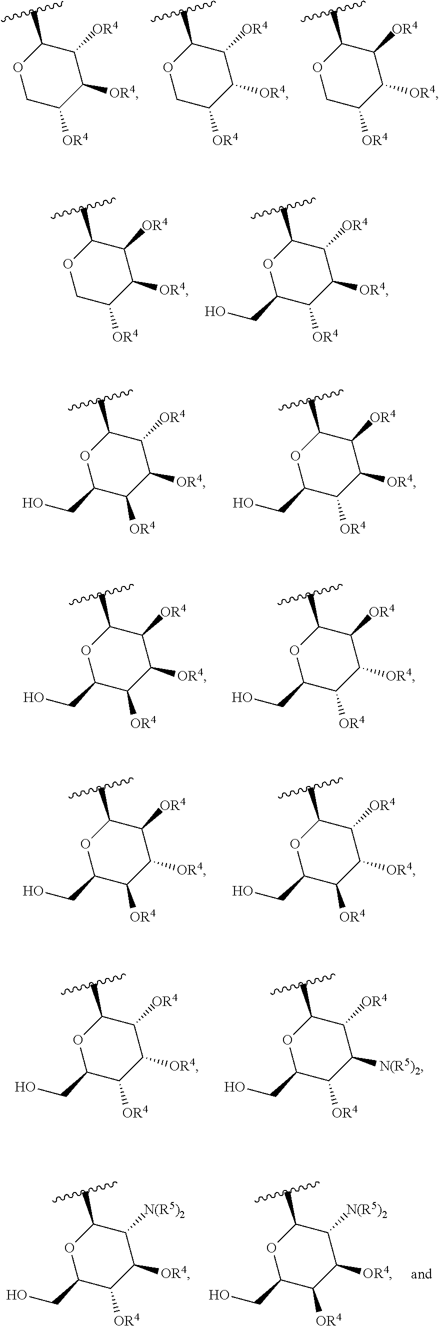 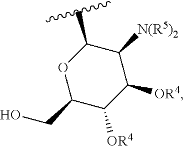
wherein:
each R⁴ is independently hydrogen or an oxygen protecting group; and
each R⁵ is independently hydrogen or a nitrogen protecting group.

57. The compound of 44, or a salt thereof, wherein R³ is

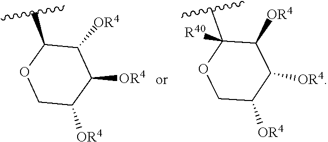

58. The compound of claim 57, or a salt thereof, wherein R³ is

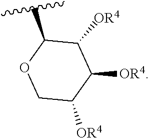

59. The compound any one of claims 39-47 or 49-58, or a salt thereof, wherein -L-R³ is

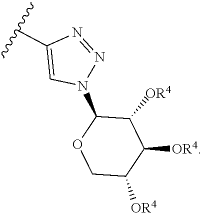

60. The compound of any one of claims 39-55 or 57, or a salt thereof, wherein -L-R³ is

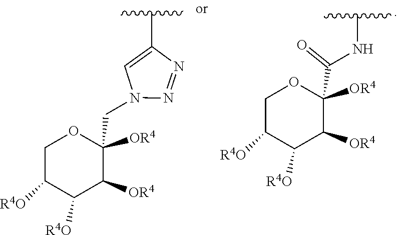

61. The compound of claim 56, or a salt thereof, according to Formula (II-a):

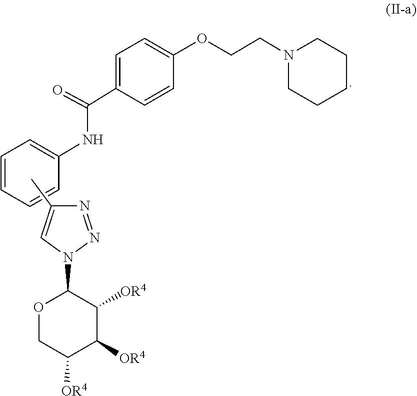

62. The compound of any one of claims 54-61, or a salt thereof, wherein R⁴ is H.

63. The compound of any one of claims 54-61, or a salt thereof, wherein R⁴ is an oxygen protecting group.

64. The compound of claim 63, or a salt thereof, wherein the oxygen protecting group is acyl.

65. The compound of any one of claims 54-61, or a salt thereof, wherein two R⁴ are joined together with the intervening atoms to form an oxygen protecting group.

66. The compound of claim 65, or a salt thereof, wherein two R⁴ are joined together with the intervening atoms to form an acetal protecting group.

67. The compound of any one of claims 39-66, or a salt thereof, wherein R^{A2} is hydrogen.

68. The compound of any one of claims 39-66, or a salt thereof, wherein R^{A2} is halogen.

69. The compound of claim 68, or a salt thereof, wherein R^{A2} is fluorine.

70. The compound of claim 39, wherein the compound is selected from the group consisting of: 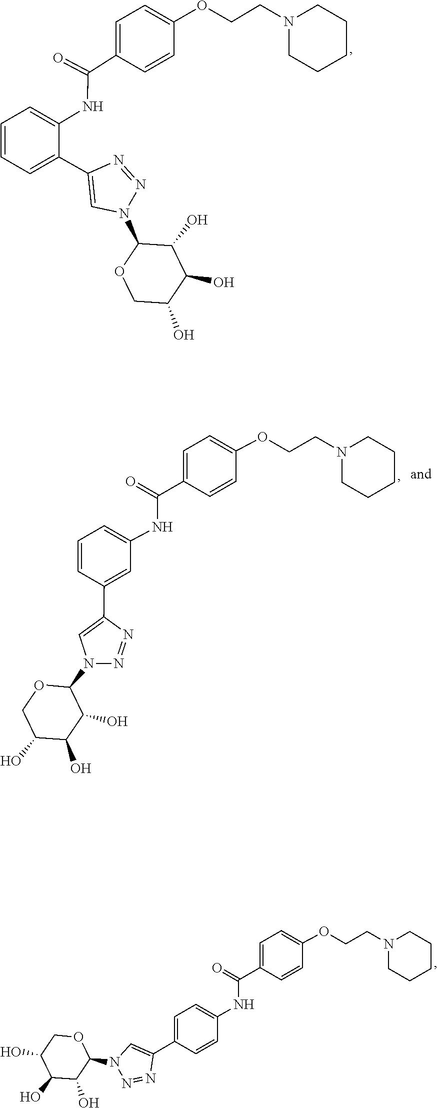 [SMILES: O=C(Nc1ccccc1-c1cn([C@@H]2OC[C@@H](O)[C@H](O)[C@H]2O)nn1)c1ccc(OCCN2CCCCC2)cc1 ; O=C(Nc1cccc(-c2cn([C@@H]3OC[C@@H](O)[C@H](O)[C@H]3O)nn2)c1)c1ccc(OCCN2CCCCC2)cc1 ; O=C(Nc1ccc(-c2cn([C@@H]3OC[C@@H](O)[C@H](O)[C@H]3O)nn2)cc1)c1ccc(OCCN2CCCCC2)cc1]
or a salt thereof.

71. The compound of claim 39, wherein the compound is selected from the group consisting of: 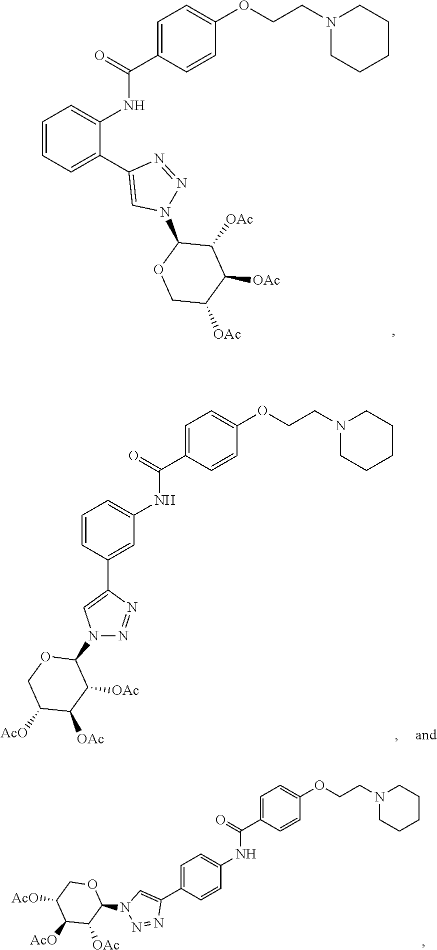 [SMILES: CC(=O)O[C@@H]1[C@@H](OC(C)=O)[C@H](OC(C)=O)CO[C@H]1n1cc(-c2ccccc2NC(=O)c2ccc(OCCN3CCCCC3)cc2)nn1 ; CC(=O)O[C@@H]1[C@@H](OC(C)=O)[C@H](OC(C)=O)CO[C@H]1n1cc(-c2cccc(NC(=O)c3ccc(OCCN4CCCCC4)cc3)c2)nn1 ; CC(=O)O[C@@H]1[C@@H](OC(C)=O)[C@H](OC(C)=O)CO[C@H]1n1cc(-c2ccc(NC(=O)c3ccc(OCCN4CCCCC4)cc3)cc2)nn1]
or a salt thereof.

72. The compound of claim 39, wherein the compound is selected from the group consisting of: 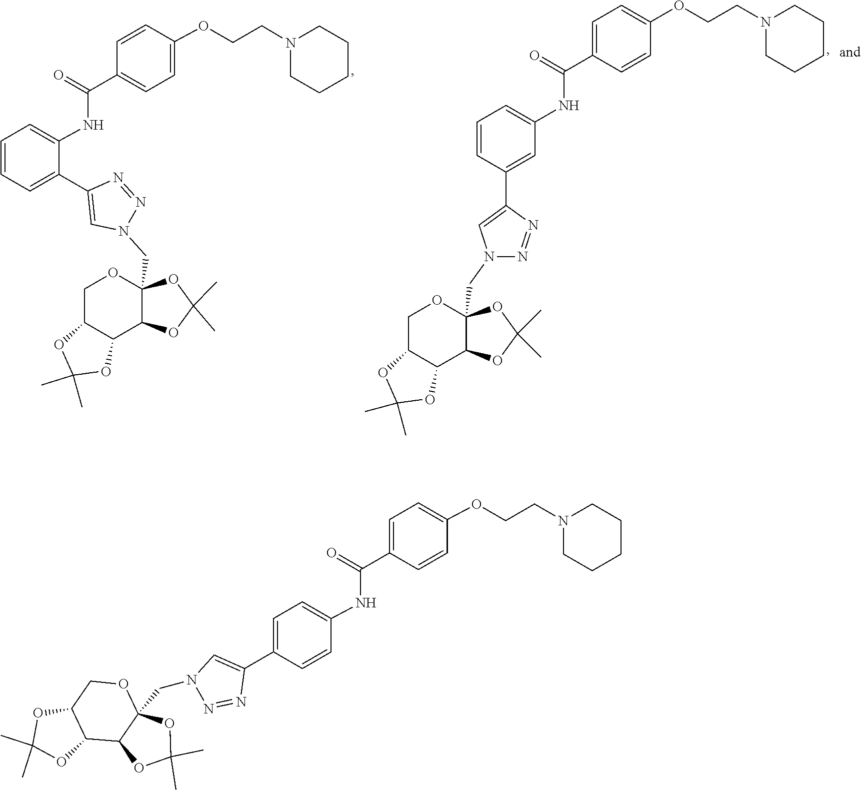
or a salt thereof.

73. The compound of claim 39, wherein the compound is selected from the group consisting of: 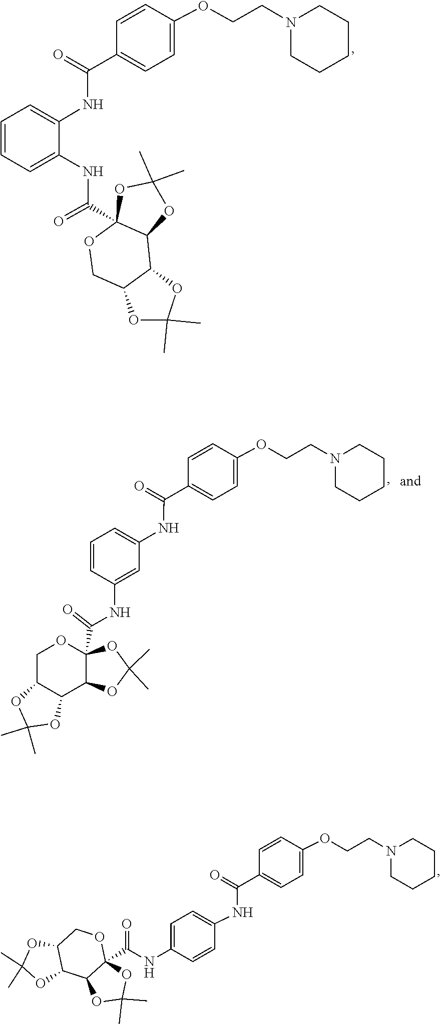 [SMILES: CC1(C)O[C@@H]2[C@@H](CO[C@@]3(C(=O)Nc4ccccc4NC(=O)c4ccc(OCCN5CCCCC5)cc4)OC(C)(C)O[C@@H]23)O1 ; CC1(C)O[C@@H]2[C@@H](CO[C@@]3(C(=O)Nc4cccc(NC(=O)c5ccc(OCCN6CCCCC6)cc5)c4)OC(C)(C)O[C@@H]23)O1 ; CC1(C)O[C@@H]2[C@@H](CO[C@@]3(C(=O)Nc4ccc(NC(=O)c5ccc(OCCN6CCCCC6)cc5)cc4)OC(C)(C)O[C@@H]23)O1]
or a salt thereof.

74. A compound of Formula (C), or a salt thereof: 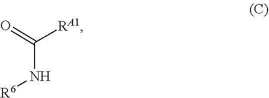
wherein:
R^{A1} is 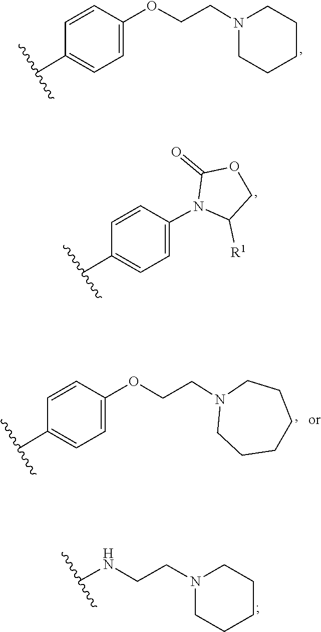
R¹ is optionally substituted alkyl or optionally substituted cycloalkyl;
R⁶ is optionally substituted 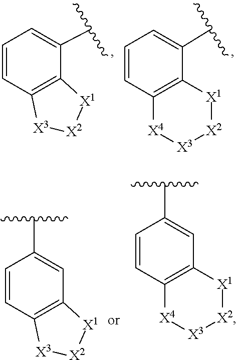
wherein:
X¹, X², X³, and X⁴ are each independently selected from —N(R⁷)—, —O—, —S—, and —C(R⁸)(R⁹)—;
each R⁷ is independently hydrogen, optionally substituted alkyl, or a nitrogen protecting group; and
R⁸ and R⁹ are each independently hydrogen, optionally substituted alkyl, or are taken together with the carbon to which they are attached form a carbonyl.

75. The compound of claim 74, wherein the compound is of Formula (III), or a salt thereof: 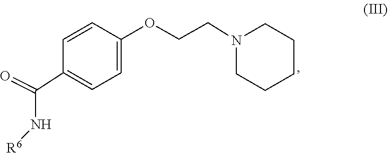
wherein:
R⁶ is 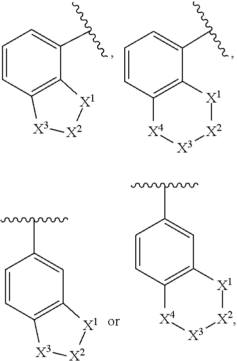
wherein:
X¹, X², X³, and X⁴ are each independently selected from —N(R⁷)—, —O—, —S—, and —C(R⁸)(R⁹)—;
each R⁷ is independently hydrogen, alkyl, or a nitrogen protecting group; and
R⁸ and R⁹ are each independently hydrogen, alkyl, or taken together with the carbon to which they are attached form a carbonyl.

76. The compound of claim 74, wherein the compound is of Formula (XIV), (XV), or (XVI), or a salt thereof:

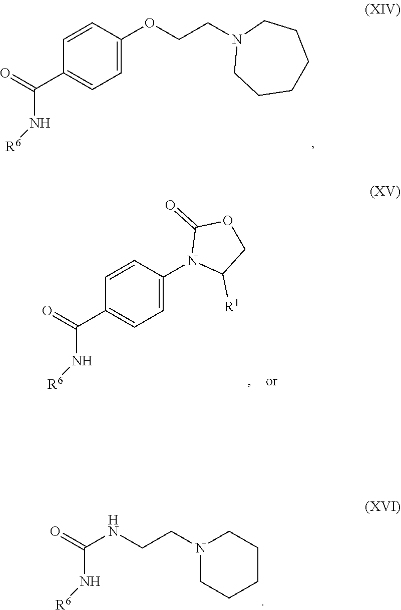

77. The compound of any one of claims 74-76, or a salt thereof, wherein R⁶ is

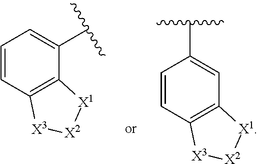

78. The compound of any one of claims 74-76, or a salt thereof, wherein R⁶ is

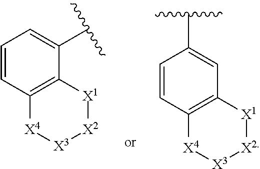

79. The compound of any one of claims 74-76, or a salt thereof, wherein R⁶ is

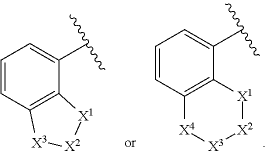

80. The compound of any one of claims 74-76, or a salt thereof, wherein R⁶ is

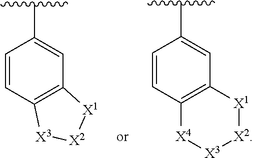

81. The compound of any one of claims 74-80, or a salt thereof, wherein at least one of X¹, X², X³, and X⁴ is —N(R⁷)—.

82. The compound of claim 81, or a salt thereof, wherein R⁷ is hydrogen.

83. The compound of any one of claims 74-82, or a salt thereof, wherein at least one of X¹, X², X³, and X⁴ is —C(R⁸)(R⁹)— and R⁸ and R⁹ taken together with the carbon to which they are attached form a carbonyl.

84. The compound of claim 74, wherein the compound is of Formula (XVII), or a salt thereof:

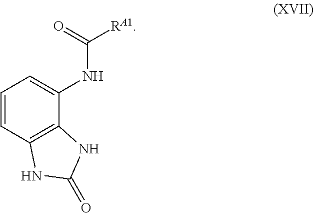

85. The compound of claim 76, or a salt thereof, wherein R¹ is C₁₋₆ alkyl.

86. The compound of claim 85, or a salt thereof, wherein R¹ is isopropyl.

87. The compound of claim 74, wherein the compound has the formula: 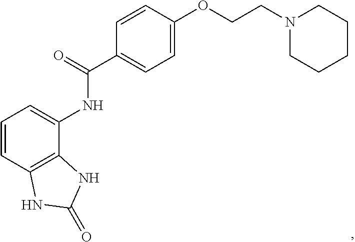 [SMILES: O=C(Nc1cccc2[nH]c(=O)[nH]c12)c1ccc(OCCN2CCCCC2)cc1]
or a salt thereof.

88. The compound of claim 74, wherein the compound has the formula: 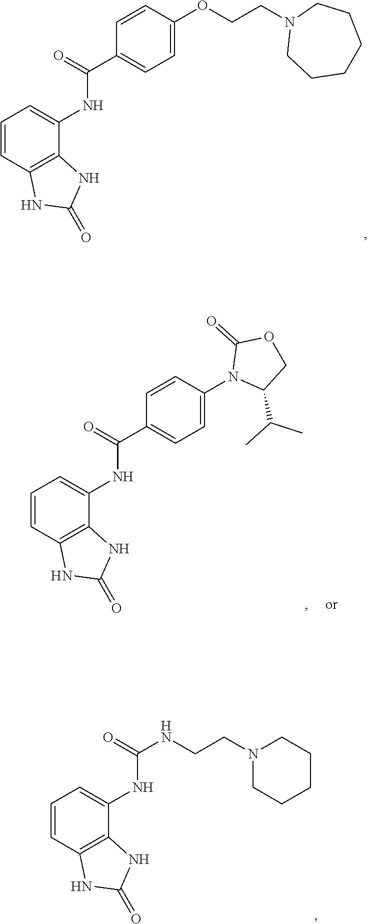 [SMILES: O=C(Nc1cccc2[nH]c(=O)[nH]c12)c1ccc(OCCN2CCCCCC2)cc1 ; CC(C)[C@H]1COC(=O)N1c1ccc(C(=O)Nc2cccc3[nH]c(=O)[nH]c23)cc1 ; O=C(NCCN1CCCCC1)Nc1cccc2[nH]c(=O)[nH]c12]
or a salt thereof.

89. A compound of Formula (D), or a salt thereof: 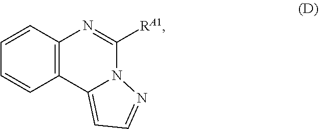
wherein:
R^{A1} is 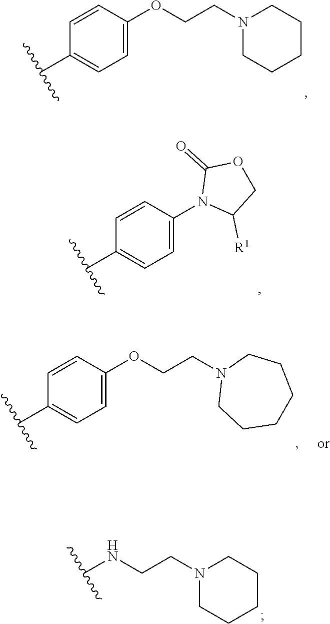
and
R¹ is optionally substituted alkyl or optionally substituted cycloalkyl.

90. The compound of claim 89, or a salt thereof, wherein R¹ is C₁₋₆ alkyl.

91. The compound of claim 90, or a salt thereof, wherein R¹ is isopropyl.

92. The compound of claim 89, wherein the compound has the formula: 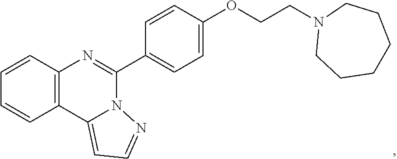 [SMILES: c1ccc2c(c1)nc(-c1ccc(OCCN3CCCCCC3)cc1)n1nccc21]
or a salt thereof.

93. The compound of claim 89, wherein the compound has the formula: 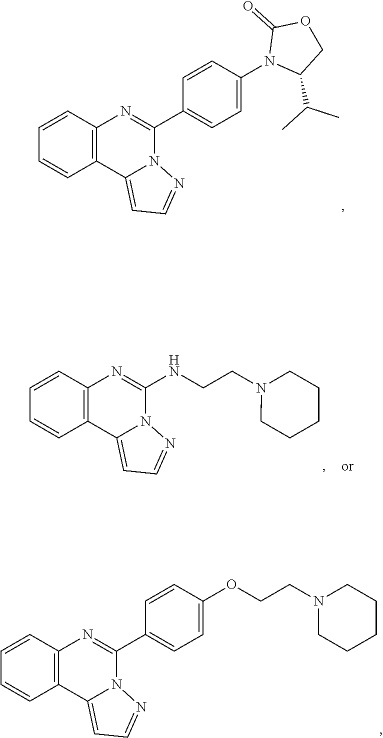 [SMILES: CC(C)[C@H]1COC(=O)N1c1ccc(-c2nc3ccccc3c3ccnn23)cc1 ; c1ccc2c(c1)nc(NCCN1CCCCC1)n1nccc21 ; c1ccc2c(c1)nc(-c1ccc(OCCN3CCCCC3)cc1)n1nccc21]
or a salt thereof.

94. A compound of Formula (E), or a salt thereof: 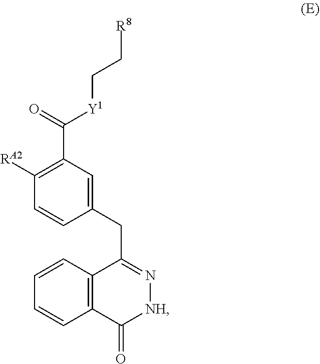
wherein:
R⁸ is 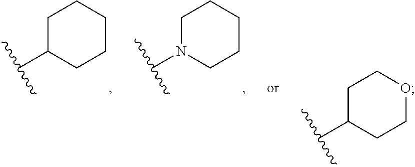 [SMILES: *C1CCCCC1 ; *N1CCCCC1 ; *C1CCOCC1]
R^{A2} is halogen;
Y¹ is —O— or —N(R⁷)—; and
R⁷ is hydrogen, alkyl, or a nitrogen protecting group.

95. The compound of claim 94, or a salt thereof, wherein R^{A2} is fluorine.

96. The compound of claim 94 or 95, or a salt thereof, wherein Y¹ is —O— or —NH—.

97. The compound of claim 94, wherein the compound has the formula: 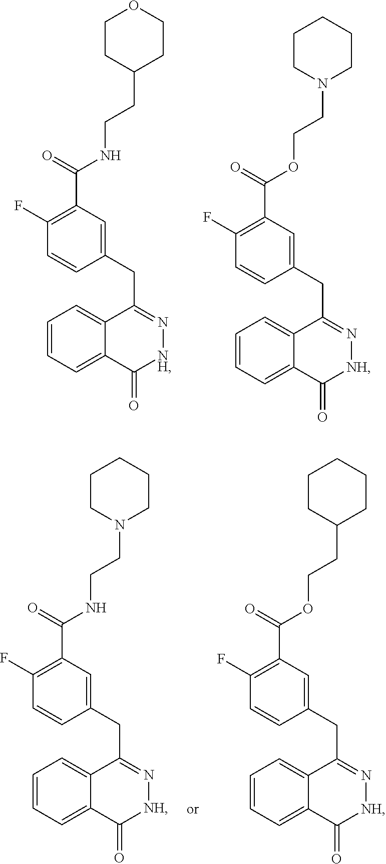
or a salt thereof.

98. A pharmaceutical composition comprising a compound of any one of the preceding claims, or a salt thereof, and a pharmaceutically acceptable carrier.

99. The pharmaceutical composition of claim 98, further comprising an additional therapeutic agent.

100. A method of inhibiting IL-6 signaling comprising administering a compound of any one of claims 1-97, or a salt thereof, or a pharmaceutical composition of any one of claims 98 or 99.

101. The method of claim 100, wherein the compound is a dual PARP/IL-6 inhibitor.

102. The method of claim 100 or 101, wherein the inhibition of IL-6 is in vitro.

103. The method of claim 100 or 101, wherein the inhibition of IL-6 is in vivo.

104. The method of any one of claims 100-103, further comprising administering the compound to a subject.

105. A method of inhibiting IL-6/gp130 comprising administering a compound of any one of claims 1-97, or a salt thereof, or a pharmaceutical composition of any one of claims 98 or 99.

106. The method of claim 105, wherein the compound is a dual PARP/IL-6 inhibitor.

107. The method of claim 105 or 106, wherein the inhibition is in vitro.

108. The method of claim 105 or 106, wherein the inhibition is in vivo.

109. The method of any one of claims 105-108, further comprising administering the compound to a subject.

110. A method of treating inflammatory disease in a subject in need thereof, the method comprising administering to the subject a compound of any one of claims 1-97, or a salt thereof, or a pharmaceutical composition of any one of claims 98 or 99.

111. The method of claim 110, wherein the inflammatory disease is fibrosis.

112. The method of claim 111, wherein the fibrosis is liver fibrosis.

113. The method of claim 110, wherein the inflammatory disease is nonalcoholic steatohepatitis (NASH).

114. A method of treating cancer in a subject in need thereof, the method comprising administering to the subject a compound of any one of claims 1-97, or a salt thereof, or a pharmaceutical composition of any one of claims 98 or 99.

115. The method of claim 114, wherein the cancer is breast cancer or pancreatic cancer.

116. The method of claim 115, wherein the breast cancer is triple negative breast cancer.

117. The method of claim 114, wherein the cancer is bone cancer or brain cancer.

118. A method of treating an autoimmune disease in a subject in need thereof, the method comprising administering to the subject a compound of any one of claims 1-97, or a salt thereof, or a pharmaceutical composition of any one of claims 98 or 99.

119. The method of any one of claims 100-118, comprising administering to the subject a therapeutically effective amount of a compound of any one of claims 1-97, or a salt thereof, or a pharmaceutical composition of any one of claims 98 or 99.

120. The method of any one of claims 100-119, wherein the subject is identified as in need of such treatment.

121. The method of any one of claims 100-120, wherein the subject has such disease or disorder.
